SEQUENCE LISTING (2 sequences)

SEQ ID NO: 1 - DNA, 20 bp, Artificial Sequence
  Description of Artificial Sequence Synthetic primer
agagtttgatcmtggctcag

SEQ ID NO: 2 - DNA, 22 bp, Artificial Sequence
  Description of Artificial Sequence Synthetic primer
tacggytaccttgttacgactt

(19) United States
(12) Patent Application Publication
(10) Pub. No.: US 2020/0268813 A1
(43) Pub. Date: Aug. 27, 2020

(54) MODULATION OF HOST IMMUNE CELL POPULATIONS USING GUT MICROBIOTA

(71) Applicant: PRESIDENT AND FELLOWS OF HARVARD COLLEGE, Boston, MA (US)

(72) Inventors: Christophe O. BENOIST, Brookline, MA (US); Naama GEVA-ZATORSKY, Brookline, MA (US); Dennis KASPER, Brookline, MA (US); Diane J. MATHIS, Brookline, MA (US); Esen SEFIK, Milford, CT (US)

(73) Assignee: PRESIDENT AND FELLOWS OF HARVARD COLLEGE, Boston, MA (US)

(21) Appl. No.: 16/486,322

(22) PCT Filed: Feb. 15, 2018
(86) PCT No.: PCT/US18/18335

§ 371 Date: Aug. 15, 2019

Related U.S. Application Data
(60) Provisional application No. 62/459,442, filed on Feb. 15, 2017.

(51) Int. Cl. A61K 35/745; A61K 35/742; A61K 35/747; A61P 37/02

(52) U.S. Cl. CPC A61K 35/745; A61P 37/02; A61K 35/747; A61K 35/742

(57) ABSTRACT

Provided herein are methods of modulating selected populations of immune cells by administering specific bacterial strains to a subject. Also provided herein are methods of promoting expansion and/or contraction of selected populations of immune cells following the administration of a bacterial strain to a subject.

### CROSS-REFERENCE TO RELATED APPLICATIONS

This application is a 35 U.S.C. § 371 National Phase Entry Application of International Application No. PCT/US2018/018335 filed Feb. 15, 2018, which designates the U.S. and claims benefit under 35 U.S.C. § 119(e) of U.S. Provisional Application No. 62/459,442 filed Feb. 15, 2017, the contents of each of which are incorporated herein by reference in their entireties.

The instant application contains a Sequence Listing which has been submitted electronically in ASCII format and is hereby incorporated by reference in its entirety. Said ASCII copy, created on Aug. 13, 2019, is named 002806-088401-PCT_SL.txt, and is 7779 bytes in size

### FIELD OF THE INVENTION

This invention relates to the immunomodulatory effect of gut microbes.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The mammalian gastrointestinal tract is inhabited by hundreds of species of symbiotic microbes, many of which have a beneficial impact on the host. The local immune system faces the daunting task of enforcing peaceful co-existence with these microbes while also imposing a staunch barrier to pathogen invasion. Maintaining this equilibrium involves both the innate and adaptive arms of the immune system as well as non-immunologic protective strategies—e.g., those involving the mucus barrier and antimicrobial peptides (AMPs). These host-protective mechanisms are counterbalanced by regulatory processes that limit the antibacterial response and prevent collateral damage from inflammation.

The gut microbiota plays an important role in educating and modulating the host immune system. There has been great interest of late in harnessing immune system-microbiota cross-talk in the intestine to therapeutic ends. A common approach has been to perform microbiome-wide association studies to search for correlations between particular microbes and particular disease conditions.

Therefore, there is a need in the art for the identification of immunomodulatory gut microbes and their use in therapeutic methods.

### SUMMARY OF THE INVENTION

Various embodiments of the present invention provide for a method for manipulating a selected population of immune cells in a subject, the method comprising administering to the subject a bacterial strain selected from the group consisting of Clostridium sordellii, Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella, Coprobacillus, Bacteroides uniformis, Clostridium perfringens, Bacteroides fragilis, Bacteroides vulgatus, Lactobacillus rhamnosus, Staphylococcus saprophyticus, Parabacteroides distasonis, Fusobacterium nucleatum, Propionibacterium granulosum, Bifidobacterium longum, Bacteroides ovatus, Bacteroides thetaiotaomicron, Enterococcus faecium, Helicobacter pylori, Ruminococcus gnavus, Peptostreptococus asaccharolyticus, Streptococcus mitis, or a combination thereof.

In various embodiments, the bacterial strain is administered to the GI tract of the subject.

In various embodiments, the manipulation comprises a change in an immune cell population in a tissue of the colon or small intestine. In some embodiments, the manipulation comprises an expansion of a monocyte population, and the bacterial strain is Clostridium sordellii. In other embodiments, the Clostridium sordellii bacterium is the species AO32.

In various embodiments, the manipulation comprises a contraction of a population of macrophages, and the bacterial strain is selected from the group consisting of Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella or a combination thereof. In various embodiments, the Acinetobacter baumannii bacterium is the species ATCC17978, the Acinetobacter lwoffii bacterium is the species F78, the Bifidobacterium breve bacterium is the species SK134, the Bacteroides dorei bacterium is the species DSM17855, the Collinsella aerofaciens bacterium is the species VPI1003, the Clostridium ramosum bacterium is the species AO31, the Lachnospiraceae bacterium is the species sp_2_1_58FAA, the Lactobacillus casei bacterium is the species AO47, and the Veillonella bacterium is the species 6_1_27. In various embodiments, the population of macrophages is CD11b+, CD11C−, F4/80+.

In various embodiments, the manipulation comprises a contraction of a population of mononuclear phagocytes, and the bacterial strain is selected from the group consisting of Acinetobacter lwoffii, Collinsella aerofaciens, Coprobacillus, and combinations thereof. In various other embodiments, the Acinetobacter lwoffii bacterium is the species F78, the Collinsella aerofaciens bacterium is the species VPI1003, and the Coprobacillus bacterium is the species 8_2_54BFAA. In various embodiments, the population of mononuclear phagocytes is CD11b+, CD11c+, F4/80+.

In various embodiments, the manipulation comprises an expansion of a population of dendritic cells, and the bacterial strain is selected from the group consisting of Bifidobacterium breve, Bacteroides uniformis, Lachnospiraceae, and combinations thereof. In various embodiments, the Bifidobacterium breve bacterium is the species SK134, the Bacteroides uniformis bacterium is the species ATCC8492, and the Lachnospiraceae bacterium is the species sp_2_1_58FAA. In various other embodiments, the population of dendritic cells is CD103+, CD11b+.

In various embodiments, the manipulation comprises a contraction of a population of CD103+, CD11b+ dendritic cells, and the bacterial strain is selected from the group consisting of Acinetobacter lwoffii_F78, Clostridium perfringens_ATCC13124, and a combination thereof. In various other embodiments, the Acinetobacter lwoffii bacterium is the species F78 and the Clostridium perfringens bacterium is the species ATCC13124. In yet other embodiments, the population of dendritic cells is CD103+, CD11b+.

In various embodiments, the manipulation comprises an expansion of a population of plasmacytoid dendritic cells, and the bacterial strain is selected from the group consisting of Bacteroides fragilis, Bacteroides vulgatus, and a combination thereof. In various other embodiments, the Bacteroides fragilis bacterium is the species NCTC9343, and the Bacteroides vulgatus bacterium is the species ATCC8482.

In various embodiments, the manipulation comprises a contraction of a population of plasmacytoid dendritic cells, and the bacterial strain is selected from the group consisting of Lactobacillus rhamnosus, Staphylococcus saprophyticus, and a combination thereof. In various other embodiments, the Lactobacillus rhamnosus bacterium is the species LMS2-1, and the Staphylococcus saprophyticus bacterium is the species ATCC15305.

In various embodiments, the manipulation comprises a contraction of a population of type 3 innate lymphoid cells, and the bacterial strain is selected from the group consisting of Coprobacillus, Parabacteroides distasonis, Veillonella, and combinations thereof. In various other embodiments, the Coprobacillus bacterium is the species 8_2_54BFAA, and the Parabacteroides distasonis bacterium is the species ATCC8503, and the Veillonella bacterium is the species 6_1_27.

In various embodiments, the manipulation comprises an expansion of a population of IL22+ innate lymphoid cells, and the bacterial strain is selected from the group consisting of Bacteroides uniformis, Lactobacillus casei, and a combination thereof. In various other embodiments, the Bacteroides uniformis bacterium is the species ATCC8492, and the Lactobacillus casei bacterium is the species AO47.

In various embodiments, the manipulation comprises a contraction of a population of IL22+ innate lymphoid cells, and the bacterial strain is selected from the group consisting of Acinetobacter lwoffii, Coprobacillus, Clostridium sordellii, Veillonella, and combinations thereof. In various other embodiments, the Acinetobacter lwoffii bacterium is the species F78, and the Coprobacillus bacterium is the species 8_2_54BFAA, the Clostridium sordellii bacterium is the species AO32, and the Veillonella bacterium is the species 6_1_27.

In various embodiments, the manipulation comprises an expansion of a population of IL22+ innate lymphoid cells, and the bacterial strain is selected from the group consisting of Acinetobacter baumannii, Bacteroides dorei, and a combination thereof. In various other embodiments, the Acinetobacter baumannii bacterium is the species ATCC17978, and the Bacteroides dorei bacterium is the species DSM17855.

In various embodiments, the manipulation comprises a contraction of a population of IL22+ innate lymphoid cells, and the bacterial strain is selected from the group consisting of Acinetobacter lwoffii, Fusobacterium nucleatum, Propionibacterium granulosum, Veillonella, and combinations thereof. In various other embodiments, the Acinetobacter lwoffii bacterium is the species F78, the Fusobacterium nucleatum bacterium is the species F0419, the Propionibacterium granulosum bacterium is the species AO42, and the Veillonella bacterium is the species 6_1_27.

In various embodiments, the manipulation comprises an expansion of a population of CD4 T cells, and the bacterial strain is selected from the group consisting of Acinetobacter lwoffii, Bifidobacterium longum, Bacteroides ovatus, Bacteroides thetaiotaomicron, Bacteroides vulgatus, Coprobacillus, Enterococcus faecium, Helicobacter pylori, Ruminococcus gnavus, Veillonella and combinations thereof. In various other embodiments, the Acinetobacter lwoffii bacterium is the species F78, the Bifidobacterium longum bacterium is the species AO44, the Bacteroides ovatus bacterium is the species ATCC8483, the Bacteroides thetaiotaomicron bacterium is the species ATCC29741, the Bacteroides vulgatus bacterium is the species ATCC8482, the Coprobacillus bacterium is the species 8_2_54BFAA, the Enterococcus faecium bacterium is the species TX1330, the Helicobacter pylori bacterium is the species ATCC700392, the Ruminococcus gnavus bacterium is the species ATCC29149, and the Veillonella bacterium is the species 6_1_27. In yet other embodiments, the population of CD4 T cells is IL10+.

In various embodiments, the manipulation comprises a contraction of a population of CD4 T cells, and the bacterial strain is selected from the group consisting of Bacteroides thetaiotaomicron, Peptostreptococus asaccharolyticus, Streptococcus mitis, and combinations thereof. In various other embodiments, the Bacteroides thetaiotaomicron bacterium is the species ATCC29741, the Peptostreptococus asaccharolyticus bacterium is the species AO33, and the Streptococcus mitis bacterium is the species F0392.

In various embodiments, the manipulation comprises a contraction of a population of CD4 T cells, and the bacterial strain is selected from the group consisting of Clostridium perfringens, Peptostreptococus asaccharolyticus, and a combination thereof. In various other embodiments, the Clostridium perfringens bacterium is the species ATCC13124, and the Peptostreptococus asaccharolyticus bacterium is the species AO33. In yet other embodiments, the population of CD4 T cells is IL17+.

In various embodiments, the contraction or expansion of the immune cell population occurs in the colon. In various other embodiments, the contraction or expansion of the immune cell population occurs in the small intestine.

Various embodiments of the present invention also provide for a method of promoting IL10 production or release by cells in the small intestine, the method comprising administering a bacterium of the genus Coprobacillus to the GI tract of the mammal. In various embodiments, the Coprobacillus bacterium is Coprobacillus species 8_2_54BFAA.

Various embodiments of the present invention also provide for a method of promoting IL22 production or release by Innate Lymphoid Cells in the small intestine or colon of a mammal, the method comprising administering Bacteroides dorei, Acinetobacter baumannii or Bifidobacterium longum cells to the GI tract of the mammal.

Various embodiments of the present invention also provide for a method of repressing IL22 production or release in a tissue of the GI tract of a mammal, the method comprising administering Acinetobacter lwoffii, Clostridium sordellii, Fusobacterium nucleatum, Propionibacterium granulosum or Veillonella bacterial cells to the GI tract of the mammal. In various embodiments, the Veillonella bacterium is Veillonella species 6 1 27. In various other embodiments, the tissue is the colon.

Various embodiments of the present invention also provide for a method of suppressing expression of a Reg3 gene in tissue of the small intestine of a mammal, the method comprising administering a composition comprising a Fusobacterium varium bacterium to the GI tract of the mammal.

Various embodiments of the present invention also provide for a method of promoting the expression of an α-defensin or Reg3 gene in tissue of the colon of a mammal, the method comprising administering a composition comprising a Parabacteroides merdae or Porphyromonas uenonsis bacterium to the GI tract of the mammal.

Various embodiments of the present invention also provide for a method of promoting expansion in a population of CD8−, CD4−, TCRγ+ T cells in a tissue of the gastrointestinal tract of a mammal, the method comprising administering a composition comprising a Fusobacterium varium bacterium to the GI tract of the mammal. In various embodiments, the tissue of the gastrointestinal tract comprises the small intestine. In various other embodiments, the tissue of the gastrointestinal tract comprises the colon.

Various embodiments of the present invention also provide for a method of reducing populations of CD4+ T cells and CD8+ T cells, or suppressing expansion of CD4+ T cells and CD8+ T cells, in a tissue of the gastrointestinal tract of a mammal, the method comprising administering a composition comprising a Fusobacterium varium bacterium to the GI tract of the mammal.

Various embodiments of the present invention also provide for a method of promoting an expansion of an immune cell population in a mammal, the method comprising administering a composition comprising a microbe selected from the group consisting of Clostridium sordellii_AO32, Bacteroides uniformis_ATCC8492, Bacteroides fragilis_NCTC9343, Bacteroides vulgatus_ATCC8482, Bifidobacterium longum_AO44, Bacteroides ovatus_ATCC8483, Bacteroides thetaiotaomicron_ATCC29741, Enterococcus faecium_TX1330, Helicobacter pylori_ATCC700392, Ruminococcus gnavus_ATCC29149, Acinetobacter baumannii_ATCC17978, Acinetobacter lwoffii_F78, Bifidobacterium breve_SK134, Bacteroides dorei_DSM17855, Lachnospiraceae_sp_2_1_58FAA, Lactobacillus casei_AO47, Veillonella_6_1_27, Coprobacillus_8_2_54BFAA or a combination thereof, to the mammal's gastrointestinal GI tract. In various embodiments, the expansion occurs at least in a tissue of the GI tract or a lymphoid tissue. In various other embodiments, the expansion occurs in small intestine (SI), colon, or mesenteric lymph nodes. In yet other embodiments, the expansion occurs in a Peyer's patch of the SI. In various embodiments, the expansion occurs in an immune cell population of the intestinal lamina propria. In various other embodiments, the expansion occurs in an immune cell population of the innate immune system.

Various embodiments of the present invention also provide for a method of promoting a contraction of an immune cell population in a mammal, the method comprising administering a composition comprising a microbe selected from the group consisting of Acinetobacter baumannii_ATCC17978, Acinetobacter lwoffii_F78, Bifidobacterium breve_SK134, Bacteroides dorei_DSM17855, Collinsella aerofaciens_VPI1003, Clostridium ramosum_AO31, Lachnospiraceae_sp_2_1_58FAA, Lactobacillus casei_AO47, Veillonella_6_1_27, Coprobacillus_8_2_54BFAA, Clostridium perfringens ATCC13124, Lactobacillus rhamnosus_LMS2-1, Staphylococcus saprophyticus_ATCC15305, Parabacteroides distasonis_ATCC8503, Fusobacterium nucleatum_F0419, Propionibacterium granulosum_AO42, Peptostreptococus asaccharolyticus_AO33, Streptococcus mitis_F0392, Clostridium sordellii_AO32, Bacteroides thetaiotaomicron_ATCC29741 or a combination thereof, to the mammal's gastrointestinal GI tract. In various embodiments, the contraction occurs at least in a tissue of the GI tract or a lymphoid tissue. In various other embodiments, the contraction occurs in small intestine (SI), colon, or mesenteric lymph nodes. In yet other embodiments, the contraction occurs in a Peyer's patch of the SI. In various embodiments, the contraction occurs in an immune cell population of the intestinal lamina propria. In various other embodiments, the contraction occurs in an immune cell population of the innate immune system.

Various embodiments of the present invention also provide for a method of administering a heterologous polypeptide to a mammal, the method comprising administering a bacterium engineered to express the heterologous polypeptide to the GI tract of the mammal. In various embodiments, the bacterium is Peptostreptococcus magnus and/or Bacteroides salanitronis.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-FIG. 1E depicts in accordance with various embodiments of the invention, the experimental design and bacterial colonization. (FIG. 1A) Four week-old GF mice were monocolonized with human gut bacteria and analyzed after two weeks for colonization, impact on the host immune system and genomic activity in the gut. (FIG. 1B) Innate and adaptive immune responses were analyzed by flow cytometry of cells extracted from SI, PPs, colons, mLNs, and SLOs. Innate cell types: Monocytes (Mono), Dendritic cells (DCs), Macrophages (MFs), Mononuclear phagocytes (MNPs) and type 3 innate lymphoid cells (ILC3s). Adaptive cell types: B cells, gamma-delta T cells (Tγδ) and alpha-beta T cells (Tαβ), subsets of Tαβ cells [CD4+(T4), CD8+(T8), CD4−CD8−(DN), RORγ+Foxp3−(proxy for TH17) and Foxp3+ regulatory T cells (Tregs)], and cytokine production (I110, I117a, I122, IFNγ). See FIG. 8 and Table 2. (FIG. 1C) Cladogram of the human gut microbiota. Microbes were identified in the HMP database except for SFB. Diamonds denote the genera included; stars mark the species. Species where more than one strain was analyzed are in bold type. The outer ring represents a bar graph of the prevalence of each genus. See Tables 1, 2 and data not shown—see supplemental materials of Geva-Zatorsky et al., Cell 2017, incorporated by reference herein below. (FIG. 1D) Average CFU per gram of fecal material. Bacteria were ordered according to phyla and rank-ordered within each phylum. (FIG. 1E) Bar graphs of CFUs in mLNs (per organ, top) and SLO (bottom). Bacteria were rank-ordered according to CFUs in mLNs. See Tables 1, 2 and data not shown—see supplemental materials of Geva-Zatorsky et al., Cell 2017, incorporated by reference herein below.

FIG. 2A-FIG. 2E depicts in accordance with various embodiments of the invention, immunomodulation by gut microbes. (FIG. 2A) Rank-ordered average frequencies (flow cytometry) of each immunocyte population (colon) for every microbe. For cell type frequency determination (y-axis) and microbe identification (x-axis) see Tables 1, 2, 3A-G and 4A-G and FIG. 8 for gating strategies. (FIG. 2B) Heatmap showing average fold changes (relative to GF) for each cell-type in the colon and SI following monocolonization. Fecal IgA levels (as fold changes relative to GF) are in bottom row. Gray-no data. (FIG. 2C) Proportion of colonic immune cell types (compared to GF) with a z-score≥2. (FIG. 2D) Example of colonization influencing the gating configuration but not frequency of cell populations. Flow cytometry plots shown are for CD11b+CD11c+ MNPs and DCs. (FIG. 2E) Cytokine responses in the SI and colon resulting from monocolonization. See FIG. 9 and Tables 3-5.

FIG. 3A-FIG. 3D depicts in accordance with various embodiments of the invention, local and systemic immunologic correlations. (FIG. 3A) Clustered heatmap of Pearson correlation coefficients (r) for immunophenotypes after monocolonization. (FIG. 3B-FIG. 3C) Average cell frequency correlations: SLO vs. colon. (FIG. 3D) Hierarchical clustering dendrogram of bacteria based on the Pearson correlation of their overall immunologic impact on the SI and colon. Values for each immunophenotype were normalized to the mean across all microbes. See also FIG. 10.

FIG. 4A-FIG. 4C depicts in accordance with various embodiments of the invention, transcriptional responses to colonization. (FIG. 4A) Mean coefficient of variation (CV) in transcripts from the colons of monocolonized mice and GF mice. Genes variable in both GF and monocolonized mice (2540); Genes more variable in monocolonized (227); and genes more variable in GF (2788). (FIG. 4B-FIG. 4C). Heatmap representation of fold changes of transcripts differentially expressed in (FIG. 4B) the colon and (FIG. 4C) SI of monocolonized and SPF mice compared to GF mice. Bacteria (columns) are clustered by hierarchical clustering; Genes (rows) are clustered by K-means clustering. Association of these transcripts with particular immune and non-immune cell types was verified in gene expression databases such as ImmGen and GNF. Enriched pathways were identified using GO. See also FIG. 11A-FIG. 11D and data not shown—see supplemental materials of Geva-Zatorsky et al., Cell 2017, incorporated by reference herein below).

FIG. 5A-FIG. 5F depicts in accordance with various embodiments of the invention, colonic plasmacytoid dendritic cells are most prolific myeloid responders to the gut microbiota. (FIG. 5A) Representative flow cytometry dot plots of a pDC ‘low inducer’, Propionibacterium granulosum (Pgran.A042) and a ‘high inducer’ Bacteroides vulgatus (Bvulg.ATCC8482). Cells were gated as CD45+CD19−CD11b−. (FIG. 5B) Frequencies of pDCs in the colon induced by monocolonization. (FIG. 5C) Pearson correlation between pDC's in SI vs. colon (p=0.0006). (FIG. 5D) Pearson correlation between colonic pDCs and Tregs (p=0.003). (FIG. 5E-FIG. 5F) Correlation coefficients were calculated between the expression value of each gene from the whole tissue transcriptome (SI, and colon) and the proportions of pDCs for each monocolonizing microbe (SI and colon). (FIG. 5E) Genes related to the interferon signature are marked. (FIG. 5F) Genes having similar expression patterns and correlating best in both the SI and colon are highlighted. The adjacent bar graph shows the enrichment of biological pathways of these highly correlating genes as analyzed by Enrichr. Most significant pathways determined by GO Molecular Function (p<0.05) Depicted gene names and the actual Enrichr adjusted p-values are shown. See also FIG. 12 and Table 9.

FIG. 6A-FIG. 6E shows in accordance with various embodiments of the invention, that antimicrobial peptides exhibit divergent patterns of expression in the small intestine and colon. (FIG. 6A) Coefficient of variation (CV) vs. mean expression in GF mice for all genes in the SI (left panel) and colon (right panel). Only genes expressed above background level are shown. Antimicrobial peptides (AMPs) are highlighted and color-coded according to the categories listed. (FIG. 6B) The CV of all expressed genes in the colons of GF vs monocolonized mice, as shown in FIG. 4A, but here with AMP genes highlighted. (FIG. 6C-FIG. 6D) Heatmaps illustrating the differential expression of AMPs in the SI (FIG. 6C) and colon (FIG. 6D) in various microbially monocolonized mice compared to GF mice. Heatmap colors represent the log 2 fold change values relative to GF mice. Only AMPs expressed above background levels are shown. (FIG. 6E) Gene programs correlated with AMP expression in the colon. For every gene expressed in the colon, its correlation with colonic AMP genes (Reg3 family and α-defensins) is plotted for GF mice vs. monocolonized mice (left panel). Top correlated genes (Spearman's rho>0.6) are highlighted in black and parsed for enrichment of biological pathways using Enrichr. Top pathways from GO Molecular Function, with corresponding adjusted p-values and gene names, are shown (right panel).

FIG. 7A-FIG. 7E depicts in accordance with various embodiments of the invention, host response to Fusobacterium varium. (FIG. 7A) Amplified gene expression preferential to F. varium (Fvari.AO16), based on the conservative gene list established in FIG. 4B-FIG. 4C. Fold change (FC) of Fvari.AO16 over GF (y-axis) was compared to the maximum induced FC by any other microbe over GF (x-axis). Top—SI, bottom—colon. (FIG. 7B) Functional analysis of genes suppressed by F. varium. STRING-db clustering and functional categories of significantly altered genes (FC≤0.5 in SI; FC≤0.67 in colon vs. GF; FDR 0.1). Genes (Mt2, Ifit2, Trim 30a, Slc5a12, Akr1c19, Adh4) from (FIG. 7A) preferentially suppressed by Fvari.AO16; The schematic shows all other suppressed genes in the Fvari.AO16 response that formed connected clusters. Functional categories determined by GO and KEGG are shown: “Retinol metabolism” FDR 2.25e-15. “Bile acid metabolism” FDR 2.6e-7. “Immune response” FDR 0.0138. (FIG. 7C) Functional analysis of genes induced by F. varium. STRING-db clustering and functional categories of significantly altered genes (SI FC≥2, colon FC≥1.5 vs. GF; FDR 0.1). Red dots—genes from (FIG. 7A) preferentially induced by Fvari.AO16-; gray dots—all other induced genes in Fvari.AO16 response that formed connected clusters. Functional categories determined by GO and KEGG: “Regulation of TRP channels” FDR 0.00313; “AA metabolism” FDR 0.0241; “Globin” FDR 3.78e-8; “Triglyceride metabolism” FDR 0.0184; “Glycerolipid metabolism” FDR 1.32e-7. (FIG. 7D) F. varium elevates DN T cell frequency. Representative flow cytometry plots of CD4 and CD8 expression in GF and Fvari.AO16, gated on CD45+CD19−TCRβ+ cells. (FIG. 7E) Frequencies of T4, T8, and DN T cells normalized to the mean frequency of all microbes in all monocolonizations. See also Tables 8 and 9.

FIG. 8A-FIG. 8C depicts in accordance with various embodiments of the invention, representative flow cytometry plots demonstrating the gating strategy for the three staining panels: lymphocytes (FIG. 8A), myeloid cells (FIG. 8B), and the cytokines (FIG. 8C). Related to FIG. 1A-FIG. 1E.

FIG. 9A-FIG. 9H depicts in accordance with various embodiments of the invention, immunomodulation following monocolonized microbe administration. (FIG. 9A-FIG. 9D) Rank-ordered average frequencies of each immunocyte population for every monocolonized microbe in SI, PP, mLN, SLO, as measured by flow cytometry. For cell-type frequency determination (y-axis) and bacterial identification (x-axis), see Tables 2, FIG. 3A-FIG. 3G, and FIG. 4A-FIG. 4G. For gating strategies, see FIG. 8A-FIG. 8C. (FIG. 9E) Representative flow cytometry plots of monocytes (Ly6c+CD11b+) in the SI (gated on CD45+CD19− cells). Monocytes include Ly6chi and Ly6clo populations, which are measured as a uniform population in the quantification. Plots here highlight that certain microbes can induce Ly6chi, Ly6clo, or both. (FIG. 9F) Representative flow cytometry plots of CD11b and CD11c expression in the SLO (gated on CD45+CD19− cells). These populations correspond to macrophages, F4/80+ mononuclear phagocytes, CD103+ DCs, and pDCs. CD11b expression is dimmer in the SLO compared to intestinal tissues. The CD11bloCD11clo population, which is largely absent in the intestines, is more pronounced in the SLO. These qualities of myeloid cells were not reflected in the quantification in FIG. 2A and FIG. 2B. (FIG. 9G) Representative flow cytometry plots of T4, T8 and DN T cells (gated on CD45+TCR+CD19− cells) in the SI. In contrast to the majority of myeloid markers, the lymphocyte markers are clearer and more consistent across tissues. Related to FIG. 2A-FIG. 2E. See also Tables 3A-G, and 4A-G. (FIG. 9H) Fecal IgA induction of individual monocolonized mice. IgA concentration quantified by ELISA (upper), % IgA quantified by flow cytometry (lower).

FIG. 10A-FIG. 10B depicts in accordance with various embodiments of the invention, correlations of immunophenotypes across tissues. (FIG. 10A) Pearson correlations were performed for each cell population assayed in the SI, colon, mLN, and SLO, and the resulting correlation coefficients were plotted as a heat map. Three correlated clusters were evident: CD11b+F4/80+ cells (which encompass CD11b+CD11c− MF and CD11b+CD11c+ MNPs), monocytes, Foxp3−RORγ+CD4+ T cells (as a proxy for T4 cells capable of 1117 production), and a Foxp3+RORγ+Helios− Treg cluster (measured separately as Foxp3+Helios− or RORγ+Helios−). (FIG. 10B) Pearson correlation of the overall immunologic impact of microbes on the SI and colon. Values for each immunophenotype were normalized to the mean across all microbes. Hierarchical clustering was performed. Related to FIG. 3A-FIG. 3D.

FIG. 11A-FIG. 11D depicts in accordance with various embodiments of the invention, volcano plot [p(−log 10) vs. Fold Change] representations of the microarray data in the colon (FIG. 11A) and the SI (FIG. 11B). (FIG. 11C, FIG. 11B) Levels of I118 transcript across the microbes studied in the colon (FIG. 11C) and in the SI (FIG. 11D). Related to FIG. 4A-FIG. 4C.

FIG. 12 depicts in accordance with various embodiments of the invention, frequencies of CD103+CD11b− DCs (top; gated on CD45+CD19− cells) and of pDCs (bottom; gated on CD45+CD19−CD1 b− cells) induced in the colon by monocolonizing microbes. Microbes were ordered according to their pDC induction level and color-coded for individual experiments. GF data are shown. Related to FIG. 5A-FIG. 5F.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “subject” refers to any animal (e.g., a mammal), including, but not limited to humans, non-human primates, and rodents, which is to be the recipient of immune cell modulation and/or of a particular treatment. Primates include, but are not limited to, chimpanzees, cynomologous monkeys, spider monkeys, and macaques, e.g., Rhesus. Rodents include, but are not limited to, mice, rats, woodchucks, ferrets, rabbits and hamsters. In various embodiments, a subject can be one who has been previously diagnosed with or identified as suffering from or having a condition in need of treatment. In various other embodiments, the subject previously diagnosed with or identified as suffering from or having a condition may or may not have undergone treatment for a condition. In yet other embodiments, a subject can also be one who has not been previously diagnosed as having a condition, but who exhibits one or more risk factors for a condition. A “subject in need” of treatment for a particular condition can be a subject having that condition, diagnosed as having that condition, or at risk of developing that condition.

Non-limiting examples of “adaptive immune system cells” include lymphocytes (such as, B cells and T cells). In some embodiments, the B and T cells can be naïve cells. In some other embodiments, the T cells are effector cells, memory cells, regulatory cells, helper cells, or cytotoxic cells. Non-limiting examples of “innate immune system cells” include leukocytes, natural killer cells (NK cells), mast cells, granulocytes, eosinophils, basophils, polymorphonuclear cells (PMNs), γδ T cells; and phagocytic cells including macrophages, neutrophils, dendritic cells (DCs).

The terms “increase” and “expansion” are used interchangeably herein, to refer to the immune cell population and/or its response which has become greater in size, amount, intensity and/or degree from a control value. The terms refer to a change relative to a reference value of at least 10%, or more, e.g., at least 20%, at least 30%, at least 40%, at least 50%, at least 60%, at least 70%, at least 80%, at least 90% or more, including, for example, at least 1-fold, at least 2-fold, at least 3-fold, at least 4-fold, at least 5-fold, at least 10-fold or more.

The terms “decrease” and “contraction” are used interchangeably herein, to refer to the immune cell population and/or its response which has become less in size, amount, intensity and/or degree from a control value. The terms refer to a change relative to a reference value of at least 10%, or more, e.g., at least 20%, at least 30%, at least 40%, at least 50%, at least 60%, at least 70%, at least 80%, at least 90% or more.

As used herein, “bacteria,” “bacterial strain” and “microbe” are used interchangeably and refer to a microorganism administered to elicit an immune response.

Germ-free (GF) mice show defects in multiple specific immunocyte populations, such as Th2 skewing of their CD4+ T cell compartments, compromised innate lymphoid cell (ILC) function; a deficiency in IgA-producing plasma cells; and, more generally, greater susceptibility to infection. The immunologic impacts of few microbial species have been elucidated: Segmented Filamentous Bacteria (SFB) elicit a robust Th17 response; a glycosphingolipid from Bacteroides fragilis inhibits invariant natural killer T cell differentiation; and specific subsets of CD4+Foxp3+ regulatory T cells (Tregs) are induced by a range of individual or groups of microbes. These changes in immunocyte profiles have readily discernible effects on both gut and extra-gut immune responses, whether protective or pathogenic.

Within the human gut reside diverse microbes coexisting with the host in a mutually advantageous relationship. Evidence has revealed the pivotal role of the gut microbiota in shaping the immune system. To date, only a few of these microbes have been shown to modulate specific immune parameters. The approach for the experiments described herein, was to use gnotobiotic colonization of GF mice with single microbial strains derived from the human gut followed by extensive immunophenotyping and transcriptomic analysis. While this reductionist experimental strategy sets aside the combinatorial effects of a complex microbiota, monocolonization renders the complexities of immune system-microbiota interactions more tractable. The numbers of colonizing bacterial species are higher and more stable over time in a monocolonized host than in a host with a diverse microbiota, and the antigenic or metabolic stimulus to the local immune system is consequently stronger. The present invention provides a robust, “sensitized” readout system that permits screening for human-derived immunomodulatory microbes and molecules.

The driving concept was that the co-evolution of the intestinal microbiota and the local immune system for millennia has resulted in a variety of presumably innocuous strategies by which various microbes manipulate immune system activities. The goal of the studies described herein in the Examples section was to begin to uncover these microbial tactics, using a compendious and performant screen.

Germ-free mice were monocolonized with 53 individual bacterial species representing all five of the major phyla, and their effects on the composition and activation of most innate and adaptive immune-system cell types as well as on intestinal tissue transcriptomes was evaluated. A synthetic overview of the extensive dataset generated and three vignettes describing the findings on particular immunomodulatory cell types or molecules are presented herein in the Examples section. The screen focused on human intestinal symbionts that were culturable and that encompassed, as widely as was practical, the genetic diversity of the human gut microbiota.

As described herein, the immunomodulatory effects of phylogenetically diverse human gut microbes were broadly identified. Surprisingly, these were independent of microbial phylogeny. Microbial diversity in the gut ensures robustness of the microbiota's ability to generate a consistent immunomodulatory impact, serving as a highly important epigenetic system. Without being bound to any particular theory, this study provides a foundation for the investigation of gut microbiota-host mutualism, highlighting key players that could identify important therapeutics.

The methods and compositions provided herein are based, at least in part, on these findings. Embodiments address the need in the art for methods of modulating a selected population of immune cells by administering a specific bacterial strain to a subject. Embodiments further provide for methods of promoting expansion and/or contraction of a selected population of immune cells following the administration of a bacterial strain to a subject.

### Method of Manipulating a Selected Population of Immune Cells

Various embodiments of the methods and compositions described herein provide for a method of manipulating a selected population of immune cells in a subject, the method comprising administering to the subject a bacterial strain selected from the group consisting of Clostridium sordellii, Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella, Coprobacillus, Bacteroides uniformis, Clostridium perfringens, Bacteroides fragilis, Bacteroides vulgatus, Lactobacillus rhamnosus, Staphylococcus saprophyticus, Parabacteroides distasonis, Fusobacterium nucleatum, Propionibacterium granulosum, Bifidobacterium longum, Bacteroides ovatus, Bacteroides thetaiotaomicron, Enterococcus faecium, Helicobacter pylori, Ruminococcus gnavus, Peptostreptococus asaccharolyticus, Streptococcus mitis, or a combination thereof. In various embodiments, the bacterial strain is administered to the GI tract of the subject. In various embodiments, the manipulation comprises a change in an immune cell population in a tissue of the colon or small intestine.

In various embodiments, the manipulation comprises an expansion of a monocyte population, and the bacterial strain is Clostridium sordellii. In various other embodiments, the Clostridium sordellii bacterium is the species AO32.

In various embodiments, the manipulation comprises a contraction of a population of macrophages, and the bacterial strain is selected from the group consisting of Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella or a combination thereof. In various other embodiments, the Acinetobacter baumannii bacterium is the species ATCC17978, the Acinetobacter lwoffii bacterium is the species F78, the Bifidobacterium breve bacterium is the species SK134, the Bacteroides dorei bacterium is the species DSM17855, the Collinsella aerofaciens bacterium is the species VPI1003, the Clostridium ramosum bacterium is the species AO31, the Lachnospiraceae bacterium is the species sp_2_1_58FAA, the Lactobacillus casei bacterium is the species AO47, and the Veillonella bacterium is the species 6_1_27. In some other embodiments, the population of macrophages is CD11b+, CD11C−, F4/80+.

In various embodiments, the manipulation comprises a contraction of a population of mononuclear phagocytes, and the bacterial strain is selected from the group consisting of Acinetobacter lwoffii, Collinsella aerofaciens, Coprobacillus, and combinations thereof. In various other embodiments, the Acinetobacter lwoffii bacterium is the species F78, the Collinsella aerofaciens bacterium is the species VPI1003, and the Coprobacillus bacterium is the species 8_2_54BFAA. In some other embodiments, the population of mononuclear phagocytes is CD11b+, CD11c+, F4/80+.

In various embodiments, the manipulation comprises an expansion of a population of dendritic cells, and the bacterial strain is selected from the group consisting of Bifidobacterium breve, Bacteroides uniformis, Lachnospiraceae, and combinations thereof. In various other embodiments, the Bifidobacterium breve bacterium is the species SK134, the Bacteroides uniformis bacterium is the species ATCC8492, and the Lachnospiraceae bacterium is the species sp2158FAA. In some other embodiments, the population of dendritic cells is CD103+, CD11b+.

In various embodiments, the manipulation comprises a contraction of a population of CD103+, CD11b+ dendritic cells, and the bacterial strain is selected from the group consisting of Acinetobacter lwoffii_F78, Clostridium perfringens_ATCC13124, and a combination thereof. In various other embodiments, the Acinetobacter lwoffii bacterium is the species F78 and the Clostridium perfringens bacterium is the species ATCC13124. In some other embodiments, the population of dendritic cells is CD103+, CD11b+.

In various embodiments, the manipulation comprises an expansion of a population of plasmacytoid dendritic cells, and the bacterial strain is selected from the group consisting of Bacteroides fragilis, Bacteroides vulgatus, and a combination thereof. In various other embodiments, the Bacteroides fragilis bacterium is the species NCTC9343, and the Bacteroides vulgatus bacterium is the species ATCC8482.

In various embodiments, the manipulation comprises a contraction of a population of plasmacytoid dendritic cells, and the bacterial strain is selected from the group consisting of Lactobacillus rhamnosus, Staphylococcus saprophyticus, and a combination thereof. In various other embodiments, the Lactobacillus rhamnosus bacterium is the species LMS2-1, and the Staphylococcus saprophyticus bacterium is the species ATCC15305.

In various embodiments, the manipulation comprises a contraction of a population of type 3 innate lymphoid cells, and the bacterial strain is selected from the group consisting of Coprobacillus, Parabacteroides distasonis, Veillonella, and combinations thereof. In various other embodiments, the Coprobacillus bacterium is the species 8_2_54BFAA, and the Parabacteroides distasonis bacterium is the species ATCC8503, and the Veillonella bacterium is the species 6_1_27.

In various embodiments, the manipulation comprises an expansion of a population of IL22+ innate lymphoid cells, and the bacterial strain is selected from the group consisting of Bacteroides uniformis, Lactobacillus casei, and a combination thereof. In various other embodiments, the Bacteroides uniformis bacterium is the species ATCC8492, and the Lactobacillus casei bacterium is the species AO47.

In various embodiments, the manipulation comprises a contraction of a population of IL22+ innate lymphoid cells, and the bacterial strain is selected from the group consisting of Acinetobacter lwoffii, Coprobacillus, Clostridium sordellii, Veillonella, and combinations thereof. In various other embodiments, the Acinetobacter lwoffii bacterium is the species F78, and the Coprobacillus bacterium is the species 8_2_54BFAA, the Clostridium sordellii bacterium is the species AO32, and the Veillonella bacterium is the species 6_1_27.

In various embodiments, the manipulation comprises an expansion of a population of IL22+ innate lymphoid cells, and the bacterial strain is selected from the group consisting of Acinetobacter baumannii, Bacteroides dorei, and a combination thereof. In various other embodiments, the Acinetobacter baumannii bacterium is the species ATCC17978, and the Bacteroides dorei bacterium is the species DSM17855.

In various embodiments, the manipulation comprises a contraction of a population of IL22+ innate lymphoid cells, and the bacterial strain is selected from the group consisting of Acinetobacter lwoffii, Fusobacterium nucleatum, Propionibacterium granulosum, Veillonella, and combinations thereof. In various other embodiments, the Acinetobacter lwoffii bacterium is the species F78, the Fusobacterium nucleatum bacterium is the species F0419, the Propionibacterium granulosum bacterium is the species AO42, and the Veillonella bacterium is the species 6_1_27.

In various embodiments, the manipulation comprises an expansion of a population of CD4 T cells, and the bacterial strain is selected from the group consisting of Acinetobacter lwoffii, Bifidobacterium longum, Bacteroides ovatus, Bacteroides thetaiotaomicron, Bacteroides vulgatus, Coprobacillus, Enterococcus faecium, Helicobacter pylori, Ruminococcus gnavus, Veillonella and combinations thereof. In various other embodiments, the Acinetobacter lwoffii bacterium is the species F78, the Bifidobacterium longum bacterium is the species AO44, the Bacteroides ovatus bacterium is the species ATCC8483, the Bacteroides thetaiotaomicron bacterium is the species ATCC29741, the Bacteroides vulgatus bacterium is the species ATCC8482, the Coprobacillus bacterium is the species 8_2_54BFAA, the Enterococcus faecium bacterium is the species TX1330, the Helicobacter pylori bacterium is the species ATCC700392, the Ruminococcus gnavus bacterium is the species ATCC29149, and the Veillonella bacterium is the species 6_1_27. In yet other embodiments, the population of CD4 T cells is IL10+.

In various embodiments, the manipulation comprises a contraction of a population of CD4 T cells, and the bacterial strain is selected from the group consisting of Bacteroides thetaiotaomicron, Peptostreptococus asaccharolyticus, Streptococcus mitis, and combinations thereof. In various other embodiments, the Bacteroides thetaiotaomicron bacterium is the species ATCC29741, the Peptostreptococus asaccharolyticus bacterium is the species AO33, and the Streptococcus mitis bacterium is the species F0392.

In various embodiments, the manipulation comprises a contraction of a population of CD4 T cells, and the bacterial strain is selected from the group consisting of Clostridium perfringens, Peptostreptococus asaccharolyticus, and a combination thereof. In various other embodiments, the Clostridium perfringens bacterium is the species ATCC13124, and the Peptostreptococus asaccharolyticus bacterium is the species AO33. In some embodiments, the population of CD4 T cells is IL17+.

In various embodiments, the contraction or expansion of the immune cell population occurs in the GI tract. In various embodiments, the contraction or expansion of the immune cell population occurs in the colon and the small intestine. In various other embodiments, the contraction or expansion of the immune cell population occurs in the colon. In various other embodiments, the contraction or expansion of the immune cell population occurs in the small intestine.

Various embodiments of the technology described herein also provide for a method of promoting IL10 production or release by cells in the small intestine, the method comprising administering a bacterium of the genus Coprobacillus to the GI tract of the mammal. In some embodiments, the Coprobacillus bacterium is Coprobacillus species 8 2 54BFAA.

Various embodiments also provide for a method of promoting IL22 production or release by Innate Lymphoid Cells in the small intestine or colon of a mammal, the method comprising administering Bacteroides dorei, Acinetobacter baumannii or Bifidobacterium longum cells to the GI tract of the mammal.

Various embodiments also provide for a method of repressing IL22 production or release in a tissue of the GI tract of a mammal, the method comprising administering Acinetobacter lwoffii, Clostridium sordellii, Fusobacterium nucleatum, Propionibacterium granulosum or Veillonella bacterial cells to the GI tract of the mammal. In some embodiments, the Veillonella bacterium is Veillonella species 6 1 27. In various other embodiments, the tissue is the colon.

Various embodiments also provide for a method of suppressing expression of a Reg3 gene in tissue of the small intestine of a mammal, the method comprising administering a composition comprising a Fusobacterium varium bacterium to the GI tract of the mammal.

Various embodiments also provide for a method of promoting the expression of an α-defensin or Reg3 gene in tissue of the colon of a mammal, the method comprising administering a composition comprising a Parabacteroides merdae or Porphyromonas uenonsis bacterium to the GI tract of the mammal.

Various embodiments also provide for a method of promoting expansion in a population of CD8−, CD4−, TCRγ+ T cells in a tissue of the gastrointestinal tract of a mammal, the method comprising administering a composition comprising a Fusobacterium varium bacterium to the GI tract of the mammal. In various embodiments, the tissue of the gastrointestinal tract comprises the small intestine. In various other embodiments, the tissue of the gastrointestinal tract comprises the colon.

Various embodiments also provide for a method of reducing populations of CD4+ T cells and CD8+ T cells, or suppressing expansion of CD4+ T cells and CD8+ T cells, in a tissue of the gastrointestinal tract of a mammal, the method comprising administering a composition comprising a Fusobacterium varium bacterium to the GI tract of the mammal.

Various embodiments also provide for a method of promoting an expansion of an immune cell population in a mammal, the method comprising administering a composition comprising a microbe selected from the group consisting of Clostridium sordellii_AO32, Bacteroides uniformis_ATCC8492, Bacteroides fragilis_NCTC9343, Bacteroides vulgatus_ATCC8482, Bifidobacterium longum_AO44, Bacteroides ovatus_ATCC8483, Bacteroides thetaiotaomicron_ATCC29741, Enterococcus faecium_TX1330, Helicobacter pylori_ATCC700392, Ruminococcus gnavus_ATCC29149, Acinetobacter baumannii_ATCC17978, Acinetobacter lwoffii_F78, Bifidobacterium breve_SK134, Bacteroides dorei_DSM17855, Lachnospiraceae_sp_2_1_58FAA, Lactobacillus casei_AO47, Veillonella_6_1_27, Coprobacillus_8_2_54BFAA or a combination thereof, to the mammal's gastrointestinal GI tract. In various embodiments, the expansion occurs at least in a tissue of the GI tract or a lymphoid tissue. In various other embodiments, the expansion occurs in small intestine (SI), colon, or mesenteric lymph nodes. In other embodiments, the expansion occurs in a Peyer's patch of the SI. In various other embodiments, the increase occurs in an immune cell population of the intestinal lamina propria. In some other embodiments, the increase occurs in an immune cell population of the innate immune system.

Various embodiments also provide for a method of promoting a contraction of an immune cell population in a mammal, the method comprising administering a composition comprising a microbe selected from the group consisting of Acinetobacter baumannii_ATCC17978, Acinetobacter lwoffii_F78, Bifidobacterium breve_SK134, Bacteroides dorei_DSM17855, Collinsella aerofaciens_VPI1003, Clostridium ramosum_AO31, Lachnospiraceaesp_2_1_58FAA, Lactobacillus casei_AO47, Veillonella_6_1_27, Coprobacillus_8_2_54BFAA, Clostridium perfringens_ATCC13124, Lactobacillus rhamnosus_LMS2-1, Staphylococcus saprophyticus_ATCC15305, Parabacteroides distasonis_ATCC8503, Fusobacterium nucleatum_F0419, Propionibacterium granulosum_AO42, Peptostreptococus asaccharolyticus_AO33, Streptococcus mitis_F0392, Clostridium sordellii_AO32, Bacteroides thetaiotaomicron_ATCC29741 or a combination thereof, to the mammal's gastrointestinal GI tract. In various embodiments, the contraction occurs at least in a tissue of the GI tract or a lymphoid tissue. In various other embodiments, the contraction occurs in small intestine (SI), colon, or mesenteric lymph nodes. In some embodiments, the contraction occurs in a Peyer's patch of the SI. In various other embodiments, the contraction occurs in an immune cell population of the intestinal lamina propria. In other embodiments, the contraction occurs in an immune cell population of the innate immune system.

In various embodiments, the method comprises the manipulation of a selected population of immune cells. In some embodiments, the immune cells are cells from the innate and/or the adaptive immune system. In various embodiment, the cells of the innate immune system include, but are not limited to, white blood cells (WBCs), leukocytes, natural killer cells (NK cells), mast cells, granulocytes, eosinophils, basophils, polymorphonuclear cells (PMNs), γδ T cells; and the phagocytic cells include macrophages, neutrophils, dendritic cells (DCs). In various embodiments, the cells of the adaptive immune system include, but are not limited to white blood cells, lymphocytes (such as, B cells and T cells). In some embodiments, the B and T cells can be naïve cells. In some other embodiments, the T cells are effector cells, memory cells, regulatory cells, helper cells, or cytotoxic cells. In various embodiments, the immune cell populations manipulated are monocytes, macrophages (MF), mononuclear phagocytes (MPN), dendritic cells (DC), plasmocytoid dendritic cells (pDC), type 3 innate lymphoid cells (ILC3), innate lymphoid cells (ILC), and/or CD4+ T-cells (T4).

In various embodiments, the manipulation of a selected population of immune cells comprises cell expansion and/or contraction. In various other embodiments, cell expansion and/or contraction occurs in the GI tract. In some other embodiments, cell expansion and/or contraction occurs in the colon and/or small intestine of the subject.

Various embodiments also provide for a method of administering a heterologous polypeptide to a mammal, the method comprising administering a bacterium engineered to express the heterologous polypeptide to the GI tract of the mammal. In various embodiments, the bacterium is Peptostreptococcus magnus and/or Bacteroides salanitronis.

These bacterial species can provide ways to deliver a heterologous polypeptide without provoking a significant immune cell response triggered by the bacterium itself. That is their lack of significant impact on the cell populations examined renders them useful for delivery of a biologic with minimal impact of the delivering microbe. Methods of engineering these species to express a given biologic, e.g., from a recombinant vector construct, are known to those of ordinary skill in the art.

Promoting and/or Suppressing Gene Expression

Various embodiments provide for a method of suppressing expression of a Reg3 gene in tissue of the small intestine of a mammal, the method comprising administering a composition comprising a Fusobacterium varium bacterium to the GI tract of the mammal.

Various embodiments also provide for a method of promoting the expression of an α-defensin or Reg3 gene in tissue of the colon of a mammal, the method comprising administering a composition comprising a Parabacteroides merdae or Porphyromonas uenonsis bacterium to the GI tract of the mammal.

The promotion and/or suppression of gene expression can be assessed from measuring nucleic acid and/or protein levels derived from a biological sample using any of various techniques and/or methods well-known in the art. In various embodiments, methods/systems to detect nucleic acids include but are not limited to northern blot, reverse transcription PCR, real-time PCR, serial analysis of gene expression (SAGE), DNA microarray, tiling array, RNA-Seq, or a combination thereof. In various other embodiments, the gene expression levels for genes in the Reg3 and/or α-defensin families are assayed. In various other embodiments, the gene expression levels for genes for Paneth cell-derived products such as, but not limited to Ang4 are assayed. In various embodiments, methods and systems to detect protein expression include, but are not limited to ELISA, immunohistochemistry, western blot, flow cytometry, fluorescence in situ hybridization (FISH), radioimmuno assays, and affinity purification. Once the expression levels have been determined, the resulting data can be analyzed using various algorithms, based on well-known methods used by those skilled in the art. In various other embodiments, the protein levels for genes in the Reg3 and/or α-defensin families are assayed. In various other embodiments, the protein levels for genes for Paneth cell-derived products such as, but not limited to Ang4 are assayed.

In various embodiments, the biological sample can be a tissue of the large and/or small intestine. In various other embodiments, the large intestine sample comprises the cecum, colon (the ascending colon, the transverse colon, the descending colon, and the sigmoid colon), rectum and/or the anal canal. In yet other embodiments, the small intestine sample comprises the duodenum, jejunum, and/or the ileum.

### Promoting Treg Expansion

Various embodiments of the present invention provide for a method of promoting an expansion of a population of Treg cells in a mammal, the method comprising administering bacterial cells to the GI tract of the mammal. In various embodiments, the expansion occurs in a population in the GI tract of the mammal. In various embodiments, the expansion occurs in the colon and/or small intestine of the GI tract of the mammal. In various other embodiments, the expansion comprises expansion of RORγ+ Tregs in the small intestine or colon. In other embodiments, the expansion comprises expansion of RORγ− Treg cells in the small intestine or colon. In various other embodiments, the expansion comprises expansion of Helios+ Treg cells in the small intestine or colon. In yet other embodiments, the bacterial cells can be one or more of the following genus Clostridium, Bacteroides and Fusobacterium. In various embodiments, the bacterial cells can be one or more of C. ramosum, B. thetaiotaomicron, F. varium, B. vulgatus, B. adolescentis and B. uniformis.

Various embodiments also provide for a method of promoting an expansion of a population of RORγ+ Helios− Treg cells in a mammal, the method comprising administering a composition comprising a single bacterial cell species to the GI tract of the mammal. In various embodiments, the expansion comprises expansion of RORγ+Helios− Tregs in the small intestine or colon. In yet other embodiments, the bacterial cells can be one or more of the following genus Clostridium, Bacteroides and Fusobacterium. In various embodiments, the bacterial cells can be one or more of C. ramosum, B. thetaiotaomicron, F. varium, B. vulgatus, B. adolescentis and B. uniformis.

### Localized Delivery of Bioactive Molecules

Various embodiments of the methods and compositions described herein provide for a method of sustained, localized delivery of a bioactive molecule to the GI tract by administering a composition comprising microbes that localize in said location. In various other embodiments, localized delivery of a bioactive molecule is to the lower GI tract. In yet other embodiments, localized delivery of a bioactive molecule is to the oral cavity. In various other embodiments, localized delivery of a bioactive molecule is to the stomach. In some embodiments, the microbes are exclusive to the location of the localized delivery.

Various embodiments of the present invention also provide for a method of sustained, localized delivery of a bioactive molecule to the oral cavity of a mammal, the method comprising administering a composition comprising a Porphyromonas gingivalis, Prevotella intermedia or Prevotella melaninogenica bacterium to the mammal.

Various embodiments also provide for a method of treating an oral disease or disorder, the method comprising sustained, localized delivery of a bioactive molecule to the oral cavity of a mammal by administering a composition comprising a Porphyromonas gingivalis, Prevotella intermedia or Prevotella melaninogenica bacterium to the mammal.

In various embodiments, the bioactive molecule is expressed by the administered bacterium. In various other embodiments, the administered bacterium is engineered to express the bioactive molecule. In yet other embodiments, the bioactive molecule comprises an antibiotic, an anti-microbial peptide (AMP), an anti-inflammatory polypeptide, an antibody, and/or a cytokine. In various embodiments, the composition is administered orally.

In various embodiments, the oral disease or disorder includes, but is not limited to caries, periodontal disease, thrush, aphthous ulcer, and halitosis.

Various embodiments also provide for a method of sustained, localized delivery of a bioactive molecule to the stomach of a mammal, the method comprising administering a composition comprising a Lactobacillus johnsonii bacterium to the mammal. In various embodiments, the Lactobacillus johnsonii is of the strain AO12. In various embodiments, the bioactive molecule is expressed by the administered bacterium. In various other embodiments, the administered bacterium is engineered to express the bioactive molecule. In yet other embodiments, the bioactive molecule comprises an antibiotic, an anti-microbial peptide (AMP), an anti-inflammatory polypeptide, an antibody, and/or a cytokine.

Various embodiments also provide for a composition for sustained, localized delivery of a bioactive molecule to a tissue of the oral cavity of a mammal, the composition comprising a Porphyromonas gingivalis, Prevotella intermedia or Prevotella melaninogenica bacterium in a pharmaceutical carrier adapted for oral delivery.

Various embodiments also provide for a composition for the sustained, localized delivery of a bioactive molecule to the stomach of a mammal, the composition comprising a Lactobacillus johnsonii bacterium in a carrier adapted for oral delivery.

In various embodiments, the bacterium expresses the bioactive molecule. In various other embodiments, the bacterium is engineered to express the bioactive molecule. In some embodiments, the bioactive molecule comprises an antibiotic, an anti-microbial peptide (AMP), an anti-inflammatory polypeptide, an antibody, and/or a cytokine.

In various embodiments, the pharmaceutical carrier comprises a foodstuff. In various other embodiments, the composition is in the form of a paste, cream, ointment, gel or liquid. In some embodiments, the composition is in the form of a toothpaste, mouth spray, mouth rinse or mouthwash. In various embodiments, at least 10⁸ of the bacterium are present in the composition. In various embodiments, the composition comprises a prebiotic.

### Therapeutics

Various embodiments provide for the manipulation of immune cells by the administration of a therapeutically effective amount bacterial strain or bacterial composition which is useful for a variety of applications including, but not limited to therapeutic treatment methods, such as treating a subject with a disease. In various embodiments, the diseases treated include, but are not limited to cancer such as intestinal tumorigenesis and colorectal cancer, among others, inflammatory bowel disease such as Crohn's disease and ulcerative colitis, inflammatory bowel syndrome, and IFNγ linked diseases. The microbiome has been implicated in, and can inform the treatment of numerous disorders that affect tissues and systems other than the small intestine and colon. These include, for example, caries, periodontal disease, systemic immune disorders such as Multiple Sclerosis, rheumatoid arthritis, psoriasis, systemic lupus erythematosus, asthma and diabetes, among others, metabolic syndrome, obesity, food allergy, anxiety, depression, obsessive-compulsive disorder, and autism spectrum disorders, among others. The methods of use can be in vitro, ex vivo, or in vivo methods.

Terms such as “treating” or “treatment” or “to treat” or “alleviating” or “to alleviate” refer to therapeutic treatment and/or prophylactic or preventative measures, wherein the object is to prevent or slow down (lessen) the pathologic condition, prevent the pathologic condition, pursue or obtain good overall survival, improve quality of life, reduce at least one symptom, as an adjunct to include with other treatments, or lower the chances of the individual developing the condition even if the treatment is ultimately unsuccessful. Thus, those in need of treatment include those already with the disorder; those prone to have the disorder; and those in whom the disorder is to be prevented. In some embodiments, “treating” refers to administration to an individual lacking a diagnosable disease (e.g. subclinical symptoms) for the purpose of e.g., improving quality of life, reduction of non-disease related systemic inflammation, reducing sub-clinical symptoms of e.g., irritable bowel syndrome, or for replacement of an appropriate microbiome following treatment of a subject with short-course antibotics.

The term “therapeutically effective amount” refers to an amount of a bacterial strain or bacterial composition effective to “treat” a disease or disorder in a subject, which can reduce the severity of disease symptoms.

In various embodiments, the administration of the selected bacterial strain or bacterial composition is therapeutic. In some embodiments, the administration of the selected bacterial strain or bacterial composition is therapeutic due to expansion of an immune cell population. In other embodiments, the administration of the selected bacterial strain or bacterial composition is therapeutic due to contraction of an immune cell population. In other embodiments, the administration of the selected bacterial strain provides a prophylactic or preventative benefit.

In various embodiments, the administration of different bacterial strains has different effects on the immune population. In various other embodiments, the administration of closely related bacterial strains does not result in similar effects on the immune population.

### Dosage and Administration

Various embodiments provide for the administration of a bacterial strain to a subject for the manipulation of an immune population. In various embodiments, the subject is administered a composition of two or more bacterial strains.

In various embodiments, the bacterial strain or bacterial composition can be formulated for delivery via any route of administration. “Route of administration” can refer to any administration pathway known in the art, although it is preferred to administer to the GI tract via an oral route or, e.g., a rectal route.

Via the enteral route, the bacterial strain or bacterial composition can be administered in the form of tablets, gel capsules, sugar-coated tablets, syrups, suspensions, solutions, powders, granules, emulsions, microspheres or nanospheres or lipid vesicles or polymer vesicles allowing controlled release. In various embodiments, the bacterial strain or bacterial composition can be administered in the form of tablets, capsules, granules, spheres or vesicles that comprise an enteric coating. The enteric coating can be a polymer barrier that aids in the prevention of dissolution or disintegration in the gastric environment. In various embodiments, the enteric coating can include, but is not limited to a coating that is water-miscible or acid-resistant. In other embodiments, the bacterial strain or bacterial composition comprises of one or more coatings. In yet other embodiments, the coating can be a controlled-release coating. In various embodiments, the enteric coating material can include, but is not limited to, fatty acids, waxes, shellac, plastics, and plant fibers.

The bacterial strains or bacterial composition administered, according to the invention can also contain any pharmaceutically acceptable carrier. “Pharmaceutically acceptable carrier” as used herein refers to a pharmaceutically acceptable material, composition, or vehicle that is involved in carrying or transporting the bacterial strain or the bacterial composition of interest into the subject. For example, the carrier can be a liquid or solid filler, diluent, excipient, solvent, or encapsulating material, or a combination thereof. Each component of the carrier must be “pharmaceutically acceptable” in that it must be compatible with the other ingredients of the formulation. The bacterial strain or bacterial composition can be mixed with carriers which are pharmaceutically acceptable and in amounts suitable for use in the therapeutic methods described herein. It must also be suitable for use in contact with any tissues or organs with which it may come in contact, meaning that it must not carry a risk of toxicity, irritation, allergic response, immunogenicity, or any other complication that excessively outweighs its therapeutic benefits. Physiologically tolerable carriers are well known in the art. Such carriers can be solid, liquid, or semisolid. Suitable carriers are, for example, starch, glucose, lactose, sucrose, gelatin, malt, rice, flour, chalk, silica gel, talc, sodium chloride, dried skim milk, water, saline, dextrose, mannitol, polysorbate, vegetable oils such as cottonseed oil, and water:oil emulsions or the like and combinations thereof. In various embodiments, the carrier is of an edible nature, such as, but not limited to foodstuffs such as food or beverages. In various embodiments, the bacterial strain or bacterial composition is administered with a prebiotic. As used herein, a “prebiotic” refers to an ingredient that allows or promotes specific changes, both in the composition and/or activity in the gastrointestinal microbiota that may (or may not) confer benefits upon the host. In some embodiments, a prebiotic can include, but is not limited to, one or more of the following: amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligofructose-enriched inulin, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides. In other embodiments, the prebiotic is obtained from plant-derived complex carbohydrates, oligosaccharides or polysaccharides.

In various embodiments, the prebiotic is useful for the survival, colonization and persistence of the bacterial strain or bacterial composition administered. In various embodiments, the prebiotic is indigestible or poorly digested by humans and serves as a food source for bacteria. In various other embodiments, the prebiotics can be purified or chemically or enzymatically synthesized. In some embodiments, the bacterial strain or bacterial composition comprises at least one prebiotic. In various embodiments, the prebiotic is administered prior to, simultaneously or subsequently to the administration of the bacterial strain or bacterial composition. In various embodiments, the prebiotic aids in the growth or maintenance of the bacterial strain or bacterial composition administered.

The bacterial strain or bacterial compositions according to the methods and compositions described herein can be delivered in an effective amount to manipulate the immune cells and/or be supplement or therapeutic for the subject.

The precise effective amount is that amount of the bacterial strain or bacterial composition that will yield the most effective results in terms of efficacy of immunomodulation and/or treatment in a given subject. The amount of the bacterial strain or bacterial composition used in the methods and compositions described herein that will be effective in the treatment of a particular disorder or condition will depend on the nature of the disorder or condition, and can be determined by one of skill in the art with standard clinical techniques. This amount will vary depending upon a variety of factors, including but not limited to the characteristics of the bacterial strain (including biological activity), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the art will be able to determine an effective amount through routine experimentation, for instance, by monitoring a subject's response to administration of a bacterial strain or bacterial composition and adjusting the dosage accordingly.

Typical dosages of an effective bacterial strain or bacterial composition can be as indicated to the skilled artisan by the in vitro responses or responses in animal models. Such dosages typically can be reduced by up to about one order of magnitude in amount without losing the effective biological activity of the bacterial strain or bacterial composition. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biological samples obtained, or the responses observed in the appropriate animal models.

In various embodiments, the dosage of the bacterial strain or bacterial composition is in the range of about 10¹ to about 10¹³ cells or colony-forming units (CFUs). The dosage of the bacterial strain or bacterial composition administered to the subject can range from about 10¹-10² CFU/g, 10²-10⁴ CFU/g, 10⁴-10⁶ CFU/g, 10⁶-10⁸ CFU/g, 10⁸-10¹⁰ CFU/g, 10¹⁰-10¹³ CFU/g or a combination thereof. In certain embodiments, the dosage is 10⁹-10¹² CFU/g.

For the treatment of a disease, the appropriate dosage of the bacterial strain or bacterial composition of the present invention depends on the type of disease to be treated, the severity and course of the disease, the responsiveness of the disease, whether the bacterial strain or bacterial composition is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates. The bacterial strain or bacterial composition can be administered one time or over a series of treatments lasting from several days to several months, or until a cure is effected or a diminution of the disease state is achieved (e.g., treatment or amelioration of IBD). The duration of treatment depends upon the subject's clinical progress and responsiveness to therapy.

The bacterial strain or bacterial composition described herein is useful, for example, in a variety of applications including, but not limited to, modulation of the immune cell population in a subject and/or therapeutic treatment for various diseases, discussed herein. The methods of use can be in vitro, ex vivo, or in vivo methods.

The present invention may be as described in any one of the following numbered paragraphs:

1. A method for manipulating a selected population of immune cells in a subject, the method comprising administering to the subject a bacterial strain selected from the group consisting of Clostridium sordellii, Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella, Coprobacillus, Bacteroides uniformis, Clostridium perfringens, Bacteroides fragilis, Bacteroides vulgatus, Lactobacillus rhamnosus, Staphylococcus saprophyticus, Parabacteroides distasonis, Fusobacterium nucleatum, Propionibacterium granulosum, Bifidobacterium longum, Bacteroides ovatus, Bacteroides thetaiotaomicron, Enterococcus faecium, Helicobacter pylori, Ruminococcus gnavus, Peptostreptococus asaccharolyticus, Streptococcus mitis, or a combination thereof.

2. The method of paragraph 1, wherein the bacterial strain is administered to the GI tract of the subject.

3. The method of paragraph 2, wherein the manipulation comprises a change in an immune cell population in a tissue of the colon or small intestine.

4. The method of any one of paragraphs 1-3, wherein the manipulation comprises an expansion of a monocyte population, and the bacterial strain is Clostridium sordellii.

5. The method of paragraph 4, wherein the Clostridium sordellii bacterium is the species AO32.

6. The method of any one of paragraphs 1-5, wherein the manipulation comprises a contraction of a population of macrophages, and the bacterial strain is selected from the group consisting of Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella or a combination thereof.

7. The method of paragraph 6, wherein the Acinetobacter baumannii bacterium is the species ATCC7978, the Acinetobacter lwoffii bacterium is the species F78, the Bifidobacterium breve bacterium is the species SK134, the Bacteroides dorei bacterium is the species DSM17855, the Collinsella aerofaciens bacterium is the species VPI1003, the Clostridium ramosum bacterium is the species AO31, the Lachnospiraceae bacterium is the species sp_2_1_58FAA, the Lactobacillus casei bacterium is the species AO47, and the Veillonella bacterium is the species 6_1_27.

8. The method of paragraph 5, wherein the population of macrophages is CD11b+, CD11C−, F4/80+.

9. The method of any one of paragraphs 1-8, wherein the manipulation comprises a contraction of a population of mononuclear phagocytes, and the bacterial strain is selected from the group consisting of Acinetobacter lwoffii, Collinsella aerofaciens, Coprobacillus, and combinations thereof.

10. The method of paragraph 9, wherein the Acinetobacter lwoffii bacterium is the species F78, the Collinsella aerofaciens bacterium is the species VPI1003, and the Coprobacillus bacterium is the species 8_2_54BFAA.

11. The method of paragraph 7, wherein the population of mononuclear phagocytes is CD11b+, CD11c+, F4/80+.

12. The method of any one of paragraphs 1-11, wherein the manipulation comprises an expansion of a population of dendritic cells, and the bacterial strain is selected from the group consisting of Bifidobacterium breve, Bacteroides uniformis, Lachnospiraceae, and combinations thereof.

13. The method of paragraph 12, wherein the Bifidobacterium breve bacterium is the species SK134, the Bacteroides uniformis bacterium is the species ATCC8492, and the Lachnospiraceae bacterium is the species sp_2_1_58FAA.

14. The method of paragraph 9, wherein the population of dendritic cells is CD103+, CD11b+.

15. The method of any one of paragraphs 1-14, wherein the manipulation comprises a contraction of a population of CD103+, CD11b+ dendritic cells, and the bacterial strain is selected from the group consisting of Acinetobacter lwoffii_F78, Clostridium perfringens_ATCC13124, and a combination thereof.

16. The method of paragraph 15, wherein the Acinetobacter lwoffii bacterium is the species F78 and the Clostridium perfringens bacterium is the species ATCC13124.

17. The method of paragraph 11, wherein the population of dendritic cells is CD103+, CD11b+.

18. The method of any one of paragraphs 1-17, wherein the manipulation comprises an expansion of a population of plasmacytoid dendritic cells, and the bacterial strain is selected from the group consisting of Bacteroides fragilis, Bacteroides vulgatus, and a combination thereof.

19. The method of paragraph 18, wherein the Bacteroides fragilis bacterium is the species NCTC9343, and the Bacteroides vulgatus bacterium is the species ATCC8482.

20. The method of any one of paragraphs 1-19, wherein the manipulation comprises a contraction of a population of plasmacytoid dendritic cells, and the bacterial strain is selected from the group consisting of Lactobacillus rhamnosus, Staphylococcus saprophyticus, and a combination thereof.

21. The method of paragraph 20, wherein the Lactobacillus rhamnosus bacterium is the species LMS2-1, and the Staphylococcus saprophyticus bacterium is the species ATCC15305.

22. The method of any one of paragraphs 1-21, wherein the manipulation comprises a contraction of a population of type 3 innate lymphoid cells, and the bacterial strain is selected from the group consisting of Coprobacillus, Parabacteroides distasonis, Veillonella, and combinations thereof.

23. The method of paragraph 22, wherein the Coprobacillus bacterium is the species 8_2_54BFAA, and the Parabacteroides distasonis bacterium is the species ATCC8503, and the Veillonella bacterium is the species 6_1_27.

24. The method of any one of paragraphs 1-23, wherein the manipulation comprises an expansion of a population of IL22+ innate lymphoid cells, and the bacterial strain is selected from the group consisting of Bacteroides uniformis, Lactobacillus casei, and a combination thereof.

25. The method of paragraph 24, wherein the Bacteroides uniformis bacterium is the species ATCC8492, and the Lactobacillus casei bacterium is the species AO47.

26. The method of any one of paragraphs 1-25, wherein the manipulation comprises a contraction of a population of IL22+ innate lymphoid cells, and the bacterial strain is selected from the group consisting of Acinetobacter lwoffii, Coprobacillus, Clostridium sordellii, Veillonella, and combinations thereof.

27. The method of paragraph 26, wherein the Acinetobacter lwoffii bacterium is the species F78, and the Coprobacillus bacterium is the species 8_2_54BFAA, the Clostridium sordellii bacterium is the species AO32, and the Veillonella bacterium is the species 6_1_27.

28. The method of any one of paragraphs 1-27, wherein the manipulation comprises an expansion of a population of IL22+ innate lymphoid cells, and the bacterial strain is selected from the group consisting of Acinetobacter baumannii, Bacteroides dorei, and a combination thereof.

29. The method of paragraph 28, wherein the Acinetobacter baumannii bacterium is the species ATCC17978, and the Bacteroides dorei bacterium is the species DSM17855.

30. The method of any one of paragraphs 1-29, wherein the manipulation comprises a contraction of a population of IL22+ innate lymphoid cells, and the bacterial strain is selected from the group consisting of Acinetobacter lwoffii, Fusobacterium nucleatum, Propionibacterium granulosum, Veillonella, and combinations thereof.

31. The method of paragraph 30, wherein the Acinetobacter lwoffii bacterium is the species F78, the Fusobacterium nucleatum bacterium is the species F0419, the Propionibacterium granulosum bacterium is the species AO42, and the Veillonella bacterium is the species 6_1_27.

32. The method of any one of paragraphs 1-31, wherein the manipulation comprises an expansion of a population of CD4 T cells, and the bacterial strain is selected from the group consisting of Acinetobacter lwoffii, Bifidobacterium longum, Bacteroides ovatus, Bacteroides thetaiotaomicron, Bacteroides vulgatus, Coprobacillus, Enterococcus faecium, Helicobacter pylori, Ruminococcus gnavus, Veillonella and combinations thereof.

33. The method of paragraph 32, wherein the Acinetobacter lwoffii bacterium is the species F78, the Bifidobacterium longum bacterium is the species AO44, the Bacteroides ovatus bacterium is the species ATCC8483, the Bacteroides thetaiotaomicron bacterium is the species ATCC29741, the Bacteroides vulgatus bacterium is the species ATCC8482, the Coprobacillus bacterium is the species 8_2_54BFAA, the Enterococcus faecium bacterium is the species TX1330, the Helicobacter pylori bacterium is the species ATCC700392, the Ruminococcus gnavus bacterium is the species ATCC29149, and the Veillonella bacterium is the species 6_1_27.

34. The method of paragraph 20, wherein the population of CD4 T cells is IL10+.

35. The method of any one of paragraphs 1-34, wherein the manipulation comprises a contraction of a population of CD4 T cells, and the bacterial strain is selected from the group consisting of Bacteroides thetaiotaomicron, Peptostreptococus asaccharolyticus, Streptococcus mitis, and combinations thereof.

36. The method of paragraph 35, wherein the Bacteroides thetaiotaomicron bacterium is the species ATCC29741, the Peptostreptococus asaccharolyticus bacterium is the species AO33, and the Streptococcus mitis bacterium is the species F0392.

37. The method of any one of paragraphs 1-36, wherein the manipulation comprises a contraction of a population of CD4 T cells, and the bacterial strain is selected from the group consisting of Clostridium perfringens, Peptostreptococus asaccharolyticus, and a combination thereof.

38. The method of paragraph 37, wherein the Clostridium perfringens bacterium is the species ATCC13124, and the Peptostreptococus asaccharolyticus bacterium is the species AO33.

39. The method of paragraph 22 or 23, wherein the population of CD4 T cells is IL17+.

40. The method of any one of paragraphs 4-17 or 20-22 wherein the contraction or expansion of the immune cell population occurs in the colon.

41. The method of any one of paragraphs 18, 19, 23 or 24 wherein the contraction or expansion of the immune cell population occurs in the small intestine.

42. A method of promoting IL10 production or release by cells in the small intestine, the method comprising administering a bacterium of the genus Coprobacillus to the GI tract of the mammal.

43. The method of paragraph 42, wherein the Coprobacillus bacterium is Coprobacillus species 8 2 54BFAA.

44. A method of promoting IL22 production or release by Innate Lymphoid Cells in the small intestine or colon of a mammal, the method comprising administering Bacteroides dorei, Acinetobacter baumannii or Bifidobacterium longum cells to the GI tract of the mammal.

45. A method of repressing IL22 production or release in a tissue of the GI tract of a mammal, the method comprising administering Acinetobacter lwoffii, Clostridium sordellii, Fusobacterium nucleatum, Propionibacterium granulosum or Veillonella bacterial cells to the GI tract of the mammal.

46. The method of paragraph 45, wherein the Veillonella bacterium is Veillonella species 6 1 27.

47. The method of paragraph 46, wherein the tissue is the colon.

48. A method of suppressing expression of a Reg3 gene in tissue of the small intestine of a mammal, the method comprising administering a composition comprising a Fusobacterium varium bacterium to the GI tract of the mammal.

49. A method of promoting the expression of an α-defensin or Reg3 gene in tissue of the colon of a mammal, the method comprising administering a composition comprising a Parabacteroides merdae or Porphyromonas uenonsis bacterium to the GI tract of the mammal.

50. A method of promoting expansion in a population of CD8−, CD4−, TCRγ+ T cells in a tissue of the gastrointestinal tract of a mammal, the method comprising administering a composition comprising a Fusobacterium varium bacterium to the GI tract of the mammal.

51. The method of paragraph 50, wherein the tissue of the gastrointestinal tract comprises the small intestine.

52. The method of paragraph 50 or 51, wherein the tissue of the gastrointestinal tract comprises the colon.

53. A method of reducing populations of CD4+ T cells and CD8+ T cells, or suppressing expansion of CD4+ T cells and CD8+ T cells, in a tissue of the gastrointestinal tract of a mammal, the method comprising administering a composition comprising a Fusobacterium varium bacterium to the GI tract of the mammal.

54. A method of promoting an expansion of an immune cell population in a mammal, the method comprising administering a composition comprising a microbe selected from the group consisting of Clostridium sordellii_AO32, Bacteroides uniformis_ATCC8492, Bacteroides fragilis_NCTC9343, Bacteroides vulgatus_ATCC8482, Bifidobacterium longum_AO44, Bacteroides ovatus_ATCC8483, Bacteroides thetaiotaomicron_ATCC29741, Enterococcus faecium_TX1330, Helicobacter pylori_ATCC700392, Ruminococcus gnavus_ATCC29149, Acinetobacter baumannii_ATCC17978, Acinetobacter lwoffii_F78, Bifidobacterium breve_SK134, Bacteroides dorei_DSM17855, Lachnospiraceae_sp_2_1_58FAA, Lactobacillus casei_AO47, Veillonella_6_1_27, Coprobacillus_8_2_54BFAA or a combination thereof, to the mammal's gastrointestinal GI tract.

55. The method of paragraph 54, wherein the expansion occurs at least in a tissue of the GI tract or a lymphoid tissue.

56. The method of paragraph 55, wherein the expansion occurs in small intestine (SI), colon, or mesenteric lymph nodes.

57. The method of paragraph 56, wherein the expansion occurs in a Peyer's patch of the SI.

58. The method of any one of paragraphs 54-57, wherein the expansion occurs in an immune cell population of the intestinal lamina propria.

59. The method of any one of paragraphs 54-58, wherein the expansion occurs in an immune cell population of the innate immune system.

60. A method of promoting a contraction of an immune cell population in a mammal, the method comprising administering a composition comprising a microbe selected from the group consisting of Acinetobacter baumannii_ATCC17978, Acinetobacter lwoffii_F78, Bifidobacterium breve_SK134, Bacteroides dorei_DSM17855, Collinsella aerofaciens_VPI1003, Clostridium ramosum_AO31, Lachnospiraceae_sp_2_1_58FAA, Lactobacillus casei_AO47, Veillonella_6_1_27, Coprobacillus_8_2_54BFAA, Clostridium perfringens_ATCC13124, Lactobacillus rhamnosus_LMS2-1, Staphylococcus saprophyticus_ATCC15305, Parabacteroides distasonis_ATCC8503, Fusobacterium nucleatum_F0419, Propionibacterium granulosum_AO42, Peptostreptococus asaccharolyticus_AO33, Streptococcus mitis_F0392, Clostridium sordellii_AO32, Bacteroides thetaiotaomicron_ATCC29741 or a combination thereof, to the mammal's gastrointestinal GI tract.

61. The method of paragraph 60, wherein the contraction occurs at least in a tissue of the GI tract or a lymphoid tissue.

62. The method of paragraph 61, wherein the contraction occurs in small intestine (SI), colon, or mesenteric lymph nodes.

63. The method of paragraph 62, wherein the contraction occurs in a Peyer's patch of the SI.

64. The method of any one of paragraphs 60-63, wherein the contraction occurs in an immune cell population of the intestinal lamina propria.

65. The method of any one of paragraphs 60-64, wherein the contraction occurs in an immune cell population of the innate immune system.

66. A method of administering a heterologous polypeptide to a mammal, the method comprising administering a bacterium engineered to express the heterologous polypeptide to the GI tract of the mammal.

67. The method of paragraph 66, wherein the bacterium is Peptostreptococcus magnus and/or Bacteroides salanitronis.

68. A method of sustained, localized delivery of a bioactive molecule to the oral cavity of a mammal, the method comprising administering a composition comprising a Porphyromonas gingivalis, Prevotella intermedia or Prevotella melaninogenica bacterium to the mammal.

69. The method of paragraph 68, wherein the bioactive molecule is expressed by the administered bacterium.

70. The method of paragraph 68 or 69, wherein the administered bacterium is engineered to express the bioactive molecule.

71. The method of any one of paragraphs 68-70, wherein the bioactive molecule comprises an antibiotic, an anti-microbial peptide (AMP), an anti-inflammatory polypeptide, an antibody, a cytokine.

72. The method of any one of paragraphs 68-71, wherein the administering comprises oral administration.

73. A method of treating an oral disease or disorder, the method comprising sustained, localized delivery of a bioactive molecule to the oral cavity of a mammal by administering a composition comprising a Porphyromonas gingivalis, Prevotella intermedia or Prevotella melaninogenica bacterium to the mammal.

74. The method of paragraph 73, wherein the bioactive molecule is expressed by the administered bacterium.

75. The method of paragraph 73 or 74, wherein the administered bacterium is engineered to express the bioactive molecule.

76. The method of any one of paragraphs 73-75, wherein the bioactive molecule comprises an antibiotic, an anti-microbial peptide (AMP), an anti-inflammatory polypeptide, an antibody, a cytokine or a combination thereof.

77. The method of any one of paragraphs 73-76, wherein the oral disease or disorder is selected from caries, periodontal disease, thrush, aphthous ulcer, and/or halitosis.

78. A method of sustained, localized delivery of a bioactive molecule to the stomach of a mammal, the method comprising administering a composition comprising a Lactobacillus johnsonii bacterium to the mammal.

79. The method of paragraph 78, wherein the Lactobacillus johnsonii is of the strain AO12.

80. The method of paragraph 78 or 79, wherein the bioactive molecule is expressed by the administered bacterium.

81. The method of any one of paragraphs 78-80, wherein the administered bacterium is engineered to express the bioactive molecule.

82. The method of any one of paragraphs 78-81, wherein the bioactive molecule comprises an antibiotic, an anti-microbial peptide (AMP), an anti-inflammatory polypeptide, an antibody, a cytokine or combinations thereof.

83. Use of a composition comprising a bacterial strain selected from the group consisting of Clostridium sordellii, Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella, Coprobacillus, Bacteroides uniformis, Clostridium perfringens, Bacteroides fragilis, Bacteroides vulgatus, Lactobacillus rhamnosus, Staphylococcus saprophyticus, Parabacteroides distasonis, Fusobacterium nucleatum, Propionibacterium granulosum, Bifidobacterium longum, Bacteroides ovatus, Bacteroides thetaiotaomicron, Enterococcus faecium, Helicobacter pylori, Ruminococcus gnavus, Peptostreptococus asaccharolyticus, Streptococcus mitis, or a combination thereof for manipulating a selected immune cell population in an individual in need thereof.

84. Use of a composition comprising a bacterium of the genus Coprobacillus to promote IL10 production or release by cells in the small intestine of a mammal in need thereof.

85. Use of a composition comprising Bacteroides dorei, Acinetobacter baumannii or Bifidobacterium longum cells for promoting IL22 production or release by Innate Lymphoid Cells in the small intestine or colon of a mammal in need thereof.

86. Use of a compositions comprising Acinetobacter lwoffii, Clostridium sordellii, Fusobacterium nucleatum, Propionibacterium granulosum or Veillonella bacterial cells to suppress IL22 production or release in a tissue of the GI tract of a mammal in need thereof.

87. Use of a composition comprising Fusobacterium varium bacteria to suppress expression of a Reg3 gene in tissue of the small intestine of a mammal in need thereof.

88. Use of a composition comprising a Parabacteroides merdae or Porphyromonas uenonsis bacterium to promote the expression of an α-defensin or Reg3 gene in tissue of the colon of a mammal in need thereof.

89. Use of a composition comprising a Fusobacterium varium to promote expansion in a population of CD8−, CD4−, TCRγ+ T cells in a tissue of the gastrointestinal tract of a mammal in need thereof.

90. Use of a composition comprising a Fusobacterium varium bacterium to reduce populations of CD4+ T cells and CD8+ T cells, or to suppress expansion of CD4+ T cells and CD8+ T cells, in a tissue of the gastrointestinal tract of a mammal in need thereof.

91. Use of a composition comprising a microbe selected from the group consisting of Clostridium sordellii_AO32, Bacteroides uniformis_ATCC8492, Bacteroides fragilis_NCTC9343, Bacteroides vulgatus_ATCC8482, Bifidobacterium longum_AO44, Bacteroides ovatus_ATCC8483, Bacteroides thetaiotaomicron_ATCC29741, Enterococcus faecium_TX1330, Helicobacter pylori_ATCC700392, Ruminococcus gnavus_ATCC29149, Acinetobacter baumannii_ATCC17978, Acinetobacter lwoffii_F78, Bifidobacterium breve_SK134, Bacteroides dorei_DSM17855, Lachnospiraceae_sp_2_1_58FAA, Lactobacillus casei_AO47, Veillonella_6_1_27, Coprobacillus_8_2_54BFAA or a combination thereof to promote an expansion of an immune cell population in a mammal in need thereof.

92. Use of a composition comprising a microbe selected from the group consisting of Acinetobacter baumannii_ATCC17978, Acinetobacter lwoffii_F78, Bifidobacterium breve_SK134, Bacteroides dorei_DSM17855, Collinsella aerofaciens_VPI1003, Clostridium ramosum_AO31, Lachnospiraceae_sp_2_1_58FAA, Lactobacillus casei_AO47, Veillonella_6_1_27, Coprobacillus_8_2_54BFAA, Clostridium perfringens_ATCC13124, Lactobacillus rhamnosus_LMS2-1, Staphylococcus saprophyticus_ATCC15305, Parabacteroides distasonis_ATCC8503, Fusobacterium nucleatum_F0419, Propionibacterium granulosum_AO42, Peptostreptococus asaccharolyticus_AO33, Streptococcus mitis_F0392, Clostridium sordellii_AO32, Bacteroides thetaiotaomicron_ATCC29741 or a combination thereof to promote a contraction of an immune cell population in a mammal in need thereof.

93. Use of a composition comprising a bacterium engineered to express a heterologous polypeptide in the GI tract of a mammal.

94. Use of a composition comprising a Porphyromonas gingivalis, Prevotella intermedia or Prevotella melaninogenica bacterium for the purpose of sustained, localized delivery of a bioactive molecule to the oral cavity of a mammal in need thereof.

95. Use of a composition comprising a Porphyromonas gingivalis, Prevotella intermedia or Prevotella melaninogenica bacterium for treating an oral disease or disorder.

96. Use of a composition comprising a Lactobacillus johnsonii bacterium for sustained, localized delivery of a bioactive molecule to the stomach of a mammal in need thereof.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

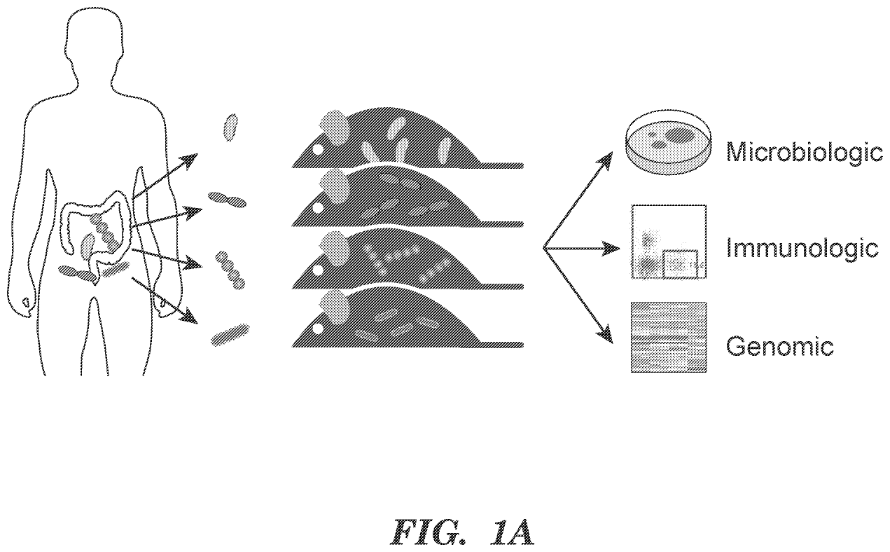

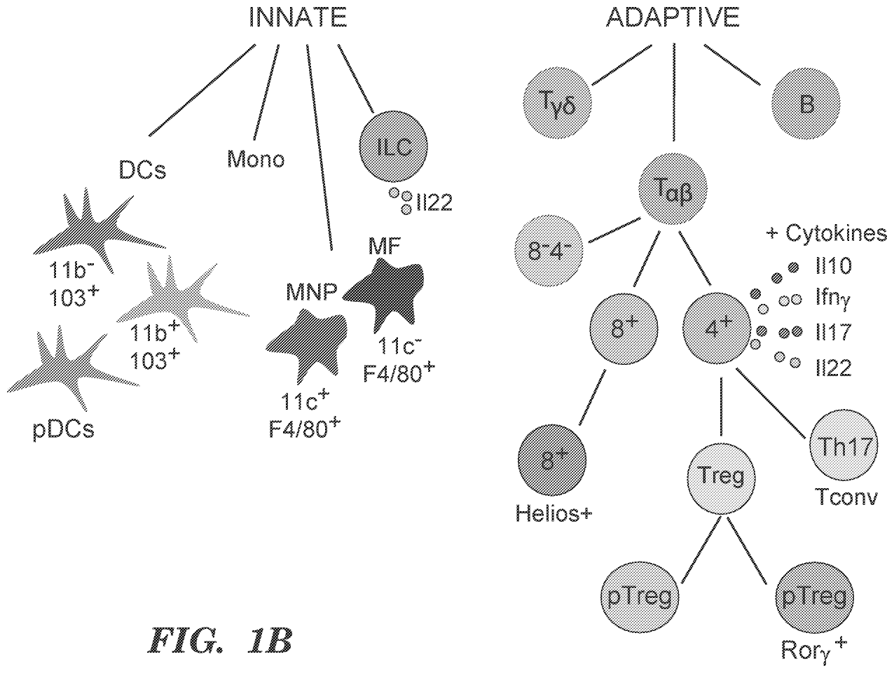

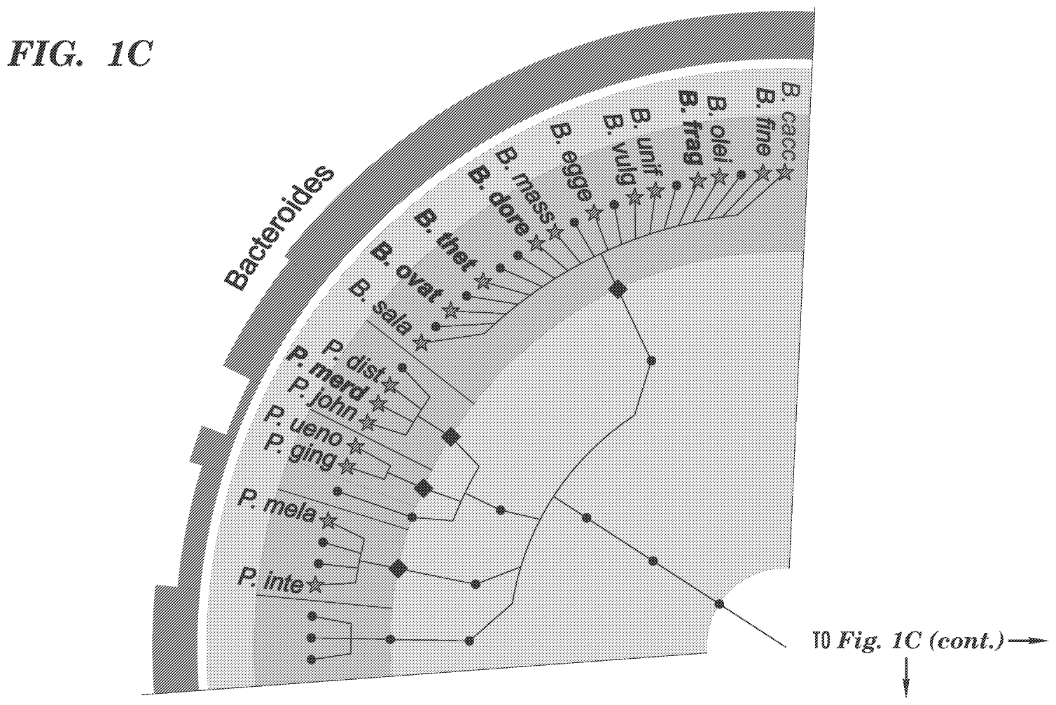

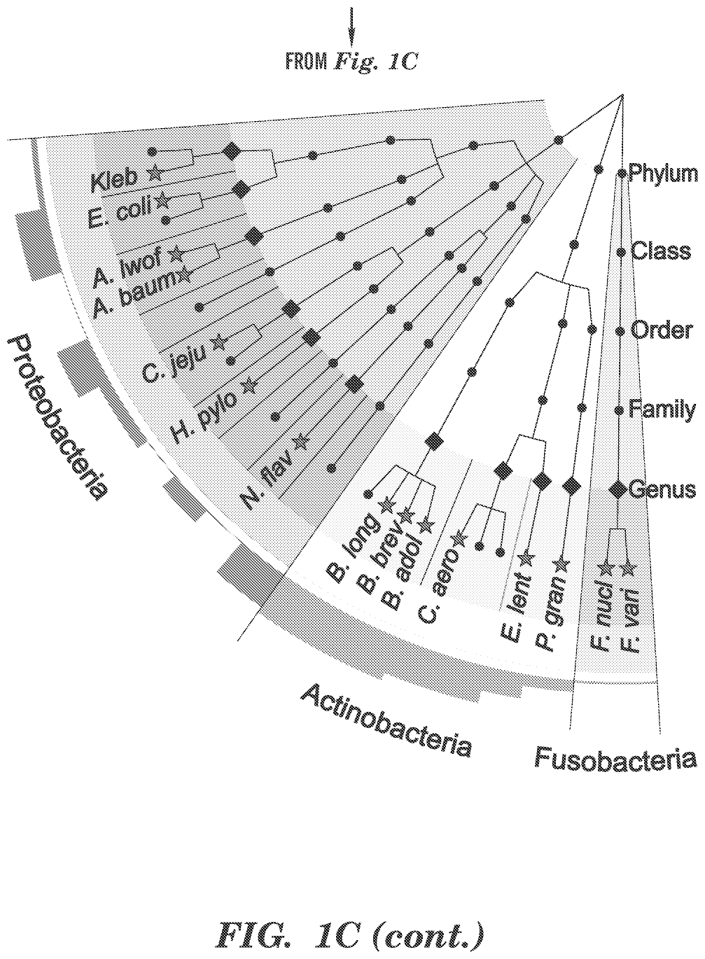

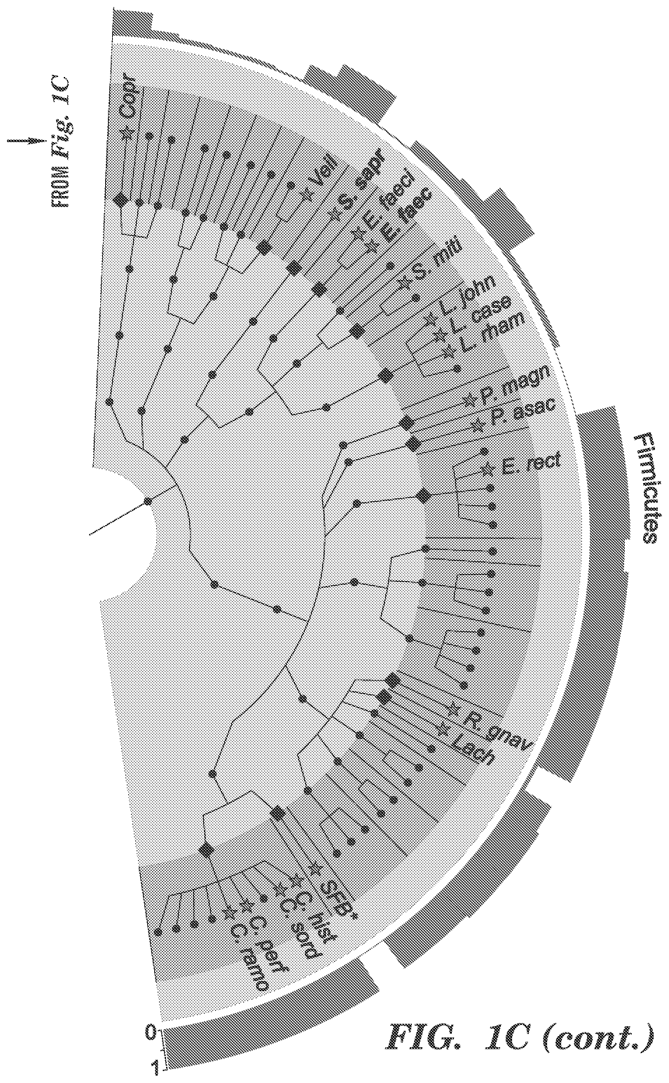

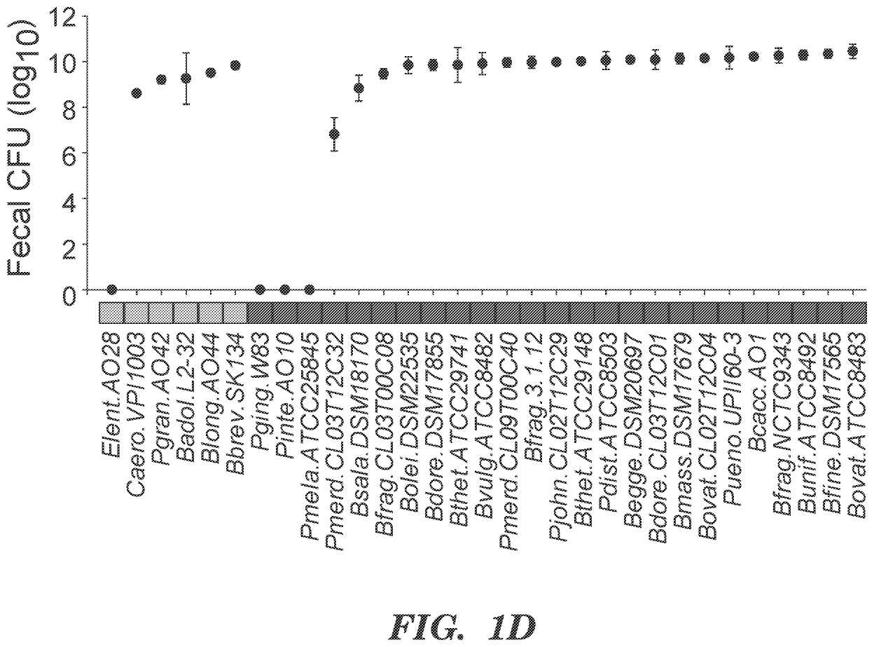

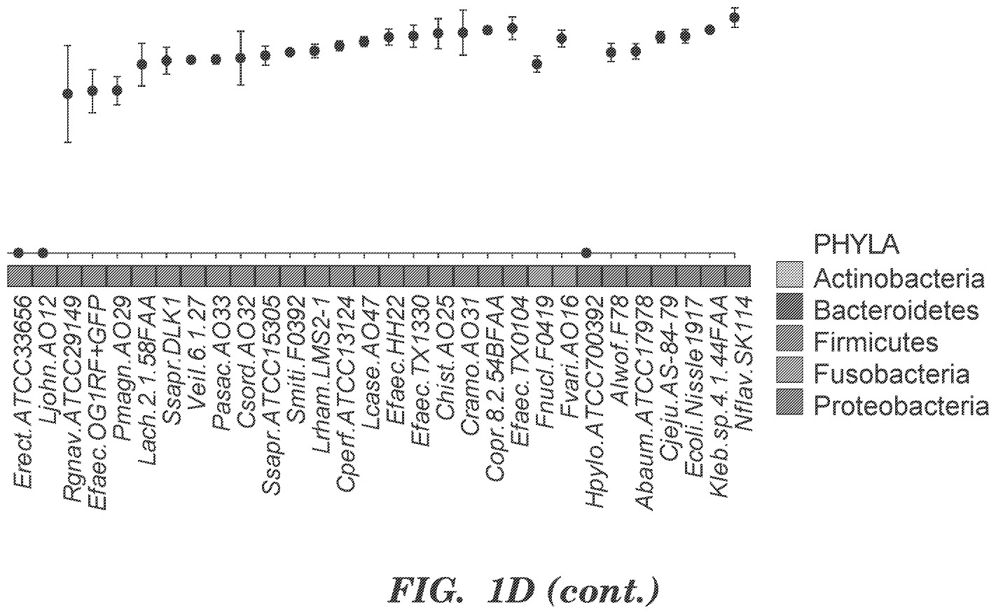

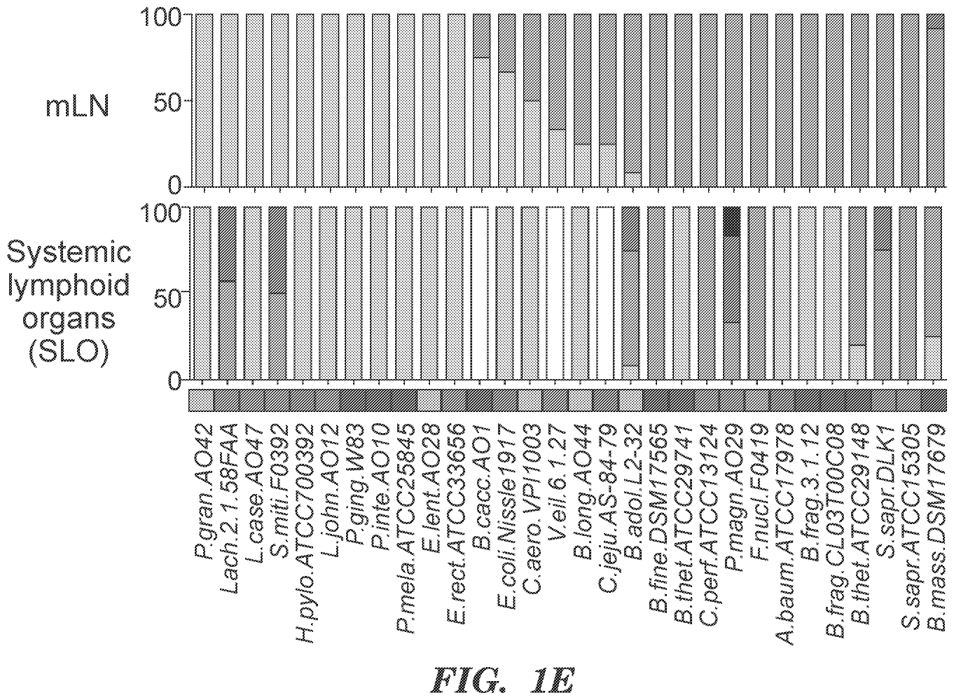

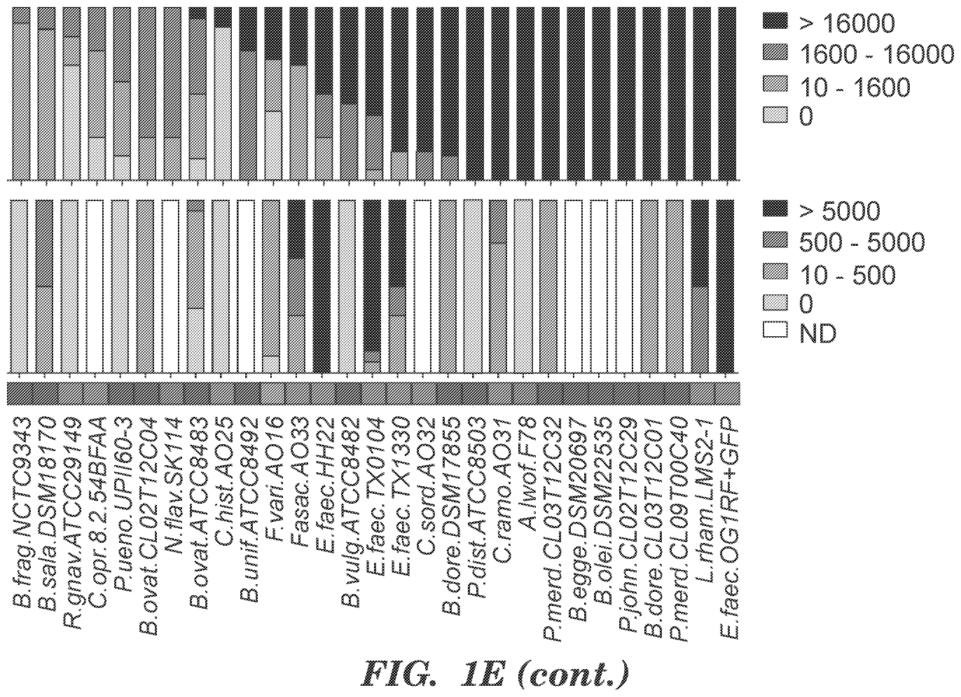

For the study described herein, a systematic screen was set up for human gut symbionts with immunomodulatory activity. GF C57BL/6 mice were bred in an isolator under rigorous microbial monitoring. At precisely 4 weeks of age, eight mice were sterilely transferred to another GF isolator, where they were colonized by gavage with one of the study's 62 bacterial strains (Table 1). Fifty-three strains spanning the known human gut species diversity were originally selected for complete analysis; nine additional strains were chosen from prototypic species for focused analysis to determine whether interesting findings were shared across a species. Mice were maintained under gnotobiotic conditions for 2 weeks, after which they were assessed by immunologic and genomic profiling of the colon and small intestine (SI) (FIG. 1A). Six week old GF mice were regularly analyzed throughout the study. Standard operating procedures were strictly followed throughout the study. All experiments included in this study were documented to ensure monocolonization only with the desired microbe (or GF status) by culture and 16S rDNA sequencing. Any suspicion of microbial contamination led that experiment to be discarded. All experiments that were documented to be free of contamination are reported. Phenotypes of interest were validated by independent repetition of the protocol. Moreover, feces from fourteen randomly chosen experiments were analyzed by deep sequencing and shown to be pure. Table 1 is a list of microbes used in this study. “Microbe_Name” includes the species name and the strain identification; “Key_Microbe_Name” and “Abbreviation” indicate short versions of the Microbe_Name used throughout the paper. “Origin” specifies the source from which the microbe can be obtained. The 16S NCBI match is provided for bacterial species that did not match their original classification.

TABLE 1
List of microbes in the present study

| Microbe_Name | Key_Microbe_Name | Bacterial Species | Abbv | Origin | Aerobic_Anaerobic | Phyla/Family/Genus | Strain_Number |
|---|---|---|---|---|---|---|---|
| Acinetobacter baumannii_ATCC17978 | Abaum.ATCC17978 | Acinetobacter | A. baum | ATCC | Aerobic | Proteobacteria/Moraxellaceae/ | ATCC17978 |
| | | baumannii | | | | Acinetobacter | |
| Acinetobacter lwoffii_F78 | Alwof.F78 | Acinetobacter | A. lwof | Harald Renz | Aerobic | Proteobacteria/Moraxellaceae/ | F78 |
| | | lwoffii | | | | Acinetobacter | |
| Bifidobacterium adolescentis_L2-32 | Badol.L2-32 | Bifidobacterium | B. adol | BEI | Anaerobic | Actinobacteria/Bifidobacteriaceae/ | L2-32 |
| | | adolescentis | | | | Bifidobacterium | |
| Bifidobacterium breve_SK134 | Bbrev.SK134 | Bifidobacterium | B. brev | BWH, Onderdonk lab | Anaerobic | Actinobacteria/Bifidobacteriaceae/ | SK134 |
| | | breve | | | | Bifidobacterium | |
| Bacteroides caccae_AO1 | Bcacc.AO1 | Bacteroides | B. cacc | Clinical isolate, | Anaerobic | Bacteroidetes/Bacteroidaceae/ | AO1 |
| | | caccae | | BWH | | Bacteroides | |
| Bacteroides dorei_CL03T12C01 | Bdore.CL03T12C01 | Bacteroides | B. dore | BEI | Anaerobic | Bacteroidetes/Bacteroidaceae/ | CL03T12C01 |
| | | dorei | | | | Bacteroides | |
| Bacteroides dorei_DSM17855 | Bdore.DSM17855 | Bacteroides | B. dore | DSMZ (Germany) | Anaerobic | Bacteroidetes/Bacteroidaceae/ | DSM17855 |
| | | dorei | | | | Bacteroides | |
| Bacteroides eggerthii_DSM20697 | Begge.DSM20697 | Bacteroides | B. egge | DSMZ (Germany) | Anaerobic | Bacteroidetes/Bacteroidaceae/ | DSM20697 |
| | | eggerthii | | | | Bacteroides | |
| Bacteroides finegoldii_DSM17565 | Bfine.DSM17565 | Bacteroides | B. fine | DSMZ (Germany) | Anaerobic | Bacteroidetes/Bacteroidaceae/ | DSM17565 |
| | | finegoldii | | | | Bacteroides | |
| Bacteroides fragilis_3_1_12 | Bfrag.3.1.12 | Bacteroides | B. frag | BEI | Anaerobic | Bacteroidetes/Bacteroidaceae/ | 3_1_12 |
| | | fragilis | | | | Bacteroides | |
| Bacteroides fragilis_CL03T00C08 | Bfrag.CL03T00C08 | Bacteroides | B. frag | BEI | Anaerobic | Bacteroidetes/Bacteroidaceae/ | CL03T00C08 |
| | | fragilis | | | | Bacteroides | |
| Bacteroides fragilis_NCTC9343 | Bfrag.NCTC9343 | Bacteroides | B. frag | ATCC | Anaerobic | Bacteroidetes/Bacteroidaceae/ | NCTC9343 |
| | | fragilis | | | | Bacteroides | |
| Bifidobacterium longum_AO44 | Blong.AO44 | Bifidobacterium | B. long | Clinical isolate, | Anaerobic | Actinobacteria/Bifidobacteriaceae/ | AO44 |
| | | longum | | BWH | | Bifidobacterium | |
| Bacteroides massiliensis_DSM17679 | Bmass.DSM17679 | Bacteroides | B. mass | DSMZ (Germany) | Anaerobic | Bacteroidetes/Bacteroidaceae/ | DSM17679 |
| | | massiliensis | | | | Bacteroides | |
| Bacteroides oleiciplenus_DSM22535 | Bolei.DSM22535 | Bacteroides | B. olei | DSMZ (Germany) | Anaerobic | Bacteroidetes/Bacteroidaceae/ | DSM22535 |
| | | oleiciplenus | | | | Bacteroides | |
| Bacteroides ovatus_ATCC8483 | Bovat.ATCC8483 | Bacteroides | B. ovat | BWH, Onderdonk | Anaerobic | Bacteroidetes/Bacteroidaceae/ | ATCC8483 |
| | | ovatus | | | | Bacteroides | |
| Bacteroides ovatus_CL02T12C04 | BovatCLO2T12C04 | Bacteroides | B. ovat | BEI | Anaerobic | Bacteroidetes/Bacteroidaceae/ | CL02T12C04 |
| | | ovatus | | | | Bacteroides | |
| Bacteroides salanitronis_DSM18170 | Bsala.DSM18170 | Bacteroides | B. sala | DSMZ (Germany) | Anaerobic | Bacteroidetes/Bacteroidaceae/ | DSM18170 |
| | | salanitronis | | | | Bacteroides | |
| Bacteroides thetaiotaomicron_ATCC29148 | Bthet.ATCC29148 | Bacteroides | B. thet | ATCC | Anaerobic | Bacteroidetes/Bacteroidaceae/ | ATCC29148 |
| | | thetaiotaomicron | | | | Bacteroides | |
| Bacteroides thetaiotaomicron_ATCC29741 | Bthet.ATCC29741 | Bacteroides | B. thet | BWH clinical, | Anaerobic | Bacteroidetes/Bacteroidaceae/ | ATCC29741 |
| | | thetaiotaomicron | | Onderdonk | | Bacteroides | |
| Bacteroides uniformis_ATCC8492 | Bunif.ATCC8492 | Bacteroides | B. unif | ATC | Anaerobic | Bacteroidetes/Bacteroidaceae/ | ATCC8492 |
| | | uniformis | | | | Bacteroides | |
| Bacteroides vulgatus_ATCC8482 | Bvulg.ATCC8482 | Bacteroides | B. vulg | BWH clinical, | Anaerobic | Bacteroidetes/Bacteroidaceae/ | ATCC8482 |
| | | vulgatus | | Onderdonk | | Bacteroides | |
| Collinsella aerofaciens_VPI1003 | Caero.VPI1003 | Collinsella | C. aero | ATCC | Anaerobic | Actinobacteria/Coriobacteriaceae/ | VPI1003 |
| | | aerofaciens | | | | Collinsella | |
| Clostridium histolyticum_AO25 | Chist.AO25 | Clostridium | C. hist | BWH clinical, | Anaerobic | Firmicutes/Clostridiaceae/ | AO25 |
| | | histolyticum | | Onderdonk | | Clostridium | |
| Campylobacter jejuni_AS-84-79 | Cjeju.AS-84-79 | Campylobacter | C. jeju | BEI | Anaerobic | Proteobacteria/Campylobacteraceae/ | AS-84-79 |
| | | jejuni | | | | Campylobacter | |
| Coprobacillus_8_2_54BFAA | Copr.8.2.54BFAA | Coprobacillus | Copr | BEI | Anaerobic | Firmicutes/Erysipelotrichaceae/ | 8_2_54BFAA |
| | | | | | | Coprobacillus | |
| Clostridium perfringens_ATCC13124 | Cperf.ATCC13124 | Clostridium | C. perf | BWH clinical, | Anaerobic | Firmicutes/Clostridiaceae/ | ATCC13124 |
| | | perfringens | | Onderdonk | | Clostridium | |
| Clostridium ramosum_AO31 | Cramo.AO31 | Clostridia | C. ramo | BWH clinical, | Anaerobic | Firmicutes/Clostridiaceae/ | AO31 |
| | | ramosum | | Onderdonk | | Clostridium | |
| Clostridium sordellii_AO32 | Csord.AO32 | Clostridium | C. sord | Clinical | Anaerobic | Firmicutes/Clostridiaceae/ | AO32 |
| | | sordellii | | isolate, BWH | | Clostridium | |
| Escherichia coli_Nissle1917 | Ecoli.Nissle1917 | Escherichia | E. coli | Mekelanos | Aerobic | Proteobacteria/Enterobacteriaceae/ | Nissle1917 |
| | | coli | | lab | | Escherichia | |
| Enterococcus faecalis_HH22 | Efaec.HH22 | Enterococcus | E. faec | BEI | Anaerobic | Firmicutes/Enterococcaceae/ | HH22 |
| | | faecalis | | | | Enterococcus | |
| Enterococcus faecalis_OG1RF | Efaec.OG1RF + GFP | Enterococcus | E. faec | ATCC | Anaerobic | Firmicutes/Enterococcaceae/ | OG1RF + GFP |
| | | faecalis | | | | Enterococcus | |
| Enterococcus faecalis_TX0104 | Efaec.TX0104 | Enterococcus | E. faec | BEI | Anaerobic | Firmicutes/Enterococcaceae/ | TX0104 |
| | | faecalis | | | | Enterococcus | |
| Enterococcus faecium_TX1330 | Efaec.TX1330 | Enterococcus | E. faeci | BEI | Anaerobic | Firmicutes/Enterococcaceae/ | TX1330 |
| | | faecium | | | | Enterococcus | |
| Eubacterium lentum_AO28 | Elent.AO28 | Eggerthella | E. lent | BWH clinical, | Anaerobic | Actinobacteria/Coriobacteriaceae/ | AO28 |
| | | lenta | | Onderdonk | | Eggerthella | |
| Eubacterium rectale_ATCC33656 | Erect.ATCC33656 | Eubacterium | E. rect | ATCC | Anaerobic | Firmicutes/Eubacteriaceae/ | ATCC33656 |
| | | rectale | | | | Eubacterium | |
| Fusobacterium varium_AO16 | Fvari.AO16 | Fusobacterium | F. vari | BWH clinical, | Anaerobic | Firmicutes/Eubacteriaceae/ | AO16 |
| | | varium | | Onderdonk | | Eubacterium | |
| Fusobacterium nucleatum_F0419 | Fnucl.F0419 | Fusobacterium | F. nucl | BEI | Anaerobic | Firmicutes/Eubacteriaceae/ | F0419 |
| | | nucleatum | | | | Eubacterium | |
| Helicobacter pylori_ ATCC700392 | Hpylo.ATCC700392 | Helicobacter | H. pylo | ATCC | | Proteobacteria/Helicobacteraceae/ | ATCC700392 |
| | | pylori | | | | Helicobacter | |
| Klebsiella_sp_4_1_44FAA | Kleb.sp.4.1.44FAA | Klebsiella | Kleb | BEI | Anaerobic | Proteobacteria/Enterobacteriaceae/ | sp. 4_1_44FAA |
| | | | | | | Klebsiella | |
| Lachnospiraceae_sp_2_1_58FAA | Lach.2.1.58FAA | Lachnospiraceae sp. | Lach | BEI | Anaerobic | Firmicutes/Lachnospiraceae/ | 2_1_58FAA |
| | | | | | | Lachnospira | |
| Lactobacillus casei_AO47 | Lcase.AO47 | Lactobacillus | L. case | BWH clinical, | Anaerobic | Firmicutes/Lactobacillaceae/ | AO47 |
| | | casei | | Onderdonk | | Lactobacillus | |
| Lactobacillus johnsonii_AO12 | Ljohn.AO12 | Lactobacillus | L. john | BWH clinical, | Anaerobic | Firmicutes/Lactobacillaceae/ | AO12 |
| | | johnsonii | | Onderdonk | | Lactobacillus | |
| Lactobacillus rhamnosus_LMS2-1 | Lrham.LMS2-1 | Lactobacillus | L. rham | BEI | Anaerobic | Firmicutes/Lactobacillaceae/ | LMS2-1 |
| | | rhamnosus | | | | Lactobacillus | |
| Neisseria flavescens_SKI14 | Nflav.SKl14 | Neisseria | N. flav | BEI | Aerobic | Proteobacteria/Neisseriaceae/ | SK114 |
| | | flavescens | | | | Neisseria | |
| Peptostreptococus asaccharolyticus_AO33 | Pasac.AO33 | Peptostreptococus | P. asac | BWH clinical, | Anaerobic | Firmicutes/Peptoniphilaceae/ | AO33 |
| | | asaccharolyticus | | Onderdonk | | Peptoniphilus | |
| Parabacteroides distasonis_ATCC8503 | Pdist.ATCC8503 | Parabacteroides | P. dist | BWH clinical, | Anaerobic | Bacteroidetes/Porphyromonadaceae/ | ATCC8503 |
| | | distasonis | | Onderdonk | | Parabacteroides | |
| Porphyromonas gingivalis_W83 | Pging.W83 | Porphyromonas | P. ging | ATCC | Anaerobic | Bacteroidetes/Porphyromonadaceae/ | W83 |
| | | gingivalis | | | | Porphyromonas | |
| Propionibacterium granulosum_AO42 | Pgran.AO4 | Propionibacterium | P. gran | BWH clinical, | Anaerobic | Actinobacteria/Propionibacteriaceae/ | AO42 |
| | | granulosum | | Onderdonk | | Propionibacterium | |
| Prevotellae intermedia_AO10 | Pinte.AO10 | Prevotellae | P. inte | BWH clinical, | Anaerobic | Bacteroidetes/Prevotellaceae/ | AO10 |
| | | intermedia | | Onderdonk | | Prevotella | |
| Peptostreptococcus magnus_AO29 | Pmagn.AO29 | Peptostreptococcus | P. magn | BWH clinical, | Anaerobic | Firmicutes/Clostridiaceae/ | AO29 |
| | | magnus | | Onderdonk | | Peptostreptococcus | |
| Prevotellae melaninogenica_ATCC25845 | Pmela.ATCC25845 | Prevotellae | P. mela | BWH clinical, | Anaerobic | Bacteroidetes/Prevotellaceae/ | ATCC25845 |
| | | melaninogenica | | Onderdonk | | Prevotella | |
| Parabacteroides merdae_CL03T12C32 | Pmerd.CL03T12C32 | Parabacteroides | P. merd | BEI | Anaerobic | Bacteroidetes/Porphyromonadaceae/ | CL03T12C32 |
| | | merdae | | | | Parabacteroides | |
| Parabacteroides merdae_CL09T00C40 | Pmerd.CL09T00C40 | Parabacteroides | P. merd | BEI | Anaerobic | Bacteroidetes/Porphyromonadaceae/ | CL09T00C40 |
| | | merdae | | | | Parabacteroides | |
| Porphyromonas uenonis_UPII60-3 | Pueno.UPII60-3 | Porphyromonas | P. ueno | BEI | Anaerobic | Bacteroidetes/Porphyromonadaceae/ | UPII60-3 |
| | | uenonis | | | | Porphyromonas | |
| Ruminococcus gnavus_ATCC29149 | Rgnav.ATCC29149 | Ruminococcus | R. gnav | ATCC | Anaerobic | Firmicutes/Lachnospiraceae/ | ATCC29149 |
| | | gnavus | | | | Blautia | |
| SFB | SFB | Candidatus | SFB | Kasper lab | Anaerobic | Firmicutes/Clostridiaceae/ | |
| | | Arthromitus | | | | Candidatus | |
| Streptococcus mitis_F0392 | Smiti.F0392 | Streptococcus | S. miti | BEI | Anaerobic | Firmicutes/Streptococcaceae/ | F0392 |
| | | mitis | | | | Streptococcus | |
| Staphylococcus saprophyticus_ATCC15305 | Ssapr.ATCC15305 | Staphylococcus | S. sapr | ATCC | Aerobic | Firmicutes/Staphylococcaceae/ | ATCC 15305 |
| | | saprophyticus | | | | Staphylococcus | |
| Staphylococcus saprophyticus_DLK1 | Ssapr.DLK1 | Staphylococcus | S. sapr | BWH clinical, | Aerobic | Firmicutes/Staphylococcaceae/ | DLK1 |
| | | saprophyticus | | Onderdonk | | Staphylococcus | |
| Veillonella_6_1_27 | Veil.6.1.27 | Veillonella | Veil | BEI | Anaerobic | Firmicutes/Veillonellaceae/ | 6_1_27 |
| | | | | | | Veillonella | |
| Parabacteroides johnsonii_CL02T12C29 | Pjohn.CL02T12C29 | Parabacteroides | P. john | BEI | Anaerobic | Bacteroidetes/Porphyromonadaceae/ | CL02T12C29 |
| | | johnsonii | | | | Parabacteroides | |

(1) All strains from this study are available from BEI/ATCC/DSMZ.; (2) Non-repository strains have now been deposited at BEI.

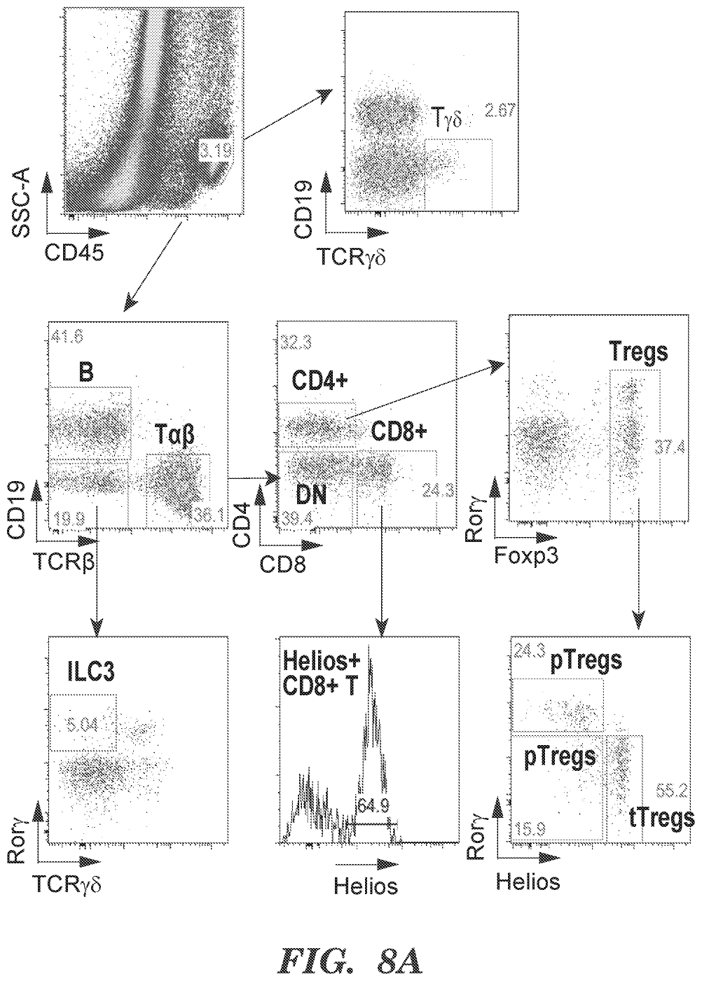

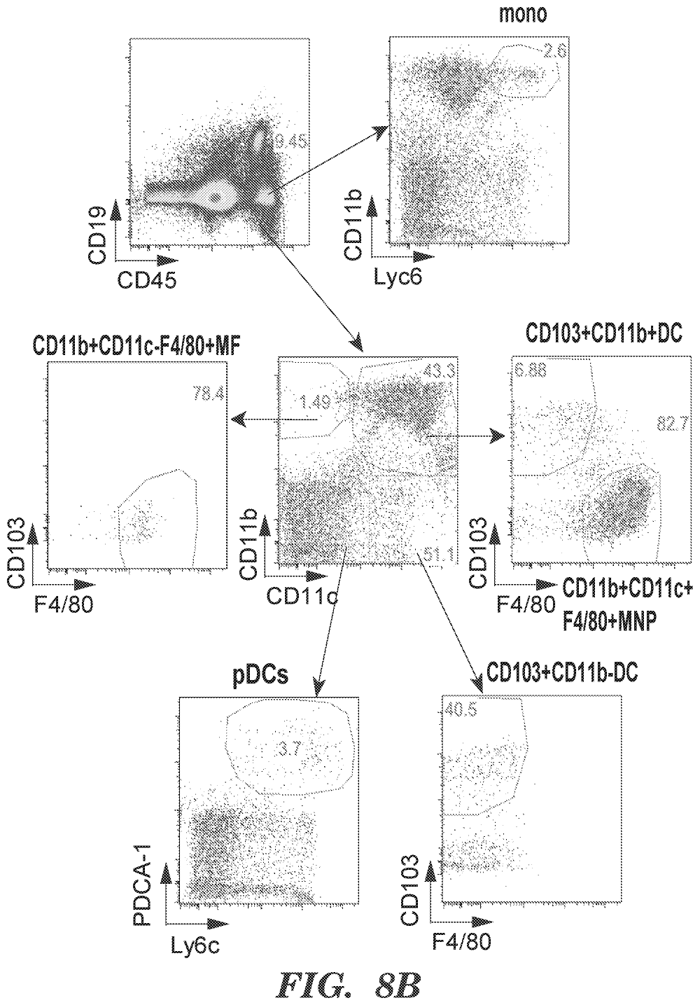

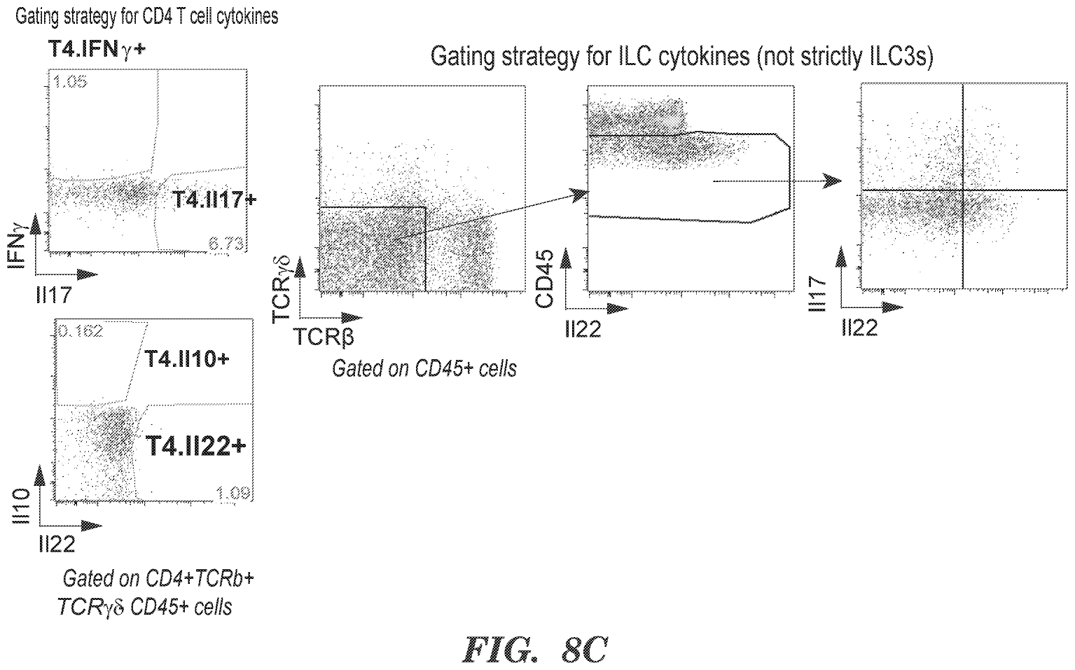

Both local and systemic effects on the immune system were examined by analyzing the proportions of 18 cell types from its innate and adaptive arms (FIG. 1B, FIG. 8, and Table 2 for all cell types, gating strategy, and phenotypic markers, respectively). Five intestinal and lymphoid tissues were examined: SI and colonic lamina propria, Peyer's patches, mesenteric lymph node (mLNs) and systemic lymphoid organs (SLO; pooled spleen and subcutaneous lymph nodes). CD4+ T-cell production of the cytokines IL10, IL17a, IL22, and IFNγ, and ILC production of IL22 were also measured. Cell specifications of cell types, their markers, and gating strategies are depicted in Table 2.

TABLE 2
Cell Specifications

| Cell Name (in figs) | Full Cell Name | Gating | Reported as % of |
|---|---|---|---|
| mono | monocytes | Ly6c+CD11b+CD45+ | CD45+CD19− |
| | | cd19− | |
| CD11b+CD11c− | CD11b+CD11C−F4/80+ | F4/80+CD103−CD11b+ | D45+CD19− |
| F4/80+MF | macrophages | CD11c−CD45+CD19− | |
| CD11b+CD11c+ | CD11b+CD11c+F4/80+ | F4/80+CD103−CD11b+ | CD45+CD19− |
| F4/80+MNP | mononuclear phagocytes | CD11c+CD45+CD19− | |
| CD103+CD11b+DC | CD103+CD11b+ | CD103+F4/80−CD11b+ | CD45+CD19− |
| | dendritic cells | CD11c+CD45+CD19 | |
| CD103+CD11b−DC | CD103+CD11b− | CD103+F4/80−CD11b− | CD45+CD19− |
| | dendritic cells | CD11c+CD45+CD19− | |
| pDC | plasmacytoid | PDCA1+Lyc6+CD11b− | CD11b−CD45+CD19− |
| | dendritic cells | CD45+cd19− | |
| ILC3 | Innate lymphocytes | Rorg+CD45+TCRb− | CD45+ |
| | type (ILC) 3 | CD19−TCRgd− | |
| B | B cells | CD19+CD45+TCRb− | CD45+ |
| Tgd | TCRab T cells | TCRb+CD45+CD19− | CD45+ |
| Tab | TCRgd T cells | TCRgd+CD45+CD19− | CD45+ |
| DN(CD8−CD4− | CD4−CD8− | CD4−CD8a−TCRb+ | TCRb+CD45+CD19− |
| TCR+) | T cells | CD19−CD45+ | |
| T8 | CD8+ T cells | CD8a+CD4−TCRb+ | TCRb+CD45+CD19− |
| | | CD19−CD45+ | |
| T8.Helios+ | Helios+ CD8+ | Helios+CD8a+CD4− | CD8a+CD4−TCRb+ |
| | T cells | TCRb+CD19−CD45+ | CD19−CD45+ |
| T4 | CD4+ T cells | CD4+CD8a−TCRb+ | TCRb+CD45+CD19− |
| | | CD19−CD45+ | |
| T4.FP-Rorg+ | Rorg+ conventional | Rorg+Foxp3−CD4+CD8a− | CD4+CD8a−TCRb+ |
| | T cells | TCRb+CD19−CD45+ | CD19−CD45+ |
| T4.FP+ | Foxp3+ regulatory | Foxp3+CD4+CD8a− | CD4+CD8a−TCRb+ |
| | T cells (Tregs) | TCRb+CD19−CD45+ | CD19−CD45+ |
| T4.FP+Helios− | peripheral Tregs | Helios−Foxp3+CD4+CD8a− | Foxp3+CD4+CD8a− |
| | | TCRb+CD19−CD45+ | TCRb+CD19−CD45+ |
| T4.FP+Rorg+ | Rorg+ peripheral | Rorg+Helios−Foxp3+CD4+ | Foxp3+CD4+CD8a− |
| Helios− | Tregs | CD8a−TCRb+CD19−CD45+ | TCRb+CD19−CD45+ |
| Cytokines | | | |
| T4.ifng+ | IFNg producing CD4+ | IFNg+CD4+TCRb+ | CD4+TCRb+TCRgd− |
| | T cells (TH1) | TCRgd−D45+ | CD45+ |
| T4.il10+ | IL10 producing CD4+ | IL10+CD4+TCRb+ | CD4+TCRb+TCRgd− |
| | T cells | TCRgd−CD45+ | CD45+ |
| T4.il17+ | IL17 producing CD4+ | IL17+CD4+TCRb+ | CD4+TCRb+TCRgd− |
| | T cells (TH17) | TCRgd−CD45+ | CD45+ |
| T4.il22+ | IL22 producing CD4+ | IL22+CD4+TCRb+ | CD4+TCRb+TCRgd− |
| | T cells | TCRgd−CD45+ | CD45+ |
| ILC.il22+ | IL22 producing ILCs | IL22+TCRb−TCRgd− | TCRb−TCRgd−CD45dim |
| | | CD45dim | |

### Microbial Selection and Colonization

Fifty-three bacterial species were selected from the Human Microbiome Project database to represent the spectrum of phyla and genera in the human gut microbiota (FIG. 1C) and covering the 5 dominant phyla: Bacteroidetes, Firmicutes, Proteobacteria, Actinobacteria, and Fusobacteria (FIG. 1C and Table 1). The selection of strains aimed to encompass genetic and phenotypic diversity rather than reflecting actual frequencies in the human intestines.

Effective gastrointestinal colonization was assessed by culture of fecal material harvested from the colon and, in some cases, from the stomach and oral cavity. Most of the strains introduced orally into GF mice successfully colonized the intestines of the recipients (10⁸ to 10¹⁰ CFU/g; FIG. 1D and data not shown—see supplemental materials of Geva-Zatorsky et al., Cell 2017, incorporated by reference herein below). Of the seven species not recovered in fecal specimens, five were recovered at other sites. Porphyromonas gingivalis, Prevotella intermedia, and Prevotella melaninogenica were found only in the oral cavity, while Helicobacter pylori and Lactobacillus johnsonii resided exclusively in the stomach. Interestingly, these are the anatomic sites in which these species are normally found in mice and humans with a complex microbiota. This existence of niche preferences even in the absence of microbial competition suggests that they derive from organ-specific physical and/or chemical properties that are intrinsically unfavorable for a certain microbe, such as acidity or the availability of particular nutrient types, rather than from competitive fitness. Only two bacteria failed to colonize any site (Eubacterium lentum and Eubacterium rectale). Colony-forming units in feces (per gram) in the mLN and SLO for all microbes in this study were assessed (data not shown—see supplemental materials of Geva-Zatorsky et al., Cell 2017, incorporated by reference herein below).

Commensal bacteria can breach intestinal barriers and can be found in small numbers in gut-draining lymph nodes or systemically. This microbial delocalization is facilitated by deficiencies in innate defenses and by myeloid cells that actively transport the bacteria, plausibly to enable antigen presentation. Because the ability of various symbionts to partake in extraintestinal delocalization is unknown, this screen was used to investigate the ability of the bacteria studied to delocalize to mLNs and caudal lymph nodes (cLNs), which drain the SI and the colon, respectively, and to the SLO. Strict precautions were taken during dissection to avoid contamination from the gut. A majority (88%) of the species that colonized the gut were detected alive in mLNs (FIG. 1E, top), with no particular preference according to phylum, genus, or aerobe/anaerobe status. A substantial proportion (47%) of gut-colonizing microbes were also found alive in the SLO (FIG. 1E, bottom).

Immunologic Changes in Response to Monocolonization with Human Gut Symbionts

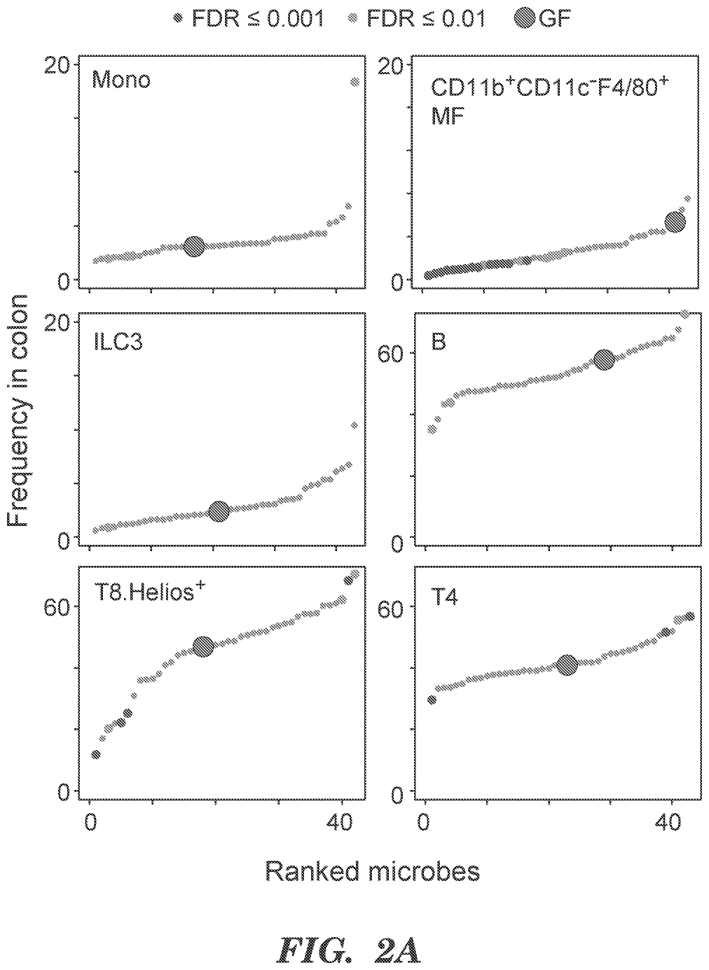

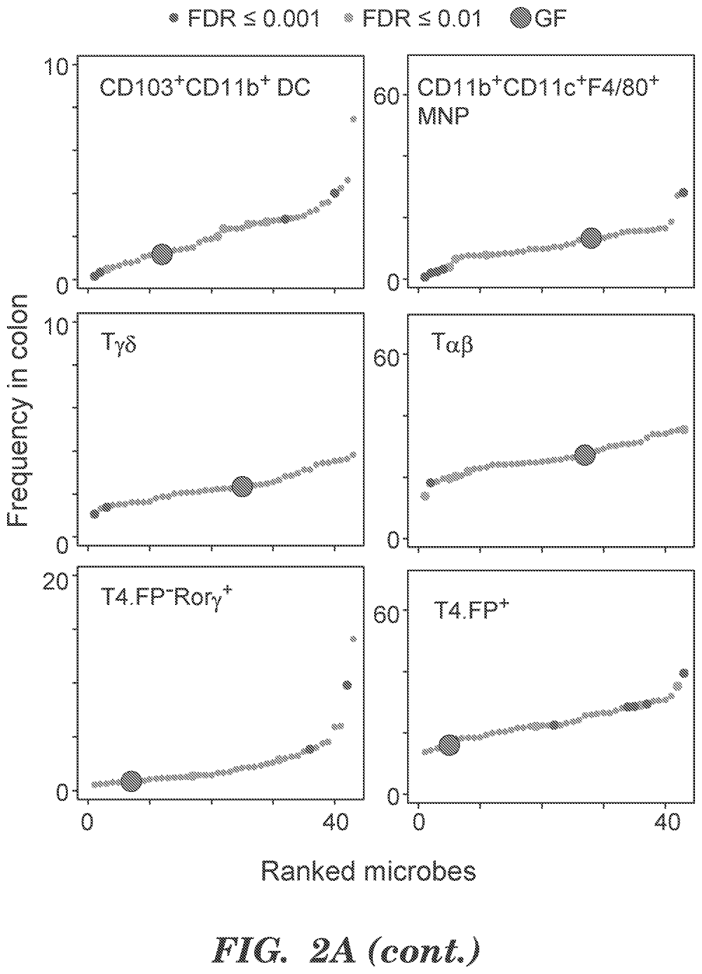

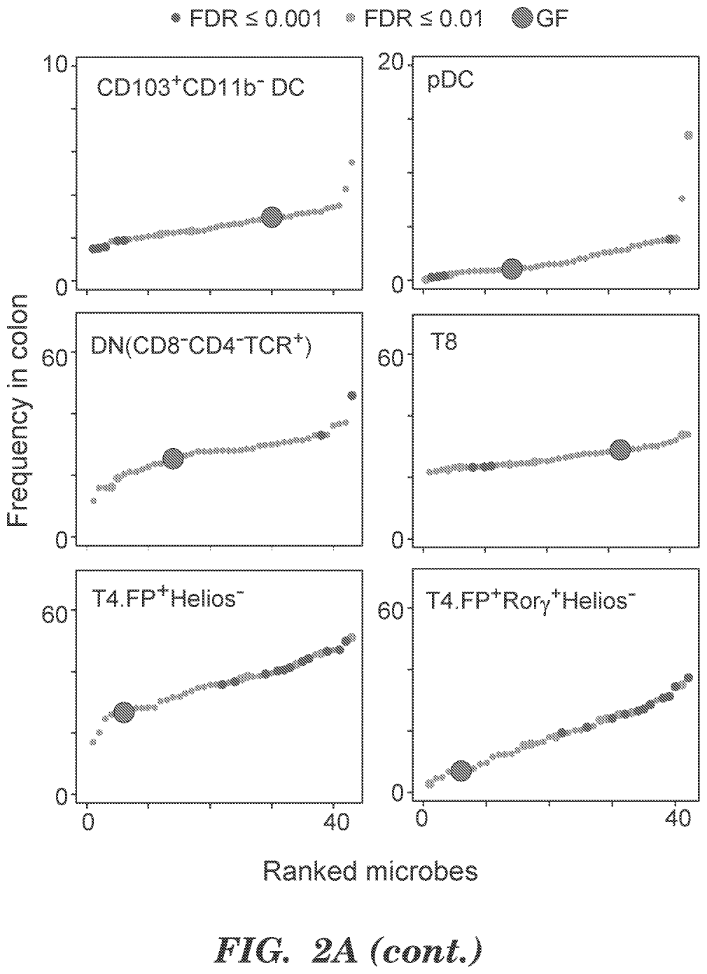

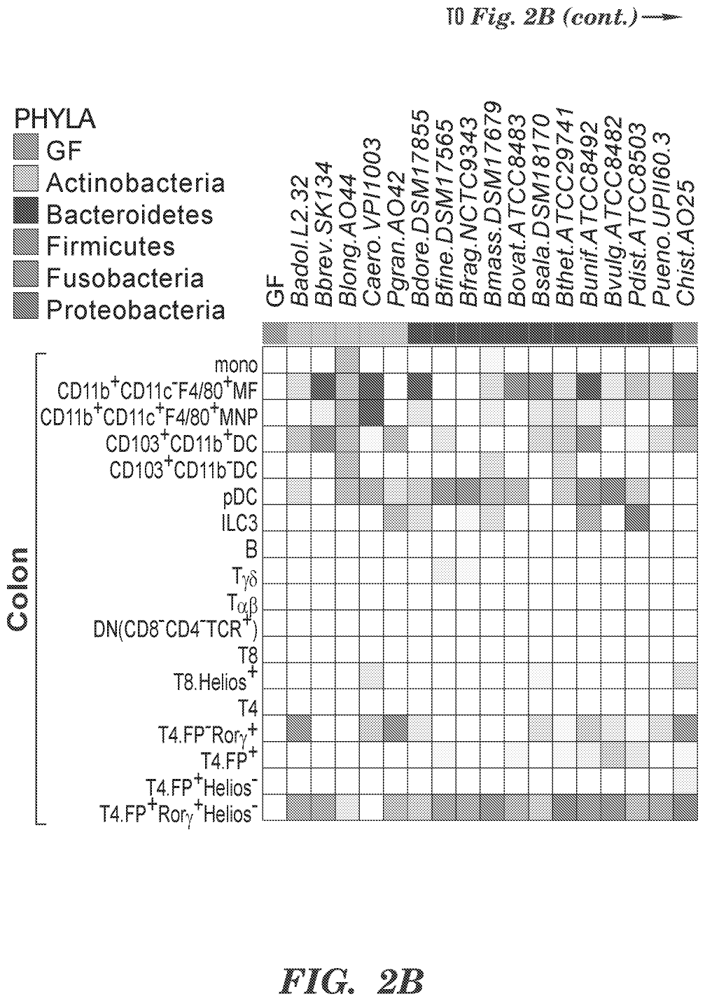

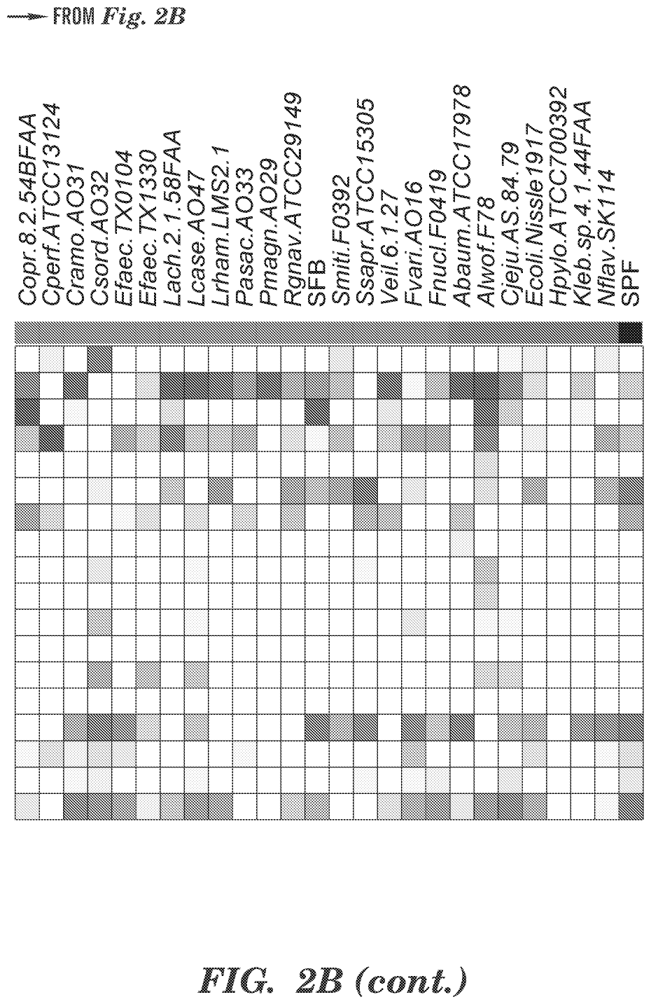

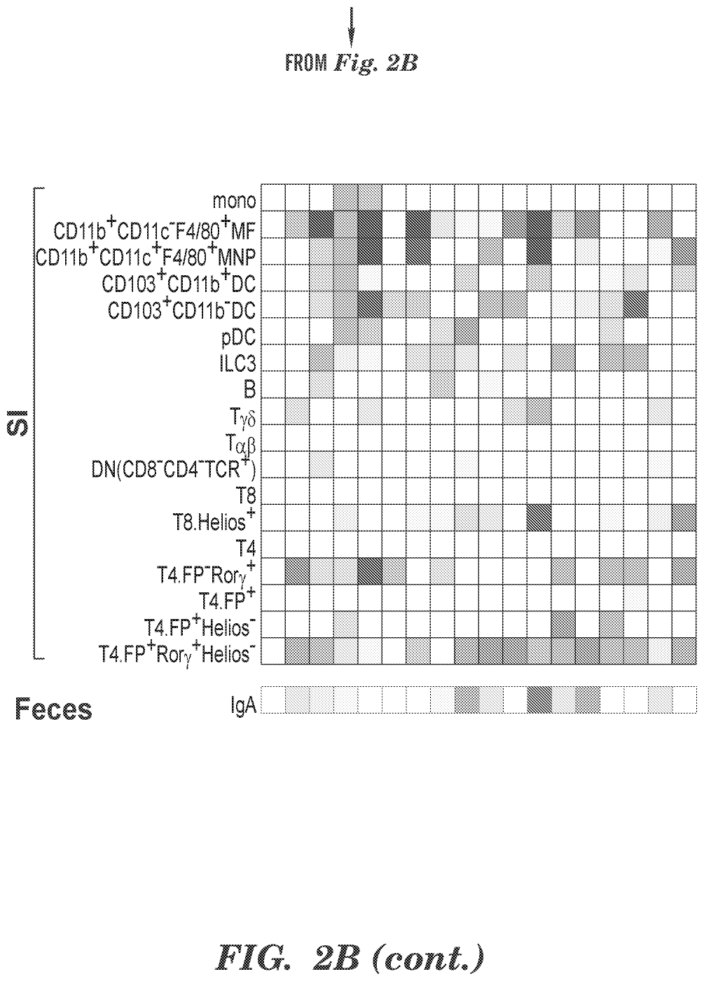

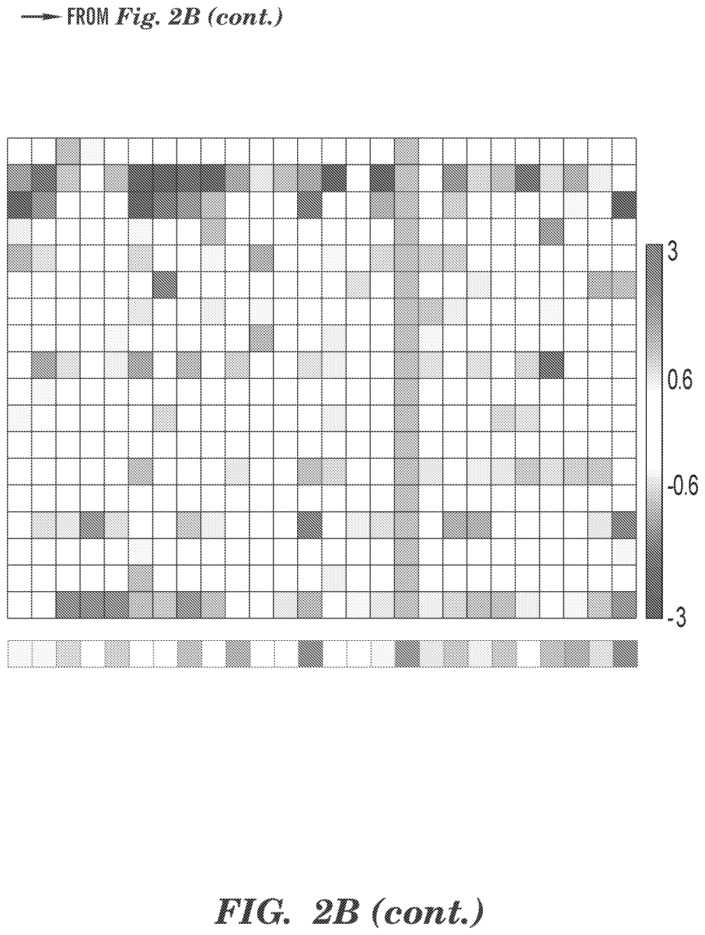

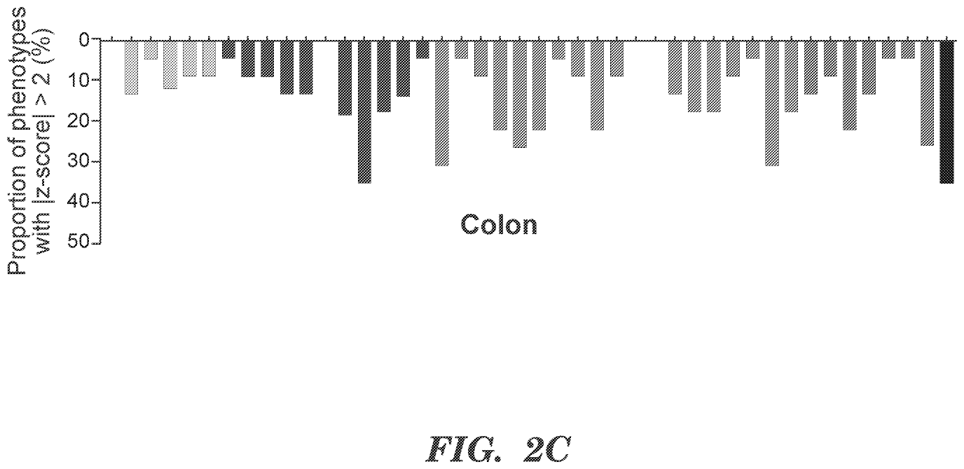

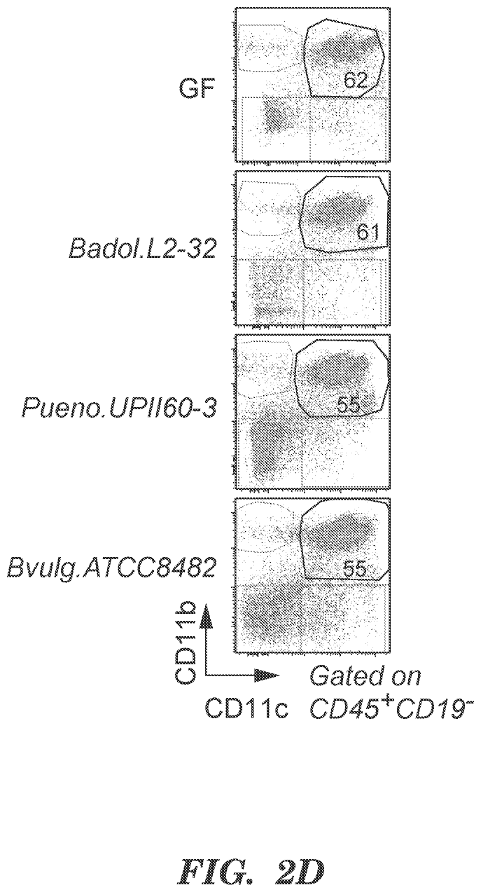

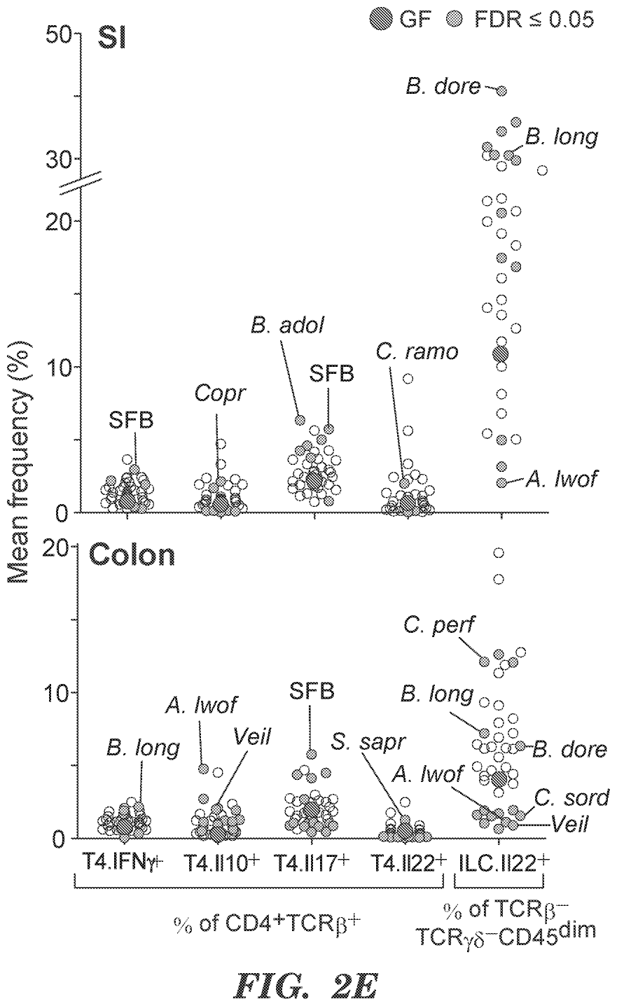

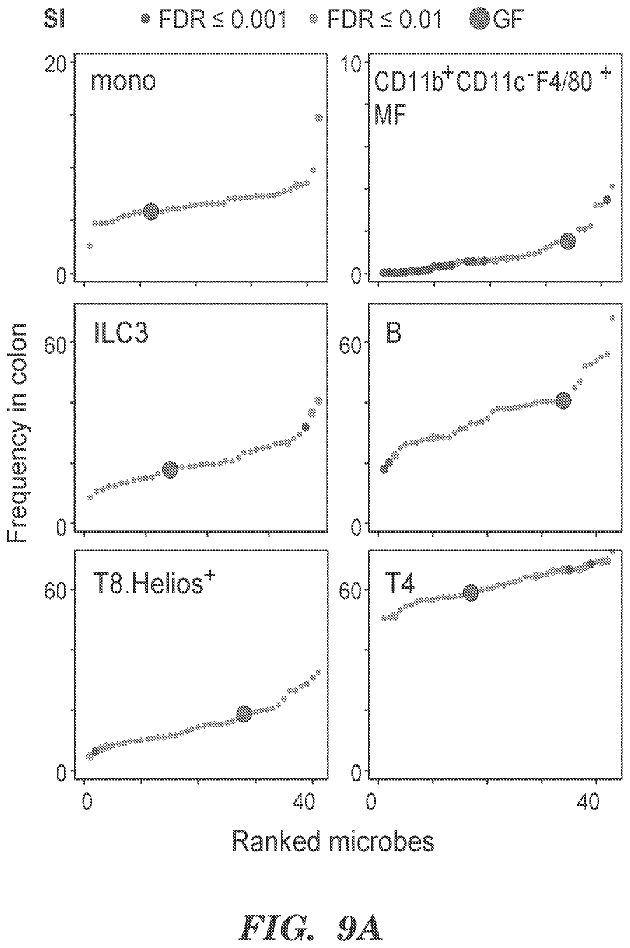

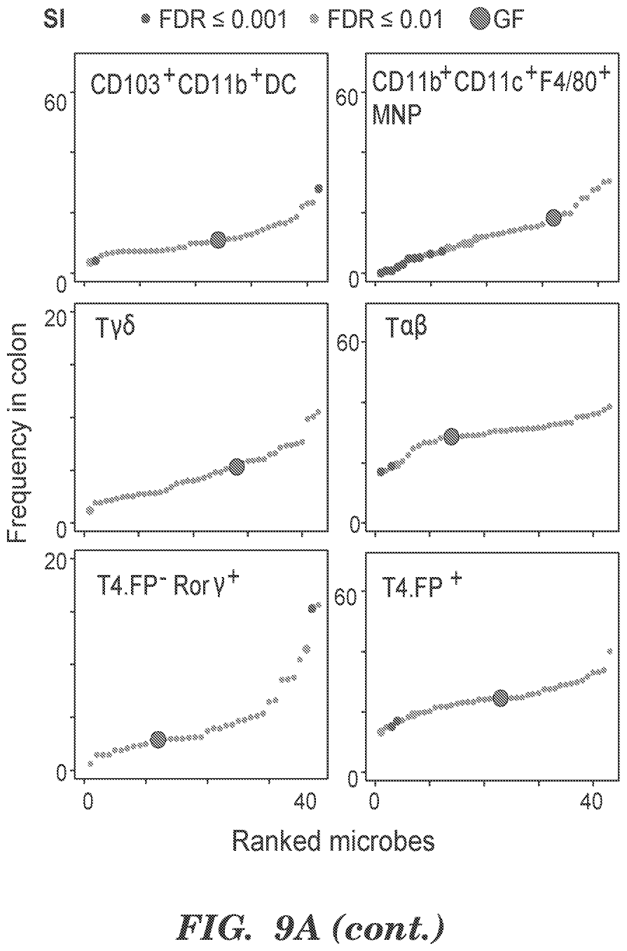

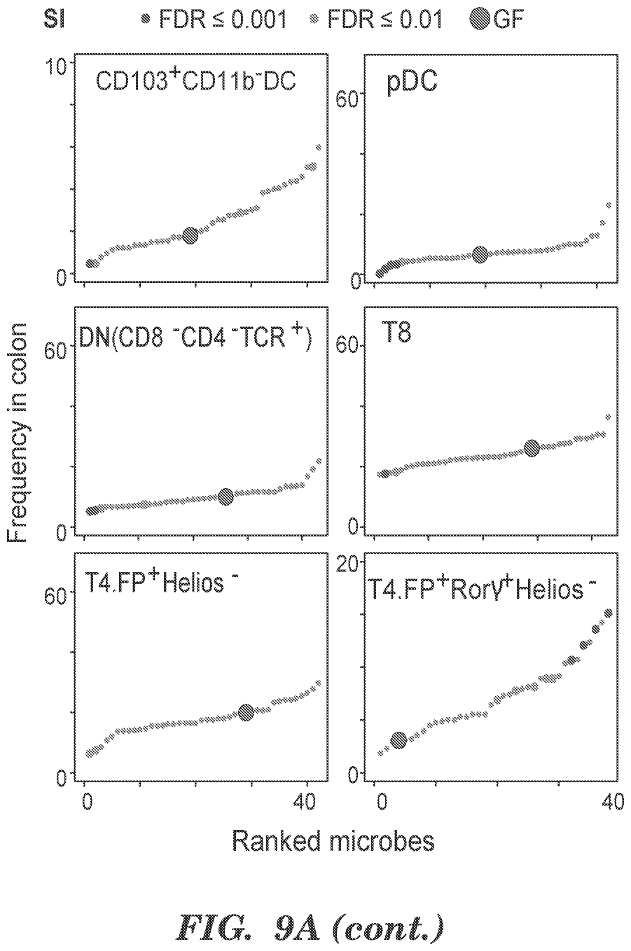

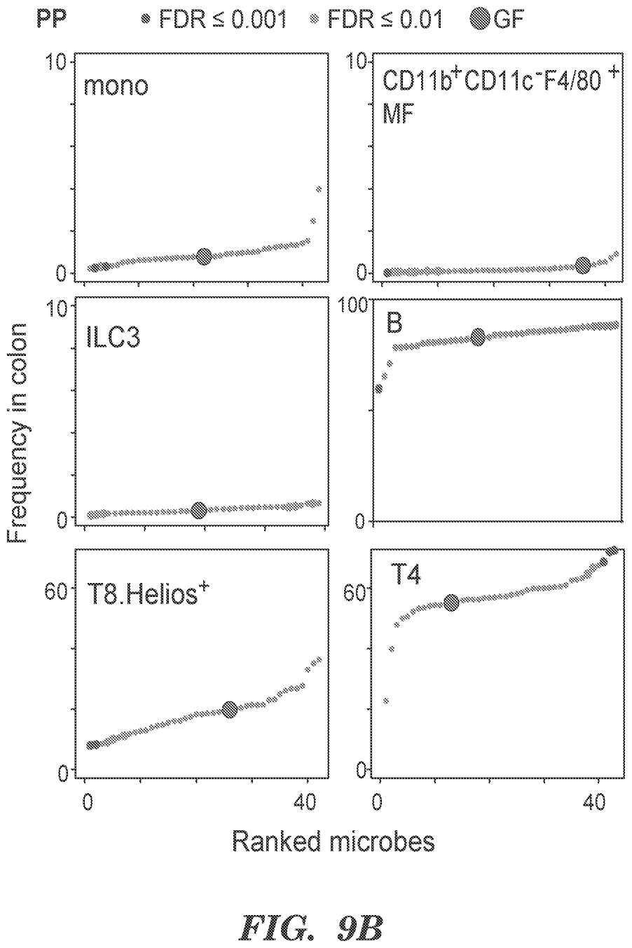

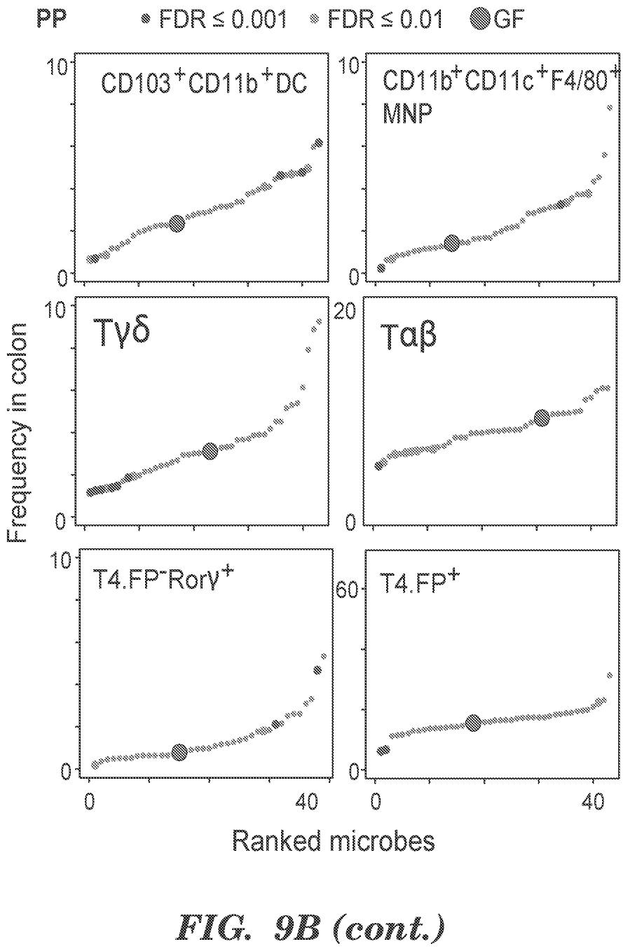

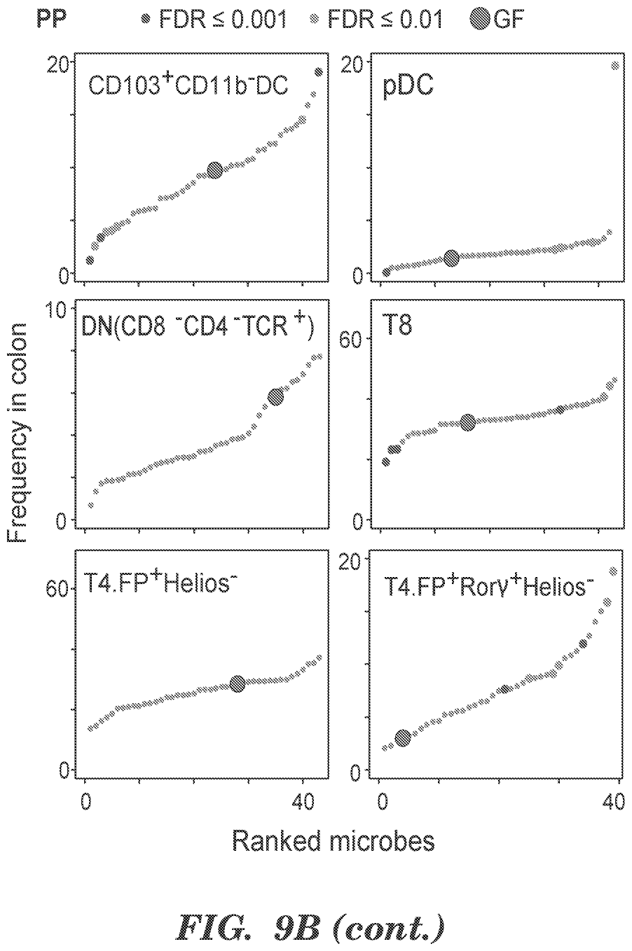

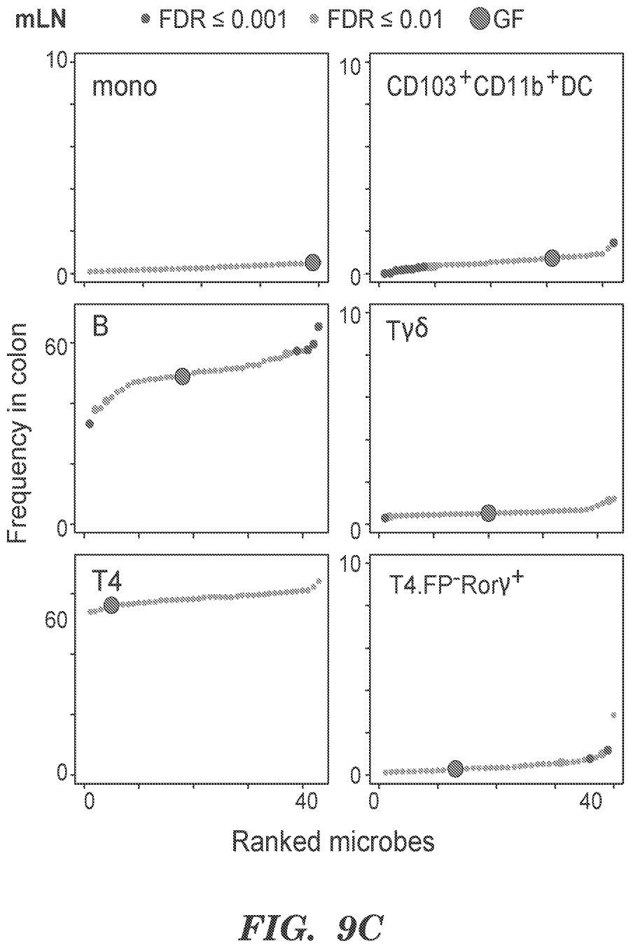

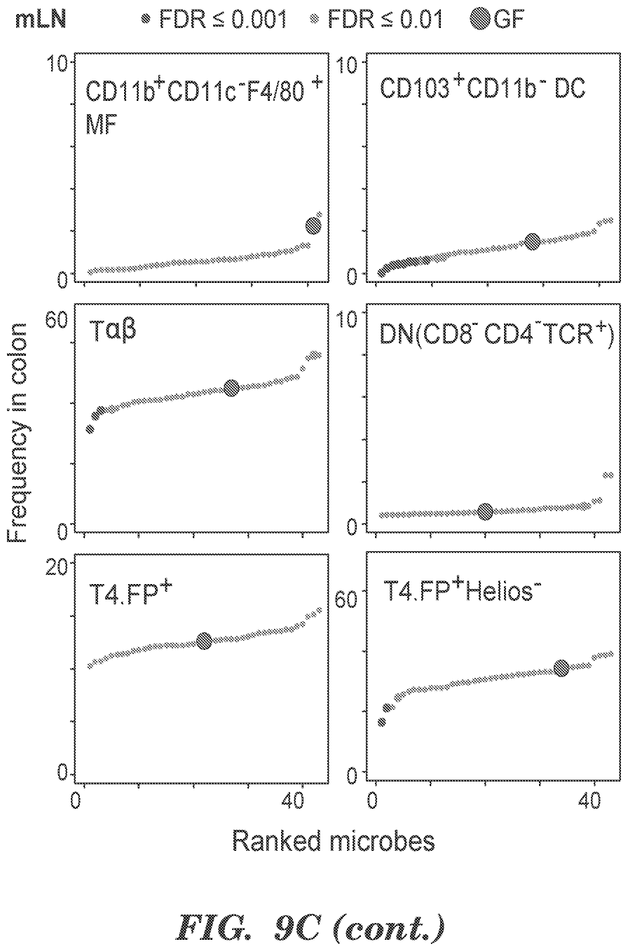

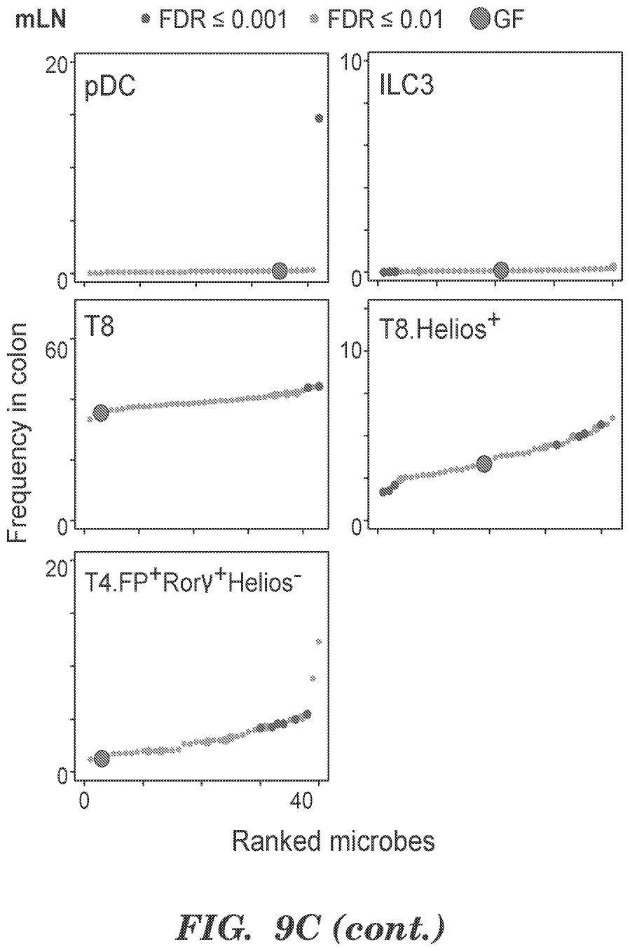

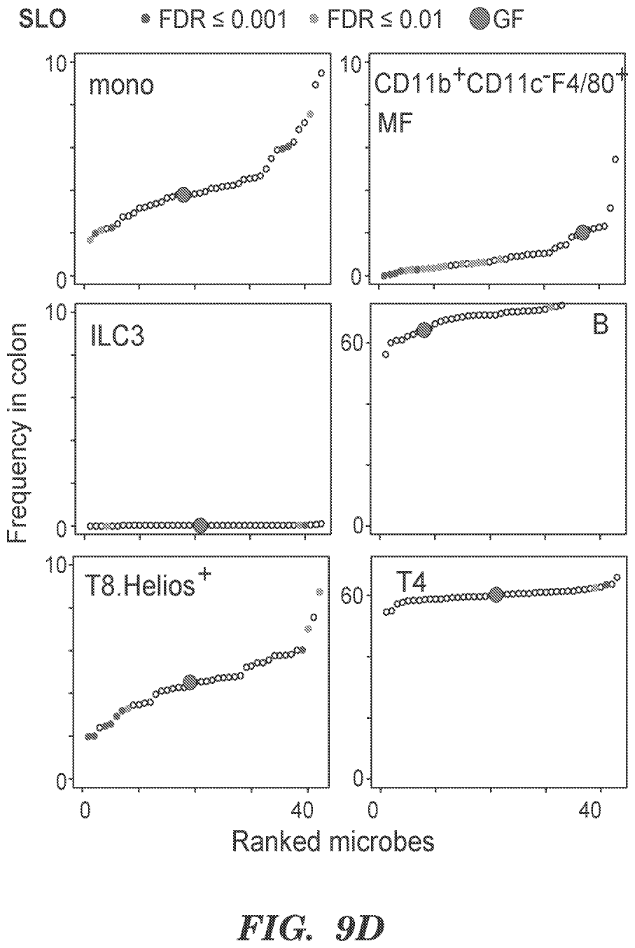

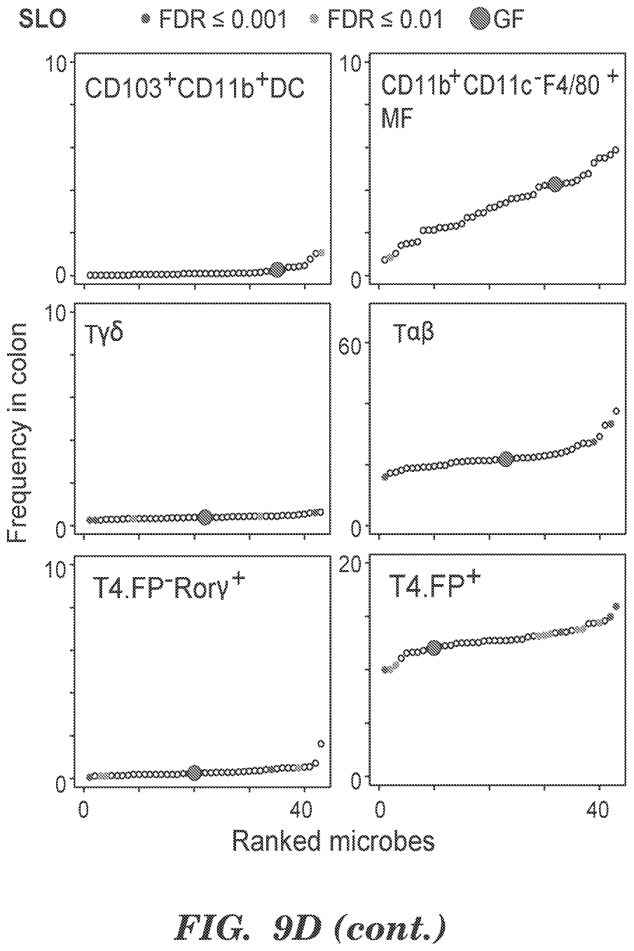

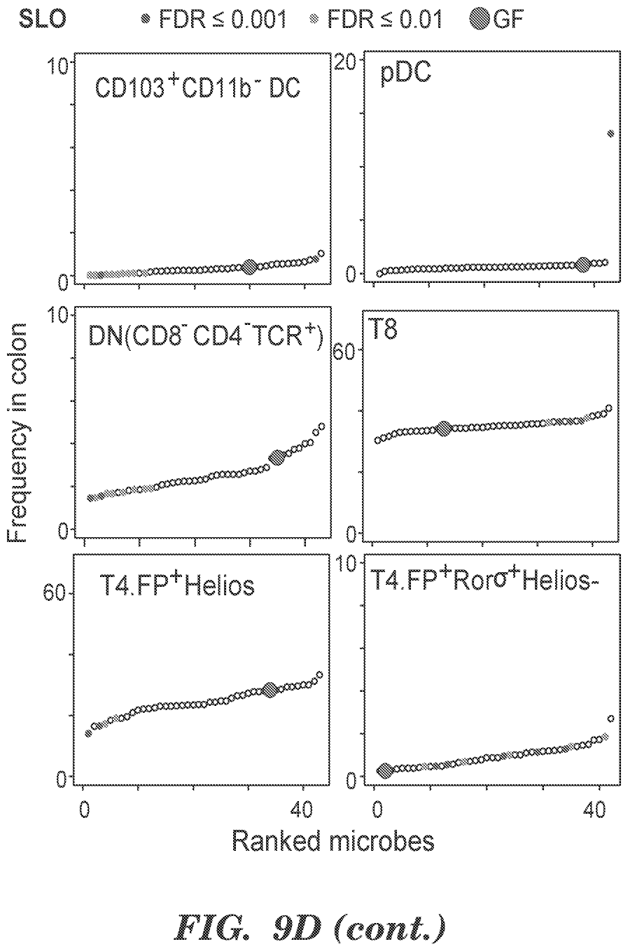

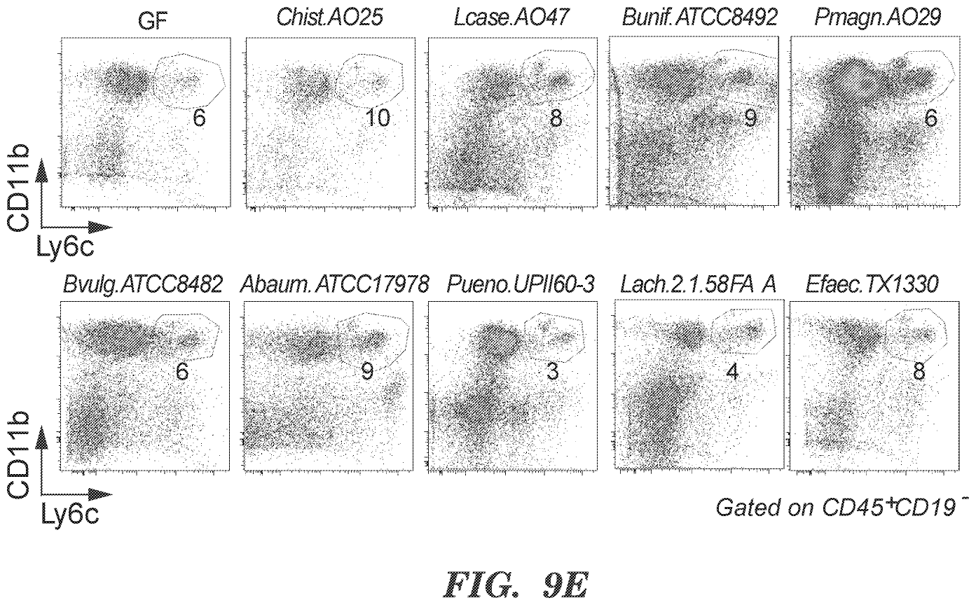

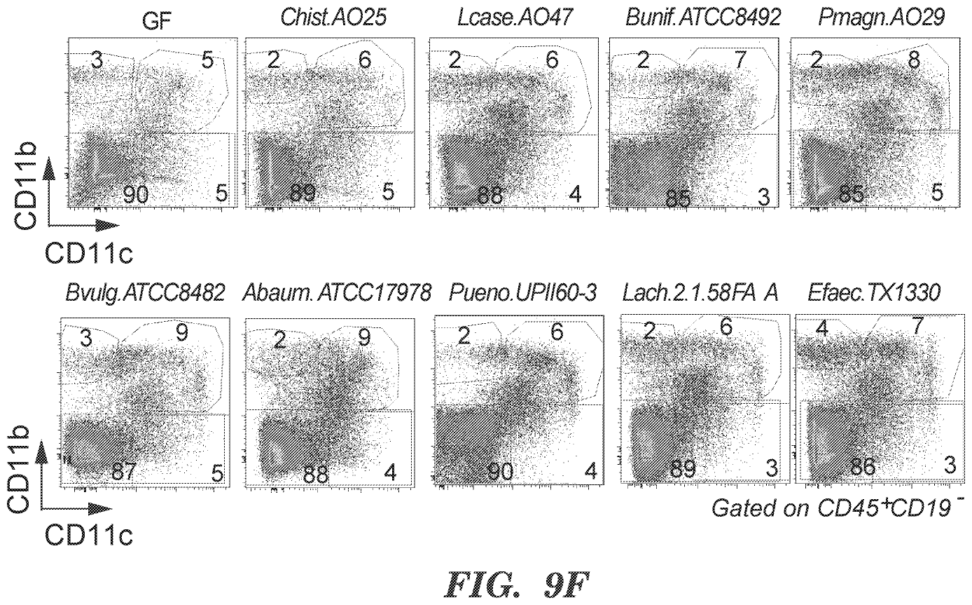

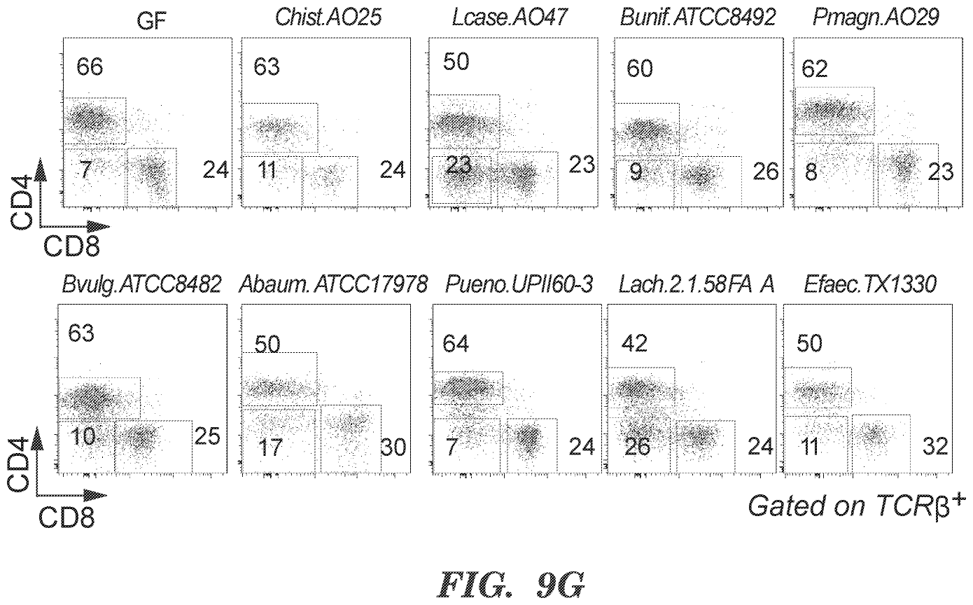

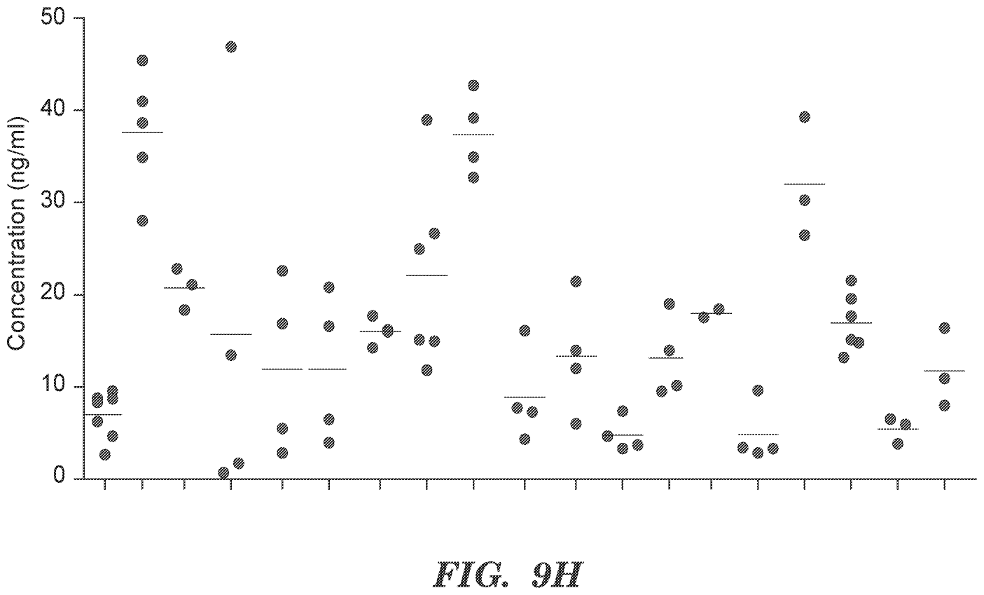

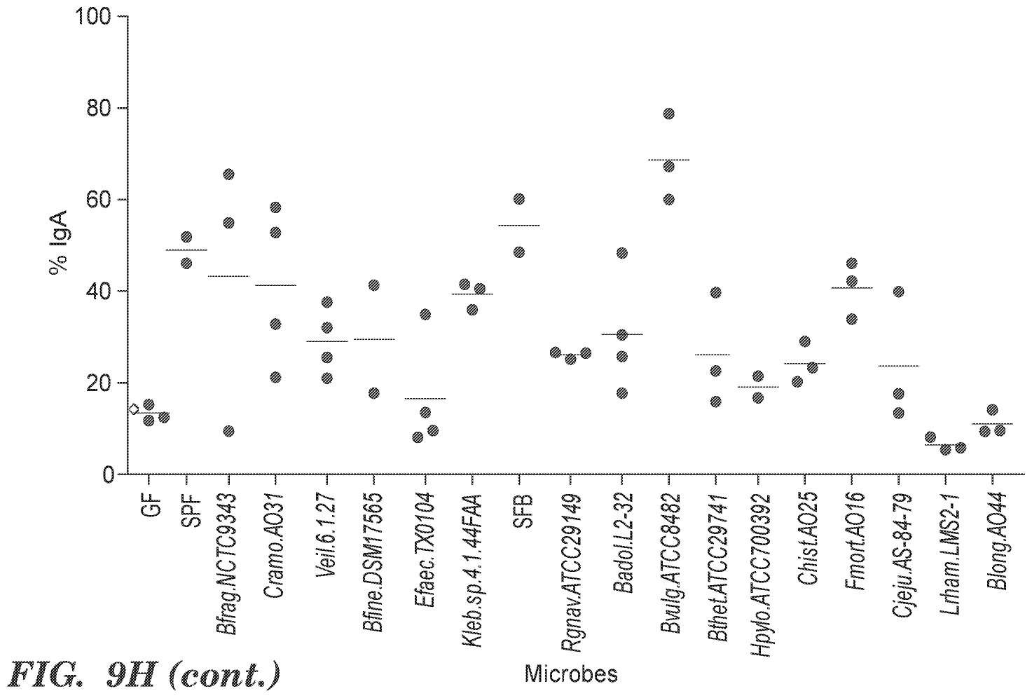

The broad screen described above generated 24, 255 individual immunophenotypes induced in local or systemic lymphoid organs by the bacteria that successfully monocolonized GF mice and for which complete data were obtained. FIG. 2A and Tables 3A-G illustrate the changes in frequencies of immunocyte populations in the colon for each microbe±standard deviations, highlighting significant changes at a False Discovery Rate (FDR) of ≤0.01. The corresponding Fold Changes (FCs) relative to GF status are summarized in the heat map in FIG. 2B and in Tables 4A-G; results in other tissues in FIGS. 9A-9B and Tables 3-5; individual mouse data which includes frequencies of all cell types per mouse across all strains of bacteria per mouse (m stands for − and p stands for +) (data not shown—see supplemental materials of Geva-Zatorsky et al., Cell 2017, incorporated by reference herein below).

Mean frequencies of all cell types across all microbes +/− standard deviations (m stands for − and p stands for +)
Table 3A

| Organ | Phylum | | Proteobacteria | Proteobacteria | Actinobacteria | Actinobacteria | Bacteroidetes |
|---|---|---|---|---|---|---|---|
| organ | Rownames (cell type in figure) | Germfree | Abaum.ATCC17978 | Alwof.F78 | Badol.L2-32 | Bbrev.SK134 | Bdore.DSM17855 |
| colon | mono.co | 3.1 +/− 1.1 | 2.65 +/− 0.4 | 2.22 +/− 0.25 | 3.33 +/− 0.69 | 3.1 +/− 0.17 | 3.31 +/− 1.37 |
| colon | CD11bpCD11cmF4/80pMF.co | 2.86 +/− 2 | 0.58 +/− 0.23 | 0.44 +/− 0.32 | 1.56 +/− 0.91 | 0.29 +/− 0.15 | 0.46 +/− 0.28 |
| colon | CD11bpCD11cpF4/80pMNP.co | 13.19 +/− 8.27 | 8.97 +/− 2.86 | 3.2 +/− 1.14 | 14.34 +/− 7.34 | 7.82 +/− 0.79 | 7.65 +/− 1.73 |
| colon | CD103pCD11bmDC.co | 1.13 +/− 0.81 | 1.4 +/− 0.42 | 0.35 +/− 0.18 | 2.81 +/− 0.53 | 4.62 +/− 1.1 | 1.07 +/− 0.34 |
| colon | CD103pCD11bpDC.co | 2.81 +/− 0.9 | 2.23 +/− 0.57 | 1.53 +/− 0.34 | 4.29 +/− 2.52 | 3.39 +/− 0.61 | 3.51 +/− 0.52 |
| colon | pDC.co | 1.02 +/− 0.58 | 1.27 +/− 1.04 | 2.04 +/− 0.68 | 2.03 +/− 1.02 | 0.96 +/− 0.29 | 2.38 +/− 0.47 |
| colon | ILC3.co | 2.22 +/− 2.1 | 5.36 +/− 2.24 | 2.7 +/− 0.96 | 3.02 +/− 1.5 | 3.53 +/− 1.41 | 1.2 +/− 0.79 |
| colon | B.co | 58.16 +/− 8.72 | 34.97 +/− 6.7 | 73.35 +/− 2.8 | 51.25 +/− 17.42 | 57 +/− 12.3 | 55.57 +/− 5.06 |
| colon | Tgd.co | 2.32 +/− 1.06 | 2.56 +/− 0.27 | 1.07 +/− 0.2 | 2.17 +/− 1 | 1.87 +/− 0.8 | 2.24 +/− 0.66 |
| colon | Tab.co | 26.99 +/− 6.63 | 34 +/− 5.23 | 13.88 +/− 3.83 | 28.67 +/− 8.65 | 24.37 +/− 10.08 | 26.37 +/− 3.63 |
| colon | DN(CD8mCD4mTCRp).co | 25.02 +/− 5.91 | 26.97 +/− 5.94 | 15.86 +/− 7.34 | 31.04 +/− 10.66 | 22.83 +/− 4.31 | 33.21 +/− 6.56 |
| colon | T8.co | 28.48 +/− 6.44 | 30.08 +/− 0.76 | 23.2 +/− 2.38 | 24.31 +/− 5.85 | 27.2 +/− 1.15 | 23.89 +/− 4.07 |
| colon | T8.Heliosp.co | 43.28 +/− 16.68 | 54.35 +/− 15.09 | 21.95 +/− 11.26 | 53.63 +/− 18.92 | 41 +/− 9.98 | 53.09 +/− 15.41 |
| colon | T4.co | 41.65 +/− 8.23 | 37.9 +/− 6.21 | 56.27 +/− 7.61 | 39.52 +/− 12.31 | 43.67 +/− 3.95 | 37.77 +/− 6.76 |
| colon | T4.FPmRorgp.co | 0.9 +/− 0.59 | 6 +/− 2.42 | 1.41 +/− 0.37 | 3.28 +/− 2.21 | 1.36 +/− 0.11 | 1.65 +/− 1.02 |
| colon | T4.FPp.co | 15.6 +/− 5.43 | 20.9 +/− 9.45 | 22.38 +/− 9.72 | 24.16 +/− 9.34 | 18.6 +/− 0.26 | 20.03 +/− 6.68 |
| colon | T4.FPpHeliosm.co | 26.66 +/− 8.07 | 28.13 +/− 6.87 | 42.32 +/− 4.21 | 36.23 +/− 10.91 | 32.83 +/− 5.4 | 36.69 +/− 3.21 |
| colon | T4.FPpRorgpHeliosm.co | 6.66 +/− 7.49 | 12.53 +/− 2.12 | 25.42 +/− 3.75 | 20.26 +/− 9.88 | 21.67 +/− 4.3 | 15.31 +/− 4.25 |
| colon | ILC.il22p.co | 4.05 +/− 2.39 | 6.45 +/− 1.69 | 1.12 +/− 0.41 | 12.61 +/− 10.35 | 6.13 +/− 2.44 | 6.34 +/− 0.69 |
| colon | T4.ifngp.co | 0.73 +/− 0.68 | 0.88 +/− 0.5 | 1.04 +/− 0.61 | 1.01 +/− 0.61 | 1.21 +/− 0.77 | 0.68 +/− 0.39 |
| colon | T4.il10p.co | 0.33 +/− 0.3 | 0.62 +/− 0.37 | 4.77 +/− 1.97 | 1.21 +/− 1.76 | 0.76 +/− 0.71 | 0.19 +/− 0.19 |
| colon | T4.il17p.co | 1.95 +/− 1.28 | 1.39 +/− 0.33 | 1.02 +/− 0.18 | 2.53 +/− 1.77 | 4.37 +/− 0.17 | 0.96 +/− 0.02 |
| colon | T4.il22p.co | 0.49 +/− 0.5 | 2.48 +/− 3.95 | 0.34 +/− 0.12 | 0.6 +/− 0.34 | | 0.14 +/− 0.17 |
| mln | mono.mln | 0.52 +/− 0.78 | 0.38 +/− 0.12 | 0.31 +/− 0.08 | 0.19 +/− 0.02 | 0.27 +/− 0.14 | 0.17 +/− 0.03 |
| mln | CD11bpCD11cpF4/80pMNP.mln | 2.25 +/− 6.53 | 0.66 +/− 0.26 | 0.83 +/− 0.05 | 0.51 +/− 0.08 | 0.23 +/− 0.17 | 0.2 +/− 0.04 |
| mln | CD103pCD11bmDC.mln | 0.74 +/− 0.42 | 0.7 +/− 0.16 | 0.41 +/− 0.03 | 0.48 +/− 0.07 | 0.65 +/− 0.25 | 0.31 +/− 0.04 |
| mln | CD103pCD11bpDC.mln | 1.5 +/− 0.8 | 1.72 +/− 0.72 | 0.94 +/− 0.11 | 1.12 +/− 0.19 | 2.47 +/− 0.97 | 1.24 +/− 0.17 |
| mln | pDC.mln | 0.26 +/− 0.31 | 0.17 +/− 0.04 | 0.11 +/− 0.04 | 0.32 +/− 0.2 | 0.11 +/− 0.02 | 0.08 +/− 0.01 |
| mln | ILC3.mln | 0.09 +/− 0.04 | 0.1 +/− 0.02 | 0.08 +/− 0.02 | 0.16 +/− 0.03 | 0.08 +/− 0.06 | 0.11 +/− 0.02 |
| mln | B.mln | 48.89 +/− 6.57 | 46.9 +/− 4.44 | 57.3 +/− 1.78 | 42.05 +/− 6.97 | 43.77 +/− 6.3 | 47.53 +/− 0.6 |
| mln | Tgd.mln | 0.52 +/− 0.13 | 0.55 +/− 0.08 | 0.48 +/− 0.04 | 0.56 +/− 0.04 | 1.19 +/− 0.59 | 0.5 +/− 0.01 |
| mln | Tab.mln | 44.99 +/− 5.52 | 45.78 +/− 4.05 | 40.35 +/− 1.93 | 54.82 +/− 7.03 | 43.6 +/− 2.26 | 48.73 +/− 0.6 |
| mln | DN(CD8mCD4mTCRp).mln | 0.58 +/− 0.24 | 0.64 +/− 0.08 | 0.45 +/− 0.03 | 0.42 +/− 0.07 | 0.75 +/− 0.23 | 0.55 +/− 0.09 |
| mln | T8.mln | 35.51 +/− 7.74 | 39.32 +/− 1.01 | 37.98 +/− 2.49 | 34.97 +/− 2.7 | 44.1 +/− 3.06 | 40.07 +/− 0.55 |
| mln | T8.Heliosp.mln | 2.69 +/− 0.75 | 2.41 +/− 0.38 | 3.4 +/− 0.59 | 1.41 +/− 0.25 | 3.21 +/− 0.99 | 1.34 +/− 0.06 |
| mln | T4.mln | 56.12 +/− 9.19 | 59.35 +/− 0.93 | 60.47 +/− 2.3 | 64.13 +/− 2.77 | 54.2 +/− 3.18 | 58.17 +/− 0.61 |
| mln | T4.FPmRorgp.mln | 0.29 +/− 0.26 | 0.65 +/− 0.22 | 0.36 +/− 0.04 | 0.52 +/− 0.14 | 0.6 +/− 0.2 | 0.12 +/− 0.03 |
| mln | T4.FPp.mln | 12.62 +/− 3.67 | 11.9 +/− 0.73 | 12.83 +/− 0.73 | 12.58 +/− 0.75 | 10.26 +/− 3.37 | 11.67 +/− 0.64 |
| mln | T4.FPpHeliosm.mln | 34.3 +/− 9.9 | 31.53 +/− 7.1 | 32.33 +/− 0.93 | 35.31 +/− 1.83 | 33.24 +/− 5.08 | 35.18 +/− 2.15 |
| mln | T4.FPpRorgpHeliosm.mln | 1.26 +/− 0.48 | 2.01 +/− 0.48 | 3.81 +/− 0.93 | 2.13 +/− 0.8 | 3 +/− 1.34 | 3.25 +/− 0.43 |
| mln | ILC.il22p.mln | 0.2 +/− 0.24 | 0.05 +/− 0.04 | 0.2 +/− 0.07 | 0.62 +/− 0.4 | 0.39 +/− 0.34 | 0.07 +/− 0.03 |
| mln | T4.ifngp.mln | 0.5 +/− 0.31 | 1.82 +/− 0.46 | 0.8 +/− 0.37 | 1.24 +/− 0.27 | 0.35 +/− 0.02 | 0.52 +/− 0.33 |
| mln | T4.il10p.mln | 0.11 +/− 0.11 | 0.09 +/− 0.05 | 0.16 +/− 0.06 | 0.02 +/− 0.01 | 0.15 +/− 0.08 | 0.01 +/− 0 |
| mln | T4.il17p.mln | 0.51 +/− 0.67 | 0.23 +/− 0.04 | 0.68 +/− 0.15 | 0.54 +/− 0.2 | 0.34 +/− 0.01 | 0.35 +/− 0.05 |
| mln | T4.il22p.mln | 0.18 +/− 0.13 | 0.18 +/− 0.03 | 0.24 +/− 0.02 | 0.42 +/− 0.16 | | 0.03 +/− 0.02 |
| pp | mono.pp | 0.79 +/− 0.3 | 0.8 +/− 0.19 | 1.15 +/− 0.36 | 1.54 +/− 0.55 | 0.41 +/− 0.15 | 0.8 +/− 0.29 |
| pp | CD11bpCD11cmF4/80pMF.pp | 0.37 +/− 0.25 | 0.11 +/− 0.07 | 0.41 +/− 0.17 | 0.18 +/− 0.11 | 0.03 +/− 0.03 | 0.07 +/− 0.02 |
| pp | CD11bpCD11cpF4/80pMNP.pp | 1.41 +/− 0.94 | 2.97 +/− 0.61 | 1.65 +/− 0.28 | 4.54 +/− 2.75 | 0.84 +/− 0.37 | 1.65 +/− 0.91 |
| pp | CD103pbmCD11bmDC.pp | 2.34 +/− 1.53 | 4.77 +/− 0.45 | 1.78 +/− 0.49 | 4.71 +/− 2.53 | 3.37 +/− 1.01 | 2.83 +/− 1.32 |
| pp | CD103pCD11bpDC.pp | 9.72 +/− 5.63 | 9.19 +/− 2.07 | 10.68 +/− 0.65 | 7.48 +/− 1.57 | 10.82 +/− 4.36 | 9.82 +/− 3.79 |
| pp | pDC.pp | 1.41 +/− 0.81 | 1.81 +/− 0.23 | 0.99 +/− 0.21 | 2.85 +/− 1.63 | 1.72 +/− 0.19 | 1.99 +/− 0.72 |
| pp | ILC3.pp | 0.33 +/− 0.18 | 0.39 +/− 0.07 | 0.47 +/− 0.14 | 0.47 +/− 0.09 | 0.27 +/− 0.02 | 0.22 +/− 0.07 |
| pp | B.pp | 82.55 +/− 5.98 | 87.73 +/− 1.86 | 87.9 +/− 1.54 | 79.1 +/− 15.67 | 86.87 +/− 3.87 | 82.77 +/− 2.46 |
| pp | Tgd.pp | 3.11 +/− 1.13 | 2.14 +/− 0.83 | 2.49 +/− 0.7 | 1.26 +/− 0.46 | 3.86 +/− 2.26 | 1.97 +/− 0.79 |
| pp | Tab.pp | 9.34 +/− 4.07 | 4.83 +/− 0.55 | 6.43 +/− 0.47 | 11.93 +/− 9.78 | 5.99 +/− 1.51 | 8.62 +/− 1.91 |
| pp | DN(CD8mCD4mTCRp).pp | 5.8 +/− 8.49 | 3.01 +/− 0.48 | 4.09 +/− 2.58 | 7.33 +/− 11.19 | 2.94 +/− 0.36 | 6.22 +/− 0.97 |
| pp | T8.pp | 32.19 +/− 2.98 | 33.35 +/− 3.51 | 33.45 +/− 3.32 | 28.69 +/− 3.64 | 27.87 +/− 8.71 | 34.03 +/− 5.56 |
| pp | T8.Heliosp.pp | 19.32 +/− 9.03 | 20.48 +/− 4.17 | 16.08 +/− 4.08 | 20.5 +/− 15.54 | 12.32 +/− 4.68 | 10.63 +/− 5.32 |
| pp | T4.pp | 55.14 +/− 9.05 | 60 +/− 3.73 | 55.6 +/− 6.74 | 60.21 +/− 10.9 | 67.43 +/− 8.39 | 56.73 +/− 6.27 |
| pp | T4.FPmRorgp.pp | 0.81 +/− 0.45 | 0.64 +/− 0.33 | 1.44 +/− 0.51 | 2.51 +/− 1.34 | 2.17 +/− 0.82 | 0.54 +/− 0.26 |
| pp | T4.FPp.pp | 15.54 +/− 4.83 | 20.98 +/− 3.15 | 19.6 +/− 1.94 | 12.99 +/− 7.49 | 14.4 +/− 0.36 | 18.83 +/− 1.42 |
| pp | T4.FPpHeliosm.pp | 28.78 +/− 18.88 | 20.5 +/− 2.51 | 26.61 +/− 3.66 | 29.22 +/− 13.36 | 27.03 +/− 3.35 | 29.89 +/− 1.53 |
| pp | T4.FPpRorgpHeliosm.pp | 3.01 +/− 1.65 | 5.23 +/− 2.27 | 9.01 +/− 3.82 | 3.42 +/− 1.9 | 15 +/− 4.09 | 8.23 +/− 3.7 |
| pp | ILC.il22p.pp | 1.21 +/− 1.45 | 1.66 +/− 1.98 | 0.91 +/− 0.45 | 2.81 +/− 1.71 | 2.9 +/− 1 | 0.32 +/− 0.09 |
| pp | T4.ifngp.pp | 0.58 +/− 0.34 | 2.23 +/− 1.09 | 0.14 +/− 0.09 | 0.07 +/− 0.04 | 1.59 +/− 2.51 | 0.16 +/− 0.16 |
| pp | T4.il10p.pp | 0.49 +/− 0.52 | 1.05 +/− 0.61 | 1.02 +/− 0.36 | 0.11 +/− 0.03 | 2.09 +/− 1.69 | 0.42 +/− 0.05 |
| pp | T4.il17p.pp | 4.75 +/− 12.38 | 0.37 +/− 0.31 | 1.83 +/− 0.64 | 1.92 +/− 0.54 | 2.97 +/− 2.78 | 1.59 +/− 0.43 |
| pp | T4.il22p.pp | 1.01 +/− 2.07 | 0.08 +/− 0.06 | 1.22 +/− 0.22 | 0.27 +/− 0.1 | | 0.24 +/− 0.03 |
| silp | mono.si | 5.73 +/− 2.53 | 7.03 +/− 1.73 | 8.34 +/− 0.64 | 7.29 +/− 2.03 | 5.67 +/− 2.15 | 6.58 +/− 1.64 |
| silp | CD11bpCD11cmF4/80pMF.si | 1.59 +/− 0.99 | 0.52 +/− 0.34 | 0.82 +/− 0.18 | 0.74 +/− 0.55 | 0.04 +/− 0.03 | 0.03 +/− 0.01 |
| silp | CD11bpCD11cpF4/80pMNP.si | 18.99 +/− 8.17 | 9.23 +/− 3.8 | 13.96 +/− 4.48 | 17.87 +/− 11.99 | 9.9 +/− 2.09 | 4.75 +/− 0.76 |
| silp | CD103pCD11bmDC.si | 10.43 +/− 7.73 | 9.93 +/− 1.99 | 7.77 +/− 4.1 | 10.98 +/− 2.02 | 21.96 +/− 6.12 | 16.44 +/− 5.77 |
| silp | CD103pCD11bpDC.si | 1.92 +/− 1.14 | 4.38 +/− 1.64 | 2.02 +/− 0.49 | 1.51 +/− 0.56 | 3.9 +/− 2.01 | 4.34 +/− 1.59 |
| silp | pDC.si | 6.32 +/− 3.03 | 7.36 +/− 2.41 | 11.19 +/− 1.85 | 9.01 +/− 2.21 | 6.26 +/− 3.25 | 6.71 +/− 2.27 |
| silp | ILC3.si | 14.79 +/− 11.85 | 26.57 +/− 14.97 | 23.72 +/− 7.09 | 17.62 +/− 2.7 | 40.61 +/− 4.79 | 29.53 +/− 3.92 |
| silp | B.si | 42.27 +/− 18.05 | 37.9 +/− 21.75 | 29.9 +/− 8.28 | 33.75 +/− 13.79 | 22.47 +/− 2.75 | 28.47 +/− 3.71 |
| silp | Tgd.si | 5.14 +/− 4.95 | 6.06 +/− 3.28 | 2.87 +/− 1.06 | 2.84 +/− 0.6 | 5.63 +/− 3.08 | 5.92 +/− 3.71 |
| silp | Tab.si | 27.67 +/− 9.6 | 24.7 +/− 5.62 | 29.05 +/− 4.42 | 30.9 +/− 9.51 | 19.33 +/− 1.88 | 32.97 +/− 2.03 |
| silp | DN(CD8mCD4mTCRp).si | 10.2 +/− 4 | 11.65 +/− 5.68 | 13.68 +/− 6.41 | 6.87 +/− 2.28 | 19.13 +/− 4.51 | 11.6 +/− 1.2 |
| silp | T8.si | 26.19 +/− 7.32 | 29.42 +/− 4.4 | 26.08 +/− 8.63 | 18.88 +/− 7.25 | 19.73 +/− 6.5 | 20.73 +/− 3.18 |
| silp | T8.Heliosp.si | 17.05 +/− 12.34 | 13.75 +/− 9.15 | 10.27 +/− 1.44 | 15.44 +/− 15.15 | 26.43 +/− 11.84 | 10.74 +/− 4.22 |
| silp | T4.si | 58.46 +/− 8.22 | 54.95 +/− 5.43 | 50.8 +/− 16.7 | 69.05 +/− 8.43 | 54.53 +/− 9.13 | 62.03 +/− 2.12 |
| silp | T4.FPmRorgp.si | 2.65 +/− 4.23 | 8.61 +/− 4.15 | 8.74 +/− 2.98 | 10.5 +/− 6.27 | 5.37 +/− 0.98 | 3.01 +/− 0.29 |
| silp | T4.FPp.si | 24.21 +/− 7.39 | 19.65 +/− 8.45 | 24.48 +/− 4.27 | 21.33 +/− 3.46 | 24.6 +/− 6.1 | 29.97 +/− 3.52 |
| silp | T4.FPpHeliosm.si | 19.89 +/− 19.74 | 14.43 +/− 4.59 | 24.1 +/− 7.94 | 20.03 +/− 2.19 | 15.64 +/− 7.19 | 16.44 +/− 2.8 |
| silp | T4.FPpRorgpHeliosm.si | 2.99 +/− 2.29 | 6.46 +/− 2.38 | 8.13 +/− 4.61 | 8.89 +/− 3.37 | 7.92 +/− 1.44 | 6.91 +/− 1.08 |
| silp | ILC.il22p.si | 10.88 +/− 8.56 | 34.34 +/− 5.31 | 2.05 +/− 1.2 | 30.58 +/− 12.85 | 30.49 +/− 7.81 | 40.81 +/− 3.99 |
| silp | T4.ifngp.si | 0.78 +/− 0.62 | 1.57 +/− 0.91 | 0.95 +/− 0.82 | 2.05 +/− 0.9 | 1.08 +/− 0.93 | 0.29 +/− 0.28 |
| silp | T4.il10p.si | 0.57 +/− 0.49 | 0.56 +/− 0.32 | 1.94 +/− 0.74 | 3.33 +/− 5.12 | 0.89 +/− 0.32 | 0.13 +/− 0.16 |
| silp | T4.il17p.si | 2.08 +/− 2.64 | 4.6 +/− 1.19 | 1.32 +/− 1.2 | 6.34 +/− 1.97 | 3.34 +/− 1.57 | 1.91 +/− 0.65 |
| silp | T4.il22p.si | 0.64 +/− 1.07 | 9.2 +/− 11.68 | 0.34 +/− 0.08 | 1.16 +/− 0.86 | | 0.8 +/− 0.18 |
| sp | mono.slo | 4.22 +/− 1.95 | 3.18 +/− 0.37 | 3.46 +/− 0.31 | 4.67 +/− 1.83 | 3.32 +/− 0.52 | 2.14 +/− 0.46 |
| sp | CD11bpCD11cmF4/80pMF.slo | 2.26 +/− 1.86 | 0.57 +/− 0.08 | 0.4 +/− 0.14 | 0.77 +/− 0.24 | 0.06 +/− 0.06 | 0.25 +/− 0.06 |
| sp | CD11bpCD11cpF4/80pMNP.slo | 4.19 +/− 4.7 | 2.11 +/− 0.32 | 2.45 +/− 0.26 | 4.15 +/− 0.8 | 1.59 +/− 0.49 | 2.12 +/− 0.67 |
| sp | CD103pCD11bmDC.slo | 0.26 +/− 0.46 | 1.09 +/− 0.24 | 0.05 +/− 0.01 | 0.13 +/− 0.02 | 0.78 +/− 0.54 | 0.11 +/− 0.01 |
| sp | CD103pCD11bpDC.slo | 0.4 +/− 0.35 | 0.41 +/− 0.06 | 0.25 +/− 0.06 | 0.76 +/− 0.61 | 0.61 +/− 0.28 | 0.21 +/− 0.03 |
| sp | pDC.slo | 0.78 +/− 1.32 | 0.61 +/− 0.17 | 0.44 +/− 0.07 | 0.64 +/− 0.18 | 0.49 +/− 0.12 | 0.49 +/− 0.03 |
| sp | ILC3.slo | 0.02 +/− 0.01 | 0.02 +/− 0.01 | 0.01 +/− 0 | 0.03 +/− 0 | 0.02 +/− 0 | 0.02 +/− 0.01 |
| sp | B.slo | 64.8 +/− 11.15 | 67.85 +/− 1.48 | 72.88 +/− 1.55 | 68.99 +/− 2.44 | 74.83 +/− 1.89 | 74 +/− 3.58 |
| sp | Tgd.slo | 0.39 +/− 0.09 | 0.4 +/− 0.03 | 0.34 +/− 0.04 | 0.39 +/− 0.08 | 0.45 +/− 0.03 | 0.3 +/− 0.06 |
| sp | Tab.slo | 21.72 +/− 5.06 | 21.32 +/− 1.3 | 22.33 +/− 1.64 | 21.26 +/− 3.47 | 19.2 +/− 2.29 | 18.98 +/− 3.88 |
| sp | DN(CD8mCD4mTCRp).slo | 3.78 +/− 2.87 | 1.9 +/− 0.16 | 4.05 +/− 1.13 | 2.56 +/− 0.5 | 1.82 +/− 0.23 | 2.26 +/− 0.35 |
| sp | T8.slo | 33.74 +/− 2.73 | 35.38 +/− 1.85 | 35.28 +/− 2.81 | 31.15 +/− 3.61 | 36.7 +/− 1.1 | 33.58 +/− 2.74 |
| sp | T8.Heliosp.slo | 4.62 +/− 1.16 | 4.55 +/− 0.54 | 5.23 +/− 0.89 | 3.48 +/− 0.88 | 6.04 +/− 0.15 | 2.01 +/− 0.28 |
| sp | T4.slo | 60.23 +/− 2.41 | 61.07 +/− 1.86 | 59.18 +/− 3.61 | 63.7 +/− 3.38 | 59.7 +/− 0.89 | 61.43 +/− 3.01 |
| sp | T4.FPmRorgp.slo | 0.29 +/− 0.21 | 0.55 +/− 0.13 | 0.12 +/− 0.03 | 0.27 +/− 0.09 | 0.2 +/− 0.03 | 0.23 +/− 0.09 |
| sp | T4.FPp.slo | 12.04 +/− 1.2 | 12.48 +/− 1.36 | 12.2 +/− 1.49 | 12.73 +/− 1.07 | 14.33 +/− 0.64 | 11.65 +/− 0.3 |
| sp | T4.FPpHeliosm.slo | 27.97 +/− 12.9 | 31.23 +/− 1.74 | 29.59 +/− 4.84 | 23.27 +/− 3.46 | 22.09 +/− 1.13 | 29.29 +/− 0.76 |
| sp | T4.FPpRorgpHeliosm.slo | 0.27 +/− 0.18 | 0.58 +/− 0.19 | 1.46 +/− 0.91 | 0.41 +/− 0.27 | 1.02 +/− 0.18 | 0.56 +/− 0.12 |
| sp | ILC.il22p.slo | 0.09 +/− 0.15 | 0.02 +/− 0.02 | 0.07 +/− 0.04 | 0.4 +/− 0.44 | 0.15 +/− 0.04 | 0.01 +/− 0.01 |
| sp | T4.ifngp.slo | 0.82 +/− 0.51 | 0.92 +/− 0.1 | 1.02 +/− 0.42 | 1.82 +/− 1.63 | 0.9 +/− 0.26 | 1.08 +/− 0.14 |
| sp | T4.il10p.slo | 0.1 +/− 0.08 | 0.3 +/− 0.1 | 0.99 +/− 0.43 | 0.55 +/− 0.73 | 0.06 +/− 0 | 0.01 +/− 0 |
| sp | T4.il17p.slo | 0.33 +/− 0.22 | 1.16 +/− 0.02 | 0.24 +/− 0.13 | 0.63 +/− 0.2 | 0.44 +/− 0.07 | 0.4 +/− 0.01 |
| sp | T4.il22p.slo | 0.19 +/− 0.2 | 0.34 +/− 0.09 | 0.13 +/− 0.03 | 0.17 +/− 0.13 | | 0.02 +/− 0 |

Table 3B

| Organ | Phylum | Bacteroidetes | Bacteroidetes | Actinobacteria | Bacteroidetes | Bacteroidetes | Bacteroidetes |
|---|---|---|---|---|---|---|---|
| organ | Rownames (cell type in figure) | Bfine.DSM17565 | Bfrag.NCTC9343 | Blong.AO44 | Bmass.DSM17679 | Bovat.ATCC8483 | Bsala.DSM18170 |
| colon | mono.co | 3.37 +/− 0.69 | 2.59 +/− 0.97 | | 1.93 +/− 0.6 | 2.48 +/− 0.33 | 3.21 +/− 0.33 |
| colon | CD11bpCD11cmF4/80pMF.co | 2.23 +/− 0.55 | 2.67 +/− 0.79 | | 1.59 +/− 0.95 | 0.94 +/− 0.6 | 0.88 +/− 0.46 |
| colon | CD11bpCD11cpF4/80pMNP.co | 9.04 +/− 1.24 | 18.77 +/− 3.51 | | 7.89 +/− 4.46 | 14.11 +/− 7.45 | 7.64 +/− 2.37 |
| colon | CD103pCD11bmDC.co | 0.64 +/− 0.07 | 0.82 +/− 0.46 | | 0.87 +/− 0.67 | 1.71 +/− 1.09 | 0.58 +/− 0.35 |
| colon | CD103pCD11bpDC.co | 2.6 +/− 0.33 | 3.54 +/− 0.98 | | 5.52 +/− 1.85 | 2.58 +/− 0.95 | 2.85 +/− 0.76 |
| colon | pDC.co | 3.72 +/− 2.13 | 4.18 +/− 3.12 | | 2.64 +/− 2.37 | 2.76 +/− 1.89 | 0.9 +/− 0.13 |
| colon | ILC3.co | 1.98 +/− 0.99 | 1.39 +/− 0.76 | 2.06 +/− 1.19 | 1.21 +/− 0.69 | 1.65 +/− 2.24 | 3.05 +/− 1.5 |
| colon | B.co | 63.17 +/− 7.07 | 42.18 +/− 14.85 | 49.77 +/− 5.98 | 51.93 +/− 12.63 | 63.06 +/− 11.75 | 47.88 +/− 9.91 |
| colon | Tgd.co | 1.38 +/− 0.37 | 3.78 +/− 1.84 | 2.19 +/− 0.66 | 2.48 +/− 1.22 | 2.04 +/− 0.76 | 3.38 +/− 0.97 |
| colon | Tab.co | 19.51 +/− 4.17 | 32.06 +/− 7.29 | 35.45 +/− 2.76 | 30.02 +/− 10.59 | 22.92 +/− 5.31 | 29.25 +/− 5.44 |
| colon | DN(CD8mCD4mTCRp).co | 21.98 +/− 3.02 | 31.54 +/− 9.71 | 23.7 +/− 9.45 | 30.22 +/− 9.77 | 27.86 +/− 6.95 | 37.12 +/− 9.34 |
| colon | T8.co | 27.6 +/− 2.61 | 29.15 +/− 4.76 | 30.07 +/− 1.23 | 30.01 +/− 4.36 | 33.77 +/− 3.13 | 26.45 +/− 3.72 |
| colon | T8.Heliosp.co | 44.94 +/− 12.31 | 58.9 +/− 20.24 | 45.23 +/− 19.53 | 55.76 +/− 19.65 | 60.87 +/− 17.08 | 70.4 +/− 14.43 |
| colon | T4.co | 44.79 +/− 5.28 | 35.84 +/− 11.12 | 41.68 +/− 10.04 | 36.67 +/− 14.65 | 34.9 +/− 8.02 | 33.3 +/− 7.5 |
| colon | T4.FPmRorgp.co | 1.17 +/− 0.55 | 1.44 +/− 0.85 | 1.11 +/− 0.64 | 1.19 +/− 0.56 | 1.18 +/− 0.79 | 1.76 +/− 1.29 |
| colon | T4.FPp.co | 26.06 +/− 11.26 | 22.89 +/− 9.58 | 18.5 +/− 5.48 | 22.5 +/− 9.36 | 26.26 +/− 7.75 | 23.56 +/− 8.57 |
| colon | T4.FPpHeliosm.co | 39.12 +/− 6.58 | 39.66 +/− 9.9 | 26.8 +/− 2.61 | 41.32 +/− 8.25 | 33.44 +/− 12.18 | 35.82 +/− 2.86 |
| colon | T4.FPpRorgpHeliosm.co | 23.48 +/− 7.42 | 20.25 +/− 5.64 | 12.18 +/− 4.21 | 27.06 +/− 8.51 | 19.42 +/− 8.25 | 15.59 +/− 3.09 |
| colon | ILC.il22p.co | 1.56 +/− 0.65 | 12.02 +/− 10.09 | 7.2 +/− 1.28 | 4.65 +/− 0.65 | 9.12 +/− 7.77 | 4.4 +/− 3.32 |
| colon | T4.ifngp.co | 1.84 +/− 0.69 | 0.52 +/− 0.47 | 2.18 +/− 0.44 | 1.35 +/− 0.68 | 1.02 +/− 0.04 | 0.62 +/− 0.21 |
| colon | T4.il10p.co | 0.5 +/− 0.28 | 0.71 +/− 0.44 | 2.35 +/− 1.51 | 0.74 +/− 0.78 | 1.47 +/− 0.7 | 0.26 +/− 0.07 |
| colon | T4.il17p.co | 1.35 +/− 0.45 | 1.49 +/− 1.5 | 1.03 +/− 0.38 | 2.7 +/− 0.98 | 2.46 +/− 1.17 | 2.15 +/− 1.01 |
| colon | T4.il22p.co | 0.36 +/− 0.63 | 0.62 +/− 0.47 | 0.2 +/− 0.21 | 0.16 +/− 0.24 | 0.09 +/− 0.08 | 0.22 +/− 0.23 |
| mln | mono.mln | 0.14 +/− 0.01 | 0.25 +/− 0.11 | | 0.16 +/− 0.06 | | 0.2 +/− 0.03 |
| mln | CD11bpCD11cpF4/80pMNP.mln | 0.18 +/− 0.06 | 0.79 +/− 0.42 | | 2.79 +/− 3.88 | 0.64 +/− 0.19 | 0.16 +/− 0.01 |
| mln | CD103pCD11bm DC.mln | 0.15 +/− 0.06 | 0.73 +/− 0.23 | | 0.8 +/− 1.03 | 0.35 +/− 0.1 | 0.18 +/− 0.02 |
| mln | CD103pCD11bp DC.mln | 1.06 +/− 0.39 | 1.28 +/− 0.41 | | 1.45 +/− 0.42 | 0.55 +/− 0.26 | 0.77 +/− 0.15 |
| mln | pDC.mln | 0.2 +/− 0 | 0.16 +/− 0.1 | | 0.14 +/− 0.06 | 0.14 +/− 0.05 | 0.22 +/− 0.04 |
| mln | ILC3.mln | 0.13 +/− 0.02 | 0.07 +/− 0.04 | 0.06 +/− 0.02 | 0.1 +/− 0.07 | 0.02 +/− 0 | 0.18 +/− 0.04 |
| mln | B.mln | 44.65 +/− 3.21 | 47.59 +/− 8.43 | 33.18 +/− 2.57 | 38.45 +/− 8.92 | 51.28 +/− 7.84 | 51.27 +/− 1.73 |
| mln | Tgd.mln | 0.46 +/− 0.05 | 0.72 +/− 0.36 | 0.42 +/− 0.05 | 0.63 +/− 0.13 | 0.37 +/− 0.08 | 0.56 +/− 0.06 |
| mln | Tab.mln | 51.63 +/− 3.19 | 46.11 +/− 10.36 | 45.75 +/− 3.48 | 56 +/− 10.59 | 46.58 +/− 8.34 | 45.07 +/− 1.75 |
| mln | DN(CD8mCD4mTCRp).mln | 0.49 +/− 0.03 | 0.89 +/− 0.86 | 0.61 +/− 0.08 | 0.52 +/− 0.08 | 2.3 +/− 2.19 | 0.51 +/− 0.12 |
| mln | T8.mln | 43.82 +/− 0.94 | 39.16 +/− 5.37 | 41.77 +/− 2.32 | 40.53 +/− 0.81 | 37.72 +/− 0.83 | 39.23 +/− 0.26 |
| mln | T8.Heliosp.mln | 2.31 +/− 0.41 | 3.02 +/− 0.76 | 4.84 +/− 1.19 | 2.35 +/− 0.41 | 4.53 +/− 0.71 | 1.95 +/− 0.14 |
| mln | T4.mln | 54.75 +/− 1.16 | 57.06 +/− 2.27 | 57.03 +/− 2.51 | 57.91 +/− 0.82 | 58.87 +/− 2.24 | 58.9 +/− 0.14 |
| mln | T4.FPmRorgp.mln | 0.3 +/− 0.07 | 0.63 +/− 0.84 | 0.33 +/− 0.06 | 0.26 +/− 0.11 | 0.16 +/− 0.03 | 0.31 +/− 0.11 |
| mln | T4.FPp.mln | 10.67 +/− 1.88 | 12.64 +/− 1.95 | 11.35 +/− 0.79 | 12.2 +/− 0.7 | 12.83 +/− 0.93 | 12.3 +/− 0.22 |
| mln | T4.FPpHeliosm.mln | 31.91 +/− 3.95 | 30.65 +/− 5.66 | 26.63 +/− 2.68 | 33.21 +/− 2.17 | 26.48 +/− 11.02 | 29.63 +/− 1.54 |
| mln | T4.FPpRorgpHeliosm.mln | 3.51 +/− 0.74 | 3.85 +/− 3.8 | 2.08 +/− 0.5 | 4.23 +/− 1.36 | 4.26 +/− 0.36 | 2.88 +/− 0.46 |
| mln | ILC.il22p.mln | 0.76 +/− 0.43 | 1.9 +/− 0.96 | 0.6 +/− 0.33 | 0.11 +/− 0.04 | 0.22 +/− 0.26 | 1.02 +/− 0.23 |
| mln | T4.ifngp.mln | 1.4 +/− 1.14 | 0.58 +/− 0.21 | 0.83 +/− 0.38 | 0.56 +/− 0.17 | 0.17 +/− 0.04 | 0.85 +/− 0.31 |
| mln | T4.il10p.mln | 0.13 +/− 0.11 | 0.15 +/− 0.06 | 1.43 +/− 0.82 | 0.19 +/− 0.04 | 0.38 +/− 0.13 | 0.3 +/− 0.17 |
| mln | T4.il17p.mln | 0.83 +/− 0.31 | 0.35 +/− 0.35 | 0.41 +/− 0.09 | 0.65 +/− 0.1 | 0.23 +/− 0.08 | 0.55 +/− 0.13 |
| mln | T4.il22p.mln | 0.1 +/− 0.02 | 0.61 +/− 0.51 | 0.07 +/− 0.02 | 0.09 +/− 0.05 | 0.13 +/− 0.04 | 0.08 +/− 0.03 |
| pp | mono.pp | 0.63 +/− 0.07 | 1.03 +/− 0.34 | | 0.66 +/− 0.28 | 0.55 +/− 0.33 | 0.98 +/− 0.26 |
| pp | CD11bpCD11cmF4/80pMF.pp | 0.19 +/− 0.02 | 0.17 +/− 0.09 | | 0.55 +/− 0.15 | 0.12 +/− 0.07 | 0.27 +/− 0.14 |
| pp | CD11bpCD11cpF4/80pMNP.pp | 0.93 +/− 0.13 | 3.79 +/− 2.49 | | 2.17 +/− 1.33 | 2.49 +/− 0.67 | 1.29 +/− 0.21 |
| pp | CD103pCD11bmDC.pp | 0.87 +/− 0.17 | 3.13 +/− 2.29 | | 4.69 +/− 1.36 | 4.64 +/− 1.77 | 1.37 +/− 0.31 |
| pp | CD103pCD11bpDC.pp | 6.07 +/− 1.52 | 10.24 +/− 2.31 | | 14.04 +/− 3.29 | 9.24 +/− 5.19 | 6.11 +/− 1.25 |
| pp | pDC.pp | 1.64 +/− 0.19 | 3.37 +/− 2.23 | | 2.48 +/− 0.94 | 2.91 +/− 1.42 | 1.96 +/− 0.41 |
| pp | ILC3.pp | 0.52 +/− 0.07 | 0.13 +/− 0.09 | 0.24 +/− 0.09 | 0.5 +/− 0.14 | 0.24 +/− 0.21 | 0.44 +/− 0.22 |
| pp | B.pp | 88.45 +/− 1.02 | 65.29 +/− 12.8 | 86.38 +/− 2.73 | 84.24 +/− 3.16 | 85.12 +/− 5.59 | 81.2 +/− 6.54 |
| pp | Tgd.pp | 1.34 +/− 0.56 | 6.21 +/− 4.88 | 3.03 +/− 1.16 | 3.67 +/− 2.08 | 3.01 +/− 1.36 | 1.72 +/− 0.83 |
| pp | Tab.pp | 8.09 +/− 0.18 | 12.93 +/− 12.76 | 6.15 +/− 0.72 | 8.28 +/− 1.72 | 8.24 +/− 2.52 | 9.45 +/− 2.1 |
| pp | DN(CD8mCD4mTCRp).pp | 1.84 +/− 0.52 | 8.22 +/− 6.38 | 2.21 +/− 0.51 | 3.82 +/− 1.43 | 6.51 +/− 5.5 | 5.74 +/− 4.9 |
| pp | T8.pp | 28.7 +/− 2.41 | 32.75 +/− 7.6 | 38.48 +/− 3.39 | 35.69 +/− 2.85 | 37.17 +/− 6.52 | 25.92 +/− 5.51 |
| pp | T8.Heliosp.pp | 11.79 +/− 4.88 | 29.57 +/− 17.17 | 19.05 +/− 2.43 | 19.52 +/− 5.36 | 26.84 +/− 12.87 | 8.22 +/− 1.03 |
| pp | T4.pp | 66.78 +/− 3.22 | 53.94 +/− 7.5 | 53.6 +/− 4.63 | 54.94 +/− 2.67 | 39.87 +/− 12.88 | 56.18 +/− 7.96 |
| pp | T4.FPmRorgp.pp | 0.83 +/− 0.57 | 0.57 +/− 0.28 | 1.22 +/− 0.26 | 1 +/− 0.72 | 0.63 +/− 0.47 | 0.64 +/− 0.41 |
| pp | T4.FPp.pp | 14.15 +/− 1.26 | 16.3 +/− 4.12 | 13.8 +/− 1.44 | 13.76 +/− 2.32 | 23.22 +/− 8.32 | 15.52 +/− 2.65 |
| pp | T4.FPpHeliosm.pp | 29.51 +/− 2.4 | 32.27 +/− 8.26 | 21.09 +/− 3.08 | 24.76 +/− 3.25 | 25.08 +/− 11.54 | 29.89 +/− 2.79 |
| pp | T4.FPpRorgpHeliosm.pp | 7.63 +/− 0.72 | 7.18 +/− 5.34 | 7.46 +/− 2.51 | 9.11 +/− 3.92 | 5.63 +/− 2.78 | 8.82 +/− 1.91 |
| pp | ILC.il22p.pp | 3.44 +/− 1.98 | 2.88 +/− 2.28 | 3.77 +/− 2.41 | 1.31 +/− 0.89 | 4.09 +/− 4.97 | 1.95 +/− 1.33 |
| pp | T4.ifngp.pp | 0.6 +/− 0.29 | 0.6 +/− 0.34 | 0.4 +/− 0.17 | 0.47 +/− 0.1 | 1.21 +/− 0.41 | 3.23 +/− 3.95 |
| pp | T4.il10p.pp | 0.3 +/− 0.14 | 0.8 +/− 0.63 | 1 +/− 0.4 | 0.45 +/− 0.2 | 2.91 +/− 1.56 | 0.28 +/− 0.08 |
| pp | T4.il17p.pp | 1.78 +/− 0.81 | 1.02 +/− 1.57 | 1.5 +/− 0.33 | 3.98 +/− 0.6 | 0.41 +/− 0.26 | 1.21 +/− 0.61 |
| pp | T4.il22p.pp | 0.2 +/− 0.13 | 0.6 +/− 0.61 | 0.25 +/− 0.17 | 0.3 +/− 0.08 | 0.11 +/− 0.12 | 0.34 +/− 0.13 |
| silp | mono, si | 7.27 +/− 0.99 | 4.86 +/− 1.85 | | 4.74 +/− 1.19 | 6.24 +/− 3.1 | 8.58 +/− 3.09 |
| silp | CD11bpCD11cmF4/80pMF.si | 0.91 +/− 0.19 | 2.63 +/− 1.4 | | 1 +/− 1.22 | 0.54 +/− 0.59 | 0.09 +/− 0.02 |
| silp | CD11bpCD11cpF4/80pMNP.si | 16.28 +/− 2.55 | 23.02 +/− 8.11 | | 8.41 +/− 9.3 | 12.67 +/− 6.93 | 0.81 +/− 0.96 |
| silp | CD103pCD11bmDC.si | 11.37 +/− 2.08 | 5.32 +/− 3.33 | | 15.89 +/− 11.08 | 7.77 +/− 2.38 | 23.48 +/− 6.35 |
| silp | CD103pCD11bpDC.si | 2.56 +/− 0.72 | 2.83 +/− 1.67 | | 5.08 +/− 1.87 | 5.04 +/− 4.54 | 1.72 +/− 0.35 |
| silp | pDC.si | 12.77 +/− 3.65 | 20.05 +/− 4.45 | | 9.91 +/− 5.26 | 7.16 +/− 3.58 | 7.39 +/− 2.9 |
| silp | ILC3.si | 32.03 +/− 3.36 | 8.41 +/− 5.38 | 26.64 +/− 1.82 | 17.99 +/− 5.8 | 28.08 +/− 6.45 | 14.65 +/− 8.17 |
| silp | B.si | 20.2 +/− 1.64 | 39.29 +/− 25.99 | 27.5 +/− 11.55 | 26.64 +/− 14.77 | 28.04 +/− 6.92 | 53.85 +/− 25.45 |
| silp | Tgd.si | 6.64 +/− 3.82 | 7.5 +/− 5.89 | 6.01 +/− 0.79 | 7.7 +/− 3.41 | 10.55 +/− 8.03 | 2.19 +/− 1.66 |
| silp | Tab.si | 28.65 +/− 3.42 | 22.01 +/− 8.57 | 18.85 +/− 2.68 | 30.61 +/− 7.6 | 31.09 +/− 10.95 | 26.8 +/− 13.65 |
| silp | DN(CD8mCD4mTCRp).si | 9.49 +/− 1.72 | 6.6 +/− 2.14 | 13.55 +/− 1.66 | 9.84 +/− 2.6 | 8.43 +/− 2.14 | 11.74 +/− 5.01 |
| silp | T8.si | 24.35 +/− 4.66 | 30.07 +/− 6.87 | 29.13 +/− 2.62 | 27.54 +/− 6.99 | 30.59 +/− 6.05 | 23 +/− 3.79 |
| silp | T8.Heliosp.si | 27.98 +/− 10.64 | 33.47 +/− 18.79 | 30.7 +/− 14.86 | 31.8 +/− 13.37 | 21.67 +/− 8.86 | 4.79 +/− 3.25 |
| silp | T4.si | 57.42 +/− 6.63 | 60.48 +/− 6.7 | 53.1 +/− 3.7 | 56.53 +/− 5.95 | 57.05 +/− 5.48 | 62.55 +/− 2.18 |
| silp | T4.FPmRorgp.si | 1.41 +/− 1.17 | 2.38 +/− 1.5 | 5.17 +/− 1.92 | 3.13 +/− 0.45 | 2.79 +/− 1.69 | 4.21 +/− 1.38 |
| silp | T4.FPp.si | 24.57 +/− 4.45 | 28.68 +/− 9.73 | 31.75 +/− 3.48 | 24.61 +/− 3.61 | 28.79 +/− 11.74 | 17.17 +/− 3.2 |
| silp | T4.FPpHeliosm.si | 16.12 +/− 3.82 | 22.13 +/− 4.36 | 10.76 +/− 3.56 | 17.97 +/− 3.84 | 15.99 +/− 10.59 | 19.78 +/− 5.93 |
| silp | T4.FPpRorgpHeliosm.si | 2.32 +/− 1.3 | 8.71 +/− 2.91 | 5.5 +/− 2.24 | 10.68 +/− 2.65 | 10.72 +/− 5.99 | 7.29 +/− 1.8 |
| silp | ILC.il22p.si | 14.6 +/− 7.43 | 20.7 +/− 7.3 | 30.53 +/− 5.13 | 20.56 +/− 3.84 | 28.79 +/− 8.3 | 24.03 +/− 2.47 |
| silp | T4.ifngp.si | 0.32 +/− 0.33 | 0.71 +/− 1.01 | 1.63 +/− 0.88 | 2.43 +/− 0.75 | 2.42 +/− 0.74 | 1.47 +/− 0.52 |
| silp | T4.il10p.si | 0.29 +/− 0.09 | 1.52 +/− 1.44 | 0.89 +/− 0.4 | 0.86 +/− 0.27 | 1.71 +/− 0.22 | 0.41 +/− 0.17 |
| silp | T4.il17p.si | 4.26 +/− 0.49 | 1.98 +/− 0.74 | 2.05 +/− 0.47 | 2.94 +/− 0.58 | 3.64 +/− 2.41 | 2.16 +/− 0.97 |
| silp | T4.il22p.si | 0.18 +/− 0.13 | 0.84 +/− 0.62 | 1.26 +/− 1.76 | 0.69 +/− 0.42 | 0.66 +/− 0.27 | 2.61 +/− 2.02 |
| sp | mono.slo | 1.69 +/− 0.59 | 7.59 +/− 2.98 | | 2.25 +/− 0.35 | 3.53 +/− 0.89 | 4.17 +/− 0.86 |
| sp | CD11bpCD11cmF4/80pMF.slo | 0.3 +/− 0.02 | 2.77 +/− 1.43 | | 0.23 +/− 0.06 | 0.7 +/− 0.37 | 0.6 +/− 0.1 |
| sp | CD11bpCD11cpF4/80pMNP.slo | 0.86 +/− 0.74 | 4.56 +/− 1.41 | | 2.13 +/− 0.69 | 4.01 +/− 1.03 | 2.25 +/− 0.32 |
| sp | CD103pCD11bmDC.slo | 0.08 +/− 0.09 | 0.12 +/− 0.04 | | 0.44 +/− 0.26 | 0.06 +/− 0.05 | 0.07 +/− 0.01 |
| sp | CD103pCD11bpDC.slo | 0.32 +/− 0.11 | 0.35 +/− 0.15 | | 0.34 +/− 0.07 | 0.18 +/− 0.18 | 0.22 +/− 0.04 |
| sp | pDC.slo | 0.3 +/− 0.04 | 0.59 +/− 0.21 | | 0.53 +/− 0.18 | 0.43 +/− 0.17 | 0.74 +/− 0.03 |
| sp | ILC3.slo | 0.02 +/− 0.01 | 0.04 +/− 0.02 | 0.02 +/− 0 | 0.02 +/− 0.01 | 0.02 +/− 0.01 | 0.02 +/− 0.01 |
| sp | B.slo | 60.82 +/− 3.7 | 66.13 +/− 6.34 | 63.65 +/− 1.33 | 56.11 +/− 17.25 | 59.95 +/− 14.06 | 70.11 +/− 3.03 |
| sp | Tgd.slo | 0.48 +/− 0.1 | 0.62 +/− 0.42 | 0.26 +/− 0.04 | 0.6 +/− 0.23 | 0.43 +/− 0.06 | 0.52 +/− 0.14 |
| sp | Tab.slo | 33.36 +/− 3.12 | 23.82 +/− 5.04 | 20.9 +/− 1.13 | 37.48 +/− 17.66 | 32.95 +/− 15.26 | 22.14 +/− 2.72 |
| sp | DN(CD8mCD4mTCRp).slo | 1.45 +/− 0.24 | 3.12 +/− 1.65 | 2.65 +/− 0.18 | 1.87 +/− 0.66 | 2.21 +/− 1.17 | 2.48 +/− 1.39 |
| sp | T8.slo | 35.71 +/− 1.32 | 36.36 +/− 4.06 | 35.82 +/− 1.2 | 36.63 +/− 1.52 | 34.53 +/− 1.66 | 34.24 +/− 2.13 |
| sp | T8.Heliosp.slo | 3.59 +/− 0.66 | 5.57 +/− 2.38 | 5.78 +/− 1.53 | 3.35 +/− 0.72 | 6.01 +/− 1.96 | 2.57 +/− 0.32 |
| sp | T4.slo | 61.39 +/− 1.36 | 58.87 +/− 3.86 | 59.5 +/− 0.97 | 59.75 +/− 1.98 | 60.2 +/− 3.01 | 60.5 +/− 2.64 |
| sp | T4.FPmRorgp.slo | 0.49 +/− 0.39 | 0.3 +/− 0.15 | 0.19 +/− 0.12 | 0.36 +/− 0.25 | 0.19 +/− 0.11 | 0.2 +/− 0.09 |
| sp | T4.FPp.slo | 10.44 +/− 0.98 | 13.11 +/− 2.63 | 12.75 +/− 0.6 | 11.65 +/− 1.01 | 11.92 +/− 0.67 | 12.7 +/− 1.36 |
| sp | T4.FPpHeliosm.sio | 27.74 +/− 3.24 | 22.9 +/− 5.41 | 24.3 +/− 0.53 | 23.55 +/− 1.54 | 21.99 +/− 8.75 | 30.09 +/− 1.44 |
| sp | T4.FPpRorgpHeliosm.slo | 0.7 +/− 0.29 | 1.54 +/− 1.93 | 0.75 +/− 0.25 | 1.4 +/− 1.04 | 1.21 +/− 1.26 | 0.46 +/− 0.14 |
| sp | ILC.il22p.slo | 0.18 +/− 0.04 | 0.62 +/− 0.48 | 0.22 +/− 0.06 | 0.01 +/− 0 | 0.07 +/− 0.04 | 0.06 +/− 0.03 |
| sp | T4.ifngp.slo | 1.34 +/− 0.37 | 1.12 +/− 0.61 | 0.7 +/− 0.14 | 1.04 +/− 0.45 | 0.53 +/− 0.08 | 1.63 +/− 0.6 |
| sp | T4.il10p.slo | 0.24 +/− 0.09 | 0.12 +/− 0.03 | 0.26 +/− 0.17 | 0.14 +/− 0.04 | 0.3 +/− 0.08 | 0.11 +/− 0.04 |
| sp | T4.il17p.slo | 1.38 +/− 0.67 | 0.18 +/− 0.15 | 0.18 +/− 0.03 | 1.02 +/− 0.56 | 0.12 +/− 0.02 | 0.33 +/− 0.16 |
| sp | T4.il22p.slo | 0.17 +/− 0.04 | 0.24 +/− 0.19 | 0.03 +/− 0.01 | 0.08 +/− 0.05 | 0.08 +/− 0.02 | 0.15 +/− 0.18 |

Table 3C

| Organ | Phylum | Bacteroidetes | Bacteroidetes | Bacteroidetes | Actinobacteria | Firmicutes | Proteobacteria |
|---|---|---|---|---|---|---|---|
| organ | Rownames (cell type in figure) | Bthet.ATCC29741 | Bunif.ATCC8492 | Bvulg.ATCC8482 | Caero.VPI1003 | Chist.AO25 | Cjeju.AS-84-79 |
| colon | mono.co | 3 +/− 0.7 | 4.29 +/− 2.17 | 3 +/− 1.1 | 2.21 +/− 0.25 | 4 +/− 0.95 | 5.2 +/− 1.17 |
| colon | CD11bpCD11cmF4/80pMF.co | 1.52 +/− 0.37 | 0.34 +/− 0.18 | 1.61 +/− 0.26 | 0.2 +/− 0.2 | 0.94 +/− 1.24 | 0.89 +/− 0.27 |
| colon | CD11bpCD11cpF4/80pMNP.co | 27.28 +/− 6.6 | 8.04 +/− 3.43 | 23.04 +/− 9.66 | 0.62 +/− 0.42 | 4.65 +/− 3.87 | 6.52 +/− 1.19 |
| colon | CD103pCD11bmDC.co | 2.56 +/− 0.45 | 4.25 +/− 5.43 | 1.16 +/− 0.49 | 1.88 +/− 0.63 | 3 +/− 1.93 | 1.44 +/− 0.32 |
| colon | CD103pCD11bpDC.co | 1.48 +/− 0.19 | 2.09 +/− 0.78 | 2.53 +/− 0.75 | 3.23 +/− 0.33 | 3.21 +/− 0.76 | 2.81 +/− 0.28 |
| colon | pDC.co | 2.36 +/− 1.01 | 3.82 +/− 0.16 | 5.57 +/− 5.2 | 0.43 +/− 0.04 | 1.23 +/− 0.52 | 1.17 +/− 0.29 |
| colon | ILC3.co | 2.46 +/− 1.76 | 6.53 +/− 8.38 | 1.76 +/− 1.06 | 2.18 +/− 0.26 | 3.39 +/− 1.4 | 2.31 +/− 0.43 |
| colon | B.co | 56.88 +/− 10.6 | 49.58 +/− 13.8 | 50.91 +/− 11.68 | 62.25 +/− 9.63 | 60.47 +/− 17.5 | 72.4 +/− 5.01 |
| colon | Tgd.co | 3.04 +/− 1.53 | 2.39 +/− 1.04 | 2.45 +/− 0.48 | 1.88 +/− 0.58 | 1.85 +/− 1.3 | 1.63 +/− 0.33 |
| colon | Tab.co | 24.16 +/− 5.69 | 24.68 +/− 5.23 | 27.61 +/− 6.06 | 25.35 +/− 8.12 | 22.53 +/− 12.19 | 18.55 +/− 4.09 |
| colon | DN(CD8mCD4mTCRp).co | 24.82 +/− 10.49 | 28.49 +/− 11.15 | 28.68 +/− 7.35 | 27.85 +/− 5 | 22.99 +/− 12.18 | 15.85 +/− 3.47 |
| colon | T8.co | 27.43 +/− 6.49 | 24.62 +/− 4.01 | 27.62 +/− 5.12 | 32.05 +/− 1.93 | 23.44 +/− 2.66 | 26.2 +/− 2.45 |
| colon | T8.Heliosp.co | 55.65 +/− 11.8 | 43.96 +/− 17.02 | 62.08 +/− 5.18 | 25.3 +/− 4.29 | 23.55 +/− 21.57 | 22.18 +/− 4.67 |
| colon | T4.co | 44.01 +/− 9.02 | 41.69 +/− 8.49 | 38.59 +/− 4.57 | 37.3 +/− 5.05 | 48.56 +/− 13.72 | 55.5 +/− 4.39 |
| colon | T4.FPmRorgp.co | 1.53 +/− 1.24 | 2.14 +/− 1.64 | 1.67 +/− 0.71 | 2.12 +/− 0.67 | 3.43 +/− 2.27 | 2.34 +/− 0.79 |
| colon | T4.FPp.co | 26.56 +/− 11.53 | 27.22 +/− 5.67 | 35.23 +/− 11.22 | 18.07 +/− 2.69 | 26.38 +/− 8.29 | 20.27 +/− 2.88 |
| colon | T4.FPpHeliosm.co | 36.15 +/− 9.46 | 42.76 +/− 6.4 | 39.37 +/− 13.22 | 31.66 +/− 2.61 | 48.27 +/− 7.7 | 50.92 +/− 6.06 |
| colon | T4.FPpRorgpHeliosm.co | 31.64 +/− 7.34 | 26.09 +/− 7.05 | 30.82 +/− 6.59 | 9.19 +/− 1.79 | 32.2 +/− 9.37 | 34.98 +/− 6.86 |
| colon | ILC.il22p.co | | 19.58 +/− 13.91 | 1.95 +/− 0.48 | 6.91 +/− 1.92 | 7.93 +/− 4.87 | 12.07 +/− 4.17 |
| colon | T4.ifngp.co | 1.37 +/− 1.69 | 0.75 +/− 0.32 | 1.3 +/− 0.51 | 0.05 +/− 0.1 | 1.9 +/− 0.86 | 1.86 +/− 0.86 |
| colon | T4.il10p.co | 2.71 +/− 1.02 | 0.99 +/− 0.43 | 1.92 +/− 0.61 | 0 +/− 0 | 1.32 +/− 0.47 | 1.29 +/− 0.15 |
| colon | T4.il17p.co | 0.46 +/− 0.2 | 1.87 +/− 0.79 | 1.84 +/− 1.24 | 0.83 +/− 0.25 | 1.26 +/− 0.65 | 2.69 +/− 0.25 |
| colon | T4.il22p.co | 0.04 +/− 0.08 | 1.01 +/− 0.81 | 0.14 +/− 0.1 | 1.76 +/− 1.23 | 0.78 +/− 0.54 | 0.65 +/− 0.14 |
| mln | mono.mln | 0.22 +/− 0.03 | 0.2 +/− 0.06 | 0.19 +/− 0.07 | 0.3 +/− 0.02 | 0.24 +/− 0.03 | 0.42 +/− 0.13 |
| mln | CD11bpCD11cpF4/80pMNP.mln | 1.01 +/− 0.18 | 0.56 +/− 0.13 | 1.04 +/− 0.21 | 0.28 +/− 0.1 | 0.44 +/− 0.4 | 0.57 +/− 0.02 |
| mln | CD103pCD11bmDC.mln | 0.42 +/− 0.08 | 0.58 +/− 0.2 | 0.01 +/− 0 | 0.83 +/− 0.29 | 0.58 +/− 0.14 | 0.6 +/− 0.07 |
| mln | CD103pCD11bpD C.mln | 0.44 +/− 0.09 | 1.42 +/− 0.47 | 0.02 +/− 0.01 | 1.54 +/− 0.59 | 1.76 +/− 0.57 | 1.57 +/− 0.18 |
| mln | pDC.mln | 0.24 +/− 0.08 | 0.21 +/− 0.07 | 0.19 +/− 0.06 | 0.12 +/− 0.02 | 0.14 +/− 0.02 | 0.34 +/− 0.08 |
| mln | ILC3.mln | | 0.08 +/− 0.02 | | 0.08 +/− 0.04 | 0.08 +/− 0.03 | 0.08 +/− 0.03 |
| mln | B.mln | 50.78 +/− 1.76 | 48.43 +/− 3.22 | 54 +/− 3.95 | 40.88 +/− 2.22 | 51.45 +/− 3.49 | 57.75 +/− 2.04 |
| mln | Tgd.mln | 0.54 +/− 0.04 | 0.63 +/− 0.05 | 0.41 +/− 0.06 | 0.47 +/− 0.05 | 0.56 +/− 0.1 | 1.13 +/− 0.15 |
| mln | Tab.mln | 44.4 +/− 1.78 | 47.18 +/− 2.94 | 43.88 +/− 4.11 | 43.32 +/− 1.43 | 44.9 +/− 3.97 | 37.88 +/− 2.07 |
| mln | DN(CD8mCD4mTCRp).mln | 0.41 +/− 0.05 | 0.49 +/− 0.03 | 0.57 +/− 0.07 | 0.49 +/− 0.09 | 0.65 +/− 0.09 | 0.82 +/− 0.13 |
| mln | T8.mln | 40.38 +/− 1.32 | 37.4 +/− 1.54 | 37.93 +/− 1.79 | 37.8 +/− 2.77 | 38.18 +/− 1.35 | 41.88 +/− 0.49 |
| mln | T8.Heliosp.mln | 4.54 +/− 2.21 | 2.74 +/− 0.23 | 3.98 +/− 0.11 | 2.13 +/− 0.24 | 3.43 +/− 0.31 | 4.1 +/− 0.38 |
| mln | T4.mln | 58.07 +/− 1.14 | 57.9 +/− 3.21 | 60.78 +/− 1.75 | 60.37 +/− 2.87 | 60.52 +/− 1.5 | 56.33 +/− 0.42 |
| mln | T4.FPmRorgp.mln | | 0.84 +/− 0.25 | | 0.56 +/− 0.12 | 0.64 +/− 0.29 | 0.45 +/− 0.08 |
| mln | T4.FPp.mln | 13.75 +/− 0.51 | 11.4 +/− 1.06 | 14.03 +/− 0.22 | 13.38 +/− 0.9 | 13.12 +/− 0.95 | 13.03 +/− 0.63 |
| mln | T4.FPpHeliosm.mln | 21.45 +/− 11.59 | 34.69 +/− 2.39 | 21.23 +/− 1.8 | 27.69 +/− 0.85 | 33.11 +/− 1.51 | 27.77 +/− 1.45 |
| mln | T4. FPpRorgpHelio sm.mln | | 3.38 +/− 0.91 | | 1.79 +/− 0.34 | 4.5 +/− 1.27 | 5.14 +/− 1.17 |
| mln | ILC.il22p.mln | | 0.92 +/− 0.13 | 0.19 +/− 0.06 | 0.53 +/− 0.45 | 0.28 +/− 0.19 | 0.25 +/− 0.1 |
| mln | T4.ifngp.mln | 0.4 +/− 0.13 | 0.73 +/− 0.23 | 0.73 +/− 0.09 | 0.56 +/− 0.22 | 1.09 +/− 0.22 | 1.63 +/− 0.33 |
| mln | T4.il10p.mln | 1.12 +/− 0.28 | 0.33 +/− 0.06 | 0.43 +/− 0.06 | 0.09 +/− 0.07 | 0.22 +/− 0.09 | 0.48 +/− 0.07 |
| mln | T4.il17p.mln | 0.28 +/− 0.08 | 0.56 +/− 0.12 | 1.11 +/− 0.15 | 0.3 +/− 0.03 | 0.47 +/− 0.11 | 1.06 +/− 0.09 |
| mln | T4.il22p.mln | 0.04 +/− 0.07 | 0.17 +/− 0.06 | 0.07 +/− 0.03 | 0.14 +/− 0.09 | 0.21 +/− 0.12 | 0.11 +/− 0.06 |
| pp | mono.pp | 0.7 +/− 0.14 | 0.92 +/− 0.35 | 0.33 +/− 0.04 | 1.21 +/− 0.38 | 0.77 +/− 0.12 | 0.83 +/− 0.26 |
| pp | CD11bpCD11cmF4/80pMF.pp | 0.27 +/− 0.07 | 0.08 +/− 0.03 | 0.13 +/− 0.01 | 0.1 +/− 0.05 | 0.08 +/− 0.03 | 0.18 +/− 0.05 |
| pp | CD11bpCD11cpF4/80pMNP.pp | 3.73 +/− 1.19 | 3.11 +/− 1.16 | 3.73 +/− 0.93 | 3.25 +/− 0.35 | 1.64 +/− 0.11 | 1.99 +/− 0.39 |
| pp | CD103pCD11bmDC.pp | 4.1 +/− 0.49 | 4.96 +/− 0.59 | 2.3 +/− 0.33 | 3.21 +/− 0.7 | 4.63 +/− 0.16 | 2.23 +/− 0.53 |
| pp | CD103pCD11bpDC.pp | 7.8 +/− 2.33 | 13.52 +/− 2.89 | 12.21 +/− 3.17 | 10.25 +/− 2.25 | 14.51 +/− 0.27 | 5.83 +/− 0.43 |
| pp | pDC.pp | 2.98 +/− 1.15 | 1.34 +/− 0.42 | 2.37 +/− 0.37 | 1.67 +/− 0.35 | 1.77 +/− 0.2 | 1.03 +/− 0.5 |
| pp | ILC3.pp | | 0.5 +/− 0.02 | | 0.21 +/− 0.16 | 0.45 +/− 0.07 | 0.39 +/− 0.07 |
| pp | B.pp | 82.18 +/− 3.36 | 85.6 +/− 2.06 | 80.8 +/− 3.63 | 78.75 +/− 14.8 | 85.98 +/− 2.92 | 88 +/− 0.88 |
| pp | Tgd.pp | 5.39 +/− 2.09 | 5.17 +/− 1.66 | 6.12 +/− 2.1 | 2.93 +/− 1.76 | 4.52 +/− 1.78 | 1.94 +/− 0.45 |
| pp | Tab.pp | 8.13 +/− 0.91 | 6.07 +/− 1.09 | 9.8 +/− 1.17 | 12.23 +/− 12.34 | 5.94 +/− 1.1 | 7.11 +/− 0.54 |
| pp | DN(CD8mCD4mTCRp).pp | 2.18 +/− 0.64 | 3.51 +/− 0.79 | 2.16 +/− 0.44 | 2.81 +/− 0.75 | 2.94 +/− 0.75 | 1.87 +/− 1.42 |
| pp | T8.pp | 38.03 +/− 7.33 | 39.38 +/− 4.22 | 44.28 +/− 2.96 | 37.9 +/− 3.41 | 37.57 +/− 5.08 | 32.8 +/− 0.94 |
| pp | T8.Heliosp.pp | 23.13 +/− 4.51 | 26.82 +/− 8.04 | 36.33 +/− 8.4 | 18.66 +/− 12.68 | 19.1 +/− 8.64 | 12.94 +/− 3.28 |
| pp | T4.pp | 54.25 +/− 7.99 | 52.4 +/− 2.9 | 50 +/− 3.41 | 57.18 +/− 4.03 | 54.13 +/− 4.64 | 62.65 +/− 3.14 |
| pp | T4.FPmRorgp.pp | | 1.18 +/− 0.73 | | 1.77 +/− 1.02 | 2.61 +/− 1.4 | 0.61 +/− 0.26 |
| pp | T4.FPp.pp | 15.75 +/− 2.68 | 16.47 +/− 1.91 | 19.42 +/− 4.16 | 16.98 +/− 3.92 | 22.4 +/− 2.71 | 15.77 +/− 0.53 |
| pp | T4.FPpHeliosm.pp | 13.73 +/− 0.88 | 30.94 +/− 1 | 16.25 +/− 1.73 | 21.85 +/− 1.83 | 35.09 +/− 4.98 | 27.66 +/− 3.61 |
| pp | T4.FPpRorgpHeliosm.pp | | 10.86 +/− 2.78 | | 6.51 +/− 3.59 | 12.71 +/− 3.22 | 10.59 +/− 3.07 |
| pp | ILC.il22p.pp | | 1.59 +/− 0.8 | 0.53 +/− 0.09 | 2.2 +/− 1.28 | 0.87 +/− 0.64 | 1.19 +/− 0.29 |
| pp | T4.ifngp.pp | 0.79 +/− 0.88 | 0.28 +/− 0.14 | 0.62 +/− 0.22 | 0.06 +/− 0.02 | 0.64 +/− 0.19 | 0.18 +/− 0.13 |
| pp | T4.il10p.pp | 2.65 +/− 1.22 | 1.16 +/− 0.54 | 0.76 +/− 0.51 | 0.13 +/− 0.09 | 0.37 +/− 0.11 | 0.46 +/− 0.11 |
| pp | T4.il17p.pp | 1.01 +/− 0.36 | 1.99 +/− 0.26 | 0.61 +/− 0.25 | 0.61 +/− 0.52 | 0.55 +/− 0.57 | 10.46 +/− 4.3 |
| pp | T4.il22p.pp | 1.77 +/− 0.67 | 0.26 +/− 0.15 | 0.37 +/− 0.28 | 0.21 +/− 0.19 | 0.1 +/− 0.12 | 1.05 +/− 0.54 |
| silp | mono.si | 7.21 +/− 1.51 | 5.76 +/− 0.91 | 4.81 +/− 1.78 | 2.62 +/− 1.47 | 7.38 +/− 0.5 | 6.63 +/− 1.59 |
| silp | CD11bpCD11cmF4/80pMF.si | 3.46 +/− 0.25 | 0.55 +/− 0.19 | 2.07 +/− 1.42 | 0 +/− 0 | 1.48 +/− 2.27 | 0.7 +/− 0.25 |
| silp | CD11bpCD11cpF4/80pMNP.si | 30.55 +/− 10.51 | 11.49 +/− 1.64 | 28.13 +/− 5.55 | 0.02 +/− 0.01 | 6.62 +/− 6.25 | 15.28 +/− 1.09 |
| silp | CD103pCD11bmDC.si | 12.62 +/− 1.37 | 14.38 +/− 5.98 | 6.72 +/− 3.99 | 6.46 +/− 5.08 | 23.15 +/− 10.94 | 10.35 +/− 0.6 |
| silp | CD103pCD11bpDC.si | 1.24 +/− 0.38 | 1.14 +/− 0.34 | 3.83 +/− 3.42 | 0.47 +/− 0.2 | 2.57 +/− 2.11 | 3.01 +/− 0.97 |
| silp | pDC.si | 7.78 +/− 1.77 | 9.61 +/− 3.47 | 12.93 +/− 5.01 | 3.13 +/− 0.76 | 5.4 +/− 1.66 | 5.47 +/− 2.88 |
| silp | ILC3.si | | 15.03 +/− 8.67 | | 25.07 +/− 7.31 | 18.85 +/− 9.36 | 18.48 +/− 3.6 |
| silp | B.si | 37.92 +/− 10.59 | 44.85 +/− 21.84 | 38.27 +/− 19.34 | 39.13 +/− 19.15 | 33.31 +/− 9.46 | 40.3 +/− 6.96 |
| silp | Tgd.si | 3.75 +/− 1.94 | 4.81 +/− 3.98 | 7.17 +/− 4.4 | 9.89 +/− 10.5 | 5.12 +/− 1.12 | 4.5 +/− 0.76 |
| silp | Tab.si | 31.35 +/− 9.16 | 22.52 +/− 5.63 | 31.05 +/− 8.85 | 36.03 +/− 15.89 | 29.18 +/− 6.55 | 36.37 +/− 6.89 |
| silp | DN(CD8mCD4mTCRp).si | 11.34 +/− 3.68 | 12.58 +/− 5.22 | 8.46 +/− 3.15 | 7.33 +/− 5.06 | 11.53 +/− 5.87 | 5.17 +/− 0.84 |
| silp | T8.si | 18.08 +/− 4.39 | 23.83 +/− 1.4 | 25.03 +/− 4.88 | 17.28 +/− 11.93 | 21.3 +/− 5.55 | 22.9 +/− 3.33 |
| silp | T8.Heliosp.si | 15.47 +/− 1.92 | 19.16 +/− 8.52 | 28.8 +/− 12.87 | 19.98 +/− 5.57 | 6.31 +/− 0.57 | 9.83 +/− 3.31 |
| silp | T4.si | 69.52 +/− 3.22 | 56.68 +/− 6.92 | 64.05 +/− 4.89 | 72.45 +/− 17.74 | 59.97 +/− 5.68 | 69.05 +/− 4.1 |
| silp | T4.FPmRorgp.si | | 3.12 +/− 2.18 | | 0.61 +/− 0.37 | 8.56 +/− 7.3 | 2.96 +/− 0.98 |
| silp | T4.FPp.si | 27.5 +/− 7.4 | 22.75 +/− 1.96 | 33.95 +/− 10.05 | 32.98 +/− 8.82 | 21.78 +/− 6.92 | 27.68 +/− 2.16 |
| silp | T4.FPpHeliosm.si | 7.45 +/− 2 | 27.84 +/− 10.38 | 8.61 +/− 1.28 | | 16.42 +/− 4.1 | 18.1 +/− 2.66 |
| silp | T4.FPpRorgpHeliosm.si | | 10.36 +/− 5.54 | | 5.02 +/− 1.31 | 8.91 +/− 3.57 | 8.14 +/− 1.37 |
| silp | ILC.il22p.si | | 25.63 +/− 17.82 | 10.02 +/− 3.68 | 21.55 +/− 8.77 | 20.68 +/− 10.04 | 29.72 +/− 1.33 |
| silp | T4.ifngp.si | 2.1 +/− 3.4 | 1.52 +/− 1.01 | 2.37 +/− 1.72 | 0.28 +/− 0.09 | 1.39 +/− 0.71 | 0.79 +/− 0.32 |
| silp | T4.il10p.si | 1.23 +/− 0.38 | 0.76 +/− 0.43 | 2.39 +/− 2.66 | 0.15 +/− 0.14 | 1.1 +/− 0.43 | 0.74 +/− 0.19 |
| silp | T4.il17p.si | 2.62 +/− 1.87 | 1.27 +/− 0.84 | 2.13 +/− 1.38 | 1.72 +/− 1.03 | 2.77 +/− 2.39 | 4.27 +/− 1.88 |
| silp | T4.il22p.si | 5.62 +/− 7.29 | 1.36 +/− 0.65 | 0.11 +/− 0.19 | 0.71 +/− 0.46 | 2.28 +/− 2.16 | 0.21 +/− 0.24 |
| sp | mono.slo | 2.2 +/− 0.67 | 4.11 +/− 1.18 | 4.35 +/− 0.53 | 3 64 +/− 0.4 | 3.24 +/− 1.8 | 1.99 +/− 0.34 |
| sp | CD11bpCD11cmF4/80pMF.slo | 0.99 +/− 0.25 | 0.66 +/− 0.16 | 1.16 +/− 0.83 | 0.28 +/− 0.06 | 0.64 +/− 0.41 | 0.49 +/− 0.28 |
| sp | CD11bpCD11cpF4/80pMNP.slo | 4.21 +/− 1.29 | 4.36 +/− 1.08 | 4.77 +/− 1.16 | 3.18 +/− 0.69 | 2.82 +/− 0.84 | 2.31 +/− 0.57 |
| sp | CD103pCD11bmDC.slo | 0.04 +/− 0.01 | 0.13 +/− 0.05 | 0.08 +/− 0.08 | 0.16 +/− 0.02 | 0.19 +/− 0.13 | 0.06 +/− 0.01 |
| sp | CD103pCD11bpDC.slo | 0.04 +/− 0.04 | 0.42 +/− 0.06 | 0.26 +/− 0.3 | 0.51 +/− 0.14 | 0.62 +/− 0.07 | 0.32 +/− 0.05 |
| sp | pDC.slo | 0.77 +/− 0.52 | 0.62 +/− 0.21 | 0.7 +/− 0.32 | 0.66 +/− 0.19 | 0.4 +/− 0.18 | 0.21 +/− 0.07 |
| sp | ILC3.slo | 0.04 +/− 0.03 | 0.02 +/− 0.01 | 0.02 +/− 0 | 0.02 +/− 0.01 | 0.02 +/− 0 | 0.03 +/− 0.01 |
| sp | B.slo | 68.39 +/− 5.27 | 69.08 +/− 1.54 | 68.83 +/− 3.36 | 70.35 +/− 2.48 | 68.69 +/− 4.64 | 74.55 +/− 1.14 |
| sp | Tgd.slo | 0.49 +/− 0.11 | 0.45 +/− 0.02 | 0.34 +/− 0.01 | 0.31 +/− 0.03 | 0.43 +/− 0.07 | 0.48 +/− 0.13 |
| sp | Tab.slo | 19.91 +/− 2.73 | 21.31 +/− 2.34 | 23.5 +/− 1.26 | 19.48 +/− 2.89 | 23.72 +/− 4.8 | 18.87 +/− 1.49 |
| sp | DN(CD8mCD4mTCRp).slo | 1.94 +/− 0.88 | 3.32 +/− 1.52 | 1.85 +/− 0.16 | 4.01 +/− 1.32 | 2.07 +/− 0.44 | 2.56 +/− 0.38 |
| sp | T8.slo | 30.14 +/− 7.53 | 36.21 +/− 1.17 | 33.48 +/− 3.18 | 34.93 +/− 1.4 | 36.65 +/− 0.72 | 36.7 +/− 0.62 |
| sp | T8.Heliosp.slo | 7.26 +/− 8.7 | 3.97 +/− 2.09 | 4.11 +/− 1.53 | 1.98 +/− 0.25 | 4.96 +/− 0.9 | 5.56 +/− 0.62 |
| sp | T4.slo | 54.66 +/− 16.19 | 58.39 +/− 2.28 | 62.75 +/− 2.84 | 59.63 +/− 2.1 | 60.29 +/− 2.05 | 58.82 +/− 0.71 |
| sp | T4.FPmRorgp.slo | 0.36 +/− 0.24 | 0.29 +/− 0.08 | 0.12 +/− 0.04 | 0.34 +/− 0.24 | 0.25 +/− 0.1 | 0.26 +/− 0.03 |
| sp | T4.FPp.slo | 12.11 +/− 1.99 | 13.69 +/− 1.95 | 12.47 +/− 2.5 | 12.82 +/− 0.33 | 11.96 +/− 1.29 | 13.23 +/− 0.5 |
| sp | T4.FPpHeliosm.slo | 20.1 +/− 8.51 | 25.43 +/− 3.33 | 16.58 +/− 0.86 | 22.94 +/− 0.91 | 22.6 +/− 5.16 | 19.05 +/− 0.67 |
| sp | T4.FPpRorgpHeliosm.slo | 1.08 +/− 0.68 | 0.95 +/− 0.19 | 0.8 +/− 0.32 | 1.74 +/− 0.96 | 1.48 +/− 2.31 | 1.22 +/− 0.35 |
| sp | ILC.il22p.slo | | 0.15 +/− 0.07 | 0 +/− 0 | 0.24 +/− 0.18 | 0.13 +/− 0.05 | 0.26 +/− 0.19 |
| sp | T4.ifngp.slo | 0.14 +/− 0.12 | 0.46 +/− 0.15 | 0.32 +/− 0.07 | 0.49 +/− 0.17 | 0.69 +/− 0.29 | 1.57 +/− 0.13 |
| sp | T4.il10p.slo | 0.87 +/− 0.5 | 0.18 +/− 0.08 | 1.02 +/− 0.86 | 0.25 +/− 0.17 | 0.36 +/− 0.13 | 0.23 +/− 0.06 |
| sp | T4.il17p.slo | 0.49 +/− 0.07 | 0.24 +/− 0.13 | 0.28 +/− 0.11 | 0.17 +/− 0.1 | 0.39 +/− 0.3 | 1.14 +/− 0.43 |
| sp | T4.il22p.slo | 0.02 +/− 0.04 | 0.06 +/− 0.04 | 0.34 +/− 0.09 | 0.13 +/− 0.12 | 0.12 +/− 0.05 | 0.02 +/− 0.01 |

Table 3D

| Organ | Phylum | Firmicutes | Firmicutes | Firmicutes | Firmicutes | Proteobacteria | Firmicutes |
|---|---|---|---|---|---|---|---|
| organ | Rownames (cell type in figure) | Copr.8.2.54BFAA | Cperf.ATCC13124 | Cramo.AO31 | Csord.AO32 | Ecoli.Nissle1917 | Efaec.TX0104 |
| colon | mono.co | 3.81 +/− 0.41 | 1.91 +/− 0.55 | 3.95 +/− 0.54 | 18.33 +/− 5.29 | 5.41 +/− 0.66 | 4.3 +/− 1.47 |
| colon | CD11bpCD11cmF4/80pMF.co | 0.87 +/− 0.64 | 2.23 +/− 0.58 | 0.69 +/− 0.2 | 2.06 +/− 2 | 1.54 +/− 0.28 | 3.75 +/− 1.15 |
| colon | CD11bpCD11cpF4/80pMNP.co | 2.08 +/− 0.91 | 10.48 +/− 1.38 | 8.48 +/− 3.35 | 12.96 +/− 8.88 | 16.32 +/− 3.63 | 15.69 +/− 4.14 |
| colon | CD103pCD11bmDC.co | 2.86 +/− 1.52 | 0.17 +/− 0.05 | 0.76 +/− 0.29 | 1.13 +/− 0.4 | 2 +/− 0.18 | 3.52 +/− 2.4 |
| colon | CD103pCD11bpDC.co | 2.03 +/− 1.1 | 1.84 +/− 0.52 | 2.29 +/− 0.33 | 2.65 +/− 1.71 | 2.23 +/− 0.38 | 3.11 +/− 0.61 |
| colon | pDC.co | 1.49 +/− 0.44 | 0.92 +/− 0.44 | 1.54 +/− 0.5 | 1.77 +/− 0.64 | 3.18 +/− 0.49 | 0.9 +/− 0.35 |
| colon | ILC3.co | 0.84 +/− 0.68 | 4.82 +/− 1.76 | 3.07 +/− 2.62 | 2.12 +/− 2.41 | 3.37 +/− 0.65 | 3.76 +/− 3.47 |
| colon | B.co | 48.2 +/− 8.33 | 47.48 +/− 8.41 | 58.34 +/− 11.96 | 54.42 +/− 15.88 | 58.73 +/− 3.68 | 47.62 +/− 14.65 |
| colon | Tgd.co | 2.03 +/− 0.47 | 2.65 +/− 0.7 | 2.26 +/− 1.42 | 1.34 +/− 0.87 | 3.63 +/− 0.93 | 2.84 +/− 1.65 |
| colon | Tab.co | 34.82 +/− 6.87 | 30.2 +/− 4.12 | 23.26 +/− 7.85 | 26.9 +/− 8.29 | 21.83 +/− 1.7 | 25.99 +/− 6.04 |
| colon | DN(CD8mCD4mTCRp).co | 28.07 +/− 3.71 | 32.95 +/− 4.89 | 28 +/− 9.04 | 11.59 +/− 7.52 | 31.9 +/− 4.03 | 27.98 +/− 10.58 |
| colon | T8.co | 28.82 +/− 3.04 | 23.45 +/− 1.33 | 28.35 +/− 6.99 | 24.55 +/− 7.3 | 22 +/− 4.76 | 28.59 +/− 3.94 |
| colon | T8.Heliosp.co | 57.53 +/− 9.26 | 44 +/− 15.13 | 47.5 +/− 21.69 | 17.1 +/− 16.66 | 60.1 +/− 5.73 | 52.7 +/− 13.6 |
| colon | T4.co | 39.8 +/− 2.05 | 40.87 +/− 6.03 | 40.79 +/− 12.9 | 50.48 +/− 10.21 | 42.1 +/− 6.66 | 40.29 +/− 7.79 |
| colon | T4.FPmRorgp.co | 0.78 +/− 0.14 | 0.93 +/− 0.44 | 2.87 +/− 0.55 | 5.93 +/− 4.99 | 2.51 +/− 1.75 | 3.87 +/− 1.82 |
| colon | T4.FPp.co | 26.52 +/− 10.1 | 30.32 +/− 8.17 | 28.11 +/− 11.23 | 32.02 +/− 15.37 | 30.5 +/− 3.86 | 28.55 +/− 8.5 |
| colon | T4.FPpHeliosm.co | 40.6 +/− 3.27 | 34.65 +/− 6.03 | 45.77 +/− 10.05 | 46.96 +/− 13.13 | 38.53 +/− 6.34 | 41.7 +/− 7.9 |
| colon | T4.FPpRorgpHeliosm.co | 12.4 +/− 4.56 | 9.7 +/− 3.64 | 34.45 +/− 6.06 | 29.89 +/− 21.6 | 20.2 +/− 5.46 | 23.85 +/− 7.71 |
| colon | ILC.il22p.co | 1.04 +/− 0.41 | 12.12 +/− 2.49 | 9.33 +/− 5.22 | 0.9 +/− 1.13 | 6.16 +/− 2.32 | 6.79 +/− 4.14 |
| colon | T4.ifngp.co | 1.23 +/− 0.23 | 1.09 +/− 0.13 | 0.58 +/− 0.23 | 1.55 +/− 0.35 | 1.12 +/− 0.17 | 1.03 +/− 0.64 |
| colon | T4.il10p.co | 1.98 +/− 0.42 | 0.5 +/− 0.15 | 0.38 +/− 0.12 | 0.71 +/− 0.26 | 0.38 +/− 0.18 | 0.95 +/− 1.45 |
| colon | T4.il17p.co | 2.16 +/− 0.6 | 0.85 +/− 0.18 | 1.56 +/− 0.21 | 1.33 +/− 0.51 | 2.98 +/− 1.21 | 2.04 +/− 0.91 |
| colon | T4.il22p.co | 0.21 +/− 0.12 | 0.07 +/− 0.02 | 0.21 +/− 0.19 | 0.59 +/− 0.49 | 0.33 +/− 0.18 | 0.29 +/− 0.25 |
| mln | mono.mln | 0.33 +/− 0.05 | 0.14 +/− 0.03 | 0.49 +/− 0.06 | 0.55 +/− 0.51 | 0.42 +/− 0.06 | 0.49 +/− 0.32 |
| mln | CD11bpCD11cpF4/80pMNP.mln | 0.17 +/− 0.06 | 0.18 +/− 0.03 | 0.41 +/− 0.12 | 0.61 +/− 0.27 | 0.72 +/− 0.27 | 1.19 +/− 0.77 |
| mln | CD103pCD11bmDC.mln | 0.42 +/− 0.15 | 0.01 +/− 0.01 | 0.44 +/− 0.1 | 0.47 +/− 0.09 | 0.62 +/− 0.16 | 0.7 +/− 0.25 |
| mln | CD103pCD11bpDC.mln | 0.25 +/− 0.07 | 0.39 +/− 0.14 | 1.27 +/− 0.13 | 1.2 +/− 0.27 | 1.29 +/− 0.16 | 1.88 +/− 1.12 |
| mln | pDC.mln | 0.18 +/− 0.05 | 0.25 +/− 0 | 0.15 +/− 0.05 | 0.22 +/− 0.04 | 0.17 +/− 0.03 | 0.16 +/− 0.12 |
| mln | ILC3.mln | 0.06 +/− 0.01 | 0.26 +/− 0.06 | 0.11 +/− 0.01 | 0.1 +/− 0.05 | 0.07 +/− 0.01 | 0.09 +/− 0.06 |
| mln | B.mln | 59.53 +/− 0.6 | 50.05 +/− 3.23 | 55.03 +/− 5.52 | 45.95 +/− 3.78 | 56.33 +/− 1.21 | 51.71 +/− 4.12 |
| mln | Tgd.mln | 0.48 +/− 0.02 | 0.42 +/− 0.04 | 0.42 +/− 0.07 | 0.67 +/− 0.3 | 0.42 +/− 0.08 | 0.88 +/− 0.31 |
| mln | Tab.mln | 35.75 +/− 0.54 | 42.95 +/− 2.82 | 41.25 +/− 5.26 | 40.88 +/− 6.74 | 37.5 +/− 0.87 | 41.86 +/− 2.8 |
| mln | DN(CD8mCD4mTCRp).mln | 0.85 +/− 0.09 | 0.61 +/− 0.09 | 0.55 +/− 0.05 | 0.73 +/− 0.31 | 0.79 +/− 0.06 | 0.76 +/− 0.18 |
| mln | T8.mln | 38.6 +/− 1.42 | 40.57 +/− 1.2 | 42.12 +/− 2 | 37.65 +/− 0.77 | 36.03 +/− 1.46 | 38.53 +/− 2.42 |
| mln | T8.Heliosp.mln | 2.58 +/− 0.25 | 2.04 +/− 0.05 | 3.57 +/− 0.73 | 2.49 +/− 0.78 | 4.33 +/− 0.32 | 4.08 +/− 0.87 |
| mln | T4.mln | 58.68 +/− 1.24 | 57.88 +/− 1.31 | 56.6 +/− 2.01 | 59.43 +/− 1.13 | 62.27 +/− 1.31 | 60.04 +/− 2.53 |
| mln | T4.FPmRorgp.mln | 0.19 +/− 0.07 | 0.15 +/− 0.03 | 0.42 +/− 0.03 | 0.72 +/− 0.35 | 0.36 +/− 0.08 | 0.67 +/− 0.2 |
| mln | T4.FPp.mln | 14.23 +/− 0.26 | 12.15 +/− 1.62 | 11.82 +/− 0.46 | 12.17 +/− 0.99 | 12.03 +/− 0.25 | 12.84 +/− 1.83 |
| mln | T4.FPpHeliosm.mln | 27.23 +/− 1.18 | 38.45 +/− 3.58 | 33.04 +/− 0.83 | 31.61 +/− 3.03 | 30.67 +/− 1.61 | 29.53 +/− 1.36 |
| mln | T4.FPpRorgpHeliosm.mln | 2.05 +/− 0.74 | 2.68 +/− 0.72 | 4.59 +/− 0.46 | 3.99 +/− 1.81 | 2.67 +/− 1.28 | 4.57 +/− 1.18 |
| mln | ILC.il22p.mln | 0.03 +/− 0.01 | 0.18 +/− 0.13 | 0.21 +/− 0.13 | 0.4 +/− 0.27 | 0.99 +/− 0.15 | 0.33 +/− 0.31 |
| mln | T4.ifngp.mln | 0.76 +/− 0.19 | 0.29 +/− 0.04 | 1.3 +/− 0.8 | 2.23 +/− 0.47 | 0.9 +/− 0.07 | 1.75 +/− 2.72 |
| mln | T4.il10p.mln | 0.67 +/− 0.18 | 0.17 +/− 0.18 | 0.15 +/− 0.02 | 0.23 +/− 0.05 | 0.45 +/− 0.26 | 0.42 +/− 0.33 |
| mln | T4.il17p.mln | 0.73 +/− 0.05 | 0.68 +/− 0.27 | 0.4 +/− 0.14 | 0.66 +/− 0.14 | 0.87 +/− 0.11 | 0.67 +/− 0.23 |
| mln | T4.il22p.mln | 0.09 +/− 0.04 | 0.02 +/− 0.01 | 0.19 +/− 0.11 | 0.1 +/− 0.06 | 0.09 +/− 0.04 | 0.13 +/− 0.09 |
| pp | mono.pp | 0.7 +/− 0.12 | 0.35 +/− 0.15 | 1.25 +/− 0.49 | 1.34 +/− 1.08 | 0.58 +/− 0.19 | 1.41 +/− 0.67 |
| pp | CD11bpCD11cmF4/80pMF.pp | 0.18 +/− 0.09 | 0.13 +/− 0.06 | 0.19 +/− 0.11 | 0.28 +/− 0.2 | 0.07 +/− 0.03 | 0.52 +/− 0.29 |
| pp | CD11bpCD11cpF4/80pMNP.pp | 0.67 +/− 0.15 | 1.17 +/− 0.38 | 3.16 +/− 0.66 | 3.02 +/− 1.14 | 1.87 +/− 0.08 | 2.85 +/− 1.61 |
| pp | CD103pCD11bmDC.pp | 1.95 +/− 0.28 | 0.68 +/− 0.15 | 2.84 +/− 0.1 | 3.4 +/− 1.03 | 3.96 +/− 1.08 | 3.81 +/− 1.78 |
| pp | CD103pCD11bpDC.pp | 4.73 +/− 2.02 | 3.37 +/− 1.11 | 11.72 +/− 2.38 | 13.08 +/− 5.14 | 10.28 +/− 3.65 | 9.73 +/− 5.58 |
| pp | pDC.pp | 2.25 +/− 0.24 | 0.76 +/− 0.25 | 1.75 +/− 0.27 | 1.7 +/− 0.32 | 2.18 +/− 0.23 | 2.52 +/− 1.46 |
| pp | ILC3.pp | 0.1 +/− 0.03 | 0.66 +/− 0.14 | 0.34 +/− 0.09 | 0.49 +/− 0.12 | 0.39 +/− 0.13 | 0.43 +/− 0.42 |
| pp | B.pp | 84.5 +/− 0.96 | 84.57 +/− 0.38 | 81.98 +/− 3.97 | 86.1 +/− 2.66 | 78.53 +/− 6.45 | 80.47 +/− 5.25 |
| pp | Tgd.pp | 4.18 +/− 1.2 | 1.3 +/− 0.29 | 3.31 +/− 1.27 | 3.9 +/− 1.87 | 7.9 +/− 4.54 | 4.79 +/− 2.73 |
| pp | Tab.pp | 6.39 +/− 0.27 | 11.1 +/− 0.44 | 9.86 +/− 2.55 | 6.27 +/− 0.49 | 9.77 +/− 1.61 | 8.74 +/− 2.21 |
| pp | DN(CD8mCD4mTCRp)pp | 3.23 +/− 2.03 | 1.73 +/− 0.28 | 2.48 +/− 0.59 | 5.35 +/− 0.24 | 2.93 +/− 0.41 | 3.73 +/− 3.56 |
| pp | T8.pp | 29.37 +/− 2.43 | 32.13 +/− 1.16 | 29.7 +/− 3.07 | 33.1 +/− 4.74 | 46.33 +/− 12.31 | 30.74 +/− 5.87 |
| pp | T8.Heliosp.pp | 21.55 +/− 6.91 | 9.02 +/− 3.39 | 18.25 +/− 6.1 | 23.07 +/− 7.76 | 35.17 +/− 9.56 | 28.68 +/− 6.58 |
| pp | T4.pp | 60.93 +/− 3.8 | 63.33 +/− 1.7 | 64.65 +/− 3.25 | 58.15 +/− 5.53 | 47.97 +/− 12.63 | 58.41 +/− 9.02 |
| pp | T4.FPmRorgp.pp | 0.46 +/− 0.22 | 0.52 +/− 0.28 | 0.93 +/− 0.3 | 2.13 +/− 0.23 | 0.65 +/− 0.37 | 1.86 +/− 0.69 |
| pp | T4.FPp.pp | 13.67 +/− 1.98 | 14.5 +/− 0.66 | 17.33 +/− 1.79 | 18.93 +/− 1.81 | 16.63 +/− 1.6 | 17.77 +/− 2.3 |
| pp | T4.FPpHeliosm.pp | 26.37 +/− 3.43 | 31.93 +/− 2.94 | 37.13 +/− 5.29 | 25 +/− 4.87 | 24.16 +/− 0.69 | 30.62 +/− 6.16 |
| pp | T4.FPpRorgpHeliosm.pp | 3.9 +/− 1.81 | 4.56 +/− 0.73 | 18.8 +/− 4.56 | 14.05 +/− 2.57 | 7.93 +/− 1.24 | 11.22 +/− 5.49 |
| pp | ILC.il22p.pp | 0.49 +/− 0.81 | 17 +/− 5.33 | 0.7 +/− 0.15 | 1.51 +/− 1.92 | 0.66 +/− 0.11 | 1.23 +/− 1.1 |
| pp | T4.ifngp.pp | 0.18 +/− 0.06 | 0.71 +/− 0.15 | 0.17 +/− 0.08 | 1.64 +/− 1.33 | 0.97 +/− 0.72 | 0.52 +/− 0.44 |
| pp | T4.il10p.pp | 2.16 +/− 1.85 | 0.17 +/− 0.01 | 0.77 +/− 0.1 | 1.12 +/− 1.11 | 0.46 +/− 0.19 | 0.54 +/− 0.59 |
| pp | T4.il17p.pp | 4.17 +/− 1.52 | 1.26 +/− 0.27 | 1.37 +/− 0.29 | 2.07 +/− 0.55 | 4.16 +/− 0.27 | 2.5 +/− 2.61 |
| pp | T4.il22p.pp | 0.49 +/− 0.84 | 0.15 +/− 0.04 | 0.43 +/− 0.08 | 0.36 +/− 0.39 | 0.56 +/− 0.32 | 0.47 +/− 0.37 |
| silp | mono.si | 7.33 +/− 1.08 | 4.75 +/− 1.94 | 14.74 +/− 3.35 | 9.76 +/− 5.74 | 6.63 +/− 1.63 | 7.23 +/− 2.58 |
| silp | CD11bpCD11cmF4/80pMF.si | 0.55 +/− 0.16 | 0.36 +/− 0.18 | 0.74 +/− 0.49 | 2.1 +/− 2.4 | 0.37 +/− 0.18 | 4.11 +/− 1.84 |
| silp | CD11bpCD11cpF4/80pMNP.si | 0.86 +/− 0.35 | 6.39 +/− 1.43 | 19.81 +/− 5.69 | 15.33 +/− 9.19 | 14.12 +/− 3.72 | 19.73 +/− 2.67 |
| silp | CD103pCD11bmDC.si | 18.51 +/− 3.06 | 9.94 +/− 3.22 | 7.23 +/− 3.17 | 7.3 +/− 2.56 | 7.63 +/− 1.73 | 7.18 +/− 2.4 |
| silp | CD103pCD11bpDC.si | 0.78 +/− 0.54 | 4.01 +/− 1.49 | 1.55 +/− 0.62 | 1.54 +/− 0.67 | 1.71 +/− 0.52 | 1.5 +/− 1.96 |
| silp | pDC.si | 4.78 +/− 1.23 | 8.54 +/− 1.3 | 5.77 +/− 0.65 | 5.37 +/− 3.76 | 7.61 +/− 0.95 | 5.45 +/− 1.54 |
| silp | ILC3.si | 13.74 +/− 5.37 | 12.23 +/− 5.18 | 19.62 +/− 0.32 | 12.32 +/− 11.35 | 10.71 +/− 2.23 | 12.39 +/− 8.47 |
| silp | B.si | 38.05 +/− 15.79 | 67.88 +/− 10.4 | 31.45 +/− 4.55 | 56.12 +/− 23.98 | 37.07 +/− 3.76 | 26.67 +/− 11.88 |
| silp | Tgd.si | 3.45 +/− 1.17 | 1.92 +/− 0.94 | 2.75 +/− 0.69 | 4.03 +/− 2.82 | 2.58 +/− 0.36 | 9.11 +/− 6.24 |
| silp | Tab.si | 3 5.4 +/− 11.49 | 17.35 +/− 4.86 | 38.68 +/− 5.14 | 20.52 +/− 11.09 | 33.23 +/− 1.4 | 33.97 +/− 7.73 |
| silp | DN(CD8mCD4mTCRp).si | 16.8 +/− 3.53 | 6.75 +/− 2.17 | 13.47 +/− 3.24 | 9.94 +/− 5.04 | 5.48 +/− 1.07 | 8.84 +/− 5.92 |
| silp | T8.si | 17.52 +/− 1.3 | 30.67 +/− 1.63 | 21 +/− 2.55 | 22.23 +/− 3.16 | 26.77 +/− 1.46 | 21.69 +/− 6.46 |
| silp | T8.Heliosp.si | 12.25 +/− 2.25 | 11.11 +/− 6.51 | 14.7 +/− 1.84 | 11.21 +/− 6.72 | 7.88 +/− 0.86 | 25.25 +/− 14.88 |
| silp | T4.si | 59.7 +/− 4.76 | 60.5 +/− 3.3 | 63.08 +/− 3.23 | 68.43 +/− 1.86 | 66.47 +/− 0.29 | 62.2 +/− 9.15 |
| silp | T4.FPmRorgp.si | 1.9 +/− 0.84 | 1.48 +/− 0.62 | 5.02 +/− 0.95 | 11.47 +/− 2.47 | 3.07 +/− 0.69 | 5.31 +/− 3.68 |
| silp | T4.FPp.si | 29.05 +/− 6.2 | 16.95 +/− 1.79 | 27.45 +/− 6.5 | 23.33 +/− 7.79 | 19.97 +/− 1.3 | 23.56 +/− 5.47 |
| silp | T4.FPpHeliosm.si | 20.69 +/− 3.72 | 20.88 +/− 7.39 | 24.54 +/− 2.05 | 25.48 +/− 8.29 | 18.47 +/− 1.9 | 22.56 +/− 6.33 |
| silp | T4.FPpRorgpHeliosm.si | 3.56 +/− 0.8 | 4.79 +/− 2.58 | 15.1 +/− 1.6 | 14.25 +/− 5.13 | 5.3 +/− 1.17 | 14.6 +/− 7.09 |
| silp | ILC.il22p.si | 5.43 +/− 3.5 | 23.86 +/− 15.06 | 24.51 +/− 5.73 | 14.06 +/− 15.99 | 16.86 +/− 1.59 | 28.22 +/− 15.07 |
| silp | T4.ifngp.si | 0.69 +/− 0.31 | 1.77 +/− 0.5 | 0.82 +/− 0.48 | 1.27 +/− 0.26 | 0.71 +/− 0.28 | 0.93 +/− 0.73 |
| silp | T4.il10p.si | 2.14 +/− 0.51 | 0.5 +/− 0.28 | 0.53 +/− 0.05 | 0.94 +/− 0.34 | 0.69 +/− 0.24 | 0.75 +/− 0.35 |
| silp | T4.il17p.si | 2.84 +/− 1.81 | 0.81 +/− 0.19 | 1.67 +/− 0.23 | 2.48 +/− 2.12 | 2.34 +/− 0.17 | 7.71 +/− 4.25 |
| silp | T4.il22p.si | 0.13 +/− 0.05 | 0.38 +/− 0.13 | 2.01 +/− 0.81 | 0.42 +/− 0.57 | 0.82 +/− 0.34 | 0.8 +/− 0.51 |
| sp | mono.slo | 2.95 +/− 0.32 | 2.78 +/− 0.6 | 4.58 +/− 0.95 | 9.46 +/− 5.89 | 7.55 +/− 0.92 | 8.93 +/− 10.41 |
| sp | CD11bpCD11cmF4/80pMF.slo | 0.79 +/− 0.42 | 0.63 +/− 0.16 | 0.36 +/− 0.16 | 2.32 +/− 1.46 | 1.44 +/− 0.41 | 3.18 +/− 3.52 |
| sp | CD11bpCD11cpF4/80pMNP.slo | 1.48 +/− 0.8 | 1.42 +/− 0.36 | 3.21 +/− 2.08 | 5.28 +/− 3.57 | 4.7 +/− 0.56 | 5.88 +/− 2.89 |
| sp | CD103pCD11bmDC.slo | 0.12 +/− 0.13 | 0.02 +/− 0.01 | 1.05 +/− 1.84 | 0.1 +/− 0.08 | 0.04 +/− 0.02 | 0.05 +/− 0.02 |
| sp | CD103pCD11bpDC.slo | 0.13 +/− 0.09 | 0.18 +/− 0.02 | 0.58 +/− 0.5 | 0.45 +/− 0.24 | 0.11 +/− 0.04 | 0.56 +/− 0.62 |
| sp | pDC.slo | 0.3 +/− 0.13 | 0.74 +/− 0.15 | 0.33 +/− 0.03 | 0.72 +/− 0.32 | 0.75 +/− 0.15 | 0.77 +/− 0.53 |
| sp | ILC3.slo | 0.02 +/− 0.01 | 0.04 +/− 0.01 | 0.12 +/− 0.21 | 0.02 +/− 0.03 | 0.02 +/− 0 | 0.03 +/− 0.02 |
| sp | B.slo | 71 +/− 1.1 | 69.17 +/− 1.35 | 69.65 +/− 3.57 | 70.52 +/− 2.33 | 76.2 +/− 2.51 | 56.79 +/− 22.95 |
| sp | Tgd.slo | 0.38 +/− 0.04 | 0.36 +/− 0.04 | 0.64 +/− 0.25 | 0.42 +/− 0.06 | 0.27 +/− 0.02 | 0.51 +/− 0.15 |
| sp | Tab.slo | 22.4 +/− 1.34 | 20.93 +/− 0.76 | 21.69 +/− 3.18 | 21.22 +/− 1.02 | 17.53 +/− 2.31 | 31.85 +/− 18.57 |
| sp | DN(CD8mCD4mTCRp).slo | 3.74 +/− 0.71 | 2.53 +/− 0.15 | 3.56 +/− 1.08 | 2.24 +/− 0.73 | 2.08 +/− 0.04 | 2.51 +/− 1.88 |
| sp | T8.slo | 35.22 +/− 0.5 | 34.5 +/− 0.84 | 34.23 +/− 2.88 | 34.52 +/− 0.59 | 30.4 +/− 3.38 | 34.09 +/− 1.82 |
| sp | T8.Heliosp.slo | 3.19 +/− 0.27 | 3.96 +/− 0.21 | 4.82 +/− 0.84 | 2.41 +/− 0.94 | 7.01 +/− 0.63 | 4.87 +/− 2.25 |
| sp | T4.slo | 58.97 +/− 0.43 | 60.4 +/− 0.84 | 61.5 +/− 3.49 | 61.22 +/− 1 | 66 +/− 3.2 | 57.77 +/− 5.9 |
| sp | T4.FPmRorgp.slo | 0.13 +/− 0.05 | 0.13 +/− 0.06 | 0.32 +/− 0.09 | 0.19 +/− 0.14 | 0.37 +/− 0.1 | 0.38 +/− 0.16 |
| sp | T4.FPp.slo | 13.75 +/− 0.58 | 14.95 +/− 0.26 | 12.19 +/− 2.15 | 13.45 +/− 0.83 | 13.13 +/− 0.72 | 14.16 +/− 1.63 |
| sp | T4.FPpHeliosm.slo | 23.12 +/− 1 | 28.4 +/− 1.71 | 23.42 +/− 8.14 | 21.73 +/− 1.81 | 23.4 +/− 0.81 | 23.59 +/− 3.18 |
| sp | T4.FPpRorgpHeliosm.slo | 0.35 +/− 0.31 | 1.5 +/− 0.37 | 1.4 +/− 0.98 | 1.13 +/− 1.02 | 1.13 +/− 0.37 | 1.17 +/− 0.5 |
| sp | ILC.il22p.slo | 0.12 +/− 0.03 | 0.02 +/− 0.02 | 0.3 +/− 0.07 | 0.06 +/− 0.06 | 0.27 +/− 0.18 | 0.26 +/− 0.26 |
| sp | T4.ifngp.slo | 0.37 +/− 0.06 | 1.11 +/− 0.66 | 1.66 +/− 0.27 | 0.62 +/− 0.18 | 0.84 +/− 0.2 | 1.03 +/− 0.49 |
| sp | T4.il10p.slo | 0.25 +/− 0.06 | 0.11 +/− 0.04 | 0.24 +/− 0.07 | 0.6 +/− 0.37 | 3.77 +/− 2.97 | 0.22 +/− 0.21 |
| sp | T4.il17p.slo | 0.21 +/− 0.09 | 0.14 +/− 0.08 | 0.53 +/− 0.26 | 1.72 +/− 0.33 | 0.49 +/− 0.38 | 1.15 +/− 0.74 |
| sp | T4.il22p.slo | 0.15 +/− 0.15 | 0.02 +/− 0.01 | 0.07 +/− 0.02 | 0.14 +/− 0.03 | 0.16 +/− 0.1 | 0.18 +/− 0.16 |

Table 3E

| Organ | Phylum | Firmicutes | Fusobacteria | Fusobacteria | Proteobacteria | Proteobacteria | Firmicutes |
|---|---|---|---|---|---|---|---|
| organ | Rownames (cell type in figure) | Efaec.TX1330 | Fvari.AO16 | Fnucl.F0419 | Hpylo.ATCC700392 | Kleb.sp.4.1.44FAA | Lach.2.1.58FAA |
| colon | mono.co | 3.13 +/− 0.48 | 4.78 +/− 2.27 | 3.1 +/− 0.46 | 3.1 +/− 1 | 3.93 +/− 0.72 | 3.38 +/− 1.07 |
| colon | CD11bpCD11cmF4/80pMF.co | 1.57 +/− 0.86 | 1.78 +/− 1.53 | 1.27 +/− 0.28 | 2.21 +/− 0.56 | 1.39 +/− 0.98 | 0.48 +/− 0.39 |
| colon | CD11bpCD11cpF4/80pMNP.co | 11.25 +/− 2.93 | 9.96 +/− 6.98 | 12.73 +/− 3.37 | 11.5 +/− 0.75 | 8.45 +/− 0.35 | 7.2 +/− 4.88 |
| colon | CD103pCD11bmDC.c 0 | 2.64 +/− 0.65 | 3.49 +/− 1.17 | 3.61 +/− 0.8 | 0.78 +/− 0.17 | 1.13 +/− 0.18 | 7.44 +/− 10.89 |
| colon | CD103pCD11bpDC.co | 2.98 +/− 0.04 | 2.99 +/− 1.02 | 3 +/− 0.79 | 2.65 +/− 0 | 3.23 +/− 0.9 | 1.96 +/− 0.65 |
| colon | pDC.co | 0.93 +/− 0.35 | 1.92 +/− 1.28 | 1.48 +/− 0.28 | 0.8 +/− 0.24 | 1.18 +/− 0.36 | 2.58 +/− 1.97 |
| colon | ILC3.co | 1.24 +/− 0.33 | 2.79 +/− 0.98 | 2.6 +/− 1.08 | 1.51 +/− 1.13 | 1.97 +/− 0.86 | 1.67 +/− 0.45 |
| colon | B.co | 47.43 +/− 7.73 | 38.39 +/− 14.74 | 64.7 +/− 5.09 | 43.3 +/− 19.23 | 49.27 +/− 17.63 | 47.5 +/− 13.64 |
| colon | Tgd.co | 2.27 +/− 0.51 | 3.57 +/− 1.15 | 1.52 +/− 0.35 | 2.25 +/− 1.1 | 2.98 +/− 1.21 | 2.85 +/− 1.26 |
| colon | Tab.co | 34.13 +/− 5.3 | 26.16 +/− 7.3 | 20.32 +/− 2.58 | 32.8 +/− 9.05 | 35.37 +/− 13.49 | 31.14 +/− 5.92 |
| colon | DN(CD8mCD4mTCRp).co | 33.08 +/− 3.37 | 45.87 +/− 7.53 | 25.83 +/− 3.65 | 31.3 +/− 8.2 | 28.07 +/− 6.92 | 29.59 +/− 12.25 |
| colon | T8.co | 29.02 +/− 0.97 | 21.75 +/− 6.57 | 24.1 +/− 1.72 | 26.85 +/− 0.78 | 31.47 +/− 4.84 | 24.09 +/− 6.6 |
| colon | T8.Heliosp.co | | 68.39 +/− 9.84 | 30.8 +/− 10.53 | 51.3 +/− 11.74 | 56.67 +/− 16.7 | 47.77 +/− 14.96 |
| colon | T4.co | 33.67 +/− 4.76 | 29.7 +/− 4.47 | 48.32 +/− 4.1 | 39.2 +/− 7.92 | 36.17 +/− 11.72 | 40.91 +/− 10.74 |
| colon | T4.FPmRorgp.co | 1.99 +/− 0.44 | 4.53 +/− 3.23 | 2.16 +/− 0.51 | 0.75 +/− 0.09 | 3.63 +/− 1.79 | 0.64 +/− 0.32 |
| colon | T4.FPp.co | 18.4 +/− 1.99 | 39.44 +/− 7.42 | 22.13 +/− 2.27 | 23.25 +/− 7.85 | 13.8 +/− 0.75 | 21.74 +/− 6.02 |
| colon | T4.FPpHeliosm.co | 35.82 +/− 10.71 | 44.07 +/− 3.97 | 46.47 +/− 4.17 | 20.19 +/− 2.48 | 28.01 +/− 4.54 | 34.94 +/− 10.22 |
| colon | T4.FPpRorgpHeliosm.co | 11.62 +/− 5.4 | 21.22 +/− 5.15 | 25.35 +/− 7.1 | 4.69 +/− 0.22 | 6.75 +/− 3.09 | 16.46 +/− 6.51 |
| colon | ILC.il22p.co | 1.71 +/− 0.68 | 7.21 +/− 4.83 | 4.92 +/− 6.1 | 3.75 +/− 0.85 | 1.61 +/− 0.75 | 4.29 +/− 3.72 |
| colon | T4.ifngp.co | 0.53 +/− 0.19 | 0.6 +/− 0.48 | 0.67 +/− 0.12 | 1.5 +/− 0.2 | 0.84 +/− 0.46 | 0.33 +/− 0.32 |
| colon | T4.il10p.co | 4.52 +/− 1.16 | 0.54 +/− 0.28 | 0.45 +/− 0.36 | 1.6 +/− 0.21 | 0.62 +/− 0.1 | 0.48 +/− 0.33 |
| colon | T4.il17p.co | 1.79 +/− 0.23 | 1.76 +/− 0.87 | 1.23 +/− 0.37 | 0.82 +/− 0.07 | 2.75 +/− 0.22 | 1.92 +/− 1.66 |
| colon | T4.il22p.co | 0.25 +/− 0.17 | 0.1 +/− 0.12 | 0.56 +/− 0.15 | 0.17 +/− 0.04 | 0.09 +/− 0.1 | 0.07 +/− 0.11 |
| mln | mono.mln | 0.37 +/− 0.04 | 0.5 +/− 0.2 | 0.22 +/− 0.01 | 0.12 +/− 0.02 | 0.48 +/− 0.11 | 0.17 +/− 0.05 |
| mln | CD11bpCD11cpF4/80pMNP.mln | 0.73 +/− 0.46 | 0.91 +/− 0.21 | 0.37 +/− 0.02 | 0.9 +/− 0.39 | 0.31 +/− 0.09 | 0.82 +/− 0.28 |
| mln | CD103pCD11bmDC.mln | 0.89 +/− 0.16 | 0.67 +/− 0.18 | 0.8 +/− 0.21 | 0.93 +/− 0.16 | 0.43 +/− 0.08 | 0.95 +/− 0.3 |
| mln | CD103pCD11bpDC.mln | 0.71 +/− 0.2 | 0.72 +/− 0.1 | 2.01 +/− 0.55 | 1.8 +/− 0.33 | 1 +/− 0.09 | 0.55 +/− 0.16 |
| mln | pDC.mln | 0.36 +/− 0.09 | 0.16 +/− 0.07 | 0.16 +/− 0.02 | 0.08 +/− 0 | 0.27 +/− 0.05 | 0.15 +/− 0.04 |
| mln | ILC3.mln | 0.08 +/− 0.01 | 0.08 +/− 0.02 | 0.1 +/− 0.01 | 0.04 +/− 0 | 0.07 +/− 0.01 | 0.08 +/− 0.01 |
| mln | B.mln | 54.88 +/− 4.35 | 51.63 +/− 5.73 | 54.63 +/− 3.39 | 56.75 +/− 3.61 | 50.6 +/− 1.18 | 52.8 +/− 3.24 |
| mln | Tgd.mln | 0.49 +/− 0.04 | 0.58 +/− 0.14 | 0.48 +/− 0.04 | 0.31 +/− 0.02 | 0.78 +/− 0.06 | 0.56 +/− 0.05 |
| mln | Tab.mln | 39.5 +/− 4.67 | 40.61 +/− 5.58 | 42.18 +/− 3.26 | 39.25 +/− 3.75 | 45.37 +/− 1.05 | 40.93 +/− 2.6 |
| mln | DN(CD8mCD4mTCRp).mln | 0.77 +/− 0.27 | 1.13 +/− 0.55 | 0.66 +/− 0.06 | 0.5 +/− 0.03 | 0.83 +/− 0.19 | 0.5 +/− 0.05 |
| mln | T8.mln | 39.62 +/− 1.72 | 36.87 +/− 2.64 | 40.77 +/− 1.86 | 42.05 +/− 0.21 | 44.33 +/− 0.76 | 39.07 +/− 2.56 |
| mln | T8.Heliosp.mln | | 2.39 +/− 0.38 | 1.66 +/− 0.06 | 2.18 +/− 0.08 | 2.25 +/− 0.32 | 2.97 +/− 0.14 |
| mln | T4.mln | 58.12 +/− 1.6 | 59.66 +/− 2.14 | 57.32 +/− 1.86 | 56.25 +/− 0.49 | 53.97 +/− 0.61 | 58.98 +/− 2.67 |
| mln | T4.FPmRorgp.mln | 0.33 +/− 0.04 | 2.81 +/− 2.78 | 0.39 +/− 0.09 | 0.19 +/− 0 | 0.33 +/− 0.16 | 0.21 +/− 0.02 |
| mln | T4.FPp.mln | 12.92 +/− 0.4 | 12.25 +/− 1.91 | 12.33 +/− 0.36 | 14.95 +/− 1.91 | 13.5 +/− 0.7 | 13.52 +/− 0.28 |
| mln | T4.FPpHeliosm.mln | 27.31 +/− 2 | 33.24 +/− 1.5 | 34.37 +/− 1.05 | 30.39 +/− 0.01 | 32.23 +/− 0.75 | 27.38 +/− 1.15 |
| mln | T4.FPpRorgpHeliosm.mln | 1.98 +/− 0.52 | 5.5 +/− 1.42 | 3.02 +/− 0.56 | 1.23 +/− 0.08 | 1.93 +/− 0.69 | 2.01 +/− 0.29 |
| mln | ILC.il22p.mln | 0.12 +/− 0.12 | 0.71 +/− 0.44 | 0.48 +/− 0.13 | 0.35 +/− 0.19 | 0.2 +/− 0.29 | 0.05 +/− 0.02 |
| mln | T4.ifngp.mln | 0.62 +/− 0.21 | 1.24 +/− 0.33 | 0.51 +/− 0.18 | 1.78 +/− 0.57 | 0.53 +/− 0.11 | 0.33 +/− 0.19 |
| mln | T4.il10p.mln | 2.83 +/− 0.6 | 1.14 +/− 0.2 | 0.02 +/− 0.01 | 0.15 +/− 0 | 0.11 +/− 0.1 | 0.08 +/− 0.03 |
| mln | T4.il17p.mln | 0.47 +/− 0.08 | 1.42 +/− 0.51 | 0.51 +/− 0.1 | 0.15 +/− 0.01 | 0.56 +/− 0.19 | 0.55 +/− 0.04 |
| mln | T4.il22p.mln | 0.04 +/− 0.03 | 2 +/− 1.19 | 0.08 +/− 0.03 | 0.07 +/− 0 | 0.02 +/− 0.02 | 0.04 +/− 0.02 |
| pp | mono.pp | 1.27 +/− 0.46 | 0.76 +/− 0.52 | 0.64 +/− 0.08 | 0.23 +/− 0.11 | 1.04 +/− 0.2 | 0.53 +/− 0.15 |
| pp | CD11bpCD11cmF4/80pMF.pp | 0.13 +/− 0.02 | 0.14 +/− 0.08 | 0.14 +/− 0.05 | 0.14 +/− 0.04 | 0.38 +/− 0.06 | 0.05 +/− 0.02 |
| pp | CD11bpCD11cpF4/80pMNP.pp | 1.62 +/− 0.86 | 1.08 +/− 0.77 | 2.83 +/− 0.85 | 1.19 +/− 0.88 | 3.35 +/− 0.41 | 0.87 +/− 0.12 |
| pp | CD103pCD11bmDC.p | 2.66 +/− 0.85 | 1.21 +/− 0.38 | 4.11 +/− 0.68 | 2.27 +/− 0.33 | 2.39 +/− 0.07 | 2.03 +/− 0.8 |
| pp | CD103pCD11bpDC.pp | 4.93 +/− 1.6 | 1.21 +/− 0.23 | 15.89 +/− 3.23 | 7.22 +/− 1.13 | 4.04 +/− 0.69 | 5.65 +/− 2.84 |
| pp | pDC.pp | 2.1 +/− 0.71 | 0.69 +/− 0.07 | 1.96 +/− 0.51 | 0.53 +/− 0.25 | 3.34 +/− 1.41 | 0.83 +/− 0.44 |
| pp | ILC3.pp | 0.19 +/− 0.06 | 0.25 +/− 0.11 | 0.24 +/− 0.1 | 0.43 +/− 0 | 0.27 +/− 0.03 | 0.29 +/− 0.12 |
| pp | B.pp | 80.47 +/− 8.04 | 81.53 +/− 8.12 | 87.55 +/− 3.52 | 78.2 +/− 1.7 | 87.83 +/− 1.32 | 85.84 +/− 2.72 |
| pp | Tgd.pp | 2.95 +/− 1.14 | 1.39 +/− 0.6 | 3.16 +/− 1.87 | 3.64 +/− 2.41 | 2.21 +/− 1.28 | 2.43 +/− 1.15 |
| pp | Tab.pp | 8.02 +/− 1.73 | 8.15 +/− 2.3 | 5.7 +/− 1.62 | 7.52 +/− 0.63 | 7.55 +/− 0.93 | 7.93 +/− 1.62 |
| pp | DN(CD8mCD4mTCRp)pp | 4.96 +/− 1.8 | 2.76 +/− 2.67 | 3.6 +/− 0.71 | 6.17 +/− 3.98 | 2.35 +/− 0.62 | 7.72 +/− 6.65 |
| pp | T8.pp | 33.1 +/− 2.93 | 23.46 +/− 3.4 | 33.52 +/− 4.05 | 34.85 +/− 2.76 | 31.7 +/− 4.1 | 31.71 +/− 3.5 |
| pp | T8.Heliosp.pp | | 12.85 +/− 6.25 | 18.3 +/− 5.46 | 25.05 +/− 4.45 | 8.52 +/− 3.94 | 20.99 +/− 14.05 |
| pp | T4.pp | 56.13 +/− 4.71 | 68.64 +/− 4.38 | 56.97 +/− 5.31 | 22.7 +/− 5.8 | 59.9 +/− 3.97 | 57.29 +/− 10.05 |
| pp | T4.FPmRorgp.pp | 0.93 +/− 0.31 | 2.61 +/− 2.46 | 1.29 +/− 0.72 | 0.52 +/− 0.37 | 0.36 +/− 0.32 | 0.51 +/− 0.38 |
| pp | T4.FPp.pp | 17.27 +/− 5.55 | 11.34 +/− 1.85 | 17.37 +/− 3.94 | 31.35 +/− 3.89 | 15.93 +/− 0.35 | 17.89 +/− 3.03 |
| pp | T4.FPpHeliosm.pp | 17.32 +/− 3.64 | 27.84 +/− 3.61 | 24.86 +/− 1.77 | 29.63 +/− 5.71 | 20.91 +/− 1.83 | 22.1 +/− 2.8 |
| pp | T4.FPpRorgpHeliosm.pp | 3.25 +/− 2.28 | 5.5 +/− 2.94 | 9.89 +/− 1.87 | 5.93 +/− 2.04 | 4.31 +/− 1.95 | 5.31 +/− 2.01 |
| pp | ILC.il22p.pp | 0.58 +/− 0.18 | 0.48 +/− 0.35 | 1.47 +/− 0.84 | 0.94 +/− 0.03 | 1.35 +/− 0.84 | 5.55 +/− 6.51 |
| pp | T4.ifngp.pp | 0.11 +/− 0.15 | 0.09 +/− 0.07 | 0.05 +/− 0.03 | 1.74 +/− 0.3 | 0.06 +/− 0.05 | 0.29 +/− 0.08 |
| pp | T4.il10p.pp | 6.8 +/− 5.85 | 1.2 +/− 0.24 | 0.02 +/− 0.01 | 1.25 +/− 0.7 | 0.11 +/− 0.11 | 0.18 +/− 0.07 |
| pp | T4.il17p.pp | 2.61 +/− 0.92 | 0.89 +/− 0.61 | 2.18 +/− 0.66 | 0.35 +/− 0.5 | 3.95 +/− 1.35 | 9.64 +/− 3.17 |
| pp | T4.il22p.pp | 0.02 +/− 0.03 | 0.09 +/− 0.1 | 0.14 +/− 0.04 | 0.22 +/− 0.31 | 0.1 +/− 0.09 | 0.15 +/− 0.06 |
| silp | mono.si | 7.15 +/− 3.86 | | 5.43 +/− 1.31 | 6.46 +/− 0.95 | 6.03 +/− 1.22 | 5.15 +/− 1.79 |
| silp | CD11bpCD11cmF4/80pMF.si | 0.04 +/− 0.02 | | 1.49 +/− 0.53 | 3.25 +/− 0.82 | 0.63 +/− 0.18 | 0.05 +/− 0.05 |
| silp | CD11bpCD11cpF4/80pMNP.si | 2.11 +/− 2.31 | | 27.57 +/− 3.48 | 19.21 +/− 3.91 | 11.92 +/− 1.01 | 4.88 +/− 2.53 |
| silp | CD103pCD11bmDC.si | 17.6 +/− 9.07 | | 10.19 +/− 1.94 | 3.68 +/− 0.89 | 7.22 +/− 2.39 | 7.41 +/− 4.96 |
| silp | CD103pCD11bpDC.si | 0.99 +/− 0.45 | | 4.59 +/− 1.25 | 1.96 +/− 1.28 | 1.7 +/− 0.36 | 2.77 +/− 3.32 |
| silp | pDC.si | 4.33 +/− 2.16 | | 7.85 +/− 0.55 | 7.63 +/− 2.25 | 5.62 +/− 3.74 | 1.9 +/− 1 |
| silp | ILC3.si | 8.63 +/− 4.9 | | 36.52 +/− 5.48 | 24.64 +/− 11.98 | 19.43 +/− 12.64 | 20.67 +/− 5.85 |
| silp | B.si | 40.13 +/− 13.87 | | 26.2 +/− 5.31 | 52 +/− 17.68 | 40.2 +/− 23.73 | 31.66 +/− 11.46 |
| silp | Tgd.si | 1.93 +/− 1.03 | | 10.09 +/− 2.24 | 1.21 +/− 0.71 | 3.9 +/− 2.29 | 4.83 +/− 5.69 |
| silp | Tab.si | 28.47 +/− 7.9 | | 28.9 +/− 1.73 | 29.35 +/− 8.84 | 32.4 +/− 9.07 | 27.01 +/− 4.32 |
| silp | DN(CD8mCD4mTCRp).si | 7.73 +/− 3.1 | | 11.23 +/− 1.1 | 7.81 +/− 1.41 | 6.68 +/− 1.94 | 21.87 +/− 11.35 |
| silp | T8.si | 29.3 +/− 4.06 | | 27.47 +/− 6.39 | 22.5 +/− 3.54 | 21.47 +/− 3.61 | 20.87 +/− 11.56 |
| silp | T8.Heliosp.si | | | 32.6 +/− 7.23 | 9.1 +/− 5.95 | 8.47 +/− 4.28 | 20.25 +/− 12.71 |
| silp | T4.si | 57.43 +/− 5.95 | | 58 +/− 7.26 | 66.15 +/− 2.19 | 66.57 +/− 3.63 | 50.54 +/− 6.91 |
| silp | T4.FPmRorgp.si | 3.69 +/− 1.64 | | 3.96 +/− 1.56 | 1.92 +/− 0.37 | 2.98 +/− 1.48 | 2.38 +/− 0.75 |
| silp | T4.FPp.si | 15 +/− 3.28 | | 26.23 +/− 6.64 | 24.1 +/− 2.26 | 29.43 +/− 10.08 | 23.33 +/− 5.44 |
| silp | T4.FPpHeliosm.si | | | 15.51 +/− 1.85 | 19.33 +/− 5 | 17.41 +/− 4 | 26.45 +/− 10.9 |
| silp | T4.FPpRorgpHeliosm.si | | | 5.3 +/− 1.39 | 3.93 +/− 0.76 | 4.88 +/− 1.49 | 7.39 +/− 5.58 |
| silp | ILC.il22p.si | 6.81 +/− 1.02 | | 4.99 +/− 2.65 | 21.38 +/− 6.66 | 11.74 +/− 6.84 | 19.14 +/− 22.56 |
| silp | T4.ifngp.si | 1.53 +/− 0.98 | | 0.41 +/− 0.19 | 1.94 +/− 0.12 | 0.58 +/− 0.51 | 0.59 +/− 0.48 |
| silp | T4.il10p.si | 4.72 +/− 2.18 | | 0.04 +/− 0.04 | 1.94 +/− 1.35 | 0.57 +/− 0.49 | 0.42 +/− 0.32 |
| silp | T4.il17p.si | 2.46 +/− 1.14 | | 1.58 +/− 0.44 | 1.16 +/− 0.81 | 5.01 +/− 1.18 | 2.69 +/− 1.42 |
| silp | T4.il22p.si | 0.48 +/− 0.49 | | 0.34 +/− 0.03 | 0.21 +/− 0.22 | 0.07 +/− 0.12 | 0.31 +/− 0.35 |
| sp | mono.slo | 5.88 +/− 0.7 | 2.44 +/− 0.47 | 4.56 +/− 0.24 | 5.5 +/− 0.41 | 6.05 +/− 0.27 | 4.22 +/− 0.35 |
| sp | CD11bpCD11cmF4/80MF.slo | 0.72 +/− 0.08 | 0.99 +/− 0.21 | 1.05 +/− 0.21 | 2.27 +/− 0.2 | 2.13 +/− 0.36 | 0.37 +/− 0.12 |
| sp | CD11bpCD11cpF4/80pMNP.slo | 4.47 +/− 1.01 | 3.6 +/− 0.63 | 5.51 +/− 0.72 | 3.71 +/− 0.12 | 2.3 +/− 0.29 | 2.71 +/− 0.58 |
| sp | CD103pCD11bmDC.slo | 0.22 +/− 0.1 | 0.1 +/− 0.04 | 0.41 +/− 0.1 | 0.13 +/− 0.03 | 0.05 +/− 0.01 | 0.12 +/− 0.03 |
| sp | CD103pCD11bpDC.slo | 0.37 +/− 0.2 | 0.24 +/− 0.08 | 0.77 +/− 0.07 | 0.39 +/− 0.08 | 0.13 +/− 0.01 | 0.25 +/− 0.12 |
| sp | pDC.slo | 0.97 +/− 0.28 | 0.8 +/− 0.27 | 0.38 +/− 0.08 | 0.63 +/− 0.04 | 0.49 +/− 0.05 | 0.6 +/− 0.18 |
| sp | ILC3.slo | 0.12 +/− 0.06 | 0.03 +/− 0.01 | 0.02 +/− 0 | 0.02 +/− 0.01 | 0.01 +/− 0 | 0.02 +/− 0.01 |
| sp | B.slo | 71.72 +/− 1.5 | 69.09 +/− 2.4 | 71.85 +/− 0.9 | 70.7 +/− 5.09 | 70.17 +/− 2.9 | 73 +/− 2.27 |
| sp | Tgd.slo | 0.34 +/− 0.05 | 0.37 +/− 0.09 | 0.34 +/− 0.01 | 0.27 +/− 0.03 | 0.45 +/− 0.08 | 0.38 +/− 0.02 |
| sp | Tab.slo | 19.27 +/− 1.19 | 24.34 +/− 2.59 | 21.82 +/− 0.82 | 22.3 +/− 4.24 | 23.8 +/− 2.85 | 21.17 +/− 0.93 |
| sp | DN(CD8mCD4mTCRp).slo | 3.48 +/− 1.19 | 2.71 +/− 0.49 | 1.65 +/− 0.19 | 1.73 +/− 0.37 | 2.3 +/− 0.39 | 4.81 +/− 3.01 |
| sp | T8.slo | 34.1 +/− 1.5 | 33.3 +/− 1.01 | 35.13 +/− 1.55 | 41 +/− 2.55 | 35.27 +/− 1.8 | 34.43 +/− 1.76 |
| sp | T8.Heliosp.slo | | 4.14 +/− 1.72 | 2.46 +/− 0.28 | 2.92 +/− 0.07 | 5.42 +/− 0.67 | 4.62 +/− 0.69 |
| sp | T4.slo | 58.27 +/− 1.67 | 62.49 +/− 1.27 | 61.15 +/− 1.47 | 54.7 +/− 2.4 | 60.4 +/− 1.41 | 58.97 +/− 1.52 |
| sp | T4.FPmRorgp.slo | 1.63 +/− 0.83 | 0.5 +/− 0.2 | 0.3 +/− 0.09 | 0.19 +/− 0.02 | 0.2 +/− 0.1 | 0.15 +/− 0.04 |
| sp | T4.FPp.slo | 14.38 +/− 1.22 | 12.57 +/− 1 | 9.98 +/− 0.38 | 13.1 +/− 1.98 | 13.33 +/− 0.35 | 13.14 +/− 0.49 |
| sp | T4.FPpHeliosm.slo | 22.91 +/− 1.28 | 18.43 +/− 1.52 | 33.39 +/− 1.73 | 24.36 +/− 1 | 24.76 +/− 0.79 | 21.01 +/− 1.09 |
| sp | T4.FPpRorgpHeliosm.slo | 0.41 +/− 0.05 | 1.01 +/− 0.26 | 0.64 +/− 0.19 | 0.26 +/− 0.01 | 0.49 +/− 0.14 | 0.47 +/− 0.29 |
| sp | ILC.il22p.slo | 0.05 +/− 0.01 | 0.23 +/− 0.14 | 0.37 +/− 0.14 | 0.11 +/− 0.01 | 0.01 +/− 0.01 | 0.1 +/− 0.13 |
| sp | T4.ifngp.slo | 0.91 +/− 0.12 | 1.47 +/− 0.29 | 0.42 +/− 0.06 | 0.91 +/− 0.03 | 0.92 +/− 0.33 | 0.94 +/− 0.44 |
| sp | T4.il10p.slo | 1.34 +/− 0.45 | 0.25 +/− 0.05 | 0.02 +/− 0.01 | 0.17 +/− 0.06 | 0.06 +/− 0.04 | 0.06 +/− 0.03 |
| sp | T4.il17p.slo | 0.2 +/− 0.06 | 0.26 +/− 0.12 | 0.36 +/− 0.1 | 0.13 +/− 0 | 0.22 +/− 0.01 | 0.52 +/− 0.24 |
| sp | T4.il22p.slo | 0.04 +/− 0.02 | 0.49 +/− 0.45 | 0.13 +/− 0.09 | 0.05 +/− 0.01 | 0.01 +/− 0 | 0.04 +/− 0.05 |

Table 3F

| Organ | Phylum | Firmicutes | Firmicutes | Proteobacteria | Firmicutes | Bacteroidetes | Actinobacteria | Firmicutes |
|---|---|---|---|---|---|---|---|---|
| organ | Rownames (cell ty pe in figure) | Lease.AO47 | Lrham.LMS2-1 | Nflav.SK114 | Pasac.AO33 | Pdist.ATCC8503 | Pgran.AO42 | Pmagn.AO29 |
| colon | mono.co | 3.43 +/− 0.37 | 2.09 +/− 0.41 | 5.61 +/− 1.34 | 2.05 +/− 0.48 | 2.44 +/− 0.62 | 3.75 +/− 1.6 | 3.34 +/− 0.98 |
| colon | CD11bpCD11cmF4/80pMF.co | 0.5 +/− 0.21 | 0.76 +/− 0.25 | 2.01 +/− 2.05 | 1 +/− 0.45 | 1.33 +/− 0.58 | 2.03 +/− 0.35 | 0.72 +/− 0.1 |
| colon | CD11bpCD11cpF4/80pMNP.co | 13.39 +/− 2.63 | 10.57 +/− 5.35 | 14.75 +/− 3.44 | 9.74 +/− 1.37 | 16.04 +/− 5.79 | 16.47 +/− 4.62 | 15.28 +/− 0.98 |
| colon | CD103pCD11bmDC.co | 2.77 +/− 2.13 | 2.74 +/− 1.84 | 3.52 +/− 0.92 | 0.47 +/− 0.11 | 1.92 +/− 1.05 | 3.13 +/− 0.65 | 1.48 +/− 0.19 |
| colon | CD103pCD11bpDC.co | 2.32 +/− 0.14 | 3.45 +/− 0.46 | 2.99 +/− 1.03 | 2.59 +/− 0.54 | 2.16 +/− 0.35 | 2.33 +/− 0.39 | 2.42 +/− 0.51 |
| colon | pDC.co | 1.65 +/− 0.36 | 0.36 +/− 0.16 | 3.06 +/− 1.47 | 0.68 +/− 0.16 | 2.33 +/− 1.51 | 0.54 +/− 0.04 | 1.03 +/− 0.3 |
| colon | ILC3.co | 4.5 +/− 0.83 | 1.74 +/− 0.69 | 3 +/− 0.51 | 4.93 +/− 1.32 | 0.64 +/− 0.11 | 6.11 +/− 2.42 | 2.85 +/− 2.74 |
| colon | B.co | 67.43 +/− 4.15 | 54.5 +/− 12.31 | 44.25 +/− 10.25 | 53.33 +/− 2.28 | 60.25 +/− 6.92 | 52.47 +/− 12.07 | 49.43 +/− 15.3 |
| colon | Tgd.co | 1.49 +/− 0.53 | 2.08 +/− 0.55 | 3.01 +/− 0.81 | 2.1 +/− 0.46 | 3.46 +/− 0.43 | 2.42 +/− 0.52 | 2.06 +/− 1.42 |
| colon | Tab.co | 18.17 +/− 1.17 | 30.75 +/− 9.21 | 28.95 +/− 5.87 | 24.7 +/− 2.23 | 24.1 +/− 3.16 | 24 +/− 0.98 | 24.27 +/− 5.7 |
| colon | DN(CD8mCD4mTCRp).co | 16.17 +/− 2.15 | 27.77 +/− 4.01 | 37.05 +/− 4.6 | 36.23 +/− 9.43 | 30.72 +/− 3.6 | 31.47 +/− 10.49 | 21.23 +/− 5.99 |
| colon | T8.co | 22.4 +/− 2.25 | 29.2 +/− 0.93 | 25.05 +/− 4.31 | 25.2 +/− 1.82 | 30.7 +/− 0.81 | 23.33 +/− 0.31 | 29.17 +/− 4.62 |
| colon | T8.Heliosp.co | 20.27 +/− 5.73 | 48.7 +/− 10.21 | 56.65 +/− 11.1 | 48.57 +/− 15.69 | 46.27 +/− 3.92 | 41.8 +/− 10.46 | 51.67 +/− 11.07 |
| colon | T4.co | 56.7 +/− 2.62 | 38.2 +/− 4.14 | 34.95 +/− 0.78 | 33.6 +/− 8.35 | 36.3 +/− 3.16 | 39.17 +/− 9.7 | 46.36 +/− 7.13 |
| colon | T4.FPmRorgp.co | 2.19 +/− 0.47 | 1.43 +/− 0.61 | 3.8 +/− 3.03 | 0.93 +/− 0.65 | 1.47 +/− 0.38 | 3.99 +/− 1.54 | 0.65 +/− 0.25 |
| colon | T4.FPp.co | 22.5 +/− 1.51 | 15 +/− 4.92 | 25.25 +/− 4.74 | 25.67 +/− 8.75 | 28.97 +/− 4.04 | 22.33 +/− 8.83 | 18.36 +/− 9.17 |
| colon | T4.FPpHeliosm. co | 45.53 +/− 6.8 | 38.32 +/− 4.26 | 29.32 +/− 2.38 | 31.71 +/− 6.9 | 25.88 +/− 3.12 | 30.77 +/− 1.05 | 24.61 +/− 5.64 |
| colon | T4.FPpRorgpHeliosm.co | 25.93 +/− 3.91 | 23.93 +/− 4.91 | 10.87 +/− 4.85 | 7.44 +/− 2.57 | | 17.93 +/− 3.35 | 4.86 +/− 2.7 |
| colon | ILC.il22p.co | 17.77 +/− 6.72 | 1.91 +/− 0.67 | 10.93 +/− 6.5 | 1.53 +/− 0.81 | 6.28 +/− 2.93 | 5.57 +/− 2.47 | 3.14 +/− 2.29 |
| colon | T4.ifngp.co | 1.91 +/− 0.27 | 1.16 +/− 0.43 | 1.81 +/− 1.12 | 1.18 +/− 0.76 | 1.16 +/− 0.42 | 1.23 +/− 1.03 | 0.88 +/− 0.14 |
| colon | T4.il10p.co | 1.16 +/− 0.34 | 1.03 +/− 1.02 | 0.58 +/− 0.22 | 0.19 +/− 0.12 | 1.11 +/− 0.25 | 0.45 +/− 0.25 | 0.33 +/− 0.23 |
| colon | T4.il17p.co | 1.46 +/− 0.4 | 4.13 +/− 0.67 | 1.82 +/− 0.47 | 0.45 +/− 0.11 | 1.4 +/− 0.42 | 2.6 +/− 0.85 | 1.06 +/− 0.36 |
| colon | T4.il22p.co | 0.19 +/− 0.07 | 0.88 +/− 0.2 | 0.32 +/− 0.09 | 0.14 +/− 0.12 | 0.59 +/− 0.43 | 0.89 +/− 0.2 | 0.39 +/− 0.19 |
| mln | mono.mln | 0.44 +/− 0.28 | 0.12 +/− 0.05 | 0.37 +/− 0.02 | 0.18 +/− 0.03 | 0.24 +/− 0.06 | 0.35 +/− 0.38 | 0.24 +/− 0.06 |
| mln | CD11bpCD11cpF4/80pMNP.mln | 1.33 +/− 0.35 | 0.4 +/− 0.15 | 0.74 +/− 0.3 | 0.19 +/− 0.07 | 0.66 +/− 0.08 | 0.57 +/− 0.25 | 0.5 +/− 0.06 |
| mln | CD103pCD11bmDC.mln | 0.83 +/− 0.02 | 0.4 +/− 0.14 | 0.69 +/− 0.17 | 0.29 +/− 0.02 | 1.2 +/− 0.2 | 0.45 +/− 0.19 | 0.22 +/− 0.03 |
| mln | CD103pCD11bpDC.mln | 1.69 +/− 0.17 | 0.88 +/− 0.46 | 1.38 +/− 0.44 | 1.65 +/− 0.09 | 0.63 +/− 0.19 | 0.62 +/− 0.17 | 0.99 +/− 0.18 |
| mln | pDC.mln | 0.22 +/− 0.07 | 0.07 +/− 0.03 | 0.27 +/− 0.06 | 0.13 +/− 0.02 | 0.19 +/− 0.03 | 0.13 +/− 0.1 | 0.14 +/− 0.01 |
| mln | ILC3.mln | 0.15 +/− 0.02 | 0.09 +/− 0.01 | 0.07 +/− 0.04 | 0.15 +/− 0.03 | | 0.08 +/− 0.01 | 0.11 +/− 0.06 |
| mln | B.mln | 57.3 +/− 1.92 | 48.73 +/− 4.38 | 53.75 +/− 3.75 | 50.83 +/− 1.86 | 65.38 +/− 2.84 | 48.03 +/− 6.12 | 47.23 +/− 14.37 |
| mln | Tgd.mln | 0.59 +/− 0.19 | 0.65 +/− 0.45 | 0.42 +/− 0.04 | 0.47 +/− 0.06 | 0.45 +/− 0.05 | 0.68 +/− 0.08 | 0.73 +/− 0.29 |
| mln | Tab.mln | 37.77 +/− 2.21 | 44.4 +/− 1.91 | 42.75 +/− 3.61 | 44.37 +/− 2 | 31.25 +/− 2.84 | 45.03 +/− 6.5 | 47.98 +/− 12.7 |
| mln | DN(CD8mCD4mTCRp).mln | 0.74 +/− 0.14 | 1.07 +/− 0.89 | 0.62 +/− 0.01 | 0.43 +/− 0.03 | 0.48 +/− 0.1 | 0.87 +/− 0.19 | 0.68 +/− 0.16 |
| mln | T8.mln | 39.93 +/− 1.07 | 39.65 +/− 0.6 | 42.65 +/− 2.9 | 41.53 +/− 1.04 | 38.68 +/− 2.62 | 43.03 +/− 7.61 | 41.3 +/− 2.95 |
| mln | T8.Heliosp.mln | 3.09 +/− 0.17 | 2.03 +/− 0.46 | 3.56 +/− 0.02 | 3.05 +/− 0.21 | 3.61 +/− 0.84 | 3.1 +/− 1.77 | 3.15 +/− 0.44 |
| mln | T4.mln | 57.77 +/− 1.31 | 58.28 +/− 1.14 | 56.1 +/− 2.97 | 57.37 +/− 1.07 | 60.3 +/− 2.65 | 55.17 +/− 7.68 | 57 +/− 3.35 |
| mln | T4.FPmRorgp.mln | 0.62 +/− 0.1 | 0.58 +/− 0.09 | 0.37 +/− 0.13 | 0.15 +/− 0.02 | | 1.18 +/− 0.1 | 0.29 +/− 0.04 |
| mln | T4.FPp.mln | 15.53 +/− 1.55 | 10.71 +/− 2.05 | 11.85 +/− 1.2 | 10.98 +/− 0.99 | 13.72 +/− 0.46 | 12.18 +/− 5.05 | 12.77 +/− 1.3 |
| mln | T4.FPpHeliosm.mln | 32.57 +/− 2 | 30.15 +/− 2.46 | 35.72 +/− 1.36 | 39.13 +/− 3.16 | 29.02 +/− 3.99 | 34.94 +/− 8.35 | 31.05 +/− 2.3 |
| mln | T4.FPpRorgpHeliosm.mln | 4.97 +/− 0.35 | 2.83 +/− 0.71 | 1.67 +/− 0.54 | 1.99 +/− 0.23 | | 3 +/− 0.96 | 1.8 +/− 1.01 |
| mln | ILC.il22p.mln | 0.4 +/− 0.12 | 0.12 +/− 0.1 | 0.92 +/− 0.47 | 0.08 +/− 0.03 | 0.76 +/− 0.22 | 1.02 +/− 0.79 | 0.14 +/− 0.14 |
| mln | T4.ifngp.mln | 2.07 +/− 0.31 | 0.79 +/− 0.43 | 0.8 +/− 0.45 | 1.06 +/− 0.08 | 0.21 +/− 0.04 | 0.46 +/− 0.39 | 0.52 +/− 0.35 |
| mln | T4.il10p.mln | 0.33 +/− 0.12 | 0.06 +/− 0.02 | 0.14 +/− 0.08 | 0.11 +/− 0.04 | 1.42 +/− 1.38 | 0.21 +/− 0.09 | 0.09 +/− 0.04 |
| mln | T4.il17p.mln | 0.82 +/− 0.1 | 0.45 +/− 0.13 | 0.53 +/− 0.21 | 0.12 +/− 0.01 | 0.48 +/− 0.11 | 0.17 +/− 0.09 | 0.19 +/− 0.14 |
| mln | T4.il22p.mln | 0.32 +/− 0.2 | 0.42 +/− 0.2 | 0.3 +/− 0.11 | 0.05 +/− 0.03 | 0.12 +/− 0.06 | 0.13 +/− 0.06 | 0.14 +/− 0.08 |
| pp | mono.pp | 1 +/− 0.43 | 0.93 +/− 0.48 | 0.82 +/− 0.33 | 1.01 +/− 0.76 | 0.27 +/− 0.1 | 1.25 +/− 0.66 | 0.93 +/− 0.17 |
| pp | CD11bpCD11cmF4/80pMF.pp | 0.19 +/− 0.08 | 0.07 +/− 0.03 | 0.33 +/− 0.37 | 0.13 +/− 0.05 | | 0.3 +/− 0.21 | 0.19 +/− 0.03 |
| pp | CD11bpCD11cpF4/80pMNP.pp | 3.54 +/− 1.96 | 2.24 +/− 0.6 | 2.34 +/− 1.9 | 2.12 +/− 1.66 | 0.23 +/− 0.09 | 4.34 +/− 1.44 | 3.77 +/− 0.82 |
| pp | CD103pCD11bmDC.pp | 3.13 +/− 0.55 | 2.76 +/− 0.5 | 3.41 +/− 0.97 | 1.16 +/− 0.47 | 5.98 +/− 2.21 | 4.44 +/− 1.25 | 6.15 +/− 0.99 |
| pp | CD103pCD11bpDC.pp | 12.27 +/− 1.87 | 11.6 +/− 1.84 | 7.3 +/− 2.38 | 4.43 +/− 0.34 | 9.26 +/− 3.99 | 13.63 +/− 2.96 | 19.06 +/− 1.18 |
| pp | pDC.pp | 1.64 +/− 1 | 1.29 +/− 0.47 | 3.47 +/− 1.76 | 0.73 +/− 0.5 | 1.52 +/− 0.34 | 2.18 +/− 1 | 2.21 +/− 0.61 |
| pp | ILC3.pp | 0.56 +/− 0.31 | 0.36 +/− 0.06 | 0.32 +/− 0.17 | 0.65 +/− 0.27 | | 0.47 +/− 0.16 | 0.28 +/− 0.09 |
| pp | B.pp | 84.07 +/− 6.69 | 85.37 +/− 1.73 | 80.15 +/− 5.73 | 71.03 +/− 16.18 | 85.32 +/− 2.44 | 83.97 +/− 5.82 | 86.77 +/− 1.22 |
| pp | Tgd.pp | 2.35 +/− 1.62 | 3.7 +/− 1.55 | 3.56 +/− 1.34 | 1.46 +/− 0.05 | 3.92 +/− 1.34 | 4.53 +/− 3.2 | 3.27 +/− 0.53 |
| pp | Tab.pp | 6.76 +/− 2.25 | 6.59 +/− 1.15 | 8.28 +/− 0.71 | 9.75 +/− 1.89 | 8.22 +/− 1.8 | 7.55 +/− 2.51 | 6.43 +/− 0.37 |
| pp | DN(CD8mCD4mTCRp).pp | 2.7 +/− 1.08 | 3.85 +/− 1.05 | 3.33 +/− 0.98 | 6.91 +/− 6.84 | 2.63 +/− 1.56 | 3.65 +/− 1.72 | 3.33 +/− 1.04 |
| pp | T8.pp | 35.93 +/− 3.07 | 39.7 +/− 3.59 | 34.1 +/− 0.14 | 28.8 +/− 7.47 | 40.75 +/− 2.36 | 34.57 +/− 5.59 | 34.07 +/− 1.79 |
| pp | T8.Heliosp.pp | 27.83 +/− 2.49 | 15.48 +/− 6.28 | 26.65 +/− 3.18 | 8.03 +/− 1.6 | 17.42 +/− 5.64 | 21.37 +/− 8.06 | 14.32 +/− 2.45 |
| pp | T4.pp | 57.8 +/− 3.34 | 50.77 +/− 5.44 | 59.45 +/− 2.05 | 60.5 +/− 2.21 | 53.38 +/− 1.54 | 54.3 +/− 10.54 | 55.9 +/− 1.32 |
| pp | T4.FPmRorgp.pp | 3.31 +/− 1.29 | 1.84 +/− 0.74 | 1.35 +/− 0.77 | 1 +/− 0.3 | | 3.1 +/− 0.61 | 0.63 +/− 0.13 |
| pp | T4.FPp.pp | 17.37 +/− 2.64 | 18.5 +/− 3.26 | 20.05 +/− 2.62 | 11.7 +/− 1.18 | 19.9 +/− 2.26 | 18.33 +/− 2.76 | 13.15 +/− 0.62 |
| pp | T4.FPpHeliosm.pp | 33.17 +/− 5.6 | 26.63 +/− 6.96 | 34.9 +/− 10.49 | 29.31 +/− 1.31 | 28.67 +/− 3.12 | 24.18 +/− 4.3 | 20.24 +/− 1.72 |
| pp | T4.FPpRorgpHeliosm.pp | 15.87 +/− 1.6 | 11.23 +/− 5.27 | 3.8 +/− 0.11 | 7.05 +/− 1.39 | | 7.68 +/− 1.92 | 2.92 +/− 1.2 |
| pp | ILC.il22p.pp | 1.62 +/− 1.24 | 0.63 +/− 0.2 | 3.66 +/− 0.95 | 1.86 +/− 1.28 | 0.28 +/− 0.25 | 1.37 +/− 0.24 | 1.9 +/− 1.21 |
| pp | T4.ifngp.pp | 0.22 +/− 0.11 | 0.31 +/− 0.12 | 0.81 +/− 0.31 | 0.44 +/− 0.41 | 0.32 +/− 0.4 | 0.58 +/− 0.24 | 0.58 +/− 0.26 |
| pp | T4.il10p.pp | 1.34 +/− 0.84 | 0.57 +/− 0.19 | 0.38 +/− 0.33 | 0.14 +/− 0.13 | 2.29 +/− 0.81 | 0.76 +/− 0.91 | 1 +/− 0.12 |
| pp | T4.il17p.pp | 1.53 +/− 0.51 | 2.45 +/− 1.07 | 1.17 +/− 0.69 | 0.29 +/− 0.15 | 1.76 +/− 3.1 | 2.33 +/− 1.37 | 2.54 +/− 0.42 |
| pp | T4.il22p.pp | 0.37 +/− 0.14 | 0.83 +/− 0.48 | 0.54 +/− 0.5 | 1.14 +/− 0.63 | 0.21 +/− 0.29 | 0.53 +/− 0.34 | 0.26 +/− 0.18 |
| silp | mono.si | 7.9 +/− 0.79 | 6.56 +/− 2.5 | 6.81 +/− 2.2 | 6.19 +/− 0.92 | 5.77 +/− 1.25 | 7.09 +/− 1.02 | 6.54 +/− 1.02 |
| silp | CD11bpCD11cmF4/80pMF.si | 0.33 +/− 0.16 | 0.18 +/− 0.06 | 0.95 +/− 1.02 | 0.58 +/− 0.25 | 1.33 +/− 0.33 | 2.26 +/− 0.34 | 0.92 +/− 0.18 |
| silp | CD11bpCD11cpF4/80pMNP.si | 6.14 +/− 3.94 | 8.35 +/− 1.72 | 26.21 +/− 5.85 | 13 +/− 4.43 | 15.17 +/− 4.6 | 30.23 +/− 4.2 | 24.83 +/− 5.37 |
| silp | CD103pCD11bmDC.si | 13.52 +/− 7.04 | 27.91 +/− 1.95 | 11.96 +/− 3.05 | 8.5 +/− 3.78 | 5.85 +/− 4.46 | 11.42 +/− 3.03 | 15.25 +/− 5.05 |
| silp | CD103pCD11bpDC.si | 2.39 +/− 0.28 | 1.22 +/− 0.39 | 2.13 +/− 2.13 | 2.9 +/− 0.34 | 0.5 +/− 0.41 | 4.21 +/− 0.58 | 5.97 +/− 2.87 |
| silp | pDC.si | 4.15 +/− 0.26 | 4.49 +/− 0.95 | 18.15 +/− 10.18 | 6.19 +/− 0.65 | 6.67 +/− 5.62 | 10.05 +/− 5.27 | 8.07 +/− 0.58 |
| silp | ILC3.si | 11.26 +/− 3.89 | 26.27 +/− 5.62 | 17.4 +/− 4.81 | 21.6 +/− 9.32 | | 20.85 +/− 7.97 | 25.43 +/− 3.09 |
| silp | B.si | 52.63 +/− 13.71 | 28.63 +/− 8.23 | 44.58 +/− 19.95 | 46.87 +/− 24.06 | 40.2 +/− 14.34 | 40.37 +/− 18.33 | 17.93 +/− 1.3 |
| silp | Tgd.si | 2.09 +/− 0.97 | 7.38 +/− 2.49 | 4.11 +/− 3.52 | 2.47 +/− 0.67 | 5.88 +/− 2.36 | 4.12 +/− 1.02 | 6.5 +/− 2.56 |
| silp | Tab.si | 30.23 +/− 7.75 | 26.82 +/− 4.12 | 20.1 +/− 6.64 | 25.7 +/− 12.27 | 35.28 +/− 8.56 | 31.7 +/− 8.67 | 30.63 +/− 2.21 |
| silp | DN(CD8mCD4mTCRp).si | 11.64 +/− 10.01 | 13.88 +/− 2.3 | 8.33 +/− 2.94 | 9.27 +/− 2.08 | 7.55 +/− 2.91 | 7.43 +/− 0.24 | 7 +/− 1.44 |
| silp | T8.si | 26.23 +/− 2.85 | 27.98 +/− 1.89 | 28.05 +/− 7.66 | 23.2 +/− 6.98 | 23.28 +/− 2.73 | 29.8 +/− 7.01 | 21.17 +/− 3.66 |
| silp | T8.Heliosp.si | 16.41 +/− 15.12 | 23.6 +/− 9.21 | 42.6 +/− 22.91 | 9.68 +/− 1.27 | 14.33 +/− 3.76 | 13.1 +/− 3.22 | 17.23 +/− 2.94 |
| silp | T4.si | 58.73 +/− 8.13 | 51.07 +/− 2.4 | 60.58 +/− 6.16 | 61.4 +/− 3.16 | 64.8 +/− 5.32 | 57.6 +/− 5.27 | 64.05 +/− 2.4 |
| silp | T4.FPmRorgp.si | 6.47 +/− 2.63 | 4.68 +/− 1.14 | 4.83 +/− 1.25 | 2.08 +/− 1.28 | | 6.61 +/− 2.76 | 2.52 +/− 0.87 |
| silp | T4.FPp.si | 20.3 +/− 4.5 | 33.25 +/− 4.06 | 18.49 +/− 8.89 | 21.7 +/− 9.31 | 40.02 +/− 6.56 | 19.13 +/− 1.07 | 25.97 +/− 3.36 |
| silp | T4.FPpHeliosm.si | 29.86 +/− 2.41 | 13.65 +/− 1.66 | 20.11 +/− 8.04 | 14.09 +/− 4.15 | 14.63 +/− 3.4 | 15.55 +/− 1.64 | 13.87 +/− 1.46 |
| silp | T4.FPpRorgpHeliosm.si | 12.36 +/− 4.98 | 7.74 +/− 1.35 | 7.27 +/− 3.77 | 3.13 +/− 0.95 | | 4.52 +/− 1.22 | 3.2 +/− 0.75 |
| silp | ILC.il22p.si | 25.39 +/− 4.22 | 8.13 +/− 3.29 | 28.44 +/− 10.8 | 13.56 +/− 9.97 | 19.96 +/− 6.86 | 3.16 +/− 1.01 | 31.47 +/− 16.73 |
| silp | T4.ifngp.si | 2.24 +/− 1.87 | 0.81 +/− 0.4 | 1.57 +/− 1.15 | 0.67 +/− 0.51 | 1.75 +/− 0.77 | 1.05 +/− 0.56 | 0.97 +/− 0.32 |
| silp | T4.il10p.si | 1.01 +/− 0.33 | 0.59 +/− 0.25 | 0.74 +/− 0.13 | 0.1 +/− 0.08 | 0.97 +/− 0.7 | 0.76 +/− 0.72 | 0.72 +/− 0.15 |
| silp | T4.il17p.si | 3.6 +/− 1.05 | 1.85 +/− 0.82 | 2.84 +/− 1.46 | 0.76 +/− 0.37 | 3.69 +/− 1.01 | 3.13 +/− 0.54 | 2.31 +/− 0.75 |
| silp | T4.il22p.si | 1.53 +/− 0.47 | 2.44 +/− 1.24 | 1.02 +/− 0.4 | 2.31 +/− 0.92 | 3.36 +/− 3.89 | 0.6 +/− 0.31 | 0.68 +/− 0.36 |
| sp | mono.slo | 4.98 +/− 1.34 | 3.69 +/− 0.32 | 7.53 +/− 2.17 | 3.37 +/− 1.05 | 4.17 +/− 1.28 | 4.51 +/− 2.02 | 4.07 +/− 0.54 |
| sp | CD11bpCD11cmF4/80pMF.slo | 0.45 +/− 0.23 | 0.53 +/− 0.11 | 2.2 +/− 2 | 1.09 +/− 0.52 | 0.4 +/− 0.44 | 1.41 +/− 1.07 | 1.03 +/− 0.1 |
| sp | CD11bpCD11cpF4/80pMNP.slo | 3.8 +/− 1.26 | 4.29 +/− 1.39 | 4.29 +/− 1.13 | 3.33 +/− 2.15 | 2.71 +/− 1.59 | 4.76 +/− 2.41 | 4.34 +/− 0.59 |
| sp | CD103pCD11bmDC.slo | 0.11 +/− 0.01 | 0.11 +/− 0.11 | 0.1 +/− 0.02 | 0.05 +/− 0.02 | 0.3 +/− 0.18 | 0.04 +/− 0.03 | 0.08 +/− 0.01 |
| sp | CD103pCD11bpDC.slo | 0.3 +/− 0.02 | 0.08 +/− 0.04 | 0.16 +/− 0.14 | 1.05 +/− 1.18 | 0.4 +/− 0.26 | 0.08 +/− 0.07 | 0.26 +/− 0.03 |
| sp | pDC.slo | 0.68 +/− 0.21 | 0.66 +/− 0.13 | 0.59 +/− 0.14 | 0.7 +/− 0.85 | 0.73 +/− 0.46 | 0.6 +/− 0.12 | 0.58 +/− 0.06 |
| sp | ILC3.slo | 0.04 +/− 0.01 | 0.01 +/− 0.01 | 0.04 +/− 0 | 0.02 +/− 0 | 0.02 +/− 0.01 | 0.03 +/− 0.01 | 0.03 +/− 0.03 |
| sp | B.slo | 70.4 +/− 3.63 | 73.68 +/− 1.49 | 68.1 +/− 0.14 | 72.17 +/− 2.39 | 79.67 +/− 1.55 | 67.47 +/− 3.53 | 73.64 +/− 1.54 |
| sp | Tgd.slo | 0.42 +/− 0.17 | 0.35 +/− 0.04 | 0.38 +/− 0.01 | 0.38 +/− 0.01 | 0.35 +/− 0.05 | 0.46 +/− 0.06 | 0.3 +/− 0.04 |
| sp | Tab.slo | 21.67 +/− 2.17 | 20.53 +/− 1.53 | 22.9 +/− 0.57 | 19.83 +/− 2.1 | 15.95 +/− 1.66 | 22.93 +/− 0.91 | 18.09 +/− 1.43 |
| sp | DN(CD8mCD4mTCRp).slo | 3.81 +/− 1.06 | 1.49 +/− 0.13 | 2.44 +/− 0.14 | 1.65 +/− 0.32 | 2.72 +/− 0.4 | 1.73 +/− 0.26 | 2.35 +/− 0.5 |
| sp | T8.slo | 32.47 +/− 2.23 | 35.65 +/− 0.75 | 38.05 +/− 0.92 | 38.3 +/− 1.87 | 37.85 +/− 1.39 | 38.63 +/− 5.35 | 31.7 +/− 3.28 |
| sp | T8.Heliosp.slo | 5.77 +/− 0.7 | 4.23 +/− 0.92 | 3.48 +/− 0.06 | 4.75 +/− 1.08 | 5.29 +/− 1.12 | 4.28 +/− 0.35 | 4.53 +/− 0.76 |
| sp | T4.slo | 62 +/− 1.8 | 61.5 +/− 0.7 | 58.1 +/− 0.57 | 58.7 +/− 1.51 | 57.7 +/− 0.89 | 57.37 +/− 6.29 | 60.74 +/− 1.2 |
| sp | T4.FPmRorgp.slo | 0.3 +/− 0.08 | 0.19 +/− 0.03 | 0.37 +/− 0.01 | 0.11 +/− 0.05 | 0.37 +/− 0.11 | 0.73 +/− 0.12 | 0.22 +/− 0.03 |
| sp | T4.FPp.slo | 14.53 +/− 2.48 | 11.08 +/− 1.03 | 12.95 +/− 0.35 | 12.77 +/− 1.19 | 15.9 +/− 0.22 | 13.53 +/− 1.88 | 12.5 +/− 1.44 |
| sp | T4.FPpHeliosin.slo | 29.5 +/− 4.32 | 24.77 +/− 3.09 | 29.43 +/− 3.87 | 27.22 +/− 1.81 | 19.57 +/− 1.56 | 27.72 +/− 3.75 | 30.1 +/− 3.2 |
| sp | T4.FPpRorgpHeliosm.slo | 1.16 +/− 0.53 | 0.9 +/− 0.5 | 0.38 +/− 0.02 | 0.49 +/− 0.05 | | 1.02 +/− 0.44 | 1.26 +/− 1.2 |
| sp | ILC.il22p.slo | 0.22 +/− 0.02 | 0.04 +/− 0.01 | 0.31 +/− 0.16 | 0.01 +/− 0.01 | 0.07 +/− 0.01 | 0.21 +/− 0.16 | 0.08 +/− 0.03 |
| sp | T4.ifngp.slo | 1.2 +/− 0.81 | 1.29 +/− 0.29 | 1.16 +/− 0.41 | 0.76 +/− 0.19 | 0.74 +/− 0.34 | 0.75 +/− 0.42 | 0.84 +/− 0.22 |
| sp | T4.il10p.slo | 0.26 +/− 0.07 | 0.08 +/− 0.03 | 0.15 +/− 0.08 | 0.15 +/− 0.05 | 2 +/− 2.37 | 0.18 +/− 0.07 | 0.56 +/− 0.12 |
| sp | T4.il17p.slo | 0.6 +/− 0.14 | 1.64 +/− 0.59 | 0.22 +/− 0.07 | 0.2 +/− 0.17 | 0.36 +/− 0.31 | 0.53 +/− 0.3 | 0.38 +/− 0.55 |
| sp | T4.il22p.slo | 0.15 +/− 0.09 | 0.35 +/− 0.11 | 0.35 +/− 0.2 | 0.04 +/− 0.01 | 0.11 +/− 0.05 | 0.15 +/− 0.09 | 0.13 +/− 0.09 |

Table 3G

| Organ | Phylum | Bacteroidetes | Firmicutes | Firmicutes | Firmicutes | Firmicutes | Firmicutes | |
|---|---|---|---|---|---|---|---|---|
| organ | Rownames (cell type in figure) | Pueno.UPII60-3 | Rgv.ATCC29149 | SFB | Smiti.F0392 | Ssapr.ATCC15305 | Veil.6.1.27 | SPF |
| colon | mono.co | 2.48 +/− 1.01 | 3.08 +/− 0.52 | 3.84 +/− 0.77 | 1.76 +/− 0.43 | 3.08 +/− 0.52 | 3.03 +/− 0.25 | 4.09 +/− 1.06 |
| colon | CD11bpCD11cmF4/80pMF.co | 1.46 +/− 0.8 | 1.11 +/− 0.76 | 1.01 +/− 0.26 | 1.29 +/− 0.78 | 1.94 +/− 0.32 | 0.67 +/− 0.09 | 1.38 +/− 0.29 |
| colon | CD11bpCD11cpF4/80pMNP.co | 10.03 +/− 5.37 | 15.48 +/− 11.87 | 2.42 +/− 0.39 | 9.75 +/− 0.84 | 15.76 +/− 1.91 | 7.64 +/− 1.82 | 9.76 +/− 5.4 |
| colon | CD103pCD11bmDC.co | 2.36 +/− 1.49 | 2.36 +/− 0.82 | 1.86 +/− 0.66 | 2.96 +/− 0.85 | 1.25 +/− 0.37 | 2.62 +/− 0.61 | 2.88 +/− 0.6 |
| colon | CD103pCD11bpDC.co | 1.89 +/− 0.6 | 2.32 +/− 0.48 | 1.97 +/− 0.85 | 2.3 +/− 0.78 | 2.96 +/− 0.36 | 2.99 +/− 1.22 | 1.87 +/− 0.19 |
| colon | pDC.co | 1.12 +/− 0.35 | 3.47 +/− 3.08 | 2.78 +/− 0.83 | 3.65 +/− 1.04 | 0.28 +/− 0.02 | 0.81 +/− 0.26 | 13.47 +/− 1.76 |
| colon | ILC3.co | 1.63 +/− 1.01 | 5.38 +/− 1.23 | 2.47 +/− 0.38 | 1.99 +/− 0.26 | 6.41 +/− 3.45 | 1 +/− 0.21 | 6.75 +/− 3.66 |
| colon | B.co | 61.83 +/− 6.2 | 57.75 +/− 12.89 | 60.77 +/− 10.24 | 51.45 +/− 9.83 | 43.73 +/− 8.91 | 49.27 +/− 9.03 | 51.77 +/− 18.33 |
| colon | Tgd.co | 1.63 +/− 0.53 | 1.8 +/− 1.1 | 1.53 +/− 0.47 | 1.67 +/− 0.54 | 3.82 +/− 0.76 | 3.54 +/− 1.51 | 3.44 +/− 1.95 |
| colon | Tab.co | 24.96 +/− 4.06 | 20.58 +/− 5.19 | 25.55 +/− 8.34 | 30.7 +/− 8.63 | 24.57 +/− 10.98 | 33.97 +/− 6.31 | 31.38 +/− 10.61 |
| colon | DN(CD8mCD4mTCRp).co | 19.02 +/− 4.16 | 28 +/− 6.7 | 20.38 +/− 8.28 | 21.25 +/− 4.45 | 36.68 +/− 7.01 | 28.17 +/− 7.13 | 26.5 +/− 9.75 |
| colon | T8.co | 25.6 +/− 2.55 | 23.65 +/− 1.44 | 23.13 +/− 3.82 | 25.25 +/− 3.04 | 22.42 +/− 1.85 | 33.93 +/− 4.62 | 25.05 +/− 0.51 |
| colon | T8.Heliosp.co | 36.43 +/− 6.61 | 50.7 +/− 19.84 | 36.23 +/− 21.68 | 38.1 +/− 10.75 | 57.43 +/− 10.64 | 54.97 +/− 16.39 | 60.25 +/− 18.58 |
| colon | T4.co | 51.57 +/− 5.31 | 45.73 +/− 6.16 | 51.9 +/− 11.77 | 48.65 +/− 7.14 | 38 44 +/− 4 69 | 34.27 +/− 11 | 45.17 +/− 10.66 |
| colon | T4.FPmRorgp.co | 0.5 +/− 0.42 | 1.23 +/− 0.82 | 14.08 +/− 5.04 | 2.6 +/− 1.1 | 4.39 +/− 4.38 | 0.75 +/− 0.42 | 9.8 +/− 0.99 |
| colon | T4.FPp.co | 14.45 +/− 4.65 | 21.67 +/− 10.27 | 20.35 +/− 4.73 | 19.35 +/− 2.05 | 22.09 +/− 4.91 | 15.43 +/− 3.8 | 30.75 +/− 7.04 |
| colon | T4.FPpHeliosm.co | 28.17 +/− 5.25 | 35.3 +/− 1.75 | 37.45 +/− 4.34 | 33.6 +/− 1.49 | 16.93 +/− 6.33 | 38.33 +/− 4.84 | 49.8 +/− 1.58 |
| colon | T4.FPpRorgpHeliosm.co | 2.68 +/− 1.02 | 15.87 +/− 5.4 | 19.38 +/− 2.13 | 6.6 +/− 1.48 | 7.89 +/− 2.66 | 13.7 +/− 2.79 | 37.32 +/− 2.25 |
| colon | ILC.il22p.co | 3.98 +/− 2.2 | 4.24 +/− 0.24 | 4.87 +/− 0.91 | 11.34 +/− 14.63 | 8.22 +/− 5.22 | 0.65 +/− 0.32 | 11.31 +/− 8.27 |
| colon | T4.ifngp.co | 0.87 +/− 0.38 | 2.48 +/− 0.64 | 1.76 +/− 0.63 | 0.55 +/− 0.36 | 2.06 +/− 1.72 | 1.42 +/− 1.12 | 1.29 +/− 0.24 |
| colon | T4.il10p.co | 0.5 +/− 0.24 | 1.67 +/− 0.68 | 0.95 +/− 0.26 | 0.19 +/− 0.11 | 1.23 +/− 0.55 | 2.06 +/− 1.03 | 2 +/− 0.45 |
| colon | T4.il17p.co | 4.68 +/− 5.64 | 1.85 +/− 0.54 | 5.77 +/− 1.41 | 0.64 +/− 0.49 | 4.49 +/− 1.2 | 2.49 +/− 0.15 | 3.73 +/− l.22 |
| colon | T4.il22p.co | 0.21 +/− 0.15 | 0.13 +/− 0.06 | 0.67 +/− 0.4 | 0.32 +/− 0.01 | 1.26 +/− 0.32 | 0.24 +/− 0.09 | 0.81 +/− 0.52 |
| mln | mono.mln | 0.44 +/− 0.12 | 0.37 +/− 0.23 | 0.11 +/− 0.04 | 0.23 +/− 0.04 | 0.36 +/− 0.16 | 0.38 +/− 0.02 | 0.3 +/− 0.12 |
| mln | CD11bpCD11cpF4/80pMNP.mln | 0.46 +/− 0.33 | 0.58 +/− 0.44 | 0.07 +/− 0.03 | 0.68 +/− 0.14 | 1.32 +/− 0.23 | 1.08 +/− 0.21 | 0.54 +/− 0.04 |
| mln | CD103pCD11bmDC.mln | 0.6 +/− 0.33 | 0.84 +/− 0.42 | 0.21 +/− 0.1 | 1.46 +/− 0.06 | 0.54 +/− 0.1 | 0.57 +/− 0.08 | 0.32 +/− 0.04 |
| mln | CD103pCD11bpDC.mln | 0.99 +/− 0.28 | 1.87 +/− 1.37 | 0.45 +/− 0.13 | 2.52 +/− 0.61 | 1.11 +/− 0.21 | 1.21 +/− 0.29 | 2.36 +/− l.44 |
| mln | pDC.mln | 0.22 +/− 0.07 | 0.3 +/− 0.12 | 0.17 +/− 0.01 | 0.24 +/− 0.02 | 0.13 +/− 0.03 | 0.23 +/− 0.08 | 14.7 +/− 0.4 |
| mln | ILC3.mln | 0.1 +/− 0.03 | 0.1 +/− 0.01 | 0.05 +/− 0.03 | 0.11 +/− 0.02 | 0.09 +/− 0.05 | 0.08 +/− 0.03 | 0.06 +/− 0.01 |
| mln | B.mln | 52.55 +/− 4.96 | 48.05 +/− 3.71 | 48.78 +/− l.7 | 52.55 +/− 1.77 | 48.6 +/− 7.19 | 50.77 +/− 2.97 | 38.05 +/− 3.44 |
| mln | Tgd.mln | 0.49 +/− 0.07 | 0.53 +/− 0.08 | 0.51 +/− 0.09 | 0.49 +/− 0.06 | 0.66 +/− 0.05 | 0.56 +/− 0.02 | 0.55 +/− 0.09 |
| mln | Tab.mln | 42.21 +/− 4.76 | 38.43+/− 4.2 | 48.5 +/− 1.7 | 41.25 +/− 2.76 | 45.75 +/− 8.14 | 42.9 +/− 3.75 | 55.85 +/− 3.73 |
| mln | DN(CD8mCD4mTCRp).mln | 0.62 +/− 0.19 | 2.31 +/− 2.08 | 0.42 +/− 0.06 | 0.5 +/− 0.07 | 0.6 +/− 0.18 | 0.68 +/− 0.08 | 0.51 +/− 0.07 |
| mln | T8.mln | 38.6 +/− 1.7 | 36.57 +/− 1.25 | 39.8 +/− 0.29 | 37.3 +/− 1.41 | 38.83 +/− 1.19 | 38.33 +/− 1.65 | 36.65 +/− 1.11 |
| mln | T8.Heliosp.mln | 2.59 +/− 1.19 | 3.72 +/− 0.58 | 3.16 +/− 0.31 | 2.13 +/− 0.08 | 3.38 +/− 0.62 | 3.49 +/− 0.14 | 2.4 +/− 0.49 |
| mln | T4.mln | 59.77 +/− 1.74 | 59.47 +/− 2.22 | 58.98 +/− 0.36 | 60.85 +/− 1.48 | 58.93 +/− 2.91 | 59.23 +/− 1 | 61.07 +/− 1.23 |
| mln | T4.FPmRorgp.mln | 0.24 +/− 0.08 | 0.23 +/− 0.05 | 1 +/− 0.27 | 0.53 +/− 0.13 | 0.35 +/− 0.08 | 0.18 +/− 0 | 0.79 +/− 0.06 |
| mln | T4.FPp.mln | 12.68 +/− 1.19 | 11.25 +/− 0.71 | 11.47 +/− 0.22 | 15.15 +/− 0.49 | 13.44 +/− 1.69 | 12.67 +/− 0.6 | 12.2 +/− 0.43 |
| mln | T4.FPpHeliosm.mln | 31.12 +/− 4.81 | 26.63 +/− 2.23 | 37.89 +/− l.29 | 29.27 +/− 1.15 | 27.98 +/− 3.14 | 24.54 +/− 0.83 | 38.61 +/− 1.47 |
| mln | T4.FPpRorgpHeliosm.mln | 1.2 +/− 0.46 | 2.83 +/− 1.02 | 1.76 +/− 0.72 | 1.57 +/− 0.69 | 12.33 +/− 11.49 | 1.74 +/− 0.36 | 4.16 +/− 0.55 |
| mln | ILC.il22p.mln | 0.19 +/− 0.15 | 0.08 +/− 0.03 | 0.01 +/− 0.02 | 0.06 +/− 0 | 0.32 +/− 0.27 | 0.01 +/− 0.01 | 0.91 +/− 0.55 |
| mln | T4.ifngp.mln | 0.74 +/− 0.31 | 0.51 +/− 0.13 | 0.56 +/− 0.43 | 0.91 +/− 0.57 | 2.94 +/− 0.39 | 0.63 +/− 0.09 | 1.18 +/− 0.3 |
| mln | T4.il10p.mln | 0.66 +/− 0.37 | 0.26 +/− 0.04 | 0.21 +/− 0.1 | 0.03 +/− 0.02 | 0.15 +/− 0.05 | 0.54 +/− 0.06 | 0.29 +/− 0.07 |
| mln | T4.il17p.mln | 0.96 +/− 0.71 | 0.21 +/− 0.07 | 0.42 +/− 0.49 | 1.01 +/− 0.2 | 0.68 +/− 0.34 | 0.42 +/− 0.1 | 1.26 +/− 0.27 |
| mln | T4.il22p.mln | 0.09 +/− 0.07 | 0.01 +/− 0 | 0.1 +/− 0.13 | 0.13 +/− 0.04 | 0.25 +/− 0.03 | 0.04 +/− 0.01 | 0.37 +/− 0.12 |
| pp | mono.pp | 0.67 +/− 0.37 | 2.48 +/− 1.23 | 0.31 +/− 0.12 | 0.73 +/− 0.43 | 3.98 +/− 5.44 | 0.63 +/− 0.16 | 1.35 +/− 1.07 |
| pp | CD11bpCD11cmF4/80pMF.pp | 0.15 +/− 0.05 | 0.72 +/− 0.31 | 0.14 +/− 0.07 | 0.16 +/− 0.09 | 0.07 +/− 0.03 | 0.08 +/− 0.05 | 0.34 +/− 0.24 |
| pp | CD11bpCD11cpF4/80pMNP.pp | 1.25 +/− 0.43 | 7.81 +/− 3.31 | 0.63 +/− 0.27 | 1.06 +/− 0.54 | 5.61 +/− 4.28 | 1.46 +/− 0.43 | 1.46 +/− 1.52 |
| pp | CD103pCD11bmDC.pp | 3.15 +/− 1.26 | 2.11 +/− 0.23 | 0.84 +/− 0.23 | 2.93 +/− 0.37 | 3.73 +/− 0.54 | 2.27 +/− 1.21 | 1.51 +/− 1 |
| pp | CD103pCD11bpDC.pp | 8.56 +/− 3.83 | 3.91 +/− 1.07 | 5.98 +/− 1.05 | 16.91 +/− 5.22 | 7.08 +/− 2.04 | 8.18 +/− 1.73 | 10.2 +/− 3.25 |
| pp | pDC.pp | 1.99 +/− 0.46 | 2.95 +/− 0.36 | 1.18 +/− 0.4 | 1.93 +/− 0.11 | 0.5 +/− 0.3 | 2.76 +/− 0.85 | 19.65 +/− 4.69 |
| pp | ILC3.pp | 0.23 +/− 0.16 | 0.68 +/− 0.18 | 0.19 +/− 0.04 | 0.14 +/− 0 | 0.28 +/− 0.13 | 0.2 +/− 0.02 | 0.17 +/− 0.01 |
| pp | B.pp | 82.7 +/− 4.99 | 80.77 +/− 1.62 | 59.97+/− 2.2 | 81.65 +/− 4.6 | 78.18 +/− 11.23 | 87.7 +/− 2.55 | 83.83 +/− 0.73 |
| pp | Tgd.pp | 3.1 +/− 1.7 | 3.34 +/− 0.67 | 1.17 +/− 0.42 | 2.61 +/− 0.13 | 9.26 +/− 10.37 | 2.69 +/− 0.88 | 1.85 +/− 0.23 |
| pp | Tab.pp | 9.94 +/− 3.41 | 9.05 +/− 0.86 | 10.07 +/− 1.03 | 11.32 +/− 3.79 | 8.02 +/− 1.59 | 5.17 +/− 0.53 | 12.23 +/− 0.49 |
| pp | DN(CD8mCD4mTCRP)pp | 3.9 +/− 1.68 | 7.66 +/− 1.42 | 1.34 +/− 0.66 | 0.7 +/− 0.15 | 6.62 +/− 4.94 | 3.22 +/− 1.09 | 1.88 +/− 0.39 |
| pp | T8.pp | 33.23 +/− 5 | 31.83 +/− 1.21 | 19.13 +/− 1.76 | 36.3 +/− 0.57 | 35.02 +/− 4.97 | 34 +/− 5.03 | 23.15 +/− 1.51 |
| pp | T8.Heliosp.pp | 16.94 +/− 10.5 | 14.87 +/− 4.89 | 11.03 +/− 2.7 | 10.03 +/− 1.65 | 15.98 +/− 8.57 | 18.77 +/− 2.8 | 13.9 +/− 1.16 |
| pp | T4.pp | 59.41 +/− 4.4 | 58.53 +/− 1.96 | 72.65 +/− 2.89 | 59.95 +/− 0.78 | 56.21 +/− 6.92 | 60.4 +/− 5.98 | 72.13 +/− 1.38 |
| pp | T4.FPmRorgp.pp | 1.1 +/− 1.07 | 0.73 +/− 0.14 | 5.33 +/− 1.41 | 0.97 +/− 0.28 | 1.35 +/− 0.37 | 0.21 +/− 0.08 | 4.69 +/− 0.54 |
| pp | T4.FPp.pp | 16.63 +/− 4.8 | 12.1 +/− 1.23 | 6.17 +/− 0.67 | 13.1 +/− 1.98 | 16.34 +/− 3.06 | 14.4 +/− 0.87 | 6.76 +/− 0.26 |
| pp | T4.FPpHeliosm.pp | 25.27 +/− 7.43 | 29.32 +/− 3.53 | 23.24 +/− l.66 | 27.45 +/− 0.91 | 21.22 +/− 4.82 | 22.66 +/− 4.73 | 35.39 +/− 2.71 |
| pp | T4.FPpRorgpHeliosm.pp | 2.05 +/− 0.82 | 8.68 +/− 2.31 | 4.65 +/− 1.65 | 2.3 +/− 0.55 | 6.12 +/− 1.49 | 6.42 +/− 2.54 | 8.67 +/− 1.41 |
| pp | ILC.il22p.pp | 1.69 +/− 0.65 | 2.29 +/− 2.09 | 0.43 +/− 0.1 | 0.72 +/− 0.18 | 5.98 +/− 2.8 | 0.48 +/− 0.44 | 0.37 +/− 0.07 |
| pp | T4.ifngp.pp | 0.36 +/− 0.29 | 0.37 +/− 0.23 | 0.19 +/− 0.12 | 0.44 +/− 0.25 | 0.81 +/− 0.41 | 0.32 +/− 0.23 | 0.21 +/− 0.04 |
| pp | T4.il10p.pp | 0.92 +/− 1.6 | 0.85 +/− 0.69 | 0.29 +/− 0.15 | 0.13 +/− 0.03 | 0.87 +/− 0.58 | 1.11 +/− 0.3 | 0.51 +/− 0.03 |
| pp | T4.il17p.pp | 2.43 +/− 1.95 | 4.17 +/− 3.83 | 0.72 +/− 0.49 | 2.42 +/− 1.22 | 3.31 +/− 1.74 | 7.38 +/− 1.36 | 1.56 +/− 0.51 |
| pp | T4.il22p.pp | 0.72 +/− 0.66 | 0.33 +/− 0.37 | 0.2 +/− 0.17 | 0.88 +/− 0.04 | 0.98 +/− 0.58 | 0.16 +/− 0.05 | 0.4 +/− 0.07 |
| silp | mono.si | 5.52 +/− 2.79 | 8.35 +/− 3.34 | 7.53 +/− 1.5 | 4.92 +/− 0.19 | 5.84 +/− 1.66 | 6.41 +/− 2.67 | 6.13 +/− l.28 |
| silp | CD11bpCD11cmF4/80pMF.si | 0.61 +/− 0.44 | 0.69 +/− 0.53 | 0.59 +/− 0.16 | 0.12 +/− 0.1 | 1.6 +/− 0.44 | 0.1 +/− 0.06 | 1.19 +/− 0.69 |
| silp | CD11bpCD11cpF4/80pMNP.si | 12.23 +/− 8.54 | 14.56 +/− 5.56 | 5.12 +/− 2.64 | 13.26 +/− 1.29 | 22.46 +/− 2.66 | 7.33 +/− 1.5 | 2.98 +/− l.l |
| silp | CD103pCD11bmDC.si | 16.59 +/− 9.11 | 7.47 +/− 3.09 | 7.31 +/− 2.94 | 11.89 +/− 4.42 | 9.82 +/− 5.31 | 8.6 +/− 2.38 | 7.02 +/− 1.83 |
| silp | CD103pCD11bpDC.si | 2.15 +/− 1.66 | 2.75 +/− 2.73 | 1.36 +/− 0.45 | 3.12 +/− 1 | 2.92 +/− 1.23 | 4.06 +/− 2.97 | 1.36 +/− 0.37 |
| silp | pDC.si | 5.46 +/− 1.5 | 7.36 +/− 3.7 | 5.08 +/− 2.13 | 7.62 +/− 0.62 | 3.33 +/− 0.8 | 9.89 +/− 2.4 | 17.13 +/− 7.23 |
| silp | ILC3.si | 15.16 +/− 6.95 | 19.81 +/− 10 | 18.99 +/− 5.77 | 23.54 +/− 5.94 | 19.64 +/− 3.34 | 13.52 +/− 3.75 | 16.59 +/− 10.6 |
| silp | B.si | 39.14 +/− 16.32 | 33.28 +/− 14.99 | 34.85 +/− 12.62 | 24.9 +/− 10.04 | 38.5 +/− 8.17 | 28.57 +/− 8.99 | 40.65 +/− 21.17 |
| silp | Tgd.si | 2.94 +/− 1.51 | 4.29 +/− 1.83 | 2.73 +/− 0.77 | 3.12 +/− 0.88 | 5.28 +/− 1.3 | 7.33 +/− 3.13 | 4.02 +/− 2.61 |
| silp | Tab.si | 30.55 +/− 8.79 | 31.26 +/− 6.16 | 33.07 +/− 4.69 | 31.85 +/− 0.64 | 35.02 +/− 11.72 | 37.53 +/− 1.85 | 28.15 +/− 5.05 |
| silp | DN(CD8mCD4mTCRp).si | 6.53 +/− 3.2 | 8.13 +/− 3.16 | 9.14 +/− 1.48 | 6.13 +/− 0.73 | 9.41 +/− 0.38 | 11.43 +/− 3.02 | 6.82 +/− 1.96 |
| silp | T8.si | 24 +/− 6.3 | 22.84 +/− 2.23 | 18.25 +/− 2.67 | 22.3 +/− 3.11 | 36.53 +/− 8.97 | 26.53 +/− 4.2 | 23.28 +/− 2.59 |
| silp | T8.Heliosp.si | 10.52 +/− 6.5 | 15.73 +/− 7.23 | 7.25 +/− 3.4 | 8.89 +/− 1.99 | 15.47 +/− 7.31 | 19.35 +/− 9.51 | 11.77 +/− 4.1 |
| silp | T4.si | 65.14 +/− 5.24 | 66.46 +/− 3.47 | 67.42 +/− 2.78 | 65.85 +/− 1.2 | 55.92 +/− 10.42 | 56.47 +/− 5.95 | 66.72 +/− 3.13 |
| silp | T4.FPmRorgp.si | 2.27 +/− 1.83 | 2.92 +/− 0.82 | 15.3 +/− 1.3 | 4.28 +/− 2.45 | 4.77 +/− 2.42 | 1.46 +/− 0.91 | 15.65 +/− 8.48 |
| silp | T4.FPp.si | 24.9 +/− 8.35 | 25.82 +/− 6.87 | 18.33 +/− 3.14 | 22.25 +/− 7.42 | 24.78 +/− 4.97 | 30.43 +/− 6.47 | 15.2 +/− 2.11 |
| silp | T4.FPpHeliosm.si | 16.25 +/− 6.03 | 16.72 +/− 4.68 | 23.28 +/− 5.96 | 11.99 +/− 4.33 | 17.43 +/− 2.74 | 16.39 +/− 0.74 | 24.18 +/− 3.08 |
| silp | T4.FPpRorgpHeliosm.si | 1.81 +/− 0.74 | 5.51 +/− 1.83 | 8.94 +/− 2.14 | 2.96 +/− 0.15 | 5 +/− 0.89 | 5.52 +/− 1.96 | 12.13 +/− 0.62 |
| silp | ILC.il22p.si | 17.47 +/− 6.48 | 16.08 +/− 2.52 | 18.33 +/− 6.55 | 12.64 +/− 4.1 | 35.82 +/− 2.46 | 5.04 +/− 2.65 | 23.35 +/− 5.23 |
| silp | T4.ifngp.si | 1.77 +/− 1.28 | 1.39 +/− 0.71 | 2.97 +/− 0.94 | 0.69 +/− 0.2 | 3.65 +/− 2.41 | 1.08 +/− 0.29 | 2 +/− 0.93 |
| silp | T4.il10p.si | 0.55 +/− 0.28 | 1.5 +/− 0.55 | 2.3 +/− 1.42 | 0.15 +/− 0.08 | 1.89 +/− 0.67 | 0.83 +/− 0.56 | 2.07 +/− l.26 |
| silp | T4.il17p.si | 5.65 +/− 8.04 | 2.45 +/− 1.11 | 5.74 +/− 1.64 | 3.78 +/− 0.4 | 2.77 +/− 1.58 | 4.17 +/− 2.45 | 7.16 +/− 2.06 |
| silp | T4.il22p.si | 1.09 +/− 1.78 | 0.05 +/− 0.04 | 0.77 +/− 0.29 | 0.48 +/− 0.27 | 0.62 +/− 0.07 | 0.14 +/− 0.1 | 3.61 +/− 0.7 |
| sp | mono.slo | 3.88 +/− 2.09 | 6.84 +/− 3.04 | 3.21 +/− 1.92 | 2.76 +/− 0.13 | 4.33 +/− 1.62 | 3.82 +/− 0.13 | 5.93 +/− 0.2 |
| sp | CD11bpCD11cmF4/80pMF.slo | 0.91 +/− 0.63 | 1.29 +/− 0.82 | 0.11 +/− 0.03 | 0.31 +/− 0.3 | 1.88 +/− 0.46 | 0.57 +/− 0.07 | 2.2 +/− 0.32 |
| sp | CD11bpCD11cpF4/80pMNP.slo | 2.74 +/− 1.05 | 2.93 +/− 0.54 | 1.05 +/− 0.4 | 2.24 +/− 1.23 | 5.52 +/− 2.87 | 3.63 +/− 0.7 | 3.42 +/− l.13 |
| sp | CD103pCD11bmDC.slo | 0.08 +/− 0.03 | 0.1 +/− 0.05 | 0.02 +/− 0.01 | 0.3 +/− 0.03 | 0.09 +/− 0.01 | 0.1 +/− 0.03 | 0.47 +/− 0.75 |
| sp | CD103pCD11bpDC.slo | 0.23 +/− 0.13 | 0.29 +/− 0.19 | 0.07 +/− 0.01 | 0.38 +/− 0.1 | 0.25 +/− 0.04 | 0.59 +/− 0.09 | 0.56 +/− 0.46 |
| sp | pDC.slo | 0.6 +/− 0.11 | 0.89 +/− 0.27 | 0.44 +/− 0.1 | 0.69 +/− 0.13 | 0.44 +/− 0.08 | 0.58 +/− 0.2 | 13.09 +/− 1.44 |
| sp | ILC3.slo | 0.02 +/− 0.01 | 0.03 +/− 0.01 | 0.02 +/− 0 | 0.03 +/− 0.01 | 0.07 +/− 0.03 | 0.01 +/− 0 | 0.02 +/− 0.01 |
| sp | B.slo | 66.27 +/− 7.84 | 66.9 +/− 6.44 | 64.67 +/− l.46 | 69.95 +/− 0.92 | 74.3 +/− 2.35 | 68.37 +/− 3.42 | 60.75 +/− 3.45 |
| sp | Tgd.slo | 0.39 +/− 0.1 | 0.39 +/− 0.03 | 0.32 +/− 0.03 | 0.33 +/− 0.07 | 0.4 +/− 0.03 | 0.41 +/− 0.07 | 0.61 +/− 0.04 |
| sp | Tab.slo | 27.02 +/− 6.37 | 18.9 +/− 2.85 | 27.38 +/− 0.96 | 23.25 +/− 1.2 | 17.22 +/− 1.17 | 22.63 +/− 3.08 | 29.18 +/− 3.4 |
| sp | DN(CD8mCD4mTCRp).slo | 2.57 +/− 1.42 | 4.53 +/− 2.64 | 1.96 +/− 0.59 | 1.55 +/− 0.04 | 2.79 +/− 0.56 | 2.57 +/− 0.43 | 2.18 +/− 0.52 |
| sp | T8.slo | 33.18 +/− 2.16 | 34.33 +/− 1.47 | 32.95 +/− l.58 | 35.7 +/− 1.84 | 39.2 +/− 3.42 | 36.37 +/− 2.3 | 34.95 +/− 0.66 |
| sp | T8.Heliosp.slo | 3.53 +/− 1.53 | 4.77 +/− 1.02 | 4.72 +/− 1.08 | 3.46 +/− 0.45 | 7.56 +/− 0.86 | 4.74 +/− 0.38 | 4.57 +/− 0.86 |
| sp | T4.slo | 62.22 +/− 2.2 | 59.68 +/− 1.68 | 63.52 +/− 0.9 | 60.95 +/− 1.91 | 55 +/− 2.87 | 58.47 +/− 1.58 | 60.6 +/− l.11 |
| sp | T4.FPmRorgp.slo | 0.55 +/− 0.51 | 0.07 +/− 0 | 0.29 +/− 0.05 | 0.44 +/− 0.01 | 0.49 +/− 0.09 | 0.13 +/− 0.03 | 0.51 +/− 0.08 |
| sp | T4.FPp.slo | 12.28 +/− 1.07 | 12.73 +/− 1.49 | 10.02 +/− 0.68 | 14.3 +/− 0.71 | 12.55 +/− 2.03 | 12.73 +/− 0.8 | 13.5 +/− 0.39 |
| sp | T4.FPpHeliosm. slo | 25.82 +/− 2.23 | 21.22 +/− 2.56 | 28.55 +/− 1.91 | 23.44 +/− 1.18 | 19.13 +/− 3.22 | 26.56 +/− 0.82 | 26.62 +/− 1.43 |
| sp | T4.FPpRorgpHeliosm.slo | 0.72 +/− 0.73 | 0.4 +/− 0.18 | 0.28 +/− 0.11 | 0.44 +/− 0.1 | 0.88 +/− 0.32 | 1.29 +/− 0.03 | 1.87 +/− 0.49 |
| sp | ILC.il22p.slo | 0.09 +/− 0.08 | 0.01 +/− 0.01 | 0.02 +/− 0.01 | 0.62 +/− 0.79 | 0.04 +/− 0.04 | 0.15 +/− 0.19 | 0.14 +/− 0.04 |
| sp | T4.ifngp.slo | 1.08 +/− 0.33 | 0.86 +/− 0.16 | 1.89 +/− 0.22 | 0.6 +/− 0.03 | 2.9 +/− 0.5 | 0.23 +/− 0.04 | 0.85 +/− 0.31 |
| sp | T4.il10p.slo | 0.45 +/− 0.52 | 0.17 +/− 0.05 | 0.23 +/− 0.12 | 0.02 +/− 0.01 | 0.13 +/− 0.02 | 0.7 +/− 0.31 | 0.35 +/− 0.02 |
| sp | T4.il17p.slo | 0.91 +/− 0.85 | 0.27 +/− 0.14 | 0.28 +/− 0.14 | 0.48 +/− 0.07 | 0.4 +/− 0.34 | 0.58 +/− 0.44 | 0.53 +/− 0.06 |
| sp | T4.il22p.slo | 0.27 +/− 0.26 | 0.01 +/− 0.01 | 0.12 +/− 0.04 | 0.62 +/− 0.85 | 0.18 +/− 0.08 | 0.01 +/− 0.01 | 0.32 +/− 0.07 |

Table 4A: Fold change cell values compared to germ free (m stands for − and p stands for +) log2 value

| | | | Phylum | | | | |
|---|---|---|---|---|---|---|---|
| | | | Proteobacteria | Proteobacteria | Actinobacteria | Actinobacteria | Bacteroidetes |
| | | | Genus | | | | |
| | | | Acinetobacter | Acinetobacter | Bifidobacterium | Bifidobacterium | Bacteroides |
| organ | Row names (cell types) | Germfree | Abaum.ATCC17978 | Alwof.F78 | Badol.L2-32 | Bbrev.SK134 | Bdore.DSM17855 |
| colon | mono.co | 0 | −0.2232467 | −0.4835647 | 0.10335048 | 0.00046576 | 0.09508785 |
| colon | CD11bpCD11cmF4/80pMF.co | 0 | −2.3043799 | −2.6906365 | −0.8716974 | −3.3168925 | −2.6331765 |
| colon | CD11bpCD11cpF4/80pMNP.co | 0 | −0.5553447 | −2.0424124 | 0.12073619 | −0.7546892 | −0.7864186 |
| colon | CD103pCD11bmDC.co | 0 | −0.3349551 | −0.8727581 | 0.60957523 | 0.27165416 | 0.31976937 |
| colon | CD103pCD11bpDC.co | 0 | 0.31091608 | −1.7129307 | 1.3129712 | 2.03154247 | −0.0799881 |
| colon | pDC.co | 0 | 0.31115955 | 0.99575886 | 0.99150521 | −0.0872864 | 1.22198904 |
| colon | ILC3.co | 0 | 1.27221154 | 0.28026082 | 0.44255502 | 0.6678819 | −0.8851228 |
| colon | B.co | 0 | −0.7337197 | 0.33477483 | −0.1824786 | −0.0290405 | −0.0656703 |
| colon | Tgd.co | 0 | 0.14168804 | −1.1113303 | −0.0946052 | −0.308597 | −0.0474926 |
| colon | Tab.co | 0 | 0.33310977 | −0.9599372 | 0.0873182 | −0.1475555 | −0.0334727 |
| colon | DN(CD8mCD4mTCRp).co | 0 | 0.10860302 | −0.6576647 | 0.31102265 | −0.131846 | 0.40882502 |
| colon | T8.co | 0 | 0.07856494 | −0.295875 | −0.2283312 | −0.0663932 | −0.2538949 |
| colon | T8.Heliosp.co | 0 | 0.32861966 | −0.9794786 | 0.30935262 | −0.0780099 | 0.29467043 |
| colon | T4.co | 0 | −0.1362226 | 0.43404811 | −0.0756914 | 0.06808997 | −0.1411414 |
| colon | T4.FPmRorgp.co | 0 | 2.73672513 | 0.64462542 | 1.86613869 | 0.59560975 | 0.87184365 |
| colon | T4.FPp.co | 0 | 0.42158704 | 0.51997178 | 0.63082398 | 0.25330915 | 0.36010246 |
| colon | T4.FPpHeliosm.co | 0 | 0.0773189 | 0.66658276 | 0.4423314 | 0.30025336 | 0.46045436 |
| colon | T4.FPpRorgpHeliosm.co | 0 | 0.9123272 | 1.93324696 | 1.60533401 | 1.70248875 | 1.20190709 |
| colon | ILC.il22p.co | 0 | 0.67008529 | −1.859361 | 1.6376206 | 0.59783799 | 0.64458216 |
| colon | T4.ifngp.co | 0 | 0.26172346 | 0.50550331 | 0.46050333 | 0.71637794 | −0.1038945 |
| colon | T4.il10p.co | 0 | 0.918827 | 3.85782978 | 1.87765476 | 1.21359547 | −0.8305623 |
| colon | T4.il17p.co | 0 | −0.486168 | −0.9369332 | 0.37674358 | 1.16505925 | −1.0246621 |
| colon | T4.il22p.co | 0 | 2.34064946 | −0.5314965 | 0.29937625 | | −1.8386059 |
| mln | mono.mln | 0 | −0.4300267 | −0.7351029 | −1.4262648 | −0.9397649 | −1.644905 |
| mln | CD11bpCD11cpF4/80pMNP.mln | 0 | −1.7656629 | −1.4324468 | −2.1476657 | −3.3034603 | −3.4710144 |
| mln | CD103pCD11bmDC.mln | 0 | 0.19961986 | −0.6719131 | −0.4240665 | 0.72398484 | −0.2661237 |
| mln | CD103pCD11bpDC.mln | 0 | −0.0661811 | −0.833564 | −0.6106726 | −0.179266 | −1.2335431 |
| mln | pDC.mln | 0 | −0.6155705 | −1.2785355 | 0.29300649 | −1.291956 | −1.8046043 |
| mln | ILC3.mln | 0 | 0.09412218 | −0.1538053 | 0.80966879 | −0.1903312 | 0.33162149 |
| mln | B.mln | 0 | −0.0599527 | 0.22902524 | −0.2174384 | −0.1596994 | −0.0405804 |
| mln | Tgd.mln | 0 | 0.06472989 | −0.1328217 | 0.10118982 | 1.18971119 | −0.0793824 |
| mln | Tab.mln | 0 | 0.02482726 | −0.157164 | 0.28507496 | −0.0454045 | 0.11516641 |
| mln | DN(CD8mCD4mTCRp).mln | 0 | 0.12821843 | −0.3622154 | −0.4578805 | 0.36063747 | −0.0738699 |
| mln | T8.mln | 0 | 0.14738787 | 0.09702776 | −0.0217394 | 0.31275645 | 0.17435628 |
| mln | T8.Heliosp.mln | 0 | −0.1591718 | 0.33665505 | −0.9298661 | 0.25451762 | −1.0064502 |
| mln | T4.mln | 0 | 0.08083726 | 0.10792854 | 0.19250045 | −0.0501202 | 0.05178963 |
| mln | T4.FPmRorgp.mln | 0 | 1.16714899 | 0.33286896 | 0.85298031 | 1.04907599 | −1.2680351 |
| mln | T4.FPp.mln | 0 | −0.0844142 | 0.02370426 | −0.0046962 | −0.2987864 | −0.1129446 |
| mln | T4.FPpHeliosm.mln | 0 | −0.1213498 | −0.0852038 | 0.04182859 | −0.0454198 | 0.03671199 |
| mln | T4.FPpRorgpHeliosm.mln | 0 | 0.67620415 | 1.59981811 | 0.7640475 | 1.25756801 | 1.37160325 |
| mln | ILC.il22p.mln | 0 | −2.0892673 | −0.0291463 | 1.62527049 | 0.97819563 | −1.4344028 |
| mln | T4.ifngp.mln | 0 | 1.86734077 | 0.67843398 | 1.32076884 | −0.5295494 | 0.05122532 |
| mln | T4.il10p.mln | 0 | −0.397964 | 0.54137323 | −2.8073549 | 0.43104982 | −3.6374299 |
| mln | T4.il17p.mln | 0 | −1.1335409 | 0.42210608 | 0.08796294 | −0.5652571 | −0.5361946 |
| mln | T4.il22p.mln | 0 | −0.040642 | 0.42701014 | 1.21550604 | | −2.7914134 |
| pp | mono.pp | 0 | 0.01812455 | 0.54489362 | 0.96398846 | −0.9480538 | 0.01812455 |
| pp | CD11bpCD11cmF4/80pMF.pp | 0 | −1.7150964 | 0.15821158 | −1.0758438 | −3.6772944 | −2.325822 |
| pp | CD11bpCD11cpF4/80pMNP.pp | 0 | 1.07730127 | 0.2323119 | 1.68777331 | −0.7486252 | 0.23143939 |
| pp | CD103pCD11bmDC.pp | 0 | −0.0802721 | 0.13576167 | −0.3777696 | 0.15482071 | 0.01506205 |
| pp | CD103pCD11bpDC.pp | 0 | 1.03117149 | −0.3891158 | 1.01261078 | 0.52932604 | 0.27533892 |
| pp | pDC.pp | 0 | 0.36393562 | −0.5066904 | 1.01680829 | 0.29127537 | 0.49549167 |
| pp | ILC3.pp | 0 | 0.22239242 | 0.51019473 | 0.51019473 | −0.2895066 | −0.6181294 |
| pp | B.pp | 0 | 0.08771983 | 0.09057854 | −0.0615905 | 0.07352339 | 0.00377002 |
| pp | Tgd.pp | 0 | −0.5381213 | −0.3189121 | −1.3039259 | 0.31466385 | −0.6546669 |
| pp | Tab.pp | 0 | −0.9514198 | −0.5392215 | 0.35260279 | −0.6407619 | −0.1163524 |
| pp | DN(CD8mCD4mTCRp).pp | 0 | −0.9450824 | −0.5037033 | 0.33820587 | −0.9804788 | 0.10018237 |
| pp | T8.pp | 0 | 0.05112056 | 0.05548327 | −0.1662264 | −0.208017 | 0.08041139 |
| pp | T8.Heliosp.pp | 0 | 0.08354435 | −0.2655004 | 0.08509367 | −0.6493168 | −0.8623132 |
| pp | T4.pp | 0 | 0.12173454 | 0.01188096 | 0.12689504 | 0.29025086 | 0.04098423 |
| pp | T4.FPmRorgp.pp | 0 | −0.342106 | 0.83207735 | 1.63399084 | 1.41903943 | −0.598383 |
| pp | T4.FPp.pp | 0 | 0.43314857 | 0.3352578 | −0.2586762 | −0.1094534 | 0.27774059 |
| pp | T4.FPpHeliosm.pp | 0 | −0.4890513 | −0.1130101 | 0.02209088 | −0.0900943 | 0.05499176 |
| pp | T4.FPpRorgpHeliosm.pp | 0 | 0.79574045 | 1.58080395 | 0.18375362 | 2.31664789 | 1.44994836 |
| pp | ILC.il22p.pp | 0 | 0.45672152 | −0.4075121 | 1.21180191 | 1.25836082 | −1.9382024 |
| pp | T4.ifngp.pp | 0 | 1.9534677 | −2.0693861 | −2.9775936 | 1.47011241 | −1.8454901 |
| pp | T4.il10p.pp | 0 | 1.1079685 | 1.06066278 | −2.1263357 | 2.09559657 | −0.2367231 |
| pp | T4.il17p.pp | 0 | −3.667121 | −1.3787546 | −1.3078765 | −0.6797126 | −1.5792044 |
| pp | T4.il22p.pp | 0 | −3.707628 | 0.28061398 | −1.9083254 | | −2.0917715 |
| si | mono.si | 0 | 0.29371133 | 0.54074144 | 0.34713192 | −0.0156926 | 0.19886211 |
| si | CD11bpCD11cmF4/80pMF.si | 0 | −1.6218385 | −0.9578418 | −1.1008366 | −5.2379497 | −5.7722861 |
| si | CD11bpCD11cpF4/80pMNP.si | 0 | −1.0411456 | −0.4436796 | −0.0880944 | −0.9403287 | −1.9990887 |
| si | CD103pCD11bmDC.si | 0 | −0.0709014 | −0.4245707 | 0.07416662 | 1.07442948 | 0.65702453 |
| si | CD103pCD11bpDC.si | 0 | 1.18925803 | 0.0732862 | −0.3448511 | 1.02274944 | 1.17833724 |
| si | pDC.si | 0 | 0.22085818 | 0.82451173 | 0.5121274 | −0.0130769 | 0.08707319 |
| si | ILC3.si | 0 | 0.84451453 | 0.68084852 | 0.25203798 | 1.45678732 | 0.99731573 |
| si | B.si | 0 | −0.157368 | −0.4994686 | −0.3246783 | −0.9118226 | −0.5703261 |
| si | Tgd.si | 0 | 0.23867259 | −0.8395945 | −0.8557706 | 0.13325827 | 0.20470825 |
| si | Tab.si | 0 | −0.1637136 | 0.07032228 | 0.15944062 | −0.5171007 | 0.25281263 |
| si | DN(CD8mCD4mTCRp).si | 0 | 0.19257433 | 0.42395949 | −0.5687815 | 0.90805982 | 0.18612153 |
| si | T8.si | 0 | 0.16803241 | −0.0062937 | −0.4724843 | −0.4083507 | −0.3370321 |
| si | T8.Heliosp.si | 0 | −0.3102318 | −0.731615 | −0.1428627 | 0.63299157 | −0.6664889 |
| si | T4.si | 0 | −0.0892332 | −0.2024979 | 0.24029409 | −0.1001969 | 0.08570947 |
| si | T4.FPmRorgp.si | 0 | 1.69784487 | 1.71896962 | 1.98401165 | 1.01567868 | 0.18111574 |
| si | T4.FPp.si | 0 | −0.301294 | 0.01570578 | −0.1830573 | 0.02299659 | 0.30772344 |
| si | T4.FPpHeliosm.si | 0 | −0.4628269 | 0.27713495 | 0.00990404 | −0.3470267 | −0.275039 |
| si | T4.FPpRorgpHeliosm.si | 0 | 1.11068291 | 1.4417399 | 1.57074387 | 1.4041437 | 1.20868402 |
| si | ILC.il22p.si | 0 | 1.6574711 | −2.4057008 | 1.49033237 | 1.48631654 | 1.90657244 |
| si | T4.ifngp.si | 0 | 1.00461663 | 0.28748745 | 1.39337396 | 0.46413205 | −1.4424234 |
| si | T4.il10p.si | 0 | −0.0332846 | 1.76479013 | 2.54518804 | 0.64608179 | −2.1661334 |
| si | T4.il17p.si | 0 | 1.14359814 | −0.6614075 | 1.6065025 | 0.67945645 | −0.1258457 |
| si | T4.il22p.si | 0 | 3.8514843 | −0.9271323 | 0.86226998 | | 0.32509537 |
| slo | mono.slo | 0 | −0.4055988 | −0.2843437 | 0.14819881 | −0.3430463 | −0.9772478 |
| slo | CD11bpCD11cmF4/80pMF.slo | 0 | −1.9904604 | −2.4881092 | −1.5615447 | −5.3613856 | −3.1712017 |
| slo | CD11bpCD11cpF4/80pMNP.slo | 0 | −0.9893679 | −0.7768701 | −0.0155744 | −1.4009924 | −0.9825499 |
| slo | CD103pCD11bmDC.slo | 0 | 0.0390421 | −0.6701785 | 0.91669106 | 0.60520701 | −0.9400993 |
| slo | CD103pCD11bpDC.slo | 0 | 2.08480839 | −2.5081469 | −0.9447176 | 1.59991284 | −1.258533 |
| slo | pDC.slo | 0 | −0.3673158 | −0.8184526 | −0.2999855 | −0.6821312 | −0.688056 |
| slo | ILC3.slo | 0 | 0.13124453 | −0.6918777 | 0.4150375 | 0.0671142 | −0.2223924 |
| slo | B.slo | 0 | 0.06628821 | 0.16936338 | 0.09026392 | 0.20761401 | 0.19146467 |
| slo | Tgd.slo | 0 | 0.04053816 | −0.1661923 | 0 | 0.20844286 | −0.3878859 |
| slo | Tab.slo | 0 | −0.0268117 | 0.03937041 | −0.0310125 | −0.1781835 | −0.1945818 |
| slo | DN(CD8mCD4mTCRp).slo | 0 | −0.9908786 | 0.09872209 | −0.5643319 | −1.0535975 | −0.7429511 |
| slo | T8.slo | 0 | 0.06809915 | 0.06401508 | −0.1153988 | 0.12110975 | −0.0068999 |
| slo | T8.Heliosp.slo | 0 | −0.0223288 | 0.1771538 | −0.4118164 | 0.38476344 | −1.2016339 |
| slo | T4.slo | 0 | 0.01998027 | −0.0255903 | 0.08069266 | −0.0128471 | 0.02843577 |
| slo | T4.FPmRorgp.slo | 0 | 0.9328858 | −1.205675 | −0.0724085 | −0.5232467 | −0.3333228 |
| slo | T4.FPp.slo | 0 | 0.05132425 | 0.01916559 | 0.07995009 | 0.25162505 | −0.0473856 |
| slo | T4.FPpHeliosm.slo | 0 | 0.1591909 | 0.08103441 | −0.265533 | −0.3405521 | 0.06642943 |
| slo | T4.FPpRorgpHeliosm.slo | 0 | 1.10520303 | 2.42960361 | 0.5902763 | 1.91503044 | 1.04197227 |
| slo | ILC.il22p.slo | 0 | −1.9842327 | −0.2983413 | 2.1324503 | 0.67211445 | −2.8073549 |
| slo | T4.ifngp.slo | 0 | 0.15898952 | 0.30643781 | 1.1424293 | 0.13208165 | 0.38764094 |
| slo | T4.il10p.slo | 0 | 1.59931779 | 3.31324585 | 2.45943162 | −0.6896599 | −4.0588937 |
| slo | T4.il17p.slo | 0 | 1.81731317 | −0.477579 | 0.9328858 | 0.42484067 | 0.28113621 |
| slo | T4.il22p.slo | 0 | 0.82017896 | −0.5737352 | −0.1503343 | | −3 |

Table 4B: Continued - Fold change cell values compared to germ free (m stands for − and p stands for +) log2 value

Phylum

| | | | | | |
|---|---|---|---|---|---|
| Bacteroidetes | Bacteroidetes | Actinobacteria | Bacteroidetes | Bacteroidetes | Bacteroidetes |

Genus

| | | Bacteroides | Bacteroides | Bifidobacterium | Bacteroides | Bacteroides | Bacteroides |
|---|---|---|---|---|---|---|---|
| organ | Row names (cell types) | Bfine.DSM17565 | Bfrag.NCTC9343 | Blong.AO44 | Bmass.DSM17679 | Bovat.ATCC8483 | Bsala.DSM18170 |
| colon | mono.co | 0.12016379 | −0.2573624 | | −0.6830182 | −0.3205313 | 0.05080276 |
| colon | CD11bpCD11cmF4/80pMF.co | −0.3615616 | −0.0991754 | | −0.8442689 | −1.6083553 | −1.7004397 |
| colon | CD11bpCD11cpF4/80pMNP.co | −0.5450912 | 0.50957538 | | −0.7405441 | 0.09740423 | −0.7867961 |
| colon | CD103pCD11bmDC.co | −0.1143221 | 0.33328514 | | 0.97461164 | −0.121009 | 0.02141142 |
| colon | CD103pCD11bpDC.co | −0.8192027 | −0.4656636 | | −0.38516 | 0.59892622 | −0.9609889 |
| colon | pDC.co | 1.86429519 | 2.03107195 | | 1.36695517 | 1.43118069 | −0.1866078 |
| colon | ILC3.co | −0.1665173 | −0.6785919 | −0.1114213 | −0.8743608 | −0.4289683 | 0.45824957 |
| colon | B.co | 0.11932846 | −0.4635802 | −0.2246101 | −0.1635732 | 0.11676846 | −0.28073 |
| colon | Tgd.co | −0.7427913 | 0.70560553 | −0.082682 | 0.09637606 | −0.1823591 | 0.54410693 |
| colon | Tab.co | −0.4680633 | 0.24852944 | 0.39336065 | 0.15330676 | −0.2355662 | 0.11596232 |
| colon | DN(CD8mCD4mTCRp).co | −0.1871033 | 0.33398747 | −0.0780794 | 0.27234623 | 0.15533236 | 0.5693857 |
| colon | T8.co | −0.0453315 | 0.0333476 | 0.07851697 | 0.07534737 | 0.24591325 | −0.1067321 |
| colon | T8.Heliosp.co | 0.05426998 | 0.44460931 | 0.06348667 | 0.36554498 | 0.49214494 | 0.7019416 |
| colon | T4.co | 0.10465963 | −0.2168095 | 0.00076179 | −0.1836628 | −0.2552347 | −0.3228982 |
| colon | T4.FPmRorgp.co | 0.37233305 | 0.67305383 | 0.30645669 | 0.40538733 | 0.39323313 | 0.96347412 |
| colon | T4.FPp.co | 0.7400319 | 0.55261247 | 0.24553138 | 0.52820144 | 0.75106089 | 0.5945461 |
| colon | T4.FPpHeliosm.co | 0.5530869 | 0.5727187 | 0.00755509 | 0.6318772 | 0.32655219 | 0.42603321 |
| colon | T4.FPpRorgpHeliosm.co | 1.81873409 | 1.60476409 | 0.87145161 | 2.02343101 | 1.54459913 | 1.22739921 |
| colon | ILC.il22p.co | −1.3765196 | 1.56849484 | 0.82860593 | 0.19824054 | 1.17036 | 0.1188412 |
| colon | T4.ifngp.co | 1.32349966 | −0.5000455 | 1.56848944 | 0.87803838 | 0.47330208 | −0.2411868 |
| col on | T4.il10p.co | 0.58934094 | 1.10973144 | 2.83465835 | 1.17527463 | 2.15867491 | −0.3340378 |
| colon | T4.il17p.co | −0.5293625 | −0.3869091 | −0.9158099 | 0.47117209 | 0.33505768 | 0.14576537 |
| colon | T4.il22p.co | −0.4367921 | 0.33715766 | −1.271302 | −1.6605135 | −2.5103732 | −1.1618509 |
| mln | mono.mln | −1.9237644 | −1.0687128 | | −1.6624991 | | −1.3746026 |
| mln | CD11bpCD11cpF4/80pMNP.mln | −3.6365045 | −1.5198015 | | 0.30969907 | −1.8166782 | −3.8144222 |
| mln | CD103pCD11bmDC.mln | −0.4984076 | −0.2249664 | | −0.0480453 | −1.4331523 | −0.961932 |
| mln | CD103pCD11bpDC.mln | −2.3043934 | −0.0197362 | | 0.11668298 | −1.0682347 | −2.0237161 |
| mln | pDC.mln | −0.3823713 | −0.72054 | | −0.8735517 | −0.9144819 | −0.2852302 |
| mln | ILC3.mln | 0.49005085 | −0.2662801 | −0.6428434 | 0.16812276 | −2.4757334 | 0.99187212 |
| mln | B.mln | −0.1308497 | −0.039003 | −0.5594134 | −0.3465638 | 0.06897076 | 0.06871755 |
| mln | Tgd.mln | −0.1851771 | 0.46318831 | −0.3027467 | 0.27311361 | −0.5071052 | 0.10118982 |
| mln | Tab.mln | 0.19833725 | 0.03525317 | 0.02403911 | 0.31566844 | 0.05007093 | 0.00256286 |
| mln | DN(CD8mCD4mTCRp).mln | −0.2457564 | 0.61850555 | 0.06315835 | −0.1600265 | 1.98565092 | −0.1908725 |
| mln | T8.mln | 0.30369896 | 0.14124803 | 0.23458309 | 0.19078998 | 0.08715447 | 0.14375124 |
| mln | T8.Heliosp.mln | −0.2228394 | 0.16455183 | 0.84740088 | −0.1943316 | 0.75222332 | −0.467836 |
| mln | T4.mln | −0.0355275 | 0.02401743 | 0.02320808 | 0.04547586 | 0.06904813 | 0.06988117 |
| mln | T4.FPmRorgp.mln | 0.04907599 | 1.13341335 | 0.16937023 | −0.1749257 | −0.8261975 | 0.10119872 |
| mln | T4.FPp.mln | −0.241424 | 0.00297016 | −0.1526894 | −0.0483734 | 0.02460391 | −0.0367135 |
| mln | T4.FPpHeliosm.mln | −0.104384 | −0.1624677 | −0.3652766 | −0.0466357 | −0.3733724 | −0.211254 |
| mln | T4.FPpRorgpHeliosm.mln | 1.48057798 | 1.61677124 | 0.72566475 | 1.75284402 | 1.76066169 | 1.19572876 |
| mln | ILC.il22p.mln | 1.93168306 | 3.24412594 | 1.582556 | −0.9159357 | 0.14404637 | 2.35049725 |
| mln | T4.ifngp.mln | 1.49017832 | 0.22239242 | 0.74043779 | 0.15893914 | −1.5849625 | 0.76791852 |
| mln | T4.il10p.mln | 0.22606808 | 0.44057259 | 3.6714147 | 0.73953954 | 1.75869912 | 1.39721622 |
| mln | T4.il17p.mln | 0.69672846 | −0.55273 | −0.3050213 | 0.35277406 | −1.1713429 | 0.1012353 |
| mln | T4.il22p.mln | −0.9068906 | 1.74893824 | −1.4694853 | −1 | −0.4257639 | −1.1880723 |
| pp | mono.pp | −0.3237531 | 0.37528113 | | −0.2699819 | −0.5216254 | 0.30320345 |
| pp | CD11bpCD11cmF4/80pMF.pp | −1.0038939 | −1.1090106 | | 0.56538696 | −1.6526323 | −0.4799929 |
| pp | CD11bpCD11cpF4/80pMNP.pp | −0.6014506 | 1.42969204 | | 0.62138591 | 0.82307769 | −0.1262763 |
| pp | CD103pCD11bmDC.pp | −0.6788737 | 0.07463132 | | 0.53025207 | −0.0735397 | −0.6689273 |
| pp | CD103pCD11bpDC.pp | −1.4226779 | 0.42412222 | | 1.00585773 | 0.99008027 | −0.7671421 |
| pp | pDC.pp | 0.22268515 | 1.25738784 | | 0.81378119 | 1.04737182 | 0.47720632 |
| pp | ILC3.pp | 0.65048607 | −1.300233 | −0.4534329 | 0.60234458 | −0.4415097 | 0.41175492 |
| pp | B.pp | 0.09959393 | −0.3384062 | 0.06534559 | 0.02918584 | 0.04416214 | −0.0237885 |
| pp | Tgd.pp | −1.2085247 | 0.99953544 | −0.0371692 | 0.23914896 | −0.0476928 | −0.8492981 |
| pp | Tab.pp | −0.2084357 | 0.46838692 | −0.6029848 | −0.1738869 | −0.1813959 | 0.01551052 |
| pp | DN(CD8mCD4mTCRp).pp | −1.6545311 | 0.50245644 | −1.3891489 | −0.6026092 | 0.167075 | −0.0147534 |
| pp | T8.pp | −0.1654722 | 0.02475169 | 0.25739839 | 0.14887569 | 0.20746049 | −0.3122346 |
| pp | T8.Heliosp.pp | −0.7127554 | 0.61357118 | −0.0206038 | 0.01492956 | 0.47395807 | −1.2327578 |
| pp | T4.pp | 0.27610415 | −0.0319554 | −0.0409709 | −0.0054258 | −0.4680451 | 0.0267243 |
| pp | T4.FPmRorgp.pp | 0.03692657 | −0.519671 | 0.58614942 | 0.29822383 | −0.3534391 | −0.3511653 |
| pp | T4.FPp.pp | −0.1348221 | 0.06934972 | −0.170854 | −0.1748321 | 0.57959718 | −0.0010219 |
| pp | T4.FPpHeliosm.pp | 0.03638799 | 0.16537765 | −0.4485989 | −0.2166298 | −0.1982211 | 0.05504002 |
| pp | T4.FPpRorgpHeliosm.pp | 1.34219648 | 1.25374114 | 1.30873952 | 1.59768336 | 0.90289221 | 1.55037237 |
| pp | ILC.il22p.pp | 1.50537068 | 1.25087147 | 1.63721717 | 0.11226623 | 1.7555492 | 0.68579481 |
| pp | T4.ifngp.pp | 0.06380304 | 0.05175038 | −0.5271732 | −0.2786749 | 1.07694569 | 2.48811258 |
| pp | T4.il10p.pp | −0.7242373 | 0.70293397 | 1.03497614 | −0.1295596 | 2.57112833 | −0.7839435 |
| pp | T4.il17p.pp | −1.416354 | −2.216836 | −1.6613464 | −0.2565506 | −3.5240175 | −1.9720323 |
| pp | T4.il22p.pp | −2.3019892 | −0.7360099 | −2.0260474 | −1.7312406 | −3.2062303 | −1.5820972 |
| si | mono.si | 0.34396203 | −0.2384244 | | −0.272987 | 0.12322146 | 0.5820226 |
| si | CD11bpCD11cmF4/80pMF.si | −0.8021306 | 0.72838408 | | −0.6659263 | −1.5507823 | −4.138414 |
| si | CD11bpCD11cpF4/80pMNP.si | −0.2227343 | 0.27737065 | | −1.1752141 | −0.5845448 | −4.5531083 |
| si | CD103pCD11bmDC.si | 0.1254271 | −0.9717728 | | 0.60757014 | −0.4240136 | 1.17100085 |
| si | CD103pCD11bpDC.si | 0.41747877 | 0.55891717 | | 1.40447379 | 1.39306902 | −0.1613052 |
| si | pDC.si | 1.01567298 | 1.66593094 | | 0.64993662 | 0.18152577 | 0.22594429 |
| si | ILC3.si | 1.11432153 | −0.8144914 | 0.84841943 | 0.28218301 | 0.924428 | −0.0145055 |
| si | B.si | −1.0652106 | −0.1054037 | −0.6201342 | −0.6661342 | −0.5922339 | 0.3493537 |
| si | Tgd.si | 0.37097252 | 0.54701463 | 0.22767967 | 0.5834636 | 1.03825237 | −1.2297143 |
| si | Tab.si | 0.05031857 | −0.3300726 | −0.5536782 | 0.14593109 | 0.16814518 | −0.0457179 |
| si | DN(CD8mCD4mTCRp).si | −0.1029153 | −0.6270282 | 0.4101831 | −0.0518596 | −0.2742276 | 0.2039206 |
| si | T8.si | −0.1050985 | 0.19945985 | 0.15329708 | 0.07241042 | 0.22395764 | −0.1873271 |
| si | T8.Heliosp.si | 0.71478953 | 0.97347685 | 0.84889006 | 0.89967817 | 0.34608423 | −1.8315523 |
| si | T4.si | −0.0256726 | 0.04915556 | −0.1386146 | −0.0484373 | −0.0350995 | 0.09768345 |
| si | T4.FPmRorgp.si | −0.9114504 | −0.155995 | 0.96059998 | 0.23845514 | 0.07002689 | 0.66462345 |
| si | T4.FPp.si | 0.02152963 | 0.24449225 | 0.39115351 | 0.02375882 | 0.24981437 | −0.495377 |
| si | T4.FPpHeliosm.si | −0.303494 | 0.15410525 | −0.8863542 | −0.146067 | −0.3149087 | −0.0077099 |
| si | T4.FPpRorgpHeliosm.si | −0.3696157 | 1.5427072 | 0.87959042 | 1.83621183 | 1.84187423 | 1.28568665 |
| si | ILC.il22p.si | 0.42405592 | 0.92735221 | 1.48778243 | 0.91728051 | 1.40320859 | 1.14274786 |
| si | T4.ifngp.si | −1.2989913 | −0.1458509 | 1.06509503 | 1.64178314 | 1.63286474 | 0.91328837 |
| si | T4.il10p.si | −0.9848931 | 1.41882908 | 0.64931291 | 0.59839864 | 1.5849625 | −0.4859331 |
| si | T4.il17p.si | 1.03219059 | −0.072434 | −0.0202267 | 0.49469766 | 0.80646416 | 0.04969434 |
| si | T4.il22p.si | −1.8394161 | 0.39048283 | 0.98061937 | 0.11111519 | 0.05117265 | 2.03302531 |
| slo | mono.slo | −1.3193633 | 0.84837716 | | −0.9030451 | −0.2558644 | −0.0165235 |
| slo | CD11bpCD11cmF4/80pMF.slo | −2.9139266 | 0.29344572 | | −3.3035415 | −1.6956621 | −1.9091256 |
| slo | CD11bpCD11cpF4/80pMNP.slo | −2.2815347 | 0.12078985 | | −0.9750871 | −0.0626134 | −0.8973695 |
| slo | CD103pCD11bmDC.slo | −0.3210289 | −0.1798529 | | −0.2508536 | −1.1798529 | −0.8530426 |
| slo | CD103pCD11bpDC.slo | −1.6962193 | −1.1296353 | | 0.78135971 | −2.0692627 | −1.9125372 |
| slo | pDC.slo | −1.3744638 | −0.4156518 | | −0.5603004 | −0.8613244 | −0.0795388 |
| slo | ILC3.slo | −0.1443899 | 0.73696559 | 0 | −0.3923174 | −0.0703893 | 0.13124453 |
| slo | B.slo | −0.0914199 | 0.02917885 | −0.0259001 | −0.2078541 | −0.1123249 | 0.11360079 |
| slo | Tgd.slo | 0.31443356 | 0.68133856 | −0.5868325 | 0.63876414 | 0.1690942 | 0.43268253 |
| slo | Tab.slo | 0.61891601 | 0.13300477 | −0.0558559 | 0.78694666 | 0.60094491 | 0.02716995 |
| slo | DN(CD8mCD4mTCRp).slo | −1.3808774 | −0.2769795 | −0.5144642 | −1.0191902 | −0.7791334 | −0.6107359 |
| slo | T8.slo | 0.08177793 | 0.1075228 | 0.08629543 | 0.11851285 | 0.03301041 | 0.02092529 |
| slo | T8.Heliosp.slo | −0.363239 | 0.26910695 | 0.32273925 | −0.4638072 | 0.37949551 | −0.8493082 |
| slo | T4.slo | 0.0273551 | −0.0329964 | −0.0177126 | −0.0116635 | −0.0008385 | 0.00635707 |
| slo | T4.FPmRorgp.slo | 0.77381932 | 0.08330265 | −0.6129769 | 0.33598369 | −0.5824425 | −0.5451615 |
| slo | T4.FPp.slo | −0.2058703 | 0.12273202 | 0.08278169 | −0.0475094 | −0.0138473 | 0.07711294 |
| slo | T4.FPpHeliosm.slo | −0.0119125 | −0.2885957 | −0.2028052 | −0.2480919 | −0.3472951 | 0.10540385 |
| slo | T4.FPpRorgpHeliosm.slo | 1.37112159 | 2.50937331 | 1.47435701 | 2.37215025 | 2.15744949 | 0.7664739 |
| slo | ILC.il22p.slo | 1 | 2.76366839 | 1.28662123 | −3.5077946 | −0.4854268 | −0.600904 |
| slo | T4.ifngp.slo | 0.6961235 | 0.44407004 | −0.2414857 | 0.33725383 | −0.636652 | 0.98149801 |
| slo | T4.il10p.slo | 1.28095631 | 0.29865832 | 1.38956681 | 0.43295941 | 1.56559718 | 0.12432814 |
| slo | T4.il17p.slo | 2.06099063 | −0.8585276 | −0.8744691 | 1.62944494 | −1.4238077 | 0 |
| slo | T4.il22p.slo | −0.1926451 | 0.33985 | −2.4974997 | −1.2995603 | −1.2995603 | −0.4050534 |

Table 4C: Continued - Fold change cell values compared to germ free (m stands for − and p stands for +) log2 value

| | | Phylum | | | | | |
|---|---|---|---|---|---|---|---|
| | | Bacteroidetes | Bacteroidetes | Bacteroidetes | Actinobacteria | Firmicutes | Proteobacteria |
| | | Genus | | | | | |
| | | Bacteroides | Bacteroides | Bacteroides | Collinsella | Clostridium | Campylobacter |
| organ | Row names (cell types) | Bthet.ATCC29741 | Bunif.ATCC8492 | Bvulg.ATCC8482 | Caero.VPI1003 | Chist.AO25 | Cjeju.AS-84-79 |
| colon | mono.co | −0.046871 | 0.47111479 | −0.0483154 | −0.487478 | 0.36840709 | 0.74736277 |
| colon | CD11bpCD11cmF4/80pMF.co | −0.910995 | −3.0681715 | −0.8334419 | −3.8451748 | −1.6129769 | −1.6922658 |
| colon | CD11bpCD11cpF4/80pMNP.co | 1.04904818 | −0.714278 | 0.80519635 | −4.4152563 | −1.503084 | −1.0151783 |
| colon | CD103pCD11bmDC.co | −0.9293417 | −0.4300092 | −0.153202 | 0.20237057 | 0.19116773 | −0.0015416 |
| colon | CD103pCD11bpDC.co | 1.17910832 | 1.90952441 | 0.04149213 | 0.73313373 | 1.40495707 | 0.34947141 |
| colon | pDC.co | 1.20739166 | 1.90255506 | 2.4460231 | −1.242292 | 0.26726312 | 0.19387973 |
| colon | ILC3.co | 0.14751206 | 1.55586036 | −0.3341648 | −0.0249086 | 0.60859616 | 0.05920559 |
| colon | B.co | −0.0320303 | −0.2303897 | −0.1920251 | 0.0980482 | 0.05626483 | 0.31596731 |
| colon | Tgd.co | 0.39148662 | 0.04597888 | 0.07878915 | −0.2978363 | −0.3233304 | −0.5076486 |
| colon | Tab.co | −0.1601031 | −0.1293749 | 0.03281816 | −0.0904392 | −0.2607698 | −0.5410836 |
| colon | DN(CD8mCD4mTCRp).co | −0.0114633 | 0.18733724 | 0.19722946 | 0.15465906 | −0.1221483 | −0.6584836 |
| colon | T8.co | −0.054403 | −0.2099347 | −0.0444954 | 0.17027954 | −0.2812119 | −0.1204881 |
| colon | T8.Heliosp.co | 0.36274786 | 0.02262319 | 0.52056444 | −0.7745535 | −0.8781518 | −0.9646997 |
| colon | T4.co | 0.07944375 | 0.00117715 | −0.1102681 | −0.1592448 | 0.22143714 | 0.41406736 |
| colon | T4.FPmRorgp.co | 0.7645915 | 1.24826495 | 0.8918512 | 1.23538668 | 1.92852825 | 1.37789496 |
| colon | T4.FPp.co | 0.76717628 | 0.80296315 | 1.17501142 | 0.21199992 | 0.75758335 | 0.37771485 |
| colon | T4.FPpHeliosm.co | 0.43906252 | 0.68143611 | 0.5620569 | 0.2479503 | 0.85629338 | 0.93345252 |
| colon | T4.FPpRorgpHeliosm.co | 2.24857752 | 1.97038697 | 2.21092279 | 0.4650357 | 2.27407181 | 2.39337998 |
| colon | ILC.il22p.co | | 2.27217131 | −1.0584785 | 0.77011304 | 0.96833069 | 1.57460263 |
| colon | T4.ifngp.co | 0.89504898 | 0.03111053 | 0.82687748 | −3.9346738 | 1.37214745 | 1.33756722 |
| colon | T4.il10p.co | 3.04213336 | 1.58203614 | 2.54794931 | | 2 | 1.96785256 |
| colon | T4.il17p.co | −2.0846868 | −0.060531 | −0.0815471 | −1.2387869 | −0.6266846 | 0.4636692 |
| colon | T4.il22p.co | −3.7271846 | 1.03634185 | −1.7868908 | 1.84061738 | 0.67807191 | 0.39651741 |
| mln | mono.mln | −1.2038723 | −1.3673711 | −1.4262648 | −0.7824086 | −1.0983379 | −0.2969817 |
| mln | CD11bpCD11cpF4/80pMNP.mln | −1.1605024 | −2.0174093 | −1.1098269 | −2.996799 | −2.3549906 | −1.9790034 |
| mln | CD103pCD11bmDC.mln | −1.7655347 | −0.0721745 | −6.4594316 | 0.03712848 | 0.23446525 | 0.07149104 |
| mln | CD103pCD11bpDC.mln | −0.8024628 | −0.3461424 | −6.9385995 | 0.17861073 | −0.3461424 | −0.2971498 |
| mln | pDC.mln | −0.1265324 | −0.291956 | −0.4559942 | −1.0909085 | −0.863498 | 0.35459428 |
| mln | ILC3.mln | | −0.083416 | | −0.083416 | −0.1181814 | −0.1719527 |
| mln | B.mln | 0.05460844 | −0.0137578 | 0.14342279 | −0.2582912 | 0.07363314 | 0.24031105 |
| mln | Tgd.mln | 0.05148191 | 0.27311361 | −0.3617822 | −0.166481 | 0.10632398 | 1.11271608 |
| mln | Tab.mln | −0.0192054 | 0.06828989 | −0.0363335 | −0.0545662 | −0.0030172 | −0.2484867 |
| mln | DN(CD8mCD4mTCRp).mln | −0.5135094 | −0.2546164 | −0.0174874 | −0.2398798 | 0.16854483 | 0.49884406 |
| mln | T8.mln | 0.18544005 | 0.07501606 | 0.09512697 | 0.09036403 | 0.10490825 | 0.23806698 |
| mln | T8.Heliosp.mln | 0.75572153 | 0.02393466 | 0.56407439 | −0.334046 | 0.3506024 | 0.60625728 |
| mln | T4.mln | 0.04950594 | 0.04515196 | 0.11509152 | 0.10554092 | 0.10893017 | 0.00538894 |
| mln | T4.FPmRorgp.mln | | 1.53415813 | | 0.95693128 | 1.15374925 | 0.64205794 |
| mln | T4.FPp.mln | 0.12417705 | −0.1463473 | 0.15274621 | 0.08428433 | 0.05607324 | 0.04602881 |
| mln | T4.FPpHeliosm.mln | −0.6772561 | 0.01631174 | −0.692402 | −0.3089578 | −0.0508559 | −0.3047952 |
| mln | T4.FPpRorgpHeliosm.mln | | 1.42690572 | | 0.50870317 | 1.83948465 | 2.03349315 |
| mln | ILC.il22p.mln | | 2.20006486 | −0.0439433 | 1.41413553 | 0.50080205 | 0.31614574 |
| mln | T4.ifngp.mln | −0.3270067 | 0.54183525 | 0.55175072 | 0.17698918 | 1.13407575 | 1.71242341 |
| mln | T4.il10p.mln | 3.32450204 | 1.56768451 | 1.94083793 | −0.2527661 | 1 | 2.09652692 |
| mln | T4.il17p.mln | −0.888234 | 0.14802951 | 1.12741923 | −0.7579022 | −0.1027786 | 1.05423782 |
| mln | T4.il22p.mln | −2.3625701 | −0.1255309 | −1.3625701 | −0.372912 | 0.19464743 | −0.6974372 |
| pp | mono.pp | −0.1866647 | 0.21009405 | −1.2788057 | 0.61206465 | −0.0332093 | 0.07636969 |
| pp | CD11bpCD11cmF4/80pMF.pp | −0.4854268 | −2.3064567 | −1.5240481 | −1.8487748 | −2.1954254 | −1.0354295 |
| pp | CD11bpCD11cpF4/80pMNP.pp | 1.40707459 | 1.14512161 | 1.40475453 | 1.2059043 | 0.21740699 | 0.49984589 |
| pp | CD103pCD11bmDC.pp | −0.3180738 | 0.47589521 | 0.32965596 | 0.07660337 | 0.57846599 | −0.7385498 |
| pp | CD103pCD11bpDC.pp | 0.81220136 | 1.08575364 | −0.0192818 | 0.46004934 | 0.98665455 | −0.068969 |
| pp | pDC.pp | 1.08021189 | −0.0671142 | 0.75306477 | 0.24879011 | 0.32683801 | −0.4566166 |
| pp | ILC3.pp | | 0.60522134 | | −0.6520767 | 0.43133931 | 0.23359063 |
| pp | B.pp | −0.0065687 | 0.05234258 | −0.0309129 | −0.0679883 | 0.058649 | 0.09223531 |
| pp | Tgd.pp | 0.79495976 | 0.73510645 | 0.97823807 | −0.0851423 | 0.54126494 | −0.6775146 |
| pp | Tab.pp | −0.2016725 | −0.6213934 | 0.06874148 | 0.38795829 | −0.6535773 | −0.3952056 |
| pp | DN(CD8mCD4mTCRp).pp | −1.411476 | −0.7243331 | −1.426778 | −1.045234 | −0.9814609 | −1.6350822 |
| pp | T8.pp | 0.24042531 | 0.29075699 | 0.45996937 | 0.23563684 | 0.22321177 | 0.02712889 |
| pp | T8.Heliosp.pp | 0.25913427 | 0.47320529 | 0.9106437 | −0.0507567 | −0.016822 | −0.5782585 |
| pp | T4.pp | −0.0235808 | −0.0736646 | −0.1412758 | 0.05218054 | −0.0269088 | 0.18411059 |
| pp | T4.FPmRorgp.pp | | 0.54767527 | | 1.1310122 | 1.68639677 | −0.4114797 |
| pp | T4.FPp.pp | 0.01982959 | 0.08466865 | 0.32231809 | 0.12788933 | 0.52797649 | 0.02202631 |
| pp | T4.FPpHeliosm.pp | −1.0680098 | 0.10461064 | −0.824465 | −0.3973807 | 0.28612067 | −0.0570669 |
| pp | T4.FPpRorgpHeliosm.pp | | 1.85004511 | | 1.11285799 | 2.07787642 | 1.81370666 |
| pp | ILC.il22p.pp | | 0.38828398 | −1.1948592 | 0.85999252 | −0.4804319 | −0.0252373 |
| pp | T4.ifngp.pp | 0.46557705 | −1.0278668 | 0.10870626 | −3.3094372 | 0.15901134 | −1.6675723 |
| pp | T4.il10p.pp | 2.43536135 | 1.25118469 | 0.63045884 | −1.9002677 | −0.421938 | −0.0819416 |
| pp | T4.il17p.pp | −2.2281736 | −1.2540136 | −2.9613501 | −2.9684628 | −3.1133532 | 1.13885556 |
| pp | T4.il22p.pp | 0.81267174 | −1.9800612 | −1.4547783 | −2.2329476 | −3.3305584 | 0.06587513 |
| si | mono.si | 0.33081212 | 0.00653032 | −0.2524501 | −1.1303218 | 0.36424734 | 0.20913356 |
| si | CD11bpCD11cmF4/80pMF.si | 1.12712288 | −1.5401547 | 0.38584471 | −9.6302671 | −0.0969374 | −1.1811185 |
| si | CD11bpCD11cpF4/80pMNP.si | 0.68587499 | −0.7253898 | 0.56682028 | −10.043179 | −1.5198351 | −0.3140306 |
| si | CD103pCD11bmDC.si | 0.27552615 | 0.46337619 | −0.6345116 | −0.6901331 | 1.15082643 | −0.0112521 |
| si | CD103pCD11bpDC.si | −0.6335092 | −0.7500559 | 0.99736631 | −2.0173957 | 0.41972858 | 0.64940878 |
| si | pDC.si | 0.30053058 | 0.60484642 | 1.03329922 | −1.0112344 | −0.2262802 | −0.2076987 |
| si | ILC3.si | | 0.02283283 | | 0.76083472 | 0.34978193 | 0.32064027 |
| si | B.si | −0.1564547 | 0.08554214 | −0.1432012 | −0.1114751 | −0.3435672 | −0.0688218 |
| si | Tgd.si | −0.4556795 | −0.0949083 | 0.48132792 | 0.94488763 | −0.0056289 | −0.1900791 |
| si | Tab.si | 0.18029923 | −0.2967032 | 0.16638059 | 0.38083221 | 0.07666533 | 0.39474137 |
| si | DN(CD8mCD4mTCRp).si | 0.15329013 | 0.30312864 | −0.2697854 | −0.4771026 | 0.1772641 | −0.9806045 |
| si | T8.si | −0.5349653 | −0.1364848 | −0.0655909 | −0.6001918 | −0.2981075 | −0.1936134 |
| si | T8.Heliosp.si | −0.1395024 | 0.16852305 | 0.75672021 | 0.2288469 | −1.4327224 | −0.7937918 |
| si | T4.si | 0.25018466 | −0.0446139 | 0.13187214 | 0.30965926 | 0.03681831 | 0.24031498 |
| si | T4.FPmRorgp.si | | 0.23337766 | | −2.1118578 | 1.68927388 | 0.15840327 |
| si | T4.FPp.si | 0.18382854 | −0.0897365 | 0.487766 | 0.44576958 | −0.1527979 | 0.19298024 |
| si | T4.FPpHeliosm.si | −1.4165859 | 0.48520919 | −1.207478 | −0.2834902 | −0.2764441 | −0.1355897 |
| si | T4.FPpRorgpHeliosm.si | | 1.79190636 | | 0.74677204 | 1.5741497 | 1.44510965 |
| si | ILC.il22p.si | | 1.23579348 | −0.1187505 | 0.98547901 | 0.92609709 | 1.44898252 |
| si | T4.ifngp.si | 1.42815614 | 0.95967506 | 1.60151368 | −1.467779 | 0.83042176 | 0.01288951 |
| si | T4.il10p.si | 1.10962449 | 0.4150375 | 2.06495544 | −1.9356496 | 0.9445297 | 0.38240029 |
| si | T4.il17p.si | 0.33310488 | −0.7092976 | 0.03219059 | −0.2745777 | 0.41330495 | 1.03455927 |
| si | T4.il22p.si | 3.14120485 | 1.09424137 | −2.5469652 | 0.15855619 | 1.83840247 | −1.5736826 |
| slo | mono.slo | −0.9360631 | −0.0374477 | 0.04515108 | −0.2123871 | −0.3790936 | −1.0849408 |
| slo | CD11bpCD11cmF4/80pMF.slo | −1.190004 | −1.7873942 | −0.9628362 | −2.998087 | −1.8253327 | −2.2061073 |
| slo | CD11bpCD11cpF4/80pMNP.slo | 0.00789579 | 0.05769525 | 0.18608971 | −0.3969074 | −0.5705765 | −0.8569057 |
| slo | CD103pCD11bmDC.slo | −3.3620562 | 0.0495091 | −0.6306501 | 0.35816612 | 0.62866598 | −0.3210289 |
| slo | CD103pCD11bpDC.slo | −2.642448 | −1.0227201 | −1.7332135 | −0.7145978 | −0.4686185 | −2.0692627 |
| slo | pDC.slo | −0.027906 | −0.3437418 | −0.169925 | −0.2509247 | −0.9618168 | −1.9124288 |
| slo | ILC3.slo | 1 | 0.19264508 | −0.3048546 | 0 | −0.4854268 | 0.36257008 |
| slo | B.slo | 0.0776826 | 0.09210305 | 0.08703979 | 0.11848982 | 0.08403948 | 0.2021284 |
| slo | Tgd.slo | 0.3293839 | 0.22452659 | −0.2001396 | −0.320994 | 0.14900995 | 0.31743605 |
| slo | Tab.slo | −0.1257241 | −0.0276238 | 0.1133096 | −0.1576664 | 0.12669259 | −0.2028896 |
| slo | DN(CD8mCD4mTCRp).slo | −0.9645993 | −0.1909032 | −1.0292708 | 0.08261042 | −0.8716759 | −0.5665888 |
| slo | T8.slo | −0.1629037 | 0.10183686 | −0.0112022 | 0.04962912 | 0.11914283 | 0.12114906 |
| slo | T8.Heliosp.slo | 0.65094145 | −0.2196904 | −0.169691 | −1.2240578 | 0.1018016 | 0.26547688 |
| slo | T4.slo | −0.139987 | −0.0449309 | 0.0590144 | −0.0146607 | 0.00134066 | −0.0341732 |
| slo | T4.FPmRorgp.slo | 0.33198176 | 0.00503559 | −1.2895066 | 0.22814595 | −0.205675 | −0.1542468 |
| slo | T4.FPp.slo | 0.00788752 | 0.1850907 | 0.0505145 | 0.09113077 | −0.0101014 | 0.13555216 |
| slo | T4.FPpHeliosm.slo | −0.4766848 | −0.1373488 | −0.7548714 | −0.2863293 | −0.3076852 | −0.554468 |
| slo | T4.FPpRorgpHeliosm.slo | 1.9879719 | 1.81569642 | 1.55266201 | 2.67857091 | 2.45312634 | 2.17051639 |
| slo | ILC.il22p.slo | | 0.7589919 | −4.9228321 | 1.39909596 | 0.45798964 | 1.50901365 |
| slo | T4.ifngp.slo | −2.6096849 | −0.8567778 | −1.3781615 | −0.7528099 | −0.2581404 | 0.92912911 |
| slo | T4.il10p.slo | 3.1210154 | 0.87970577 | 3.35614381 | 1.33342373 | 1.84398384 | 1.169925 |
| slo | T4.il17p.slo | 0.57619229 | −0.447459 | −0.25258 | −0.9400575 | 0.25206329 | 1.78469433 |
| slo | T4.il22p.slo | −3 | −1.6076826 | 0.81164228 | −0.5737352 | −0.6901447 | −3.6780719 |

Table 4D: Continued - Fold change cell values compared to germ free (m stands for − and p stands for +) log2 value

Phylum

| | | | | | |
|---|---|---|---|---|---|
| Firmicutes | Firmicutes | Firmicutes | Firmicutes | Proteobacteria | Firmicutes |

Genus

| | | Coprobacillus | Clostridium | Clostridium | Clostridium | Escherichia | Enterococcus |
|---|---|---|---|---|---|---|---|
| organ | Row names (cell types) | Copr.8.2.54BFAA | Cperf.ATCC13124 | Cramo.AO31 | Csord.AO32 | Ecoli.Nissle1917 | Efaec.TX0104 |
| colon | mono.co | 0.29740418 | −0.6965436 | 0.35244149 | 2.5653422 | 0.80342324 | 0.4727941 |
| colon | CD11bpCD11cmF4/80pMF.co | −1.7202482 | −0.3583246 | −2.0513469 | −0.4733708 | −0.8940219 | 0.38972083 |
| colon | CD11bpCD11cpF4/80pMNP.co | −2.6622722 | −0.3312306 | −0.6377217 | −0.0251639 | 0.30745726 | 0.25056249 |
| colon | CD103pCD11bmDC.co | −0.4671562 | −0.609567 | −0.2972312 | −0.0851535 | −0.335603 | 0.14778544 |
| colon | CD103pCD11bpDC.co | 1.33942474 | −2.777061 | −0.5735276 | 0.00254893 | 0.82456349 | 1.63879598 |
| colon | pDC.co | 0.53906774 | −0.1516894 | 0.59434287 | 0.78990684 | 1.63581572 | −0.1833983 |
| colon | ILC3.co | −1.4072601 | 1.11967023 | 0.46955749 | −0.0664954 | 0.60261695 | 0.75902144 |
| colon | B.co | −0.2709693 | −0.2928345 | 0.00440873 | −0.0959716 | 0.01416887 | −0.2884651 |
| colon | Tgd.co | −0.1937133 | 0.19544552 | −0.0372288 | −0.7904793 | 0.64674367 | 0.2927309 |
| colon | Tab.co | 0.36765695 | 0.1620758 | −0.2144498 | −0.0048188 | −0.3059146 | −0.0546904 |
| colon | DN(CD8mCD4mTCRp).co | 0.16626817 | 0.397312 | 0.16240884 | −1.1104594 | 0.35054474 | 0.1614295 |
| colon | T8.co | 0.01727076 | −0.2804119 | −0.006702 | −0.2142768 | −0.3724963 | 0.00530896 |
| colon | T8.Heliosp.co | 0.41055525 | 0.02386969 | 0.13426331 | −1.3398906 | 0.47370716 | 0.28414176 |
| colon | T4.co | −0.0656882 | −0.027237 | −0.030311 | 0.27737724 | 0.01536556 | −0.0478912 |
| colon | T4.FPmRorgp.co | −0.2083017 | 0.04108719 | 1.671545 | 2.71930515 | 1.47796509 | 2.10247151 |
| colon | T4.FPp.co | 0.76538246 | 0.95854417 | 0.84927019 | 1.03687717 | 0.96689334 | 0.8713727 |
| colon | T4.FPpHeliosm.co | 0.60640882 | 0.37783722 | 0.77960363 | 0.81641629 | 0.53120129 | 0.64498117 |
| colon | T4.FPpRorgpHeliosm.co | 0.8968142 | 0.54266632 | 2.37155991 | 2.16647747 | 1.60141122 | 1.84080321 |
| colon | ILC.il22p.co | −1.9651837 | 1.57973232 | 1.20319613 | −2.1725972 | 0.60276868 | 0.74420891 |
| colon | T4.ifngp.co | 0.74128328 | 0.56649997 | −0.3422167 | 1.08120586 | 0.60448506 | 0.49298829 |
| colon | T4.il10p.co | 2.58569316 | 0.59515827 | 0.19647694 | 1.10973144 | 0.21170341 | 1.53438863 |
| colon | T4.il17p.co | 0.14643499 | −1.1906352 | −0.3197069 | −0.5541721 | 0.61212164 | 0.06377591 |
| colon | T4.il22p.co | −1.2155388 | −2.8281135 | −1.243151 | 0.25565488 | −0.5529336 | −0.7468134 |
| mln | mono.mln | −0.6318489 | −1.8513559 | −0.0657834 | 0.10251491 | −0.2901281 | −0.071648 |
| mln | CD11bpCD11cpF4/80pMNP.mln | −3.7613109 | −3.6687448 | −2.4463524 | −1.8789625 | −1.6545509 | −0.924462 |
| mln | CD103pCD11bmDC.mln | −2.60438 | −1.9358697 | −0.2328778 | −0.3204823 | −0.2092726 | 0.33038967 |
| mln | CD103pCD11bpDC.mln | −0.8162028 | −5.8231222 | −0.7356594 | −0.6531972 | −0.2404736 | −0.0702913 |
| mln | pDC.mln | −0.5256284 | −0.0792267 | −0.8337507 | −0.2718718 | −0.6583836 | −0.7572986 |
| mln | ILC3.mln | −0.6177524 | 1.52989112 | 0.31868244 | 0.2107671 | −0.2662801 | −0.0663425 |
| mln | B.mln | 0.28398582 | 0.03386017 | 0.17057734 | −0.0894764 | 0.20447041 | 0.081045 |
| mln | Tgd.mln | −0.1328217 | −0.323308 | −0.3336996 | 0.36165029 | −0.3336996 | 0.75723542 |
| mln | Tab.mln | −0.3318297 | −0.0670745 | −0.1253385 | −0.1385139 | −0.262842 | −0.1042637 |
| mln | DN(CD8mCD4mTCRp).mln | 0.55231527 | 0.06315835 | −0.081732 | 0.32738065 | 0.44696227 | 0.38556172 |
| mln | T8.mln | 0.12057864 | 0.19253333 | 0.24662023 | 0.08458934 | 0.02129656 | 0.11788509 |
| mln | T8.Heliosp.mln | −0.0619136 | −0.4011602 | 0.40993346 | −0.1103021 | 0.68776006 | 0.59990178 |
| mln | T4.mln | 0.06435947 | 0.04455382 | 0.0123901 | 0.08268353 | 0.15005814 | 0.09758557 |
| mln | T4.FPmRorgp.mln | −0.6280312 | −0.9365209 | 0.55299477 | 1.31090363 | 0.3088901 | 1.20228485 |
| mln | T4.FPp.mln | 0.17317409 | −0.0542983 | −0.0935364 | −0.0514513 | −0.0682582 | 0.02561534 |
| mln | T4.FPpHeliosm.mln | −0.3331285 | 0.16496716 | −0.0538655 | −0.1176942 | −0.1613383 | −0.2160829 |
| mln | T4.FPpRorgpHeliosm.mln | 0.70819427 | 1.09339654 | 1.87028618 | 1.66646714 | 1.08746284 | 1.86367336 |
| mln | ILC.il22p.mln | −3 | −0.1520031 | 0.09085343 | 1 | 2.30888506 | 0.70929064 |
| mln | T4.ifngp.mln | 0.60224049 | −0.8051838 | 1.38318378 | 2.16282606 | 0.86017703 | 1.80900939 |
| mln | T4.il10p.mln | 2.57850748 | 0.59352451 | 0.37255417 | 1.05062607 | 2.01282404 | 1.89654865 |
| mln | T4.il17p.mln | 0.51428971 | 0.41574592 | −0.3585266 | 0.37261281 | 0.77666248 | 0.39434056 |
| mln | T4.il22p.mln | −1.0489096 | −3.3219281 | 0.05504136 | −0.8336416 | −0.9840585 | −0.447459 |
| pp | mono.pp | −0.1763228 | −1.1640095 | 0.65439614 | 0.75940636 | −0.4526082 | 0.83292201 |
| pp | CD11bpCD11cmF4/80pMF.pp | −1.0117134 | −1.5579955 | −0.9503129 | −0.395724 | −2.4908813 | 0.47315325 |
| pp | CD11bpCD11cpF4/80pMNP.pp | −1.0800533 | −0.2622149 | 1.1658206 | 1.09946736 | 0.41170357 | 1.01528833 |
| pp | CD103pCD11bmDC.pp | −1.0404936 | −1.5267756 | 0.27021588 | 0.42859305 | 0.08082013 | 0.00163192 |
| pp | CD103pCD11bpDC.pp | −0.2606885 | −1.7840654 | 0.28297628 | 0.5421122 | 0.76207788 | 0.70750398 |
| pp | pDC.pp | 0.67755949 | −0.889576 | 0.31040623 | 0.2735837 | 0.62736807 | 0.83691107 |
| pp | ILC3.pp | −1.6938969 | 0.99781244 | 0.03455722 | 0.56736844 | 0.22613482 | 0.37851162 |
| pp | B.pp | 0.03366605 | 0.03480953 | −0.0100842 | 0.06074502 | −0.0719692 | −0.0368889 |
| pp | Tgd.pp | 0.42913523 | −1.2532207 | 0.09046522 | 0.3273061 | 1.34734258 | 0.62496783 |
| pp | Tab.pp | −0.5477729 | 0.2484475 | 0.07740105 | −0.5762653 | 0.06476122 | −0.0967372 |
| pp | DN(CD8mCD4mTCRp).pp | −0.8433769 | −1.7458663 | −1.224301 | −0.115726 | −0.9834271 | −0.6358552 |
| pp | T8.pp | −0.1319832 | −0.0024673 | −0.11606 | 0.04026469 | 0.52551716 | −0.0664527 |
| pp | T8.Heliosp.pp | 0.15736877 | −1.0987999 | −0.0821065 | 0.25594903 | 0.86386215 | 0.56987683 |
| pp | T4.pp | 0.14383043 | 0.1997535 | 0.22942413 | 0.07655046 | −0.2011918 | 0.08291266 |
| pp | T4.FPmRorgp.pp | −0.8037971 | −0.6505508 | 0.1962029 | 1.39282622 | −0.3219281 | 1.19698 |
| pp | T4.FPp.pp | −0.1840869 | −0.0995688 | 0.15733311 | 0.28477106 | 0.09852616 | 0.19359666 |
| pp | T4.FPpHeliosm.pp | −0.1258089 | 0.14991588 | 0.3675751 | −0.2027724 | −0.2520163 | 0.08970547 |
| pp | T4.FPpRorgpHeliosm.pp | 0.37212122 | 0.59879112 | 2.64241805 | 2.22215283 | 1.39707816 | 1.89737226 |
| pp | ILC.il22p.pp | −1.3201422 | 3.8114337 | −0.7825515 | 0.32216634 | −0.8734767 | 0.01893638 |
| pp | T4.ifngp.pp | −1.6675723 | 0.30222367 | −1.7327899 | 1.51557644 | 0.75442279 | −0.1395121 |
| pp | T4.il10p.pp | 2.14579413 | −1.5499864 | 0.64939211 | 1.19559236 | −0.0882006 | 0.13777171 |
| pp | T4.il17p.pp | −0.1899147 | −1.9113766 | −1.797218 | −1.1958153 | −0.1906076 | −0.9245729 |
| pp | T4.il22p.pp | −1.0525737 | −2.7455959 | −1.2262217 | −1.4905988 | −0.8348632 | −1.1133277 |
| si | mono.si | 0.35404186 | −0.2720748 | 1.36258407 | 0.76809425 | 0.20913356 | 0.3360067 |
| si | CD11bpCD11cmF4/80pMF.si | −1.5269793 | −2.1545337 | −1.0988857 | 0.40315588 | −2.1106309 | 1.37535742 |
| si | CD11bpCD11cpF4/80pMNP.si | −4.4632348 | −1.5710525 | 0.06076421 | −0.3093165 | −0.4281627 | 0.05463346 |
| si | CD103pCD11bmDC.si | 0.82772937 | −0.0694487 | −0.528717 | −0.5152059 | −0.4504308 | −0.5387329 |
| si | CD103pCD11bpDC.si | −1.2951142 | 1.06360726 | −0.3108815 | −0.313682 | −0.170583 | −0.3553922 |
| si | pDC.si | −0.4031347 | 0.43415158 | −0.1299183 | −0.2335117 | 0.26789837 | −0.2119249 |
| si | ILC3.si | −0.1062102 | −0.2742339 | 0.40745992 | −0.2636587 | −0.4663231 | −0.2556031 |
| si | B.si | −0.1516694 | 0.68331444 | −0.4265058 | 0.40905199 | −0.1894694 | −0.6641858 |
| si | Tgd.si | −0.5744671 | −1.4180369 | −0.9017383 | −0.3502234 | −0.9955126 | 0.82648909 |
| si | Tab.si | 0.35554239 | −0.6732306 | 0.48323507 | −0.4308543 | 0.26445032 | 0.29601006 |
| si | DN(CD8mCD4mTCRp).si | 0.72037208 | −0.5956852 | 0.40217493 | −0.0363953 | −0.8957555 | −0.2055586 |
| si | T8.si | −0.5796289 | 0.22805534 | −0.3186403 | −0.2367776 | 0.03144058 | −0.2717979 |
| si | T8.Heliosp.si | −0.4765669 | −0.6181392 | −0.2136306 | −0.6040476 | −1.1138136 | 0.56670623 |
| si | T4.si | 0.03038033 | 0.04960871 | 0.10974187 | 0.2271971 | 0.1852901 | 0.08958815 |
| si | T4.FPmRorgp.si | −0.4806511 | −0.8406229 | 0.92066809 | 2.11212815 | 0.21007029 | 1.00027177 |
| si | T4.FPp.si | 0.26288542 | −0.5143178 | 0.18115051 | −0.053726 | −0.2780577 | −0.0390798 |
| si | T4.FPpHeliosm.si | 0.05696571 | 0.069878 | 0.30317826 | 0.35758031 | −0.1070268 | 0.18186887 |
| si | T4.FPpRorgpHeliosm.si | 0.25286943 | 0.68 | 2.33575319 | 2.25195835 | 0.82536445 | 2.2867744 |
| si | ILC.il22p.si | −1.0034505 | 1.13244565 | 1.17127934 | 0.36887459 | 0.63165236 | 1.37466249 |
| si | T4.ifngp.si | −0.1727021 | 1.18383258 | 0.06686195 | 0.70555264 | −0.1336246 | 0.25994843 |
| si | T4.il10p.si | 1.90992466 | −0.2035334 | −0.0995357 | 0.72169884 | 0.28397374 | 0.40168795 |
| si | T4.il17p.si | 0.44672001 | −1.3680223 | −0.3196789 | 0.25109543 | 0.16969412 | 1.88731935 |
| si | T4.il22p.si | −2.2598671 | −0.7415024 | 1.65711229 | −0.600904 | 0.36784502 | 0.32690213 |
| slo | mono.slo | −0.5128628 | −0.5983734 | 0.12044483 | 1.16691744 | 0.84151716 | 1.0826428 |
| slo | CD11bpCD11cmF4/80pMF.slo | −1.5115682 | −1.8366836 | −2.6710701 | 0.03529705 | −0.6498907 | 0.4897956 |
| slo | CD11bpCD11cpF4/80pMNP.slo | −1.5007229 | −1.5654733 | −0.3829246 | 0.33351662 | 0.16413789 | 0.48901226 |
| slo | CD103pCD11bmDC.slo | −1.6474584 | −1.1396638 | 0.52996111 | 0.17909006 | −1.8401035 | 0.47924605 |
| slo | CD103pCD11bpDC.slo | −1.0931094 | −3.4150375 | 2.03479896 | −1.3275747 | −2.7905466 | −2.476438 |
| slo | pDC.slo | −1.364941 | −0.0756501 | −1.2685726 | −0.1230205 | −0.0640466 | −0.0222814 |
| slo | ILC3.slo | 0.0671142 | 1.0671142 | 2.46566357 | 0.19264508 | −0.0703893 | 0.4150375 |
| slo | B.slo | 0.1317381 | 0.09416928 | 0.10404204 | 0.1220537 | 0.23371146 | −0.1904745 |
| slo | Tgd.slo | −0.0112565 | −0.096602 | 0.72269253 | 0.13546342 | −0.5156414 | 0.41316751 |
| slo | Tab.slo | 0.04414456 | −0.0540621 | −0.0024593 | −0.0335932 | −0.3092169 | 0.5520036 |
| slo | DN(CD8mCD4mTCRp).slo | −0.0153317 | −0.5813451 | −0.0880348 | −0.7551258 | −0.8661056 | −0.5922247 |
| slo | T8.slo | 0.06192775 | 0.03192354 | 0.02041954 | 0.03296863 | −0.1506071 | 0.01488687 |
| slo | T8.Heliosp.slo | −0.537536 | −0.2226005 | 0.06110145 | −0.9415932 | 0.59976163 | 0.07627689 |
| slo | T4.slo | −0.030499 | 0.0039466 | 0.02998488 | 0.02351924 | 0.13188796 | 0.0603575 |
| slo | T4.FPmRorgp.slo | −1.1264483 | −1.1598713 | 0.15301162 | −0.6129769 | 0.35976492 | 0.39087545 |
| slo | T4.FPp.slo | 0.19171606 | 0.31242992 | 0.01798256 | 0.15989061 | 0.12548095 | 0.23420759 |
| slo | T4.FPpHeliosm.slo | −0.2746765 | 0.02201068 | −0.2562008 | −0.3645241 | −0.25768 | −0.2455825 |
| slo | T4.FPpRorgpHeliosm.slo | 0.35247919 | 2.46859774 | 2.37215025 | 2.05356025 | 2.05740231 | 2.11506765 |
| slo | ILC.il22p.slo | 0.4229427 | −1.9842327 | 1.70652448 | −0.5305147 | 1.5743544 | 1.50901365 |
| slo | T4.ifngp.slo | −1.1551191 | 0.43244055 | 1.00785736 | −0.4057298 | 0.02085861 | 0.32612401 |
| slo | T4.il10p.slo | 1.33342373 | 0.13750352 | 1.24488706 | 2.58976349 | 5.2376402 | 1.11769504 |
| slo | T4.il17p.slo | −0.6658825 | −1.2577978 | 0.67533694 | 2.37935211 | 0.57619229 | 1.79606912 |
| slo | T4.il22p.slo | −0.337035 | −3 | −1.5188733 | −0.4352154 | −0.2451125 | −0.0931094 |

Table 4E: Continued - Fold change cell values compared to germ free (m stands for − and p stands for +) log2 value

Phylum

| | | | | | |
|---|---|---|---|---|---|
| Firmicutes | Fusobacteria | Fusobacteria | Proteobacteria | Proteobacteria | Firmicutes |

Genus

| | | Enterococcus | Fusobacterium | Fusobacterium | Helicobacter | Klebsiella | Lachnospira |
|---|---|---|---|---|---|---|---|
| organ | Row names (cell types) | Efaec.TX1330 | Fvari.AO16 | Fnucl.F0419 | Hpylo.ATCC700392 | Kleb.sp.4.1.44FAA | Lach.2.1.58FAA |
| colon | mono.co | 0.0134464 | 0.62493145 | 0.0023273 | 0.0023273 | 0.3447588 | 0.12785819 |
| colon | CD11bpCD11cmF4/80pMF.co | −0.8634139 | −0.6808995 | −1.1757377 | −0.373275 | −1.0367846 | −2.583954 |
| colon | CD11bpCD11cpF4/80pMNP.co | −0.2289538 | −0.4042101 | −0.0511146 | −0.1977495 | −0.641813 | −0.8735394 |
| colon | CD103pCD11bmDC.co | 0.08525571 | 0.08912352 | 0.09394376 | −0.08352 | 0.20147754 | −0.5221503 |
| colon | CD103pCD11bpDC.co | 1.22129864 | 1.62521467 | 1.67360041 | −0.5305147 | −0.0063921 | 2.71770369 |
| colon | pDC.co | −0.1299007 | 0.91121314 | 0.53029753 | −0.3623684 | 0.20983484 | 1.33541658 |
| colon | ILC3.co | −0.8367334 | 0.33073927 | 0.22573071 | −0.5569669 | −0.1716319 | −0.4141713 |
| colon | B.co | −0.2941113 | −0.5991216 | 0.15374095 | −0.4256354 | −0.2394101 | −0.2921053 |
| colon | Tgd.co | −0.0321242 | 0.62226682 | −0.6104142 | −0.0417103 | 0.36173927 | 0.29780724 |
| colon | Tab.co | 0.33874224 | −0.0450624 | −0.4092406 | 0.28127084 | 0.38993808 | 0.20643662 |
| colon | DN(CD8mCD4mTCRp).co | 0.4031236 | 0.87455299 | 0.04580189 | 0.3231501 | 0.165857 | 0.24189943 |
| colon | T8.co | 0.02684885 | −0.3889844 | −0.2410265 | −0.0851314 | 0.14379399 | −0.2418649 |
| colon | T8.Heliosp.co | | 0.66015156 | −0.4907503 | 0.24529687 | 0.38884954 | 0.14250125 |
| colon | T4.co | −0.3071281 | −0.4880061 | 0.21431947 | −0.0876036 | −0.2037864 | −0.0258258 |
| colon | T4.FPmRorgp.co | 1.14186872 | 2.33087705 | 1.26570359 | −0.2630344 | 2.01157515 | −0.4963686 |
| colon | T4.FPp.co | 0.23771145 | 1.33770707 | 0.50376155 | 0.57531481 | −0.1772476 | 0.47856875 |
| colon | T4.FPpHeliosm.co | 0.42599294 | 0.72503461 | 0.8015306 | −0.4009665 | 0.07120327 | 0.38986251 |
| colon | T4.FPpRorgpHeliosm.co | 0.80403842 | 1.67248058 | 1.92898476 | −0.5040543 | 0.01916015 | 1.30637081 |
| colon | ILC.il22p.co | −1.2492184 | 0.83140922 | 0.27996134 | −0.1140245 | −1.3355183 | 0.08064168 |
| colon | T4.ifngp.co | −0.4697877 | −0.2932241 | −0.131619 | 1.03111053 | 0.19632574 | −1.135932 |
| colon | T4.il10p.co | 3.778886 | 0.70414542 | 0.43895613 | 2.28191242 | 0.90951925 | 0.54193807 |
| colon | T4.il17p.co | −0.1188774 | −0.144039 | −0.6625946 | −1.2457988 | 0.49975173 | −0.0171441 |
| colon | T4.il22p.co | −0.9536444 | −2.2642126 | 0.19778838 | −1.5615985 | −2.4936944 | −2.9142701 |
| mln | mono.mln | −0.4994746 | −0.0599425 | −1.2038723 | −2.0570309 | −0.1194436 | −1.6018363 |
| mln | CD11bpCD11cpF4/80pMNP.mln | −1.6305388 | −1.3050431 | −2.6088733 | −1.3289954 | −2.8602259 | −1.4621581 |
| mln | CD103pCD11bmDC.mln | −1.0731887 | −1.0490426 | 0.42608533 | 0.26848884 | −0.5767886 | −1.4541373 |
| mln | CD103pCD11bpDC.mln | 0.27895441 | −0.142019 | 0.11124909 | 0.33597283 | −0.7720179 | 0.36060856 |
| mln | pDC.mln | 0.45441703 | −0.7480208 | −0.7480208 | −1.6583836 | 0.0433926 | −0.8337507 |
| mln | ILC3.mln | −0.1903312 | −0.1181814 | 0.16812276 | −1.3464504 | −0.4096442 | −0.1181814 |
| mln | B.mln | 0.16684945 | 0.07864382 | 0.16005148 | 0.2151105 | 0.04959898 | 0.11100071 |
| mln | Tgd.mln | −0.1058546 | 0.13675101 | −0.1388834 | −0.7498964 | 0.5785116 | 0.09086638 |
| mln | Tab.mln | −0.18788 | −0.1477555 | −0.0933446 | −0.19704 | 0.01187883 | −0.1367502 |
| mln | DN(CD8mCD4mTCRp).mln | 0.39880623 | 0.95587745 | 0.19047071 | −0.2079798 | 0.52150922 | −0.2137276 |
| mln | T8.mln | 0.15806101 | 0.05446434 | 0.19962729 | 0.24408359 | 0.32035879 | 0.13818675 |
| mln | T8.Heliosp.mln | | −0.1693888 | −0.6929507 | −0.30394 | −0.2557589 | 0.14042593 |
| mln | T4.mln | 0.05054894 | 0.08830496 | 0.03075282 | 0.00346663 | −0.0563357 | 0.07171705 |
| mln | T4.FPmRorgp.mln | 0.16937023 | 3.28348093 | 0.42870066 | −0.5824686 | 0.20011375 | −0.474486 |
| mln | T4.FPp.mln | 0.03390478 | −0.0430618 | −0.0336669 | 0.24479442 | 0.09770484 | 0.10026736 |
| mln | T4.FPpHeliosm.mln | −0.3285787 | −0.0453763 | 0.00285741 | −0.1748072 | −0.0898071 | −0.3249387 |
| mln | T4.FPpRorgpHeliosm.mln | 0.65666397 | 2.12980802 | 1.26427823 | −0.0254941 | 0.61901668 | 0.67620415 |
| mln | ILC.il22p.mln | −0.7131189 | 1.82171022 | 1.26303441 | 0.79077204 | 0.01435529 | −2.1202942 |
| mln | T4.ifngp.mln | 0.31379367 | 1.31262785 | 0.04562262 | 1.83360138 | 0.08165722 | −0.5762976 |
| mln | T4.il10p.mln | 4.66075996 | 3.34999201 | −2.4150375 | 0.38246964 | −0.0931094 | −0.4150375 |
| mln | T4.il17p.mln | −0.1027786 | 1.48421159 | 0.00565764 | −1.7820685 | 0.14802951 | 0.12222032 |
| mln | T4.il22p.mln | −2.0655883 | 3.47537316 | −1.2250666 | −1.4694853 | −3.3219281 | −2.0995357 |
| pp | mono.pp | 0.67966693 | −0.0538867 | −0.3056058 | −1.7946439 | 0.39344605 | −0.5776853 |
| pp | CD11bpCD11cmF4/80pMF.pp | −1.4799929 | −1.395724 | −1.4478125 | −1.4372433 | 0.04592521 | −2.86285 |
| pp | CD11bpCD11cpF4/80pMNP.pp | 0.20501567 | −0.3893107 | 1.00919242 | −0.2390533 | 1.24835888 | −0.6879421 |
| pp | CD103pCD11bmDC.pp | −0.9792202 | −3.0034182 | 0.70951169 | −0.4284095 | −1.2653817 | −0.7838343 |
| pp | CD103pCD11bpDC.pp | 0.18637568 | −0.9472237 | 0.81431108 | −0.0426382 | 0.03177602 | −0.2055006 |
| pp | pDC.pp | 0.57880152 | −1.0331669 | 0.4779422 | −1.4014399 | 1.24576876 | −0.755528 |
| pp | ILC3.pp | −0.8117334 | −0.4238077 | −0.4534329 | 0.38522185 | −0.2948599 | −0.1963972 |
| pp | B.pp | −0.0368889 | −0.0179726 | 0.08483896 | −0.0780996 | 0.08949487 | 0.05641547 |
| pp | Tgd.pp | −0.0763004 | −1.1620503 | 0.02258218 | 0.23046513 | −0.4916644 | −0.3535079 |
| pp | Tab.pp | −0.2213378 | −0.1977714 | −0.7125722 | −0.3125404 | −0.3083282 | −0.2376293 |
| pp | DN(CD8mCD4mTCRp).pp | −0.2269191 | −1.0706133 | −0.688208 | 0.09040135 | −1.3037574 | 0.41317042 |
| pp | T8.pp | 0.04026469 | −0.4565044 | 0.05867136 | 0.11463572 | −0.0220401 | −0.0214031 |
| pp | T8.Heliosp.pp | | −0.5883262 | −0.0784754 | 0.37449151 | −1.1808859 | 0.11917686 |
| pp | T4.pp | 0.02564525 | 0.31588767 | 0.04709978 | −1.2805752 | 0.11935208 | 0.0549535 |
| pp | T4.FPmRorgp.pp | 0.20240805 | 1.68860865 | 0.67025845 | −0.6533495 | −1.1779623 | −0.6816385 |
| pp | T4.FPp.pp | 0.15241165 | −0.4538472 | 0.1614077 | 1.01289718 | 0.03649569 | 0.20322788 |
| pp | T4.FPpHeliosm.pp | −0.7327102 | −0.0478122 | −0.2108736 | 0.04219401 | −0.4604159 | −0.3809654 |
| pp | T4.FPpRorgpHeliosm.pp | 0.10975304 | 0.87023776 | 1.71529012 | 0.97754609 | 0.51844901 | 0.81874082 |
| pp | ILC.il22p.pp | −1.0695556 | −1.3231199 | 0.2747018 | −0.3670018 | 0.15462163 | 2.19550886 |
| pp | T4.ifngp.pp | −2.3992338 | −2.7580272 | −3.523562 | 1.59745345 | −3.2605276 | −0.9924925 |
| pp | T4.il10p.pp | 3.79677948 | 1.29992902 | −4.3487282 | 1.35863098 | −2.1788032 | −1.4338448 |
| pp | T4.il17p.pp | −0.8625241 | −2.4212252 | −1.1258898 | −3.7545839 | −0.2667438 | 1.0203529 |
| pp | T4.il22p.pp | −5.8044896 | −3.4825615 | −2.8763825 | −2.2128634 | −3.3597047 | −2.707628 |
| si | mono.si | 0.31834902 | | −0.0773034 | 0.1716302 | 0.07241378 | −0.1547748 |
| si | CD11bpCD11cmF4/80pMF.si | −5.3448649 | | −0.0891705 | 1.03595688 | −1.3425547 | −4.9023467 |
| si | CD11bpCD11cpF4/80pMNP.si | −3.1673446 | | 0.53791673 | 0.01639058 | −0.6723707 | −1.9607345 |
| si | CD103pCD11bmDC.si | 0.75547163 | | −0.032607 | −1.5028002 | −0.5297154 | −0.4934193 |
| si | CD103pCD11bpDC.si | −0.9621591 | | 1.25656702 | 0.03344022 | −0.1722763 | 0.52901034 |
| si | pDC.si | −0.5438733 | | 0.31345308 | 0.27149777 | −0.1689262 | −1.732481 |
| si | ILC3.si | −0.7774126 | | 1.30379309 | 0.73575585 | 0.39327372 | 0.4823168 |
| si | B.si | −0.0747768 | | −0.689999 | 0.29894577 | −0.0724062 | −0.4170413 |
| si | Tgd.si | −1.4127921 | | 0.97363702 | −2.0856382 | −0.3960617 | −0.0874277 |
| si | Tab.si | 0.04107356 | | 0.06285335 | 0.08514513 | 0.22778307 | −0.0344589 |
| si | DN(CD8mCD4mTCRp).si | −0.4002097 | | 0.13974 | −0.3846088 | −0.6102993 | 1.10101591 |
| si | T8.si | 0.16193969 | | 0.06868585 | −0.219036 | −0.2869076 | −0.3274612 |
| si | T8.Heliosp.si | | | 0.93547911 | −0.9062031 | −1.0095962 | 0.24821704 |
| si | T4.si | −0.0254465 | | −0.0112984 | 0.17839292 | 0.18745905 | −0.2098437 |
| si | T4.FPmRorgp.si | 0.47545245 | | 0.5762387 | −0.4670621 | 0.16859561 | −0.1602409 |
| si | T4.FPp.si | −0.6906406 | | 0.11577972 | −0.0065699 | 0.28183152 | −0.0535405 |
| si | T4.FPpHeliosm.si | | | −0.3587925 | −0.0407581 | −0.191982 | 0.41120588 |
| si | T4.FPpRorgpHeliosm.si | | | 0.8245476 | 0.39536905 | 0.70625324 | 1.30533685 |
| si | ILC.il22p.si | −0.6773298 | | −1.1253863 | 0.97405299 | 0.10873191 | 0.81423121 |
| si | T4.ifngp.si | 0.97575245 | | −0.934905 | 1.31078754 | −0.4324046 | −0.4150375 |
| si | T4.il10p.si | 3.04975304 | | −3.7625007 | 1.76479013 | 0.00505323 | −0.4303043 |
| si | T4.il17p.si | 0.23999548 | | −0.403311 | −0.848274 | 1.26499545 | 0.36679647 |
| si | T4.il22p.si | −0.4112677 | | −0.9015216 | −1.5736826 | −3.2066251 | −1.0297473 |
| slo | mono.slo | 0.48101941 | −0.7910185 | 0.11191842 | 0.38389899 | 0.52211772 | 0.00068439 |
| slo | CD11bpCD11cmF4/80pMF.slo | −1.6528973 | −1.1929186 | −1.1134581 | 0.00255006 | −0.0861076 | −2.6074699 |
| slo | CD11bpCD11cpF4/80pMNP.slo | 0.09136565 | −0.2188969 | 0.39449594 | −0.1758753 | −0.8669157 | −0.6268738 |
| slo | CD103pCD11bmDC.slo | −0.1044266 | −0.7226459 | 0.93750407 | −0.0475456 | −1.6474584 | −0.6759149 |
| slo | CD103pCD11bpDC.slo | −0.2518072 | −1.3706434 | 0.66888498 | −1.0342157 | −2.4150375 | −1.0811368 |
| slo | pDC.slo | 0.30151664 | 0.03098769 | −1.0582796 | −0.3205469 | −0.6674247 | −0.3744638 |
| slo | ILC3.slo | 2.46566357 | 0.46566357 | −0.3048546 | 0.13124453 | −1.2223924 | −0.3048546 |
| slo | B.slo | 0.14623442 | 0.0923119 | 0.14892755 | 0.12564961 | 0.11471152 | 0.17183586 |
| slo | Tgd.slo | −0.1661923 | −0.0494252 | −0.1703923 | −0.510308 | 0.22132415 | −0.0340363 |
| slo | Tab.slo | −0.1732327 | 0.16415794 | 0.00662578 | 0.03768925 | 0.13161119 | −0.0372004 |
| slo | DN(CD8mCD4mTCRp).slo | −0.1208248 | −0.4821517 | −1.1974461 | −1.1291401 | −0.7163977 | 0.34672455 |
| slo | T8.slo | 0.01514074 | −0.0191088 | 0.05786723 | 0.28099291 | 0.06364695 | 0.02895137 |
| slo | T8.Heliosp.slo | | −0.1588502 | −0.9078271 | −0.6633552 | 0.22946349 | −0.0018736 |
| slo | T4.slo | −0.0479238 | 0.05293183 | 0.02175084 | −0.1390372 | 0.00397048 | −0.0305724 |
| slo | T4.FPmRorgp.slo | 2.51432093 | 0.80879546 | 0.04958203 | −0.5900157 | −0.5016599 | −0.8930848 |
| slo | T4.FPp.slo | 0.25664906 | 0.06238386 | −0.2706038 | 0.12174112 | 0.14728587 | 0.12646929 |
| slo | T4.FPpHeliosm.slo | −0.2879028 | −0.6021373 | 0.25549263 | −0.1993069 | −0.1761603 | −0.4128041 |
| slo | T4.FPpRorgpHeliosm.slo | 0.60084554 | 1.89226555 | 1.23977905 | −0.054243 | 0.85154161 | 0.80659421 |
| slo | ILC.il22p.slo | −0.8353693 | 1.31238432 | 2.01576732 | 0.29956028 | −3.1858665 | 0.1646307 |
| slo | T4.ifngp.slo | 0.14004796 | 0.83411815 | −0.9791414 | 0.14797052 | 0.15742052 | 0.19461343 |
| slo | T4.il10p.slo | 3.74200621 | 1.31614574 | −2.3959287 | 0.78240857 | −0.7612131 | −0.7858752 |
| slo | T4.il17p.slo | −0.7516124 | −0.3219281 | 0.105353 | −1.3219281 | −0.5981379 | 0.66710079 |
| slo | T4.il22p.slo | −2.1586977 | 1.36340473 | −0.5188733 | −2.0931094 | −4.2630344 | −2.1255309 |

Table 4F: Continued - Fold change cell values compared to germ free (m stands for − and p stands for +) log2 value

| | | Phylum | | | | | | |
|---|---|---|---|---|---|---|---|---|
| | | Firmicutes | Firmicutes | Proteobacteria | Firmicutes | Bacteroidetes | Actinobacteria | Firmicutes |
| | | Genus | | | | | | |
| | | Lactobacillus | Lactobacillus | Neisseria | Peptoniphilus | Parabacteroides | Propionibacterium | Peptostreptococcus |
| organ | Row names (cell types) | Lcase.AO47 | Lrham.LMS2-1 | Nflav.SK114 | Pasac.AO33 | Pdist.ATCC8503 | Pgran.AO42 | Pmagn.AO29 |
| colon | mono.co | 0.14859847 | −0.5646099 | 0.8568722 | −0.5945439 | −0.3428082 | 0.27755727 | 0.11070346 |
| colon | CD11bpCD11cmF4/80pMF.co | −2.5160151 | −1.919557 | −0.5102559 | −1.5102559 | −1.1013384 | −0.4938249 | −1.9859444 |
| colon | CD11bpCD11cpF4/80pMNP.co | 0.02236445 | −0.3195777 | 0.16141451 | −0.4368652 | 0.28221737 | 0.32057076 | 0.21263756 |
| colon | CD103pCD11bmDC.co | −0.2771761 | 0.29444736 | 0.0920176 | −0.118219 | −0.3810304 | −0.2684888 | −0.2126669 |
| colon | CD103pCD11bpDC.co | 1.29176612 | 1.27657696 | 1.63633614 | −1.2761045 | 0.76650987 | 1.46994584 | 0.38897271 |
| colon | pDC.co | 0.68756914 | −1.4973336 | 1.57977718 | −0.5963 | 1.18955381 | −0.9097166 | 0.00984779 |
| colon | ILC3.co | 1.02064715 | −0.3514724 | 0.43440282 | 1.14956405 | −1.803461 | 1.45943162 | 0.36242566 |
| colon | B.co | 0.21345232 | −0.0937462 | −0.394325 | −0.124974 | 0.05095878 | −0.1486197 | −0.2347615 |
| colon | Tgd.co | −0.6363229 | −0.1585239 | 0.37572974 | −0.1391864 | 0.57788535 | 0.0609852 | −0.1689909 |
| colon | Tab.co | −0.5711842 | 0.18816143 | 0.10113837 | −0.1279139 | −0.1634517 | −0.1694507 | −0.1530131 |
| colon | DN(CD8mCD4mTCRp).co | −0.6300037 | 0.15076851 | 0.56650708 | 0.5343378 | 0.29639959 | 0.33082733 | −0.2366582 |
| colon | T8.co | −0.3465011 | 0.03591916 | −0.1851892 | −0.1766333 | 0.10819186 | −0.287628 | 0.03428774 |
| colon | T8.Heliosp.co | 1.0945725 | 0.17025832 | 0.38844213 | 0.16631284 | 0.09656798 | −0.0501309 | 0.25558136 |
| colon | T4.co | 0.44490288 | −0.1248855 | −0.253128 | −0.3100021 | −0.1984906 | −0.0888186 | 0.15439792 |
| colon | T4.FPmRorgp.co | 1.28029649 | 0.66801824 | 2.07610298 | 0.04730572 | 0.70487196 | 2.14839184 | −0.462842 |
| colon | T4.FPp.co | 0.5280091 | −0.0574344 | 0.69436748 | 0.71794257 | 0.89284297 | 0.51726116 | 0.2348073 |
| colon | T4.FPpHeliosm.co | 0.77201929 | 0.52335493 | 0.13699188 | 0.25004466 | −0.0433344 | 0.20644366 | −0.1156486 |
| colon | T4.FPpRorgpHeliosm.co | 1.96184504 | 1.84557485 | 0.70740787 | 0.16101207 | | 1.42967278 | −0.4542127 |
| colon | ILC.il22p.co | 2.1323816 | −1.086929 | 1.43070325 | −1.4035739 | 0.63269301 | 0.45894614 | −0.3682256 |
| colon | T4.ifngp.co | 1.38198491 | 0.6565369 | 1.30293458 | 0.686157 | 0.6565369 | 0.74010701 | 0.2600831 |
| colon | T4.il10p.co | 1.81672108 | 1.6520767 | 0.82293157 | −0.7694867 | 1.7582941 | 0.46460471 | 0.01309566 |
| colon | T4.il17p.co | −0.4152845 | 1.08453953 | −0.0941472 | −2.1229061 | −0.4789209 | 0.41892243 | −0.8771886 |
| colon | T4.il22p.co | −1.3441809 | 0.85126462 | −0.6328572 | −1.8386059 | 0.25565488 | 0.85452495 | −0.3479233 |
| mln | mono.mln | −0.2168114 | −2.0687128 | −0.4643328 | −1.5274115 | −1.1043367 | −0.5477029 | −1.1043367 |
| mln | CD11bpCD11cpF4/80pMNP.mln | −0.7558893 | −2.4852987 | −1.6108295 | −3.5438933 | −1.7634853 | −1.9840655 | −2.1619358 |
| mln | CD103pCD11bmDC.mln | 0.1793237 | −0.7589919 | −0.1185343 | 0.13785416 | −1.2385554 | −1.2730989 | −0.5941532 |
| mln | CD103pCD11bpDC.mln | 0.169925 | −0.8905668 | −0.0993957 | −1.353637 | 0.69923295 | −0.6970135 | −1.7553776 |
| mln | pDC.mln | −0.2852302 | −1.8836759 | 0.02727293 | −0.9781406 | −0.4942642 | −1.0676387 | −0.863498 |
| mln | ILC3.mln | 0.75308526 | 0.01611967 | −0.3464504 | 0.70417566 | | −0.1181814 | 0.23851209 |
| mlη | B.mln | 0.22900007 | −0.0048477 | 0.13675486 | 0.05625548 | 0.41922915 | −0.025484 | −0.0499591 |
| mlη | Tgd.mln | 0.16408972 | 0.30248821 | −0.323308 | −0.1695802 | −0.2104882 | 0.3702123 | 0.48108552 |
| mlη | Tab.mln | −0.2526446 | −0.0192054 | −0.0738082 | −0.0202781 | −0.5259226 | 0.00124996 | 0.09255018 |
| mlη | DN(CD8mCD4mTCRp).mln | 0.35482405 | 0.87424336 | 0.09605511 | −0.4208431 | −0.2635311 | 0.58081801 | 0.22061767 |
| mlη | T8.mln | 0.16955925 | 0.15926227 | 0.26452354 | 0.22623588 | 0.12337909 | 0.27742121 | 0.21808464 |
| mlη | T8.Heliosp.mln | 0.19813189 | −0.4061264 | 0.40224339 | 0.18072998 | 0.42239309 | 0.2027993 | 0.22774566 |
| mlη | T4.mln | 0.04183413 | 0.05449063 | −0.0003857 | 0.03180946 | 0.10374761 | −0.0246071 | 0.02257545 |
| mlη | T4.FPmRorgp.mln | 1.09886992 | 1.00746867 | 0.35645578 | −0.9950166 | | 2.02964546 | 0 |
| mlη | T4.FPp.mln | 0.30008193 | −0.2357574 | −0.0903675 | −0.2005079 | 0.12144647 | −0.0503851 | 0.01795579 |
| mlη | T4.FPpHeliosm.mln | −0.0744899 | −0.1859128 | 0.05836361 | 0.18996111 | −0.2409202 | 0.02654792 | −0.1435268 |
| mlη | T4.FPpRorgpHeliosm.mln | 1.98527997 | 1.17043542 | 0.40668571 | 0.66610525 | | 1.25756801 | 0.51996172 |
| mlη | ILC.il22p.mln | 1 | −0.7490384 | 2.20163386 | −1.3959287 | 1.929791 | 2.35049725 | −0.4941091 |
| mlη | T4.ifngp.mln | 2.054716 | 0.66021789 | 0.68925425 | 1.08848494 | −1.2526428 | −0.1271119 | 0.0595888 |
| mlη | T4.il10p.mln | 1.57202345 | −0.8073549 | 0.28010792 | −0.0524674 | 3.65923142 | 0.87914561 | −0.2527661 |
| mlη | T4.il17p.mln | 0.68795825 | −0.1713429 | 0.06376059 | −2.1460318 | −0.0786325 | −1.6164824 | −1.4065593 |
| mlη | T4.il22p.mln | 0.81192765 | 1.23606736 | 0.75607442 | −1.7369656 | −0.646363 | −0.4584301 | −0.3833286 |
| pp | mono.pp | 0.33246805 | 0.23510347 | 0.05896664 | 0.35260569 | −1.5722514 | 0.66363682 | 0.23819939 |
| pp | CD11bpCD11cmF4/80pMF.pp | −0.9503129 | −2.4691862 | −0.1865472 | −1.5240481 | | −0.3307042 | −0.9503129 |
| pp | CD11bpCD11cpF4/80pMNP.pp | 1.33050951 | 0.6698514 | 0.73532524 | 0.58769229 | −2.5952455 | 1.62238466 | 1.41938567 |
| pp | CD103pCD11bmDC.pp | 0.33660816 | 0.25561808 | −0.4133067 | −1.1351304 | −0.0693284 | 0.48801162 | 0.97128982 |
| pp | CD103pCD11bpDC.pp | 0.42458263 | 0.24019991 | 0.54592605 | −1.0080546 | 1.35648166 | 0.92746202 | 1.39810189 |
| pp | pDC.pp | 0.22004848 | −0.1240413 | 1.3000405 | −0.9536201 | 0.11232103 | 0.63001886 | 0.6484393 |
| pp | ILC3.pp | 0.77322914 | 0.105353 | −0.0671142 | 0.97797369 | | 0.51937416 | −0.2267709 |
| pp | B.pp | 0.02625421 | 0.04852856 | −0.0425657 | −0.2167788 | 0.04768339 | 0.02453705 | 0.07199462 |
| pp | Tgd.pp | −0.4005565 | 0.25129721 | 0.19479173 | −1.0890895 | 0.3346913 | 0.54317876 | 0.07511492 |
| pp | Tab.pp | −0.4678709 | −0.503324 | −0.1744095 | 0.06136195 | −0.1842 | −0.3083282 | −0.5392215 |
| pp | DN(CD8mCD4mTCRp).pp | −1.1028447 | −0.5913205 | −0.8024498 | 0.2520462 | −1.1396446 | −0.668303 | −0.8015823 |
| pp | T8.pp | 0.15878645 | 0.30257973 | 0.0832488 | −0.1605042 | 0.34027712 | 0.10283073 | 0.08214838 |
| pp | T8.Heliosp.pp | 0.52647732 | −0.3203797 | 0.46381644 | −1.2668472 | −0.1492432 | 0.14499774 | −0.4318847 |
| pp | T4.pp | 0.06786557 | −0.119114 | 0.10847289 | 0.13373122 | −0.0470398 | −0.0222517 | 0.01964436 |
| pp | T4.FPmRorgp.pp | 2.03214439 | 1.18449581 | 0.73375604 | 0.2996716 | | 1.93766988 | −0.3557164 |
| pp | T4.FPp.pp | 0.16074325 | 0.25200303 | 0.36808 | −0.409137 | 0.35724619 | 0.23892065 | −0.2404594 |
| pp | T4.FPpHeliosm.pp | 0.2048891 | −0.111926 | 0.27841111 | 0.0267247 | −0.0050728 | −0.2508227 | −0.5074641 |
| pp | T4.FPpRorgpHeliosm.pp | 2.39762384 | 1.89865786 | 0.33575671 | 1.22656177 | | 1.35142705 | −0.0457573 |
| pp | ILC.il22p.pp | 0.41801274 | −0.9519643 | 1.59643293 | 0.61599785 | −2.1075568 | 0.17375862 | 0.6467601 |
| pp | T4.ifngp.pp | −1.4125306 | −0.9053233 | 0.48542683 | −0.3992338 | −0.8275681 | 0.01993393 | 0.02240641 |
| pp | T4.il10p.pp | 1.45755293 | 0.21859419 | −0.3524901 | −1.8251662 | 2.22995902 | 0.63426542 | 1.03497614 |
| pp | T4.il17p.pp | −1.6384762 | −0.9542722 | −2.0217227 | −4.0491086 | −1.4293806 | −1.0303801 | −0.9039708 |
| pp | T4.il22p.pp | −1.4313828 | −0.2774471 | −0.9110195 | 0.17787025 | −2.2329476 | −0.9272907 | −1.9576063 |
| si | mono.si | 0.46361351 | 0.19402896 | 0.24950331 | 0.11138556 | 0.01078421 | 0.30618457 | 0.19006248 |
| si | CD11bpCD11cmF4/80pMF.si | −2.2595797 | −3.179056 | −0.7460966 | −1.4578396 | −0.2530566 | 0.51056265 | −0.7894892 |
| si | CD11bpCD11cpF4/80pMNP.si | −1.6286114 | −1.1848528 | 0.46445027 | −0.5468802 | −0.3242659 | 0.67058914 | 0.38645999 |
| si | CD103pCD11bmDC.si | 0.37522948 | 1.42033788 | 0.19815225 | −0.2951603 | −0.8341705 | 0.13150321 | 0.54833964 |
| si | CD103pCD11bpDC.si | 0.31363456 | −0.6593984 | 0.14778689 | 0.59470288 | −1.9519427 | 1.13346552 | 1.63640927 |
| si | pDC.si | −0.6075195 | −0.4931669 | 1.52297599 | −0.0295333 | 0.07823088 | 0.6693097 | 0.3536866 |
| si | ILC3.si | −0.3944461 | 0.8281294 | 0.23407513 | 0.54621949 | | 0.49524277 | 0.78146255 |
| si | B.si | 0.31640178 | −0.5622902 | 0.07692785 | 0.14897582 | −0.0723703 | −0.0664252 | −1.2375927 |
| si | Tgd.si | −1.2950729 | 0.52375735 | −0.3204541 | −1.0561342 | 0.19492558 | −0.3169507 | 0.3391285 |
| si | Tab.si | 0.12795793 | −0.0446418 | −0.4609707 | −0.106454 | 0.35047987 | 0.19631662 | 0.14654363 |
| si | DN(CD8mCD4mTCRp).si | 0.19145956 | 0.44448449 | −0.2910954 | −0.1379847 | −0.4330726 | −0.4565692 | −0.5417523 |
| si | T8.si | 0.00242183 | 0.09517716 | 0.0990398 | −0.1748362 | −0.1701798 | 0.18630294 | −0.3066671 |
| si | T8.Heliosp.si | −0.0545096 | 0.46943826 | 1.32150483 | −0.8158226 | −0.2501087 | −0.3797818 | 0.01515538 |
| si | T4.si | 0.0068449 | −0.1947374 | 0.05158659 | 0.07088873 | 0.14866738 | −0.0212827 | 0.13184961 |
| si | T4.FPmRorgp.si | 1.28537434 | 0.81803186 | 0.86236057 | −0.3508914 | | 1.31626363 | −0.0764632 |
| si | T4.FPp.si | −0.2541944 | 0.45770787 | −0.3886238 | −0.1579745 | 0.72526228 | −0.33954 | 0.10146513 |
| si | T4.FPpHeliosm.si | 0.58626765 | −0.5425745 | 0.01630181 | −0.4976362 | −0.4434616 | −0.3553548 | −0.5199304 |
| si | T4.FPpRorgpHeliosm.si | 2.0468622 | 1.3720784 | 1.28132745 | 0.06507375 | | 0.59441758 | 0.09699308 |
| si | ILC.il22p.si | 1.22205153 | −0.4212366 | 1.38576333 | 0.31693551 | 0.87483058 | −1.784669 | 1.53186 |
| si | T4.ifngp.si | 1.52388359 | 0.0580056 | 1.01197264 | −0.2301198 | 1.16498426 | 0.43295941 | 0.31004177 |
| si | T4.il10p.si | 0.82817547 | 0.04975304 | 0.38240029 | −2.5109619 | 0.75956702 | 0.42261072 | 0.33502985 |
| si | T4.il17p.si | 0.78773018 | −0.1711376 | 0.44519469 | −1.456491 | 0.82652103 | 0.58888193 | 0.15047856 |
| si | T4.il22p.si | 1.26038969 | 1.93692448 | 0.67920388 | 1.8579029 | 2.39780726 | −0.0863309 | 0.10270295 |
| slo | mono.slo | 0.24177144 | −0.1915209 | 0.83711723 | −0.3244975 | −0.0144477 | 0.09631468 | −0.0490867 |
| slo | CD11bpCD11cmF4/80pMF.slo | −2.3225663 | −2.1065717 | −0.0401134 | −1.053957 | −2.5170364 | −0.680243 | −1.1371207 |
| slo | CD11bpCD11cpF4/80pMNP.slo | −0.140536 | 0.03401938 | 0.03233733 | −0.3304732 | −0.6316659 | 0.18245419 | 0.05139743 |
| slo | CD103pCD11bmDC.slo | −0.4138388 | −2.3436777 | −1.371334 | 1.39420073 | −0.0217496 | −2.3620562 | −0.653105 |
| slo | CD103pCD11bpDC.slo | −1.179821 | −1.2055841 | −1.4301444 | −2.476438 | 0.22400167 | −2.7145978 | −1.7332135 |
| slo | pDC.slo | −0.2098565 | −0.2487338 | −0.3985494 | −0.1534369 | −0.1050739 | −0.3912814 | −0.4304741 |
| slo | ILC3.slo | 0.96523458 | −0.6918777 | 0.73696559 | 0 | −0.0703893 | 0.56187889 | 0.51457317 |
| slo | B.slo | 0.11949433 | 0.18511455 | 0.0715942 | 0.15525869 | 0.2980484 | 0.05810003 | 0.18446821 |
| slo | Tgd.slo | 0.13205682 | −0.1620045 | −0.0378682 | −0.0417103 | −0.1536652 | 0.26241135 | −0.3684554 |
| slo | Tab.slo | 0.0038569 | −0.0819075 | 0.07605783 | −0.1313868 | −0.4457333 | 0.07813533 | −0.2644962 |
| slo | DN(CD8mCD4mTCRp).slo | 0.00836347 | −1.3494492 | −0.6330309 | −1.2009478 | −0.475245 | −1.1299742 | −0.6847978 |
| slo | T8.slo | −0.0557014 | 0.07923061 | 0.17326545 | 0.18267572 | 0.16562418 | 0.19520271 | −0.0901482 |
| slo | T8.Heliosp.slo | 0.3194919 | −0.1274897 | −0.4097421 | 0.03909815 | 0.19361996 | −0.1102077 | −0.028045 |
| slo | T4.slo | 0.04169015 | 0.03000834 | −0.0520399 | −0.0372421 | −0.0620318 | −0.0703821 | 0.01211634 |
| slo | T4.FPmRorgp.slo | 0.07374834 | −0.6284899 | 0.37928747 | −1.3269813 | 0.36369062 | 1.3439544 | −0.3983201 |
| slo | T4.FPp.slo | 0.27161698 | −0.1204089 | 0.10523654 | 0.08459099 | 0.4013112 | 0.16876613 | 0.05421253 |
| slo | T4.FPpHeliosm.slo | 0.07668798 | −0.1750534 | 0.07321119 | −0.0392662 | −0.5149416 | −0.012953 | 0.10607494 |
| slo | T4.FPpRorgpHeliosm.slo | 2.1027263 | 1.73163215 | 0.47243981 | 0.8396919 | | 1.9122044 | 2.21820352 |
| slo | ILC.il22p.slo | 1.26038969 | −1.3378696 | 1.75430021 | −3.048363 | −0.3580475 | 1.22012581 | −0.1679446 |
| slo | T4.ifngp.slo | 0.5459204 | 0.64888983 | 0.48842516 | −0.1261677 | −0.1609797 | −0.1434687 | 0.02258329 |
| slo | T4.il10p.slo | 1.35614381 | −0.2688168 | 0.59454855 | 0.5849625 | 4.3219281 | 0.84799691 | 2.48284828 |
| slo | T4.il17p.slo | 0.85284631 | 2.31667237 | −0.5981379 | −0.7442704 | 0.13352367 | 0.67259678 | 0.21487837 |
| slo | T4.il22p.slo | −0.318176 | 0.86624861 | 0.87446912 | −2.1926451 | −0.7905466 | −0.337035 | −0.5188733 |

Table 4G: Continued - Fold change cell values compared to germ free (m stands for − and p stands for +) log2 value

Phylum

| | | | | | |
|---|---|---|---|---|---|
| Bacteroidetes | Firmicutes | Firmicutes | Firmicutes | Firmicutes | Firmicutes |

Genus

| | | Porphyromonas | Blautia | Candidatus | Streptococcus | Staphylococcus | Veillonella | |
|---|---|---|---|---|---|---|---|---|
| organ | Row names (cell types) | Pueno.UPII60-3 | Rgnav.ATCC29149 | SFB | Smiti.F0392 | Ssapr.ATCC15305 | Veil.6.1.27 | SPF |
| colon | mono.co | −0.3228601 | −0.0088782 | 0.3083552 | −0.8136575 | −0.0070045 | −0.0339362 | 0.40263972 |
| colon | CD11bpCD11cmF4/80pMF.co | −0.969059 | −1.3719688 | −1.5002322 | −1.1486441 | −0.5629362 | −2.0916305 | −1.0565835 |
| colon | CD11bpCD11cpF4/80pMNP.co | −0.3952608 | 0.23139849 | −2.4447362 | −0.4358288 | 0.25689434 | −0.7869848 | −0.4344975 |
| colon | CD103pCD11bmDC.co | −0.5709073 | −0.2734466 | −0.5147933 | −0.2877956 | 0.07651502 | 0.08912352 | −0.590881 |
| colon | CD103pCD11bpDC.co | 1.06179911 | 1.06241004 | 0.71459778 | 1.38653531 | 0.14778749 | 1.21196788 | 1.34846988 |
| colon | pDC.co | 0.13081484 | 1.76354047 | 1.44576402 | 1.83729157 | −1.8834373 | −0.3425435 | 3.72028275 |
| colon | ILC3.co | −0.45012 | 1.27785075 | 0.15278272 | −0.1592419 | 1.53021475 | −1.1448004 | 1.60432783 |
| colon | B.co | 0.08828121 | −0.0102065 | 0.06345179 | −0.1768314 | −0.4115441 | −0.2394101 | −0.1677747 |
| colon | Tgd.co | −0.5049942 | −0.3612359 | −0.6009352 | −0.4726513 | 0.72269253 | 0.61333621 | 0.57077331 |
| colon | Tab.co | −0.112807 | −0.3909717 | −0.0791582 | 0.18576669 | −0.1352923 | 0.33166635 | 0.21719048 |
| colon | DN(CD8mCD4mTCRp).co | −0.3954492 | 0.16246037 | −0.2961664 | −0.2355036 | 0.5518305 | 0.17098823 | 0.08297146 |
| colon | T8.co | −0.1539123 | −0.2682206 | −0.3005464 | −0.1737164 | −0.3449562 | 0.25268918 | −0.1851892 |
| colon | T8.Heliosp.co | −0.248586 | 0.22832346 | −0.2566481 | −0.1838428 | 0.40804512 | 0.34490567 | 0.47732741 |
| colon | T4.co | 0.30811164 | 0.13481515 | 0.31728633 | 0.22398974 | −0.1159247 | −0.2816426 | 0.11707226 |
| colon | T4.FPmRorgp.co | −0.8422376 | 0.44948801 | 3.96778344 | 1.53051472 | 2.28589536 | −0.2688168 | 3.44463762 |
| colon | T4.FPp.co | −0.1112458 | 0.47371667 | 0.383042 | 0.3103431 | 0.50128157 | −0.0158974 | 0.97867051 |
| colon | T4.FPpHeliosm.co | 0.07931755 | 0.40477496 | 0.49003397 | 0.33335331 | −0.6549704 | 0.52369369 | 0.90122355 |
| colon | T4.FPpRorgpHeliosm.co | −1.3137162 | 1.25299438 | 1.54125224 | −0.0134995 | 0.24551879 | 1.04123182 | 2.48715953 |
| colon | ILC.il22p.co | −0.0251347 | 0.06473379 | 0.26552396 | 1.48410409 | 1.02032372 | −2.6471526 | 1.481047 |
| colon | T4.ifngp.co | 0.24689266 | 1.75823229 | 1.26499859 | −0.4268791 | 1.49019237 | 0.95102262 | 0.8124003 |
| colon | T4.il10p.co | 0.60384051 | 2.33936269 | 1.53287399 | −0.8150493 | 1.89779949 | 2.64368078 | 2.60167484 |
| colon | T4.il17p.co | 1.26402205 | −0.07139 | 1.56656814 | −1.6141542 | 1.20610504 | 0.35489286 | 0.9386961 |
| colon | T4.il22p.co | −1.2087175 | −1.9479533 | 0.44922446 | −0.6147098 | 1.36485826 | −1.060121 | 0.71620703 |
| mln | mono.mln | −0.2331501 | −0.4876653 | −2.1910483 | −1.1470411 | −0.531447 | −0.4262648 | −0.7920587 |
| mln | CD11bpCD11cpF4/80pMNP.mln | −2.3034603 | −1.953956 | −4.926897 | −1.7206086 | −0.7700281 | −1.0595347 | −2.0622089 |
| mln | CD103pCD11bmDC.mln | −0.5912446 | 0.3219281 | −1.7427634 | 0.75059504 | −0.4370638 | −0.3096845 | 0.65584158 |
| mlη | CD103pCD11bpDC.mln | −0.3019748 | 0.19753723 | −1.8024628 | 0.99016564 | −0.4387536 | −0.3789037 | −1.2197812 |
| mlη | pDC.mln | −0.2455204 | 0.17114814 | −0.6411056 | −0.1086105 | −1.0110552 | −0.175442 | 5.80971291 |
| mlη | ILC3.mln | 0.13897641 | 0.10922907 | −0.8610236 | 0.26573356 | −0.0163018 | −0.1358834 | −0.5930904 |
| mlη | B.mln | 0.10418087 | −0.0250035 | −0.003368 | 0.10415341 | −0.0085833 | 0.05435269 | −0.3616134 |
| mlη | Tgd.mln | −0.1058546 | 0.00825185 | −0.0448252 | −0.0910879 | 0.3443723 | 0.10888422 | 0.0699952 |
| mlη | Tab.mln | −0.092182 | −0.227387 | 0.10825212 | −0.1253385 | 0.02400758 | −0.0687886 | 0.31182465 |
| mlη | DN(CD8mCD4mTCRp).mln | 0.100694 | 1.9931552 | −0.4647179 | −0.2311096 | 0.05601039 | 0.21848508 | −0.1823944 |
| mlη | T8.mln | 0.12054127 | 0.04248061 | 0.16474622 | 0.07111475 | 0.12896372 | 0.1105647 | 0.04575163 |
| mlη | T8.Heliosp.mln | −0.052984 | 0.46885944 | 0.23003383 | −0.334046 | 0.32899018 | 0.37686047 | −0.1663763 |
| mlη | T4.mln | 0.09101094 | 0.08367857 | 0.07171705 | 0.1168708 | 0.07049339 | 0.07801469 | 0.12217187 |
| mlη | T4.FPmRorgp.mln | −0.2560624 | −0.3611445 | 1.79374111 | 0.87764237 | 0.27628543 | −0.7237146 | 1.44529414 |
| mlη | T4.FPp.mln | 0.0071864 | −0.1653296 | −0.1368861 | 0.26406323 | 0.09095651 | 0.00570642 | −0.0483734 |
| mlη | T4.FPpHeliosm.mln | −0.1404635 | −0.3650599 | 0.14353639 | −0.2287931 | −0.2938737 | −0.4828549 | 0.17069594 |
| mlη | T4.FPpRorgpHeliosm.mln | −0.0610011 | 1.17247529 | 0.48837746 | 0.32100889 | 3.29491337 | 0.47273611 | 1.72670628 |
| mlη | ILC.il22p.mln | −0.0816138 | −1.4150375 | −3.8365013 | −1.8109662 | 0.67807191 | −3.9434165 | 2.19219417 |
| mlη | T4.ifngp.mln | 0.56551888 | 0.03999807 | 0.16928109 | 0.86336531 | 2.55914286 | 0.34149426 | 1.24456921 |
| mlη | T4.il10p.mln | 2.55458885 | 1.21501289 | 0.9273547 | −2.0524674 | 0.44057259 | 2.25604016 | 1.36757076 |
| mlη | T4.il17p.mln | 0.91085327 | −1.2980349 | −0.2772763 | 0.98145794 | 0.40935759 | −0.267008 | 1.30654072 |
| mlη | T4.il22p.mln | −0.9682911 | −3.9068906 | −0.9219975 | −0.4365707 | 0.45066141 | −2.0655883 | 1.02778316 |
| pp | mono.pp | −0.2395166 | 1.64742659 | −1.3329131 | −0.1098645 | 2.32956816 | −0.3191949 | 0.77334555 |
| pp | CD11bpCD11cmF4/80pMF.pp | −1.325822 | 0.96457051 | −1.4267509 | −1.259151 | −2.5129076 | −2.142958 | −0.1258844 |
| pp | CD11bpCD11cpF4/80pMNP.pp | −0.1740294 | 2.47222928 | −1.1694128 | −0.4164043 | 1.99332441 | 0.05528244 | 0.05626822 |
| pp | CD103pCD11bmDC.pp | −0.1831971 | −1.3147466 | −0.7016277 | 0.7993381 | −0.4572623 | −0.2492362 | 0.06926498 |
| pp | CD103pCD11bpDC.pp | 0.43192928 | −0.1468635 | −1.4766798 | 0.32846255 | 0.67691296 | −0.0388249 | −0.6317431 |
| pp | pDC.pp | 0.49694454 | 1.06657849 | −0.2548605 | 0.45121111 | −1.4965356 | 0.97049812 | 3.80288349 |
| pp | ILC3.pp | −0.5334322 | 1.04730572 | −0.8271634 | −1.2473811 | −0.2165751 | −0.7009863 | −0.9912828 |
| pp | B.pp | 0.00261911 | −0.0315201 | −0.460931 | −0.0158153 | −0.0785609 | 0.08729218 | 0.02211236 |
| pp | Tgd.pp | −0.0027896 | 0.10608553 | −1.4036254 | −0.251008 | 1.57517516 | −0.2095985 | −0.7444166 |
| pp | Tab.pp | 0.08978602 | −0.0461225 | 0.10752164 | 0.27676178 | −0.2211578 | −0.8530391 | 0.38772229 |
| pp | DN(CD8mCD4mTCRp).pp | −0.5708511 | 0.40210517 | −2.1103448 | −3.0524398 | 0.19080913 | −0.8505367 | −1.6258391 |
| pp | T8.pp | 0.04596336 | −0.0159998 | −0.7510632 | 0.17344661 | 0.12182092 | 0.07896938 | −0.4755107 |
| pp | T8.Heliosp.pp | −0.1899704 | −0.3783026 | −0.8090171 | −0.9454223 | −0.2745032 | −0.0421979 | −0.4752342 |
| pp | T4.pp | 0.10747758 | 0.0860463 | 0.39773902 | 0.12055583 | 0.02767423 | 0.13134463 | 0.38729549 |
| pp | T4.FPmRorgp.pp | 0.44282066 | −0.1500254 | 2.71868297 | 0.26006284 | 0.74123393 | −1.982298 | 2.53482403 |
| pp | T4.FPp.pp | 0.09835268 | −0.3605152 | −1.3317123 | −0.2460656 | 0.07262084 | −0.1095536 | −1.2010675 |
| pp | T4.FPpHeliosm.pp | −0.1871619 | 0.02687234 | −0.3079576 | −0.0680623 | −0.4392553 | −0.3449228 | 0.29868889 |
| pp | T4.FPpRorgpHeliosm.pp | −0.5518065 | 1.52795088 | 0.62543589 | −0.3886088 | 1.02376034 | 1.09300459 | 1.52578929 |
| pp | ILC.il22p.pp | 0.47740567 | 0.91788819 | −1.5005162 | −0.7501301 | 2.30273985 | −1.3411163 | −1.7302305 |
| pp | T4.ifngp.pp | −0.6596235 | −0.6360367 | −1.5749611 | −0.3827833 | 0.48900672 | −0.8365013 | −1.4394977 |
| pp | T4.il10p.pp | 0.90551313 | 0.7942298 | −0.7389338 | −1.8676015 | 0.82286567 | 1.17744502 | 0.04644892 |
| pp | T4.il17p.pp | −0.964902 | −0.1881838 | −2.7322161 | −0.9708415 | −0.5205285 | 0.63597595 | −1.6048368 |
| pp | T4.il22p.pp | −0.4845666 | −1.6080924 | −2.3019892 | −0.1914164 | −0.0377767 | −2.6615316 | −1.3233629 |
| si | mono.si | −0.053596 | 0.5429893 | 0.39328808 | −0.2204218 | 0.02594576 | 0.16243746 | 0.09592295 |
| si | CD11bpCD11cmF4/80pMF.si | −1.3799687 | −1.1935556 | −1.425696 | −3.6760708 | 0.01088147 | −4.0155573 | −0.4110986 |
| si | CD11bpCD11cpF4/80pMNP.si | −0.6344957 | −0.3829852 | −1.8914579 | −0.5180935 | 0.24215878 | −1.3737036 | −2.6720076 |
| si | CD103pCD11bmDC.si | 0.66986721 | −0.4815851 | −0.5120451 | 0.18992692 | −0.086097 | −0.2777772 | −0.569821 |
| si | CD103pCD11bpDC.si | 0.16398195 | 0.5185522 | −0.4956876 | 0.69980344 | 0.60413068 | 1.08076963 | −0.4988712 |
| si | pDC.si | −0.2095461 | 0.21948577 | −0.3155475 | 0.27093004 | −0.9237174 | 0.64716851 | 1.43921367 |
| si | ILC3.si | 0.03554304 | 0.42128952 | 0.36037966 | 0.66991827 | 0.40892966 | −0.1300237 | 0.16512777 |
| si | B.si | −0.110959 | −0.3449538 | −0.2784486 | −0.763478 | −0.1347074 | −0.5652668 | −0.0563105 |
| si | Tgd.si | −0.8067932 | −0.2593313 | −0.9106877 | −0.7190992 | 0.03879936 | 0.513168 | −0.3538086 |
| si | Tab.si | 0.14295894 | 0.17610541 | 0.25753132 | 0.20308186 | 0.33997167 | 0.43999339 | 0.02491745 |
| si | DN(CD8mCD4mTCRp).si | −0.6437324 | −0.3263212 | −0.157106 | −0.7331032 | −0.11635 | 0.16431716 | −0.5797366 |
| si | T8.si | −0.1259266 | −0.1973983 | −0.5210645 | −0.2319173 | 0.48023926 | 0.01882683 | −0.1701798 |
| si | T8.Heliosp.si | −0.6958025 | −0.1155548 | −1.2328978 | −0.9390933 | −0.1395024 | 0.18320862 | −0.5342343 |
| si | T4.si | 0.15619514 | 0.18513815 | 0.20593579 | 0.1718351 | −0.0639879 | −0.049944 | 0.19087935 |
| si | T4.FPmRorgp.si | −0.2261118 | 0.13582235 | 2.52729138 | 0.68775605 | 0.84551841 | −0.8612122 | 2.56010674 |
| si | T4.FPp.si | 0.04048472 | 0.09288592 | −0.4017899 | −0.1217977 | 0.03351489 | 0.33003348 | −0.6716267 |
| si | T4.FPpHeliosm.si | −0.2916361 | −0.2500645 | 0.22731679 | −0.7295853 | −0.1903256 | −0.2788183 | 0.28161769 |
| si | T4.FPpRorgpHeliosm.si | −0.7286291 | 0.88247136 | 1.58029228 | −0.0130825 | 0.74130018 | 0.88482422 | 2.01928493 |
| si | ILC.il22p.si | 0.6828359 | 0.56332768 | 0.75199792 | 0.2157876 | 1.71843564 | −1.1101408 | 1.10102832 |
| si | T4.ifngp.si | 1.18464651 | 0.82938123 | 1.92988809 | −0.1685385 | 2.22635044 | 0.46681115 | 1.35773244 |
| si | T4.il10p.si | −0.0489096 | 1.39207635 | 2.01071703 | −1.9551458 | 1.7255307 | 0.5334322 | 1.86059694 |
| si | T4.il17p.si | 1.43831074 | 0.23411891 | 1.4623879 | 0.85934168 | 0.41226442 | 1.00242208 | 1.78069214 |
| si | T4.il22p.si | 0.78024746 | −3.5602621 | 0.27169023 | −0.4022602 | −0.0390252 | −2.1962085 | 2.50063398 |
| slo | mono.slo | −0.1191042 | 0.69783079 | −0.3920607 | −0.608775 | 0.03950061 | −0.14196 | 0.49249947 |
| slo | CD11bpCD11cmF4/80pMF.slo | −1.3082742 | −0.8151926 | −4.3098553 | −2.8759587 | −0.263928 | −1.9803539 | −0.0381465 |
| slo | CD11bpCD11cpF4/80pMNP.slo | −0.6152263 | −0.5154094 | −1.9914197 | −0.9018649 | 0.39711236 | −0.207325 | −0.2932982 |
| slo | CD103pCD11bmDC.slo | −0.7957094 | −0.4775334 | −2.4576339 | −0.0624959 | −0.6759149 | 0.55711272 | 0.47407509 |
| slo | CD103pCD11bpDC.slo | −1.7332135 | −1.3852902 | −3.6780719 | 0.20457114 | −1.5570565 | −1.4150375 | 0.88264305 |
| slo | pDC.slo | −0.3768543 | 0.17829439 | −0.8217056 | −0.1845083 | −0.841379 | −0.440441 | 4.06308854 |
| slo | ILC3.slo | 0.0671142 | 0.3081223 | −0.3923174 | 0.3081223 | 1.7574297 | −0.6918777 | 0.19264508 |
| slo | B.slo | 0.03227355 | 0.04594561 | −0.0028748 | 0.11026345 | 0.19728219 | 0.07721842 | −0.0931762 |
| slo | Tgd.slo | 0.01116931 | 0.00373272 | −0.2615402 | −0.2130787 | 0.03689958 | 0.10102992 | 0.66963781 |
| slo | Tab.slo | 0.31468451 | −0.2009799 | 0.3335692 | 0.09794095 | −0.3355398 | 0.05913806 | 0.42544289 |
| slo | DN(CD8mCD4mTCRp).slo | −0.5559 | 0.2583245 | −0.947587 | −1.2923053 | −0.439647 | −0.5603909 | −0.7929392 |
| slo | T8.slo | −0.0243171 | 0.02496493 | −0.0343963 | 0.08129307 | 0.21618585 | 0.10795925 | 0.05066146 |
| slo | T8.Heliosp.slo | −0.3891612 | 0.04394959 | 0.0299575 | −0.4201437 | 0.70898426 | 0.03697051 | −0.016004 |
| slo | T4.slo | 0.04677714 | −0.013258 | 0.07672369 | 0.01702448 | −0.1311464 | −0.0429802 | 0.00871592 |
| slo | T4.FPmRorgp.slo | 0.94603717 | −1.9700468 | 0.01005367 | 0.62148838 | 0.78264317 | −1.1045889 | 0.82881335 |
| slo | T4.FPp.slo | 0.02847751 | 0.08085681 | −0.2645451 | 0.24829959 | 0.05974187 | 0.08085681 | 0.16513698 |
| slo | T4.FPpHeliosm.slo | −0.1154471 | −0.3984556 | 0.02966102 | −0.2551539 | −0.547968 | −0.0748424 | −0.0712613 |
| slo | T4.FPpRorgpHeliosm.slo | 1.4157028 | 0.57248699 | 0.02637542 | 0.68603528 | 1.70084917 | 2.25100631 | 2.78821568 |
| slo | ILC.il22p.slo | −0.0815299 | −3.9228321 | −2.5077946 | 2.77529371 | −1.0815299 | 0.70165873 | 0.5796682 |
| slo | T4.ifngp.slo | 0.39298426 | 0.06504353 | 1.19384828 | −0.4649134 | 1.81633128 | −1.8536105 | 0.03971763 |
| slo | T4.il10p.slo | 2.16349873 | 0.73118324 | 1.20789285 | −2.7369656 | 0.3219281 | 2.79908731 | 1.81557543 |
| slo | T4.il17p.slo | 1.46498503 | −0.3164737 | −0.2630344 | 0.55254102 | 0.28113621 | 0.82350235 | 0.68352634 |
| slo | T4.il22p.slo | 0.48649986 | −4.2630344 | −0.6660993 | 1.69348696 | −0.0931094 | −3.7776076 | 0.74595438 |

A patchwork of effects was observed. Some innate cell types varied in response to several microbes, with expansion (e.g., CD103+ dendritic cells [DCs]), contraction (e.g., both CD11b+F4/80+ subsets of macrophages and mononuclear phagocytes), or both (e.g., plasmacytoid dendritic cells [pDCs]). Type 3 ILCs (ILC3s) were affected by only a few microbes, a result consistent with earlier studies reporting microbiota-mediated alterations in IL22 production but not in overall ILC3 frequency. Most cells of the adaptive immune system seemed largely unresponsive, at least in terms of abundance, with comparatively infrequent and modest changes in the proportions of B, γδT, and αβT (T4 or T8) cells. The notable exceptions were Tregs and their subsets, which, in line with previous reports (Lathrop et al., Nature 2011; 478, 250-254; Faith et al., Sci. Transl. Med 2014; 6, 220; Sefik et al., Science 2015; 349, 993-997), were strongly induced by a number of individual microbes. These effects were distributed among the different microbes tested, with a range in the number of cell types affected by a given microbe (as judged by the proportion of cell types modified by a z-score of ≥2 relative to GF; FIG. 2C). Some microbes seemed stealth-like, affecting few or none of the immunocyte populations examined (e.g., Peptostreptococcus magnus and Bacteroides salanitronis), but others were substantially more active (Bacteroides uniformis). Microbes of the same phylum or genus provoked no obviously shared patterns of these signatures in terms of either the number of cell types affected (FIG. 2C) or the extent of change relative to GF (FIG. 2B, Tables 4A-G).

In addition to quantitative changes, some reproducible alterations in the configuration of cell populations within flow cytometry counting gates were observed with a few microbes, as illustrated by the difference in CD11c intensity in CD11b+CD11c+ mononuclear phagocytes and DCs (FIG. 2D; see also FIGS. 9E-9G and Table 5). These changes occurred independently of the quantitative perturbations measured above. Along the same lines, the induction of inflammatory or suppressive cytokines by CD4+ T cells and ILCs was assessed; because the staining panels were designed before defined markers for ILC subsets had been established, we assessed only bulk ILC populations in this instance (FIG. 2E). Only a handful of symbionts elicited deviations from GF levels in T cells, including SFB and Th17 cells, but other unprecedented associations were found, such as Coprobacillus with IL10+SI T cells and Bifidobacterium longum with colonic Th1 (T4.IFNγ+) cells (FIG. 2E). Bacterial influences on IL22 production by ILCs were far more pronounced, with significant induction by microbes such as Bacteroides dorei and B. longum in both gut tissues. Conversely, Acinetobacter lwoffii, Clostridium sordellii, and Veillonella appeared to repress IL22 production, especially in the colon, a result indicating that the microbes can have differential effects on ILC activation. Without being bound to any particular theory, these observations provide a nuanced perspective on bacterial modulation of ILCs and may explain discrepancies in studies comparing IL22 production in GF and specific pathogen-free (SPF) mice.

TABLE 5
Qualitative phenotypic changes in the immune cells

| Cell population | Observation | Tissue |
|---|---|---|
| CD11b+CD11c+ | This inclusive gate has 4 different populations | All tissues, but |
| | with different distribution of CD11b and CD11c: | especially |
| | CD11bmedCD11cmed, CD11bhiCD11chi, | intestinal tissues |
| | CD11chiCD11bmed, CD11bmedCD11med. | |
| CD11b+CD11c+ | The distribution of the CD11b+ and CD11c+ | SLO, mLN |
| | is different and the MFI is dimmer. There is | |
| | a dimmer CD11bmedCD11med population that is | |
| | largely absent in intestinal tissues. | |
| Ly6c+ monocytes | There is a Ly6chi and Ly6cmed population that | Colon and SI |
| | we gate together. Some microbes induce Ly6hi | |
| | or Ly6cmed populations to different extents. | |
| | In addition to these two populations, | |
| | Bunif.ATCC8492 and Cramo.AO31 colonized mice | |
| | has an extra high Ly6c+ population. | |
| Ly6c+ monocytes | The Ly6chi population in the SLO has a lower | SLO |
| | MFI for CD11b. | |
| CD11b+CD11c+ | Pmagn.AO29 colonized mice have a population | SI |
| | that has a very high CD11c, a unique population | |
| | not present in other colonization experiments | |
| CD11bmedCD11c− | Some tissues have a CD11bmedCD11c− | All tissues |
| | population that has not been included in our | |
| | analysis. (e.g. SLO in the Pueno.UPII60.3 | |
| | colonized mice, SI in the Lach.2.1.58FAA | |
| | colonized mice, SI in the Efaec.TX1330 | |
| | colonize mice) | |
| CD45+ | In the small intestine there are two CD45+ | SI |
| | populations: Bright and dim. For all the cells | |
| | except for ILC3 and ILC.IL22 staining, we only | |
| | gated on CD45 hi population. In other tissues | |
| | there is a compact CD45 population. | |
| Helios− pTregs | Tregs express 3 different levels of Helios: | Colon and SI |
| | negative, dim and high. Helios− gate is | |
| | based on the non Tregs that do not express | |
| | Helios as well as guidance of Rorg staining. | |
| | This predominantly corresponds to the | |
| | negative population. | |

Fecal IgA was quantitated from specimens obtained at the end of the 2-week monocolonization. All IgA levels ranged between GF and SPF. Fold change relative to GF is shown in FIG. 2C. IgA induction varied by organism and did not follow microbial phylogeny. Total IgA was measured in fecal samples by ELISA and organism-specific IgA was evaluated by flow (FIG. 9H). There was a significant correlation between total and organism specific IgA (r=0.51, p=0.025). Without being bound to any particular theory, this suggests microbes induce IgA production by acting as standard “immunogens” rather than as bystanders that boost IgA production without being direct targets themselves.

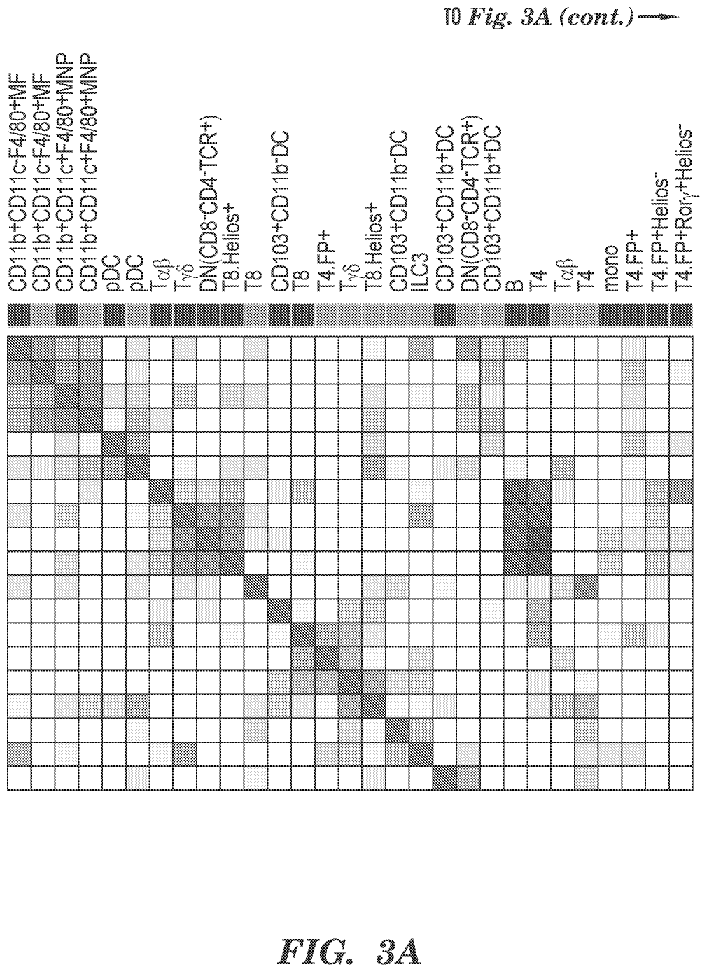

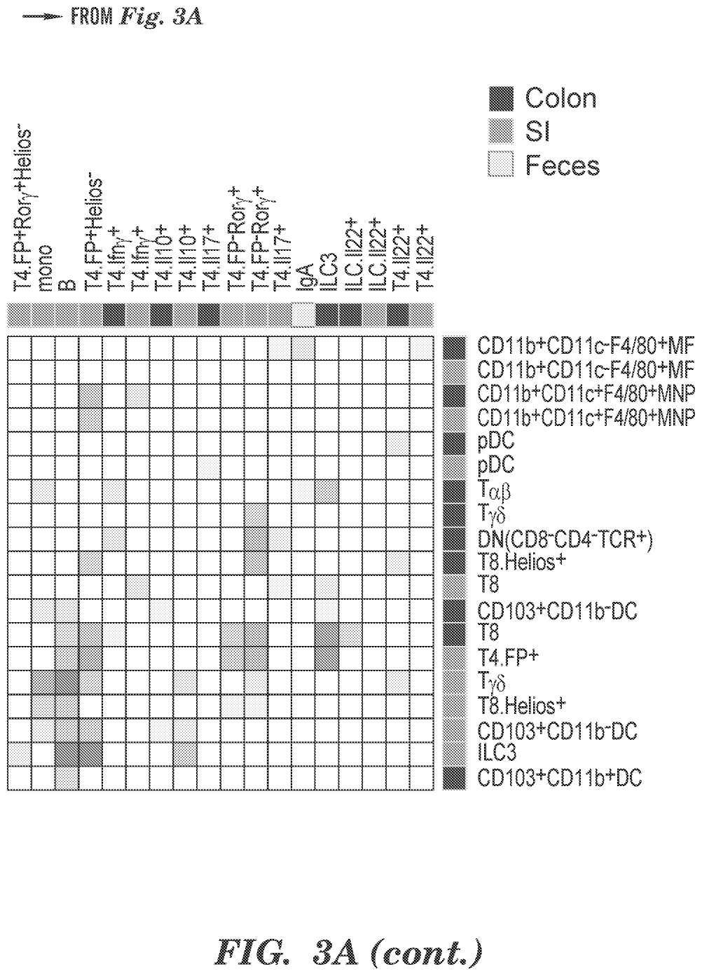

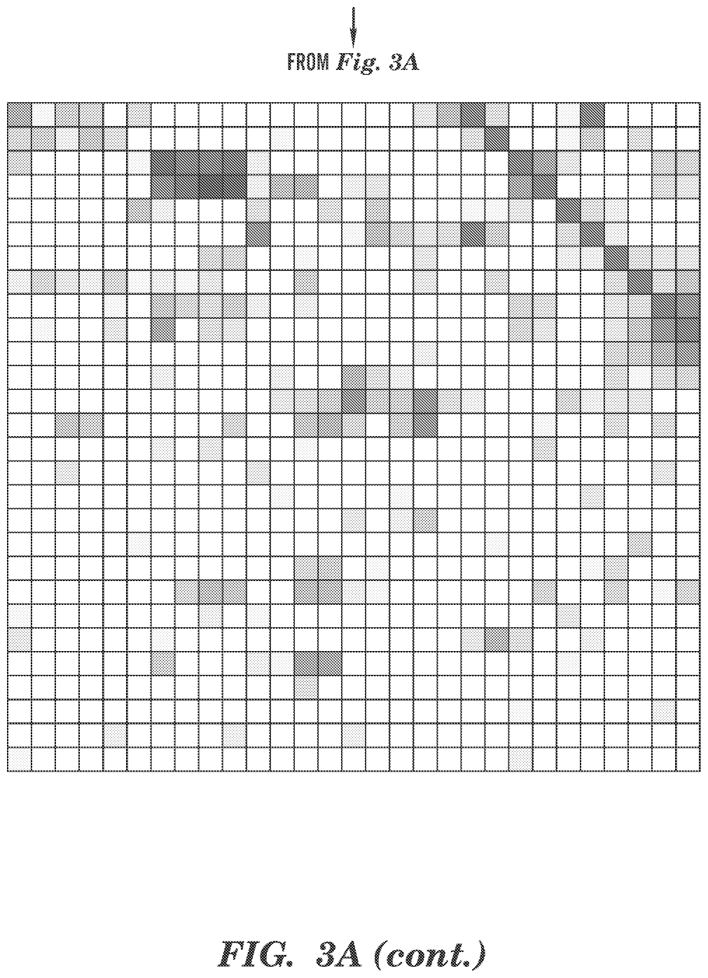

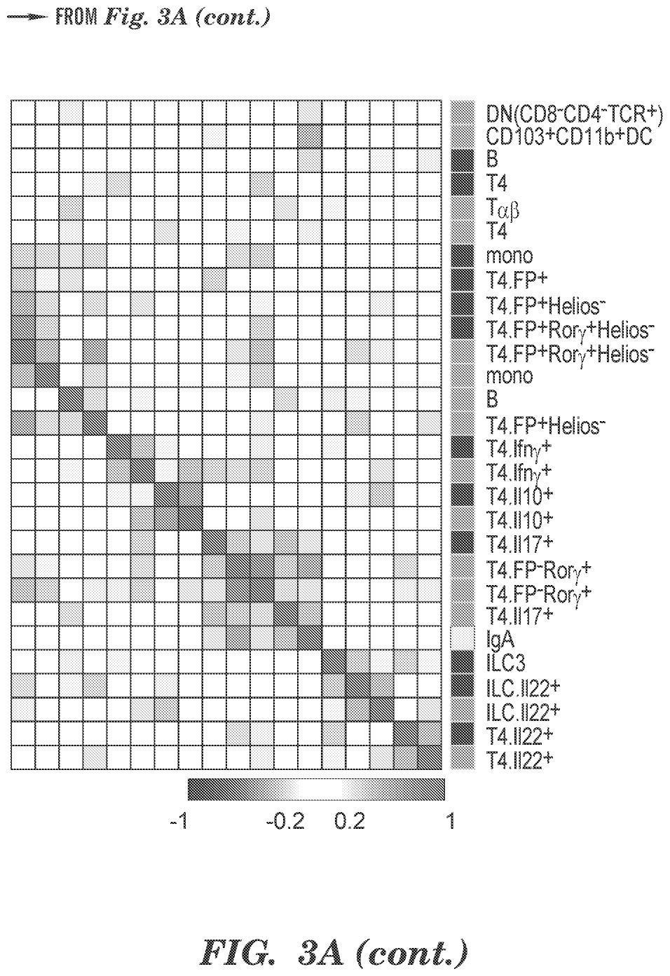

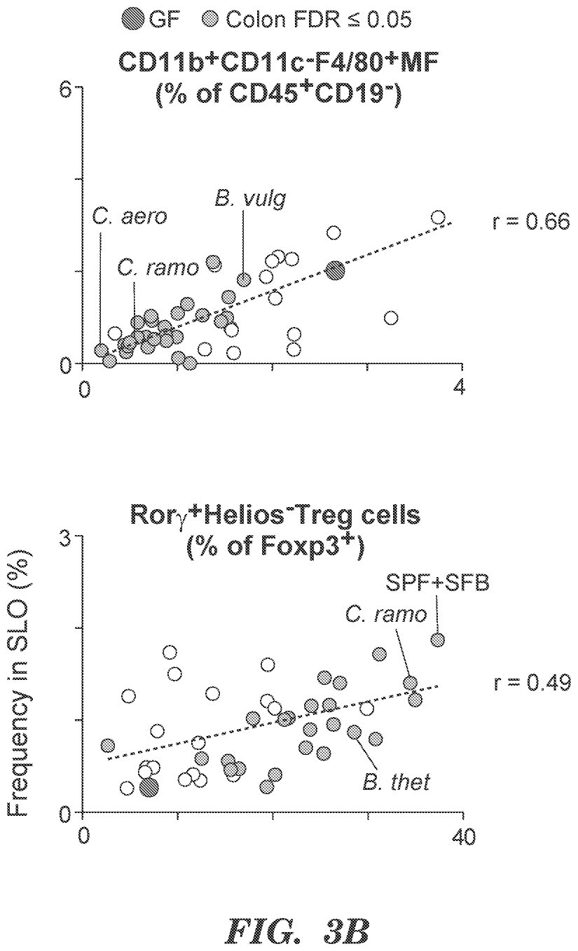

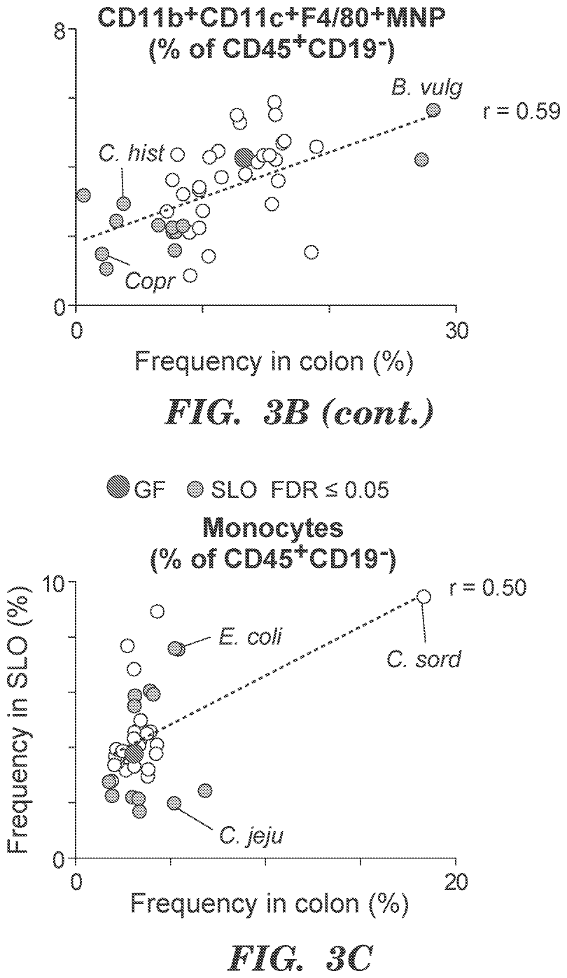

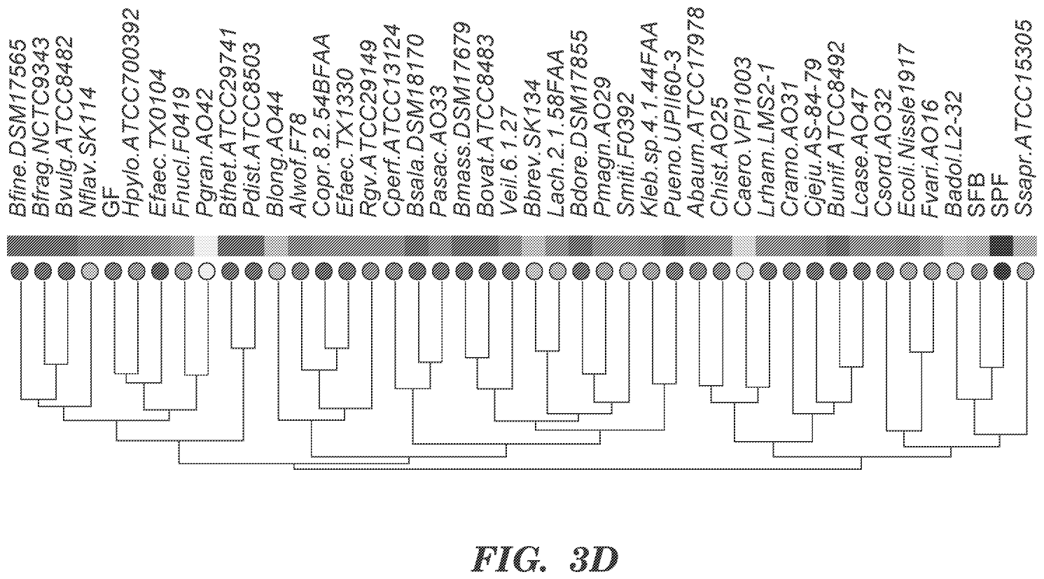

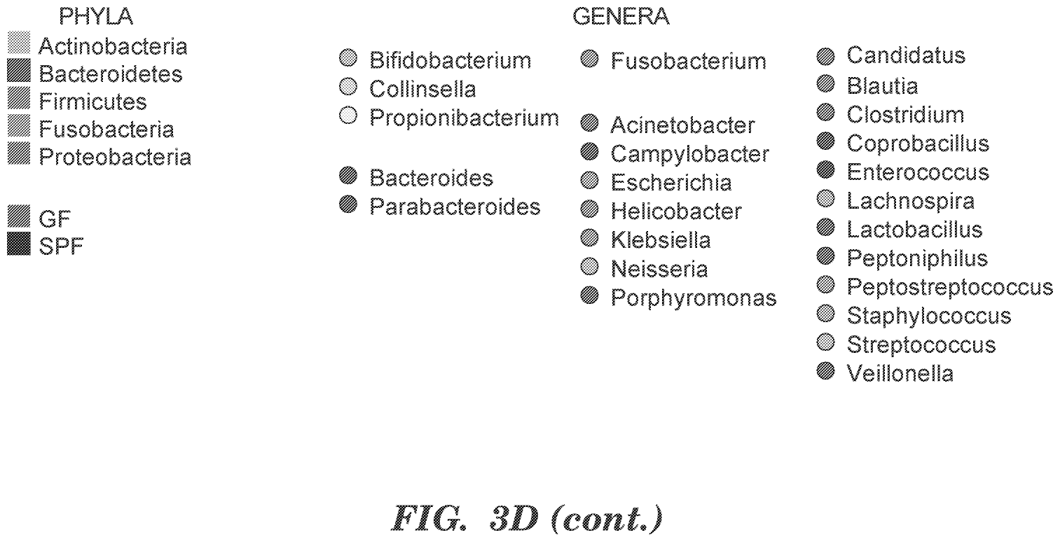

Further insight was obtained by correlating the responses induced by the set of microbes in the colon versus the SI (FIG. 3A). Many of the stronger correlations corresponded to the same cell type in the colon and SI (e.g., F4/80+ mononuclear phagocytes, IL10-producing CD4+ T cells, or RORγ+ Tregs), an observation denoting similar responses despite differences in tissue organization and microbial load in these two gut segments. Other correlated phenotypes, although expected (e.g., ILC3 frequency and the proportion of IL22 producing cells among bulk ILCs; CD4+RORγ+ T cell frequency and IL17a production), did reinforce the significance of the trends observed. Finally, some correlated traits were less anticipated (e.g., Tγδ and Helios+CD8+ T cells; CD4+ T and B lymphocytes) and may reflect common sensing pathways or integration of microbial influences by the immunologic network.

TABLE 6
Contraction or Expansion of Immune cells following microbe administration

| | | Microbe | |
|---|---|---|---|
| Tissue | Immune Cell Type | Cell Expansion | Cell Contraction |
| colon | Monocytes | Clostridium sordellii_AO32 | |
| colon | CD11b+CD11c−F4/80+MF.co | | Acinetobacter baumannii_ATCC17978, |
| | | | Acinetobacter lwoffii_F78, |
| | | | Bifidobacterium breve_SK134, |
| | | | Bacteroides dorei_DSM17855, |
| | | | Collinsella aerofaciens_VPI1003, |
| | | | Clostridium ramosum_AO31, |
| | | | Lactobacillus casei_AO47, |
| | | | Lachnospiraceae_sp_2_1_58FAA, |
| | | | Veillonella_6_1_27 |
| colon | CD11b+CD11c+F4/80+PMN.co | | Acinetobacter lwoffii_F78, |
| | | | Collinsella aerofaciens_VPI1003, |
| | | | Coprobacillus_8_2_54BFAA |
| colon | CD103+CD11b+DC.co | Bifidobacterium breve_SK134, | Acinetobacter lwoffii_F78, |
| | | Bacteroides uniformis_ATCC8492, | Clostridium perfringens_ATCC13124 |
| | | Lachnospiraceae_sp_2_1_ 58FAA | |
| colon | pDC.co | Bacteroides fragilis_NCTC9343, | Lactobacillus rhamnosus_LMS21, |
| | | Bacteroides vulgatus_ATCC8482 | Staphylococcus saprophyticus_ATCC15305 |
| colon | ILC3.co | | Coprobacillus_8_2_54BFAA, |
| | | | Parabacteroides distasonis_ATCC8503, |
| | | | Veillonella_6_1_27 |
| colon | ILC.il22p.co | Bacteroides uniformis_ATCC8492, | Acinetobacter lwoffii_F78, |
| | | Lactobacillus casei_AO47 | Coprobacillus_8_2_54BFAA, |
| | | | Clostridium sordellii_AO32, |
| | | | Veillonella_6_1_27 |
| si | ILC.il22p.si | Acinetobacter baumannii_ATCC17978, | Acinetobacter lwoffii_F78, |
| | | Bacteroides dorei_DSM17855 | Fusobacterium nucleatum_F0419, |
| | | | Propionibacterium granulosum_AO42, |
| | | | Veillonella_6_1_27 |
| colon | T4.il10p.co | Acinetobacter lwoffii_F78, | |
| | | Bifidobacterium longum_AO44, | |
| | | Bacteroides ovatus_ATCC8483, | |
| | | Bacteroides thetaiotaomicron_ATCC29741, | |
| | | Bacteroides vulgatus_ATCC848, | |
| | | Coprobacillus_8_2_54BFAA, | |
| | | Helicobacter pylori_ATCC700392, | |
| | | Enterococcus faecium_TX1330, | |
| | | Ruminococcus gnavus_ATCC29149, | |
| | | Veillonella_6_1_27 | |
| colon | T4.il17p.co | | Bacteroides thetaiotaomicron_ATCC29741, |
| | | | Peptostreptococus asaccharolyticus_AO33, |
| | | | Streptococcus mitis_F0392 |
| si | T4.il17p.si | | Clostridium perfringens_ATCC13124, |
| | | | Peptostreptococus asaccharolyticus_AO33 |

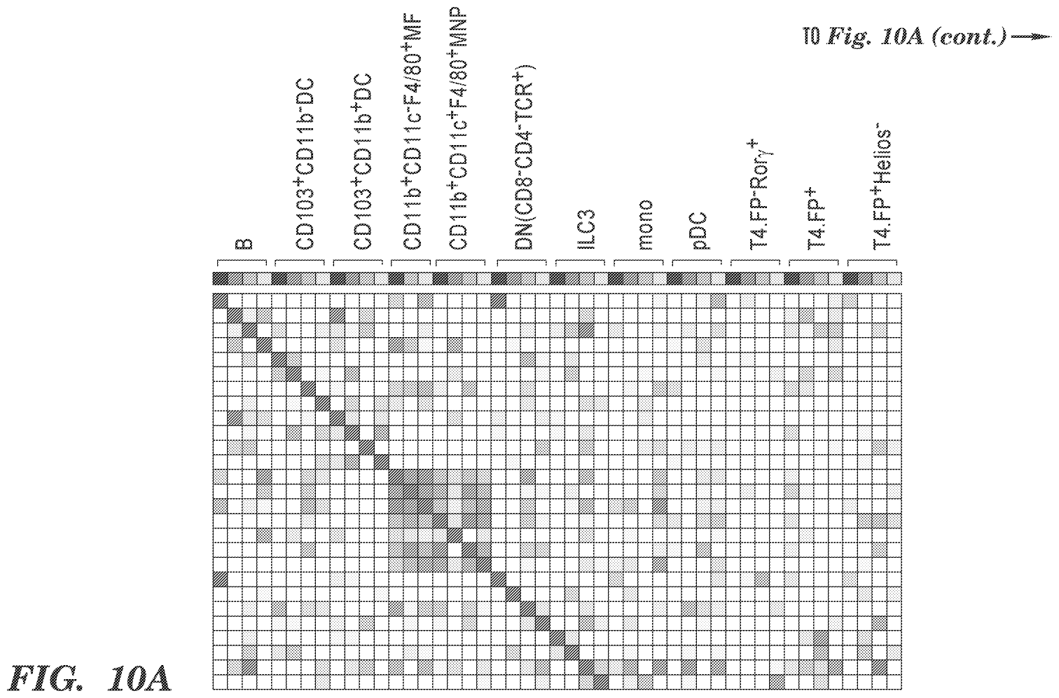

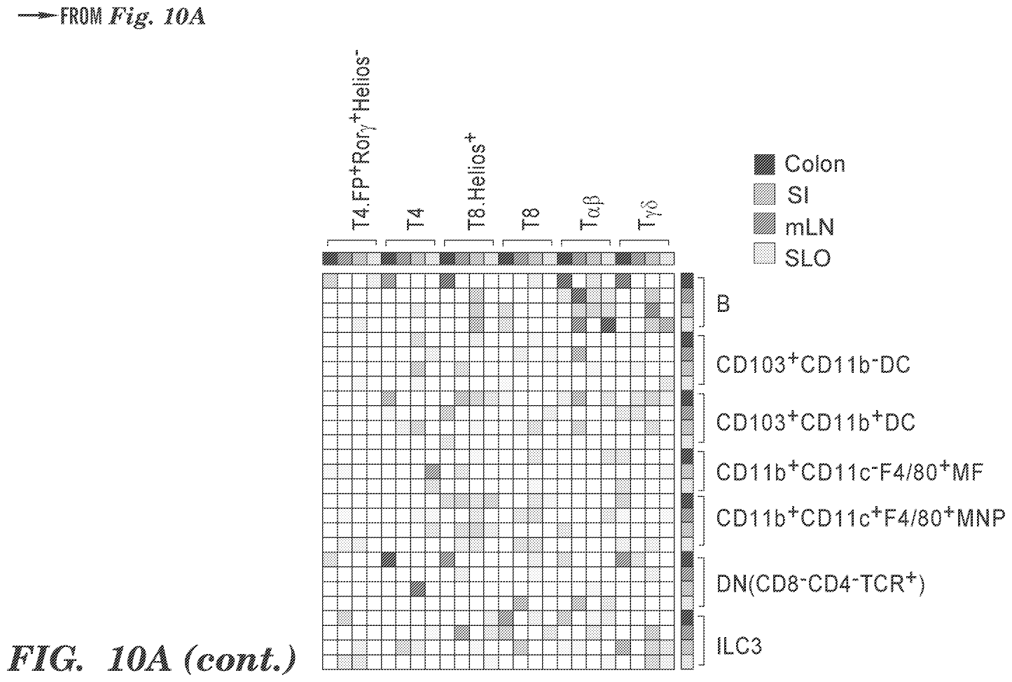

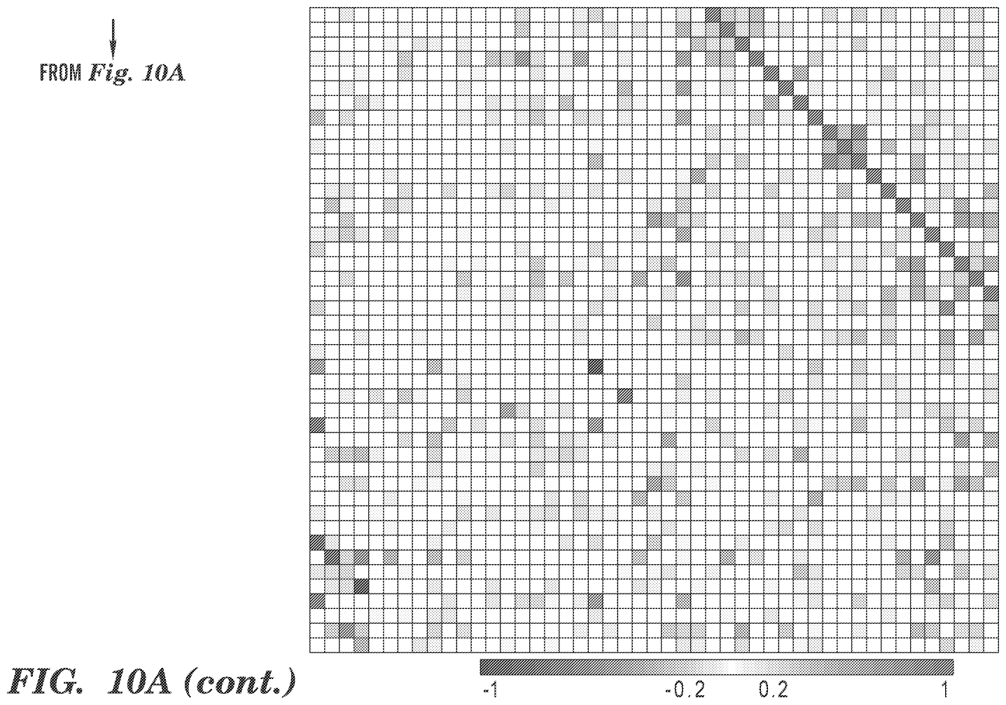

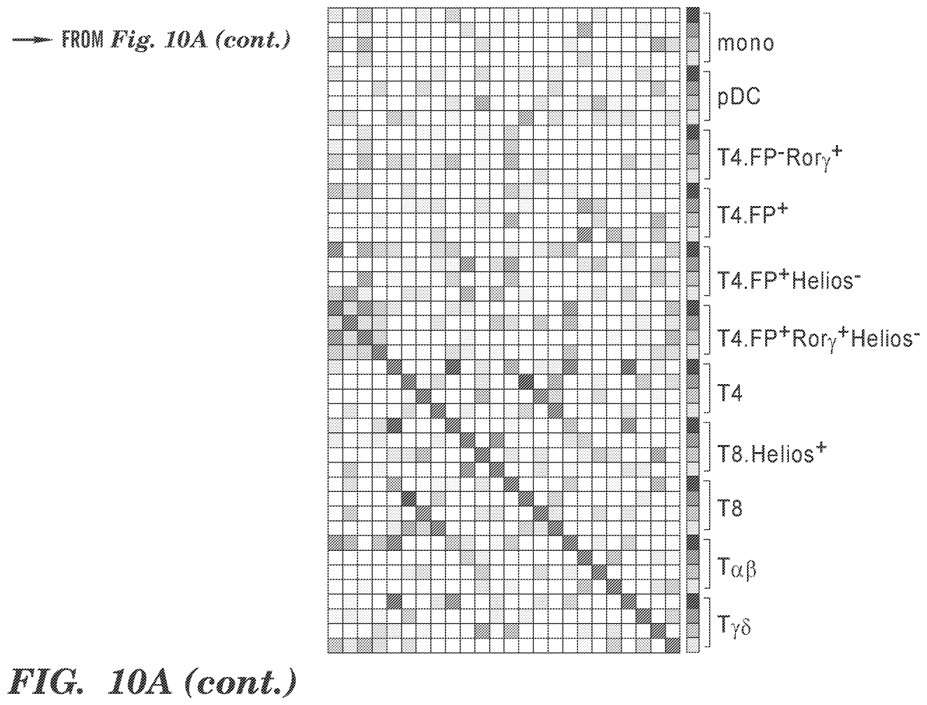

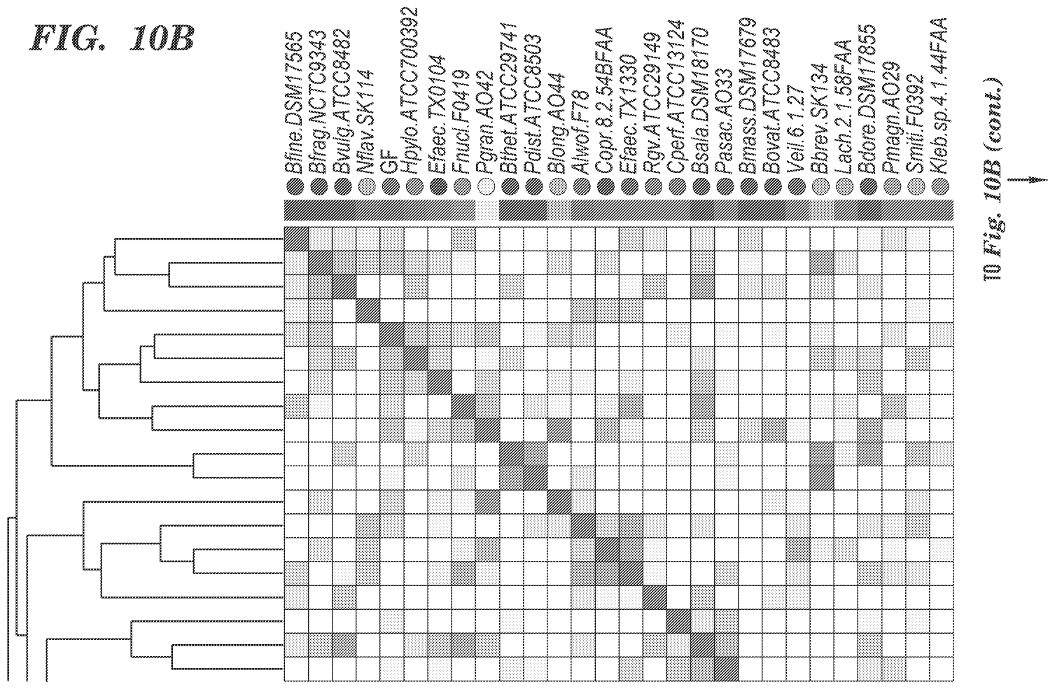

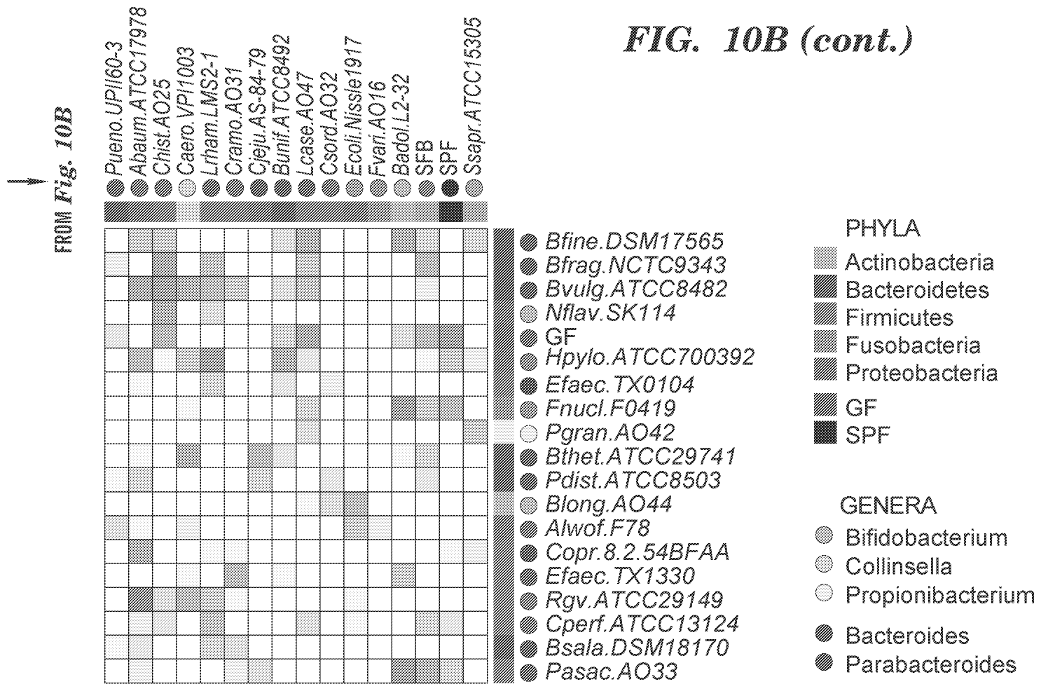

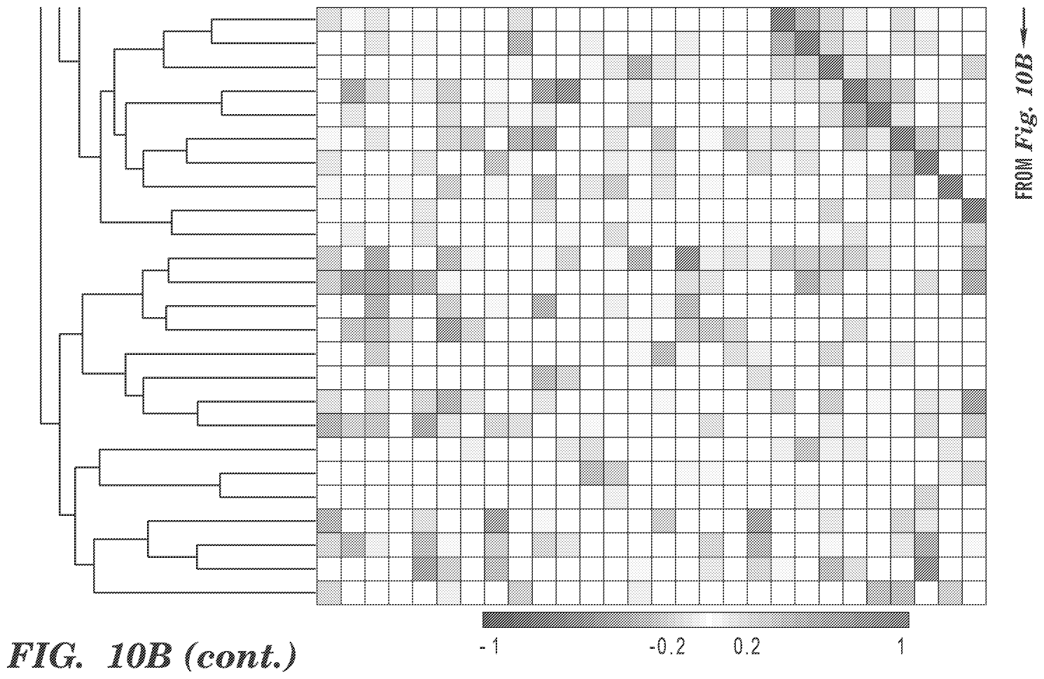

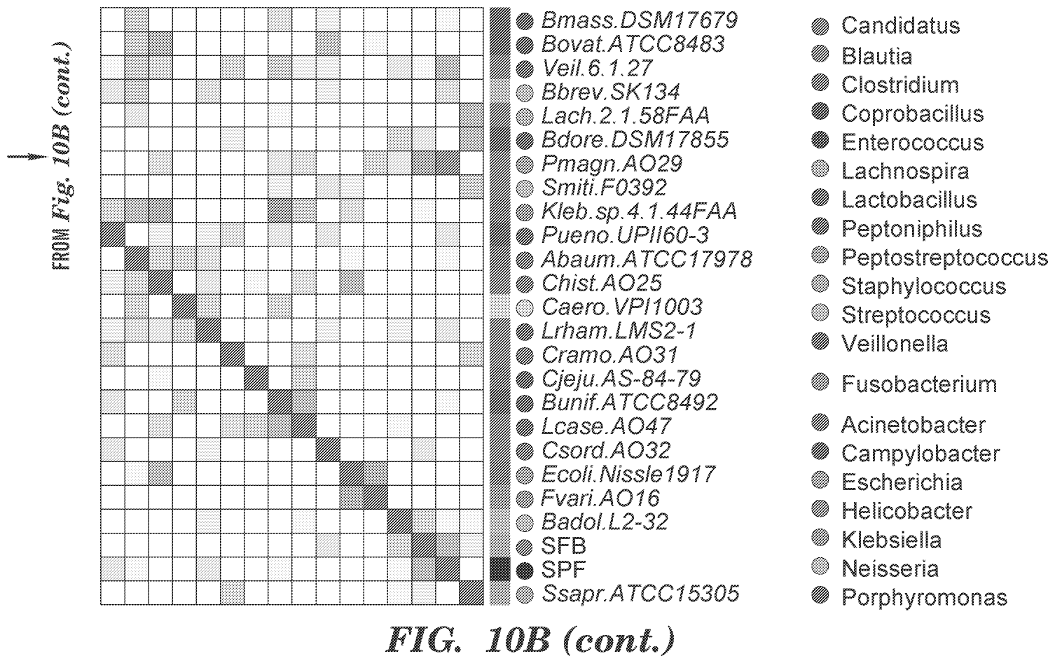

Bacteria of the same phylum or genus provoked no obviously shared patterns of these signatures in terms of either the number of cell types affected (FIG. 2C) or the extent of change relative to GF (FIG. 2B, Table 4A-G). The normalized immunophenotypic responses correlated between microbes in the SI and the colon (FIGS. 3D and 10B). The dendrogram generated by hierarchical clustering of these correlations bore testament to the true diversity of microbial functions represented by the organisms chosen for this screen. Bacterial species from the same phylum or genus largely failed to cluster together, a result pointing to a high degree of diversification in immunomodulatory properties within a phylum or genus. For seven species (nine strains total), the impact of additional strain(s) on lymphocyte populations such as Tregs was looked at. For the Bacteroides strains within the same species, quantifiable differences were found (data not shown—see supplemental materials of Geva-Zatorsky et al., Cell 2017, incorporated by reference herein below). The mean Euclidean distance between species was 0.39. Interestingly, the mean distance between strains within the same species was very similar—0.32. Without being bound to any particular theory, these results highlight the importance of strain-level information in relating microbial function to immunologic phenotypes.

### Effects of Bacterial Colonization in Systemic Lymphoid Organs

Immunocytes can migrate from the colon into the lymphatics and circulate between lymphoid organs. The inventors analyzed immunocyte populations in the mLNs and the SLO to determine whether immunologic alterations in the gut were reflected systemically. Most microbes had a limited effect on innate immunocytes in mLNs and the SLO (FIGS. 9C and 9D), although monocytes did vary markedly in the SLO. As in the intestine, adaptive immunocytes in lymphoid organs were mostly unaffected by microbial exposure. To detect more sensitively the echoes in lymphoid organs of microbe-instructed immunologic changes in the gut, the inventors correlated the immunologic phenotypes in the gut and secondary lymphoid organs (FIGS. 3B, and 10A). There was a significant correlation across all tissues for five cell types. For three of these types (the F4/80+ macrophage and mononuclear phagocyte populations and FoxP3+ Tregs), changes in the SLO were subtle but were correlated with frequencies in the gut across the set of microbes (FIG. 3B). Without being bound to any particular theory, this finding suggested a direct relationship between the two pools. The fifth cell type—the monocyte—was the exception, with equally strong induction by C. sordellii in the SLO and the intestines (FIG. 3C).

### Colonic and Small-Intestinal Transcriptomes of Monocolonized Mice

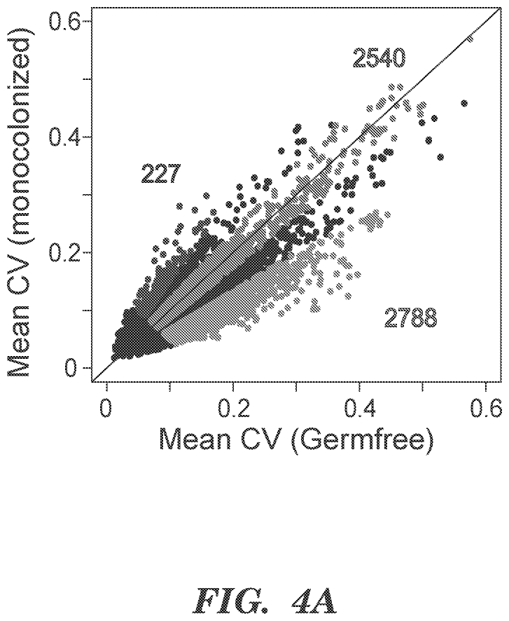

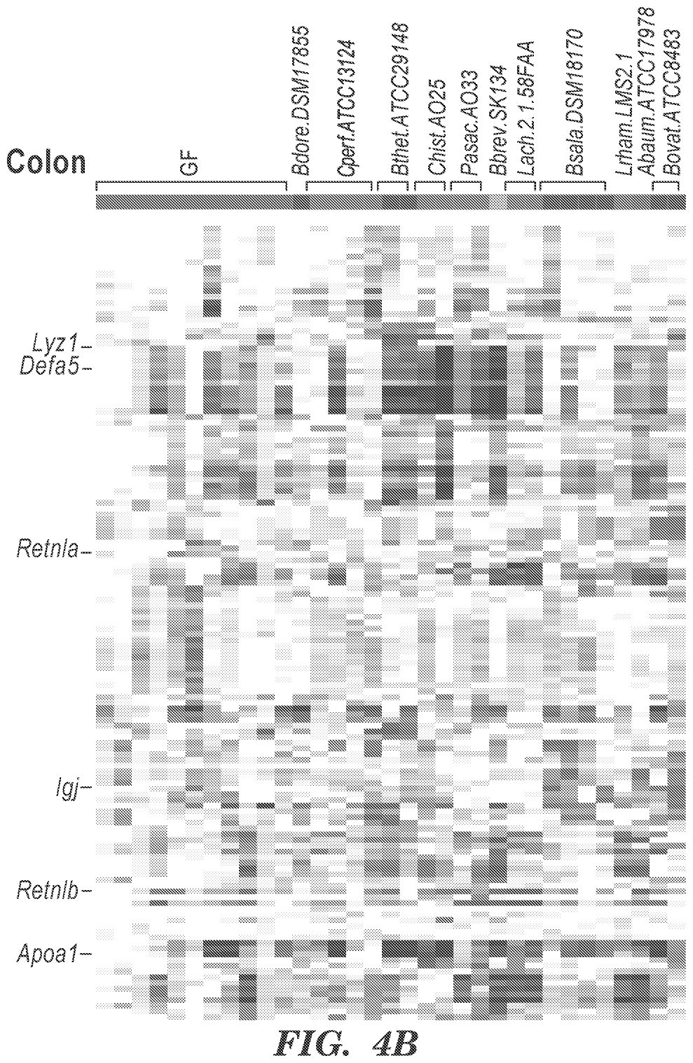

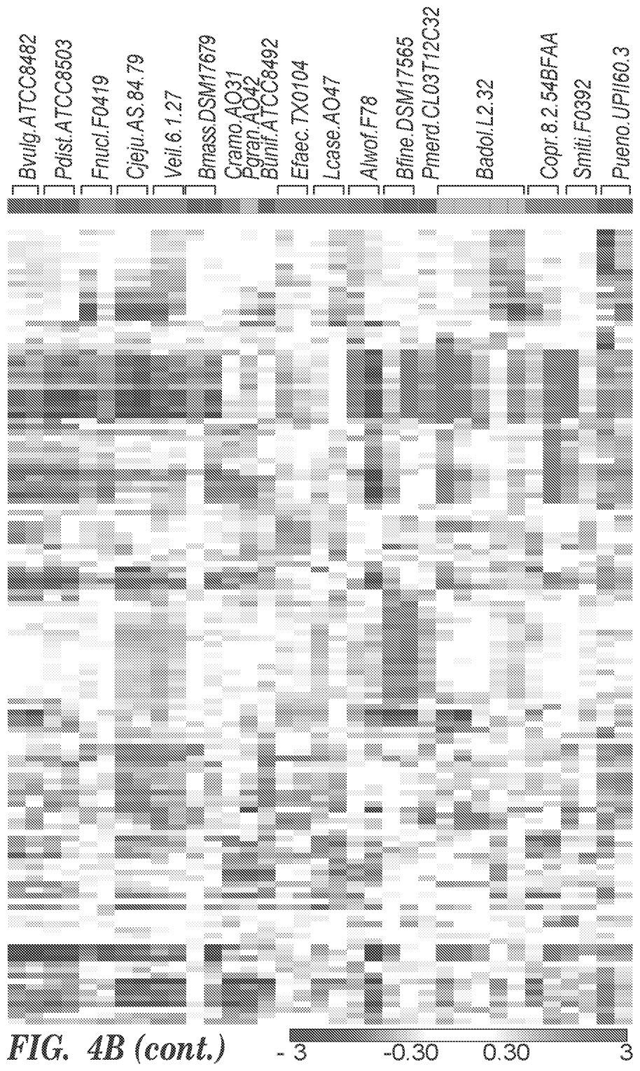

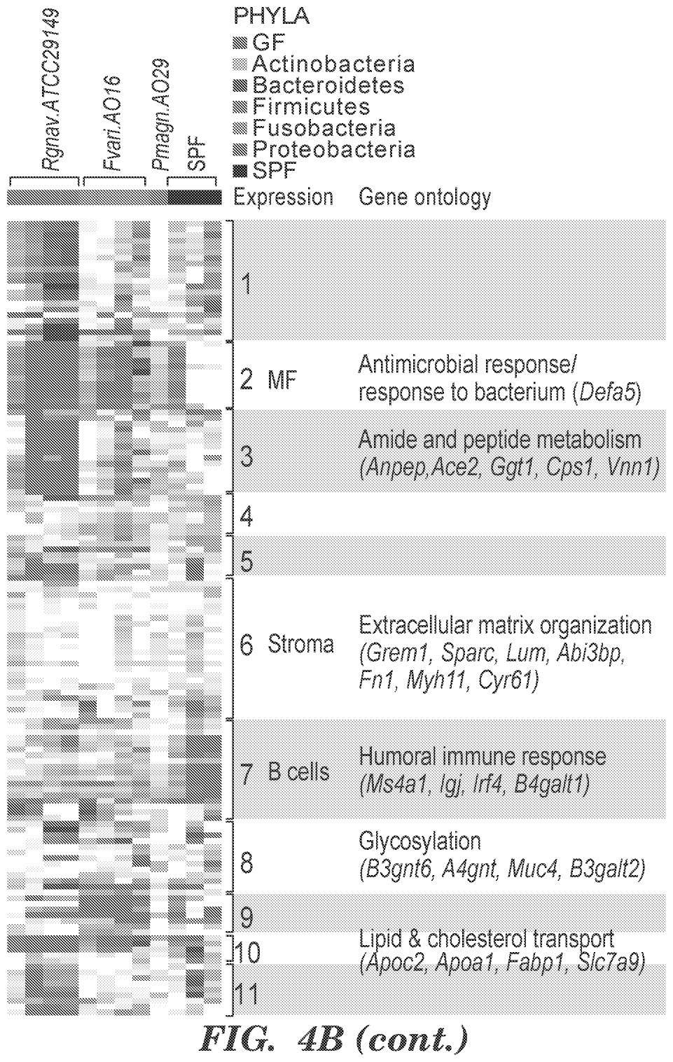

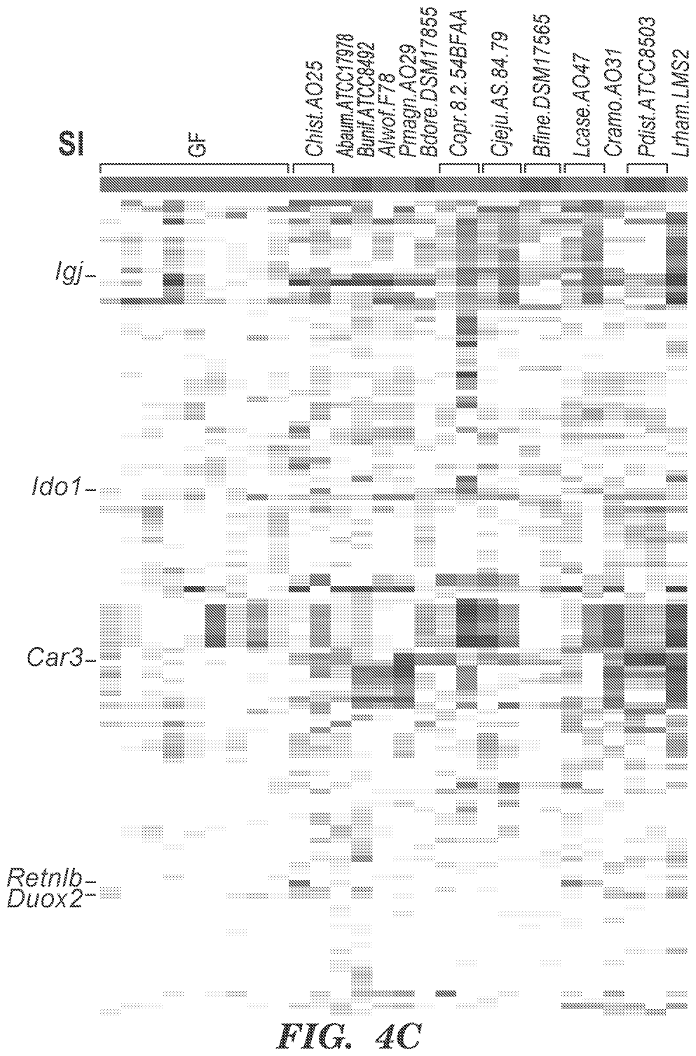

Transcriptomic changes induced by the various microbes in SI and colonic tissue were then investigated. Gene-expression profiles were generated in duplicate from whole-tissue RNA in order to capture responses in all major cell types, with controls from GF tissues included in every batch. A first observation from the compiled datasets was that there was more marked inter-individual variability in intestinal tissues than in other tissues we have recently profiled such as the fat and muscle (data not shown). Groups of variable genes appeared in the plot of gene-wise coefficients of variation (CV) (FIG. 4A): one group had the same variability in replicates of GF and monocolonized mice, but a larger group was more variable in GF colons than in microbially colonized colons, as if the presence of bacteria stabilized fluctuations in the transcriptome. Except for some B cell-specific transcripts, most of these highly variable genes could not be ascribed to fluctuations in the frequency of particular cell types.

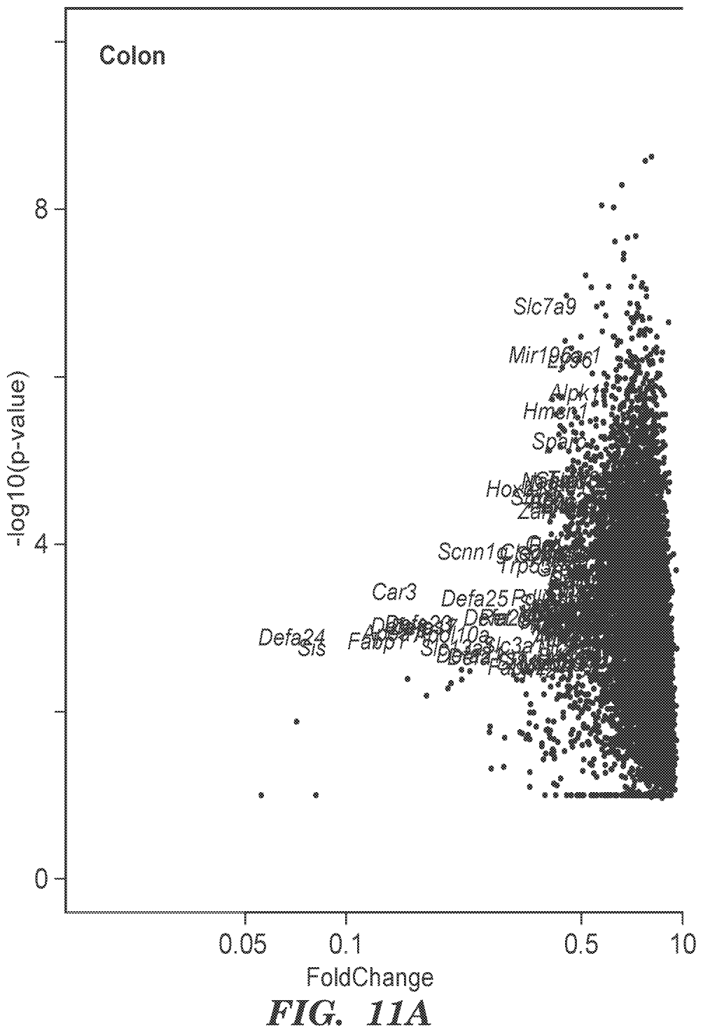

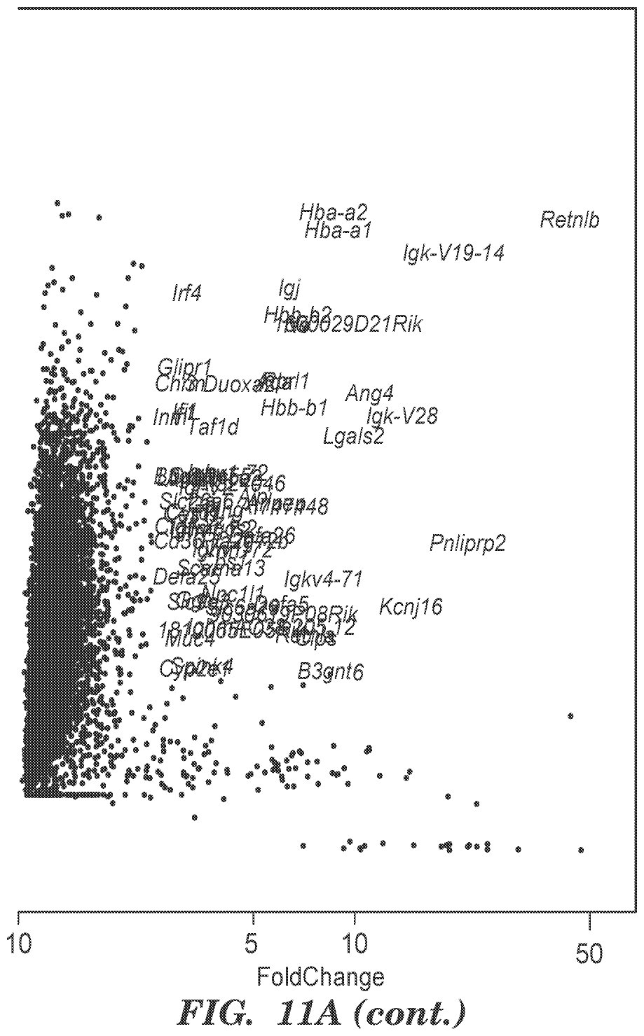

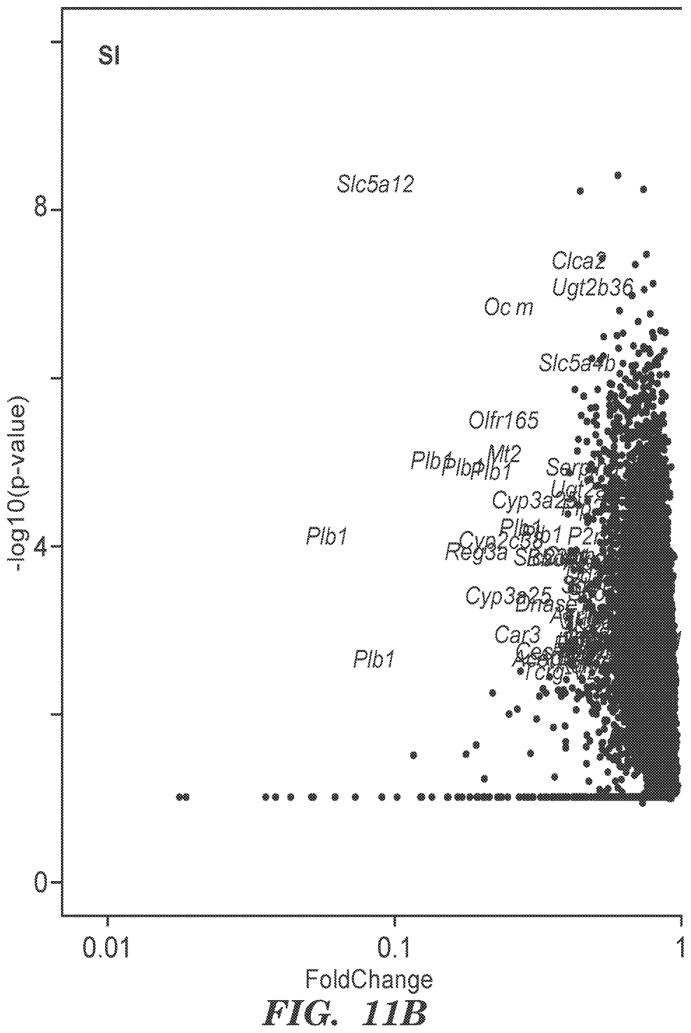

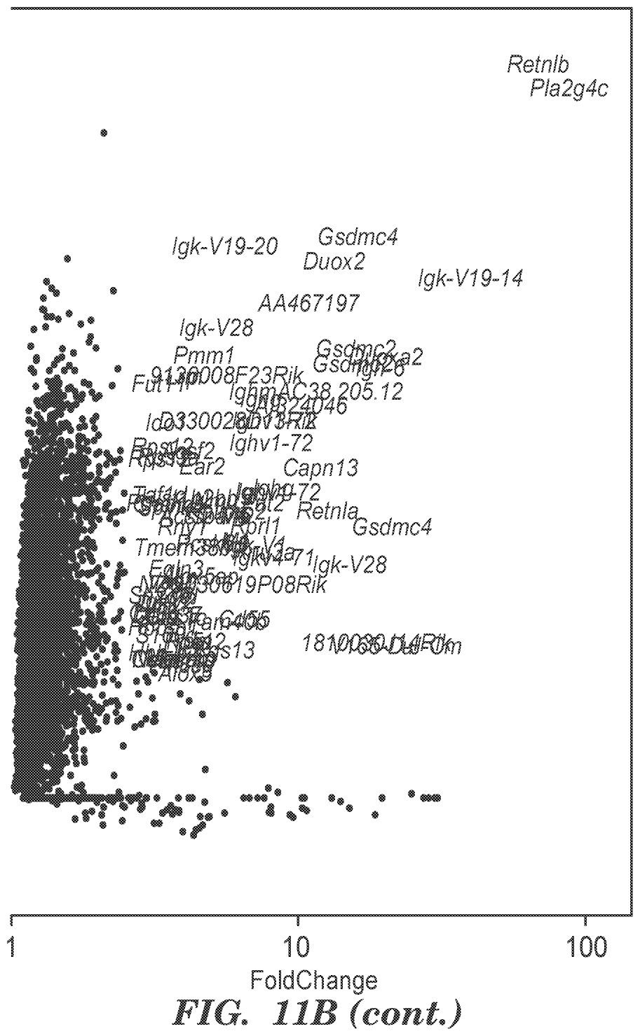

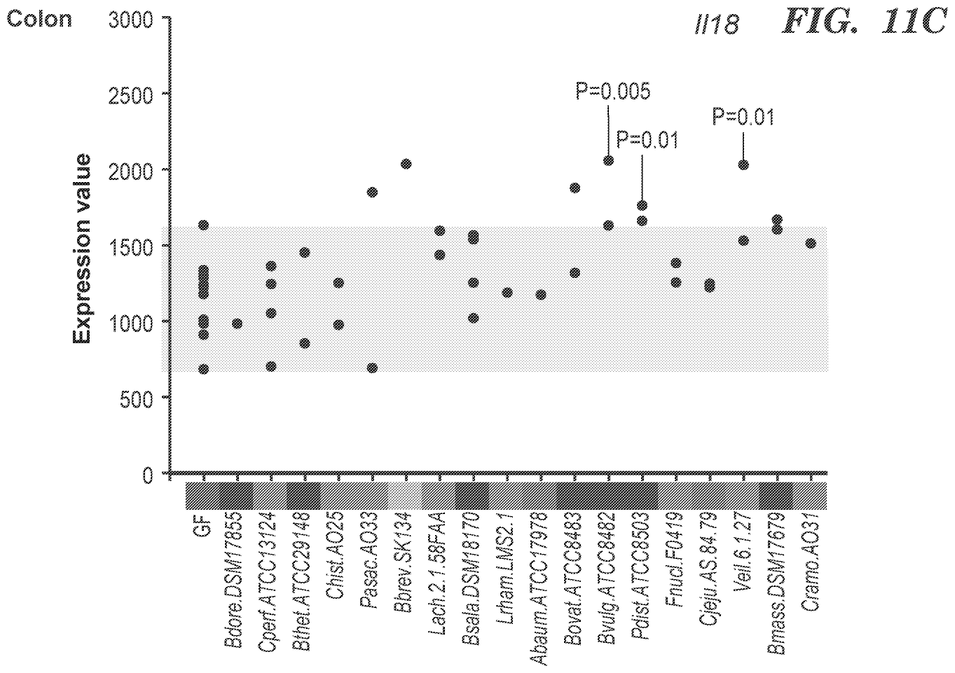

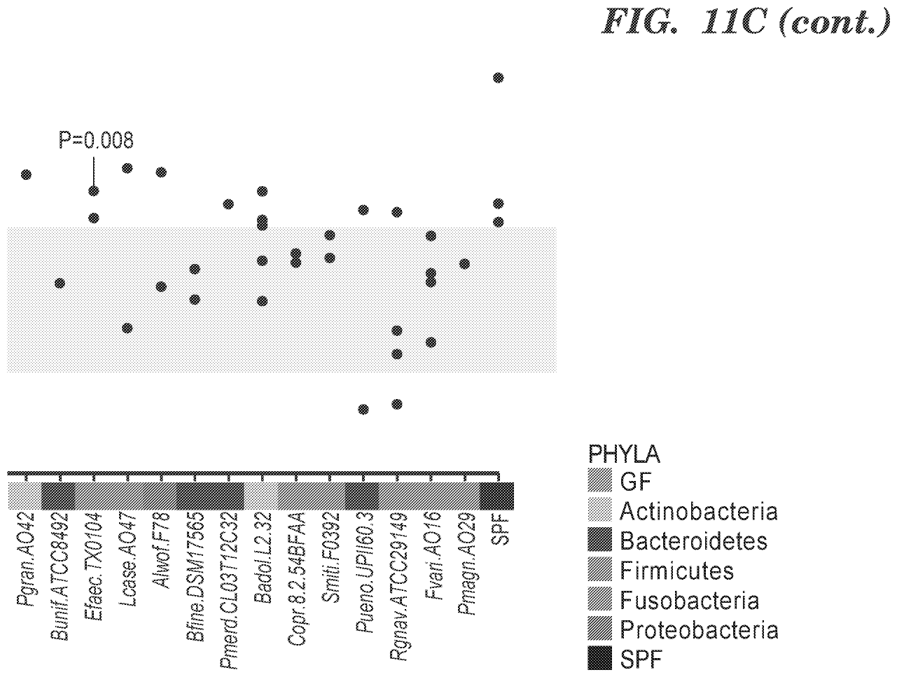

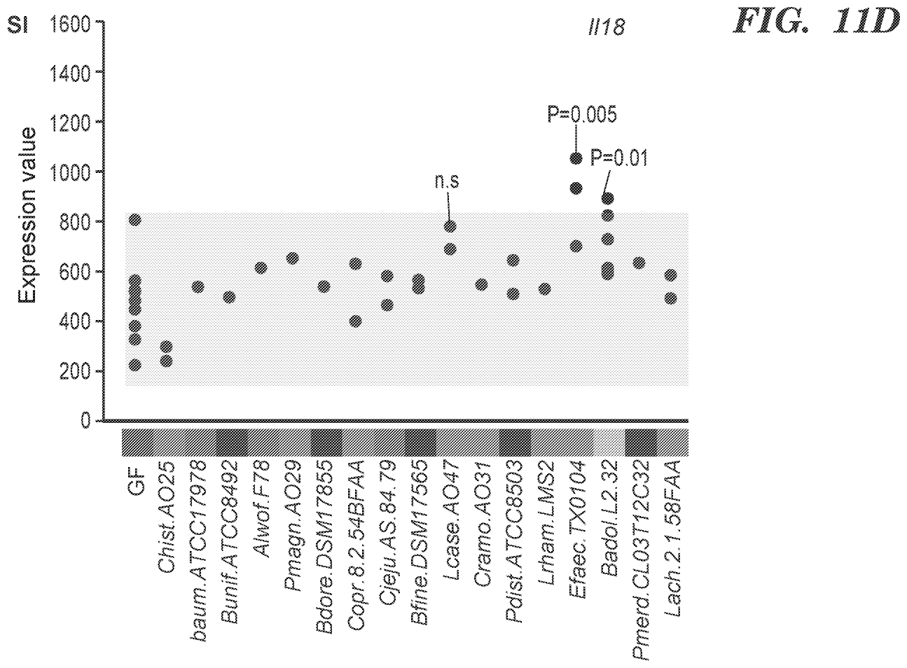

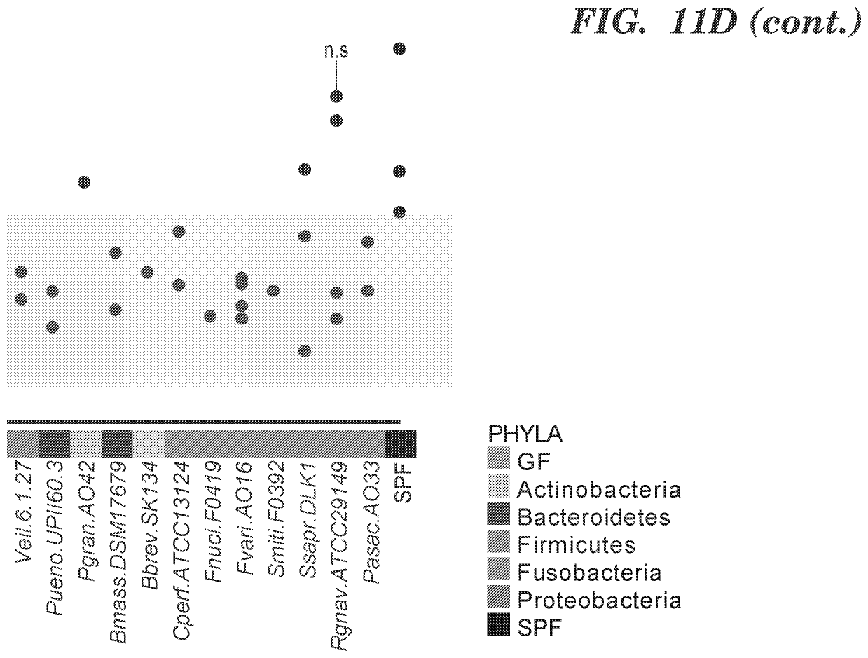

This degree of background variation made the determination of microbe-specific effects somewhat more complicated, but clear effects were apparent in volcano plot representations (FIGS. 11A and 11B). A general approach was adopted in which transcripts with an FC relative to GF>2.5 (or <0.4) and uncorrected p(−log 10)>2.5 for at least one bacteria were flagged. This selection yielded an unexpectedly small number of transcripts, indicating that symbiotic bacteria have only limited effects on the gut transcriptome in the monocolonization setting: 128 genes were up- or down-regulated in the colon and 116 in the SI, of which 20 were responsive in both colon and SI (data not shown—see supplemental materials of Geva-Zatorsky et al., Cell 2017, incorporated by reference herein below). These transcripts are displayed for each microbe in FIGS. 4B and 4C. None of them was uniformly induced by all bacteria, but >60% of these responsive transcripts were induced by some microbes and repressed by others (e.g., Defa5, Retnlb, Apoa1, and Lyz1 in the colon; Retnlb, Duox2, and Reg3a in the SI). Without being bound to any particular theory, this observation indicated that different microbes can sometimes have diametrically opposed consequences. Without wishing to be bound by theory, it appears that some bacteria can take advantage of the host's adaptive abilities as a means of out-competing other microbes, either by creating a more favorable environment for themselves or by down-regulating host metabolic pathways such as those for lipid or amine metabolism to create a hostile environment for other bacteria that require these molecules. Fold Change of colonic and small-intestinal transcripts that are most impacted by monocolonization (compared to GF) (data not shown—see supplemental materials of Geva-Zatorsky et al., Cell 2017, incorporated by reference herein below).

Some bacteria had stronger and more reproducible signatures (e.g. Fusobacterium varium in the SI, Campylobacter jejuni in the colon), while others had weaker and more variable imprints (Bacteroides salanitronis, Clostridium perfringens). None of the transcripts were uniquely induced by a single microbe, but most were induced (or repressed) by several bacteria, with no particular connection to phylum. In these respects, the diversity of transcriptional changes mirrored the alterations in immunophenotypes described above. These transcriptomic changes were grouped in co-regulated gene clusters (FIGS. 4B and 4C). Cross-referencing to gene-expression databases (ImmGen, GNF atlases) showed that some, but not all, of these clusters were predominantly expressed in particular cell types and probably corresponded to responses in those cells (e.g., stromal, macrophage, B cell, or perhaps even stem cell transcripts; FIGS. 4B and 4C). In both tissues, the responsive genes encoded a variety of functional molecules-AMPs, stress response elements (Retn, Retnla, Retnlb), hemoglobins (likely reflecting changes in vascularization), immunoglobulin-related transcripts, and enzymes and molecules involved in lipid metabolism (fat digestion and absorption, lipid processing, lipase and phospholipase activity)—with corresponding overrepresentation of Gene Ontology pathways (antimicrobial response, extracellular matrix organization, amide and amine metabolism, retinol and vitamin metabolism, and acute inflammatory response). There was also an enrichment in transcripts reported to be affected in infant mice secondary to maternal colonization. On the other hand, significant induction of inflammation-associated cytokines like IL1α, IL1β, IL6, IL22, TNF, IL12, or IFNs was not observed. (Levels of IL1a, IL22, and IL6 were below detection.) However, IL18 levels were slightly elevated in response to several different bacteria (FIGS. 11C and 11D).

### Immunomodulatory Cell Types and Transcriptional Responses

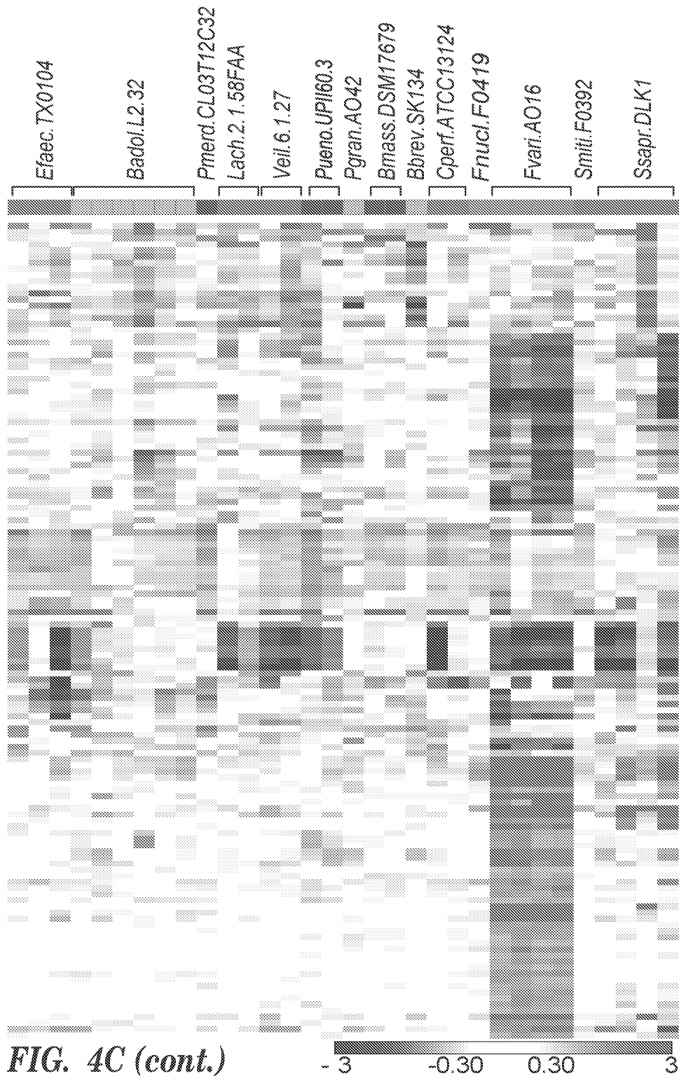

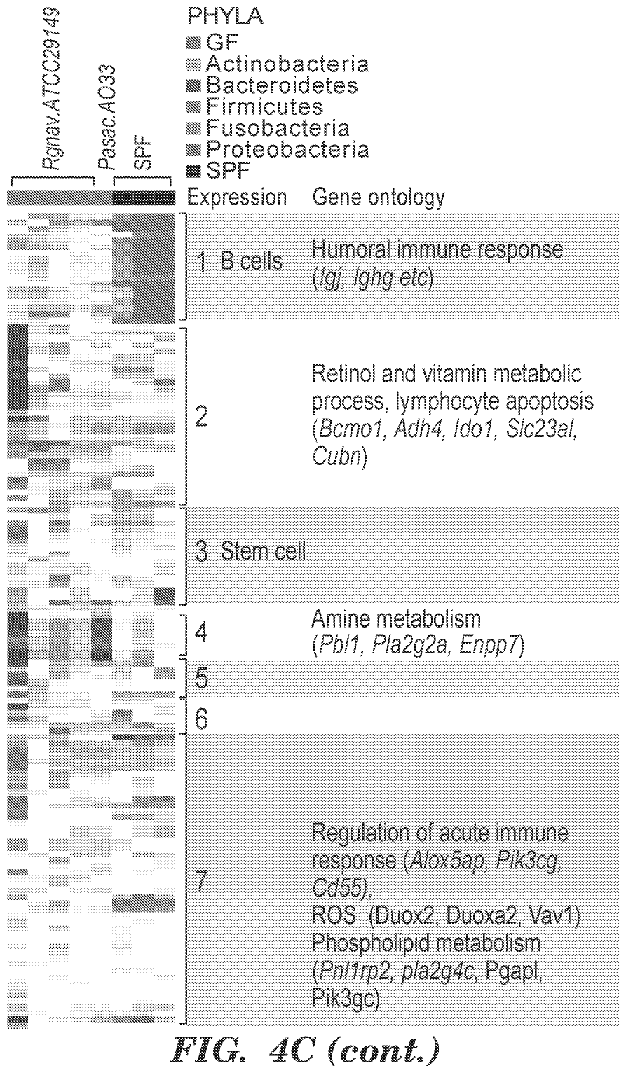

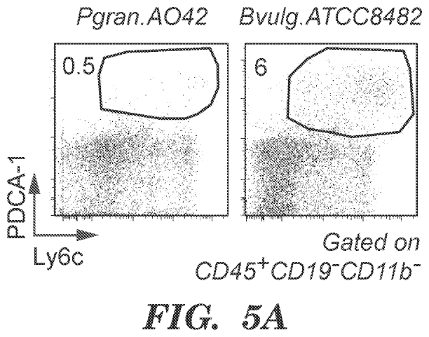

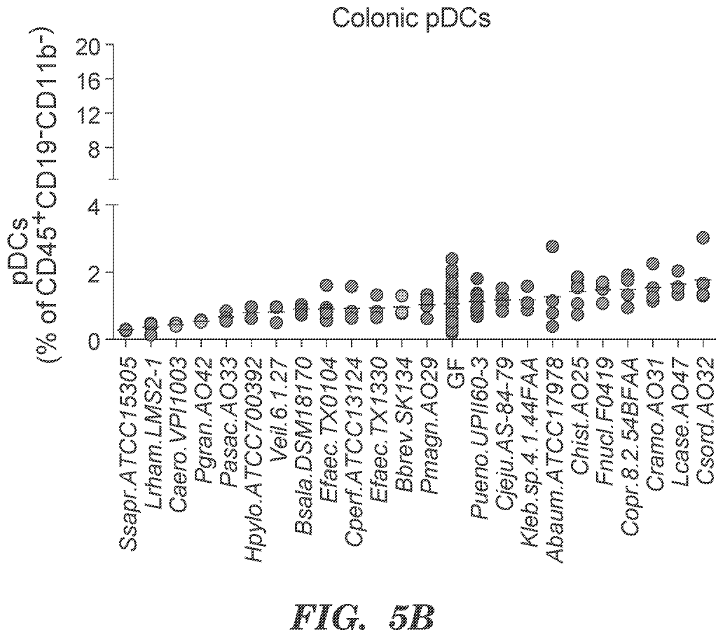

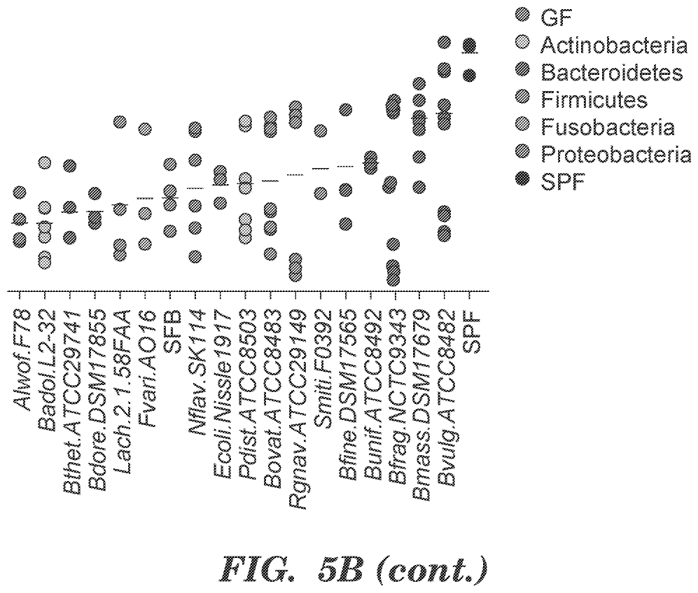

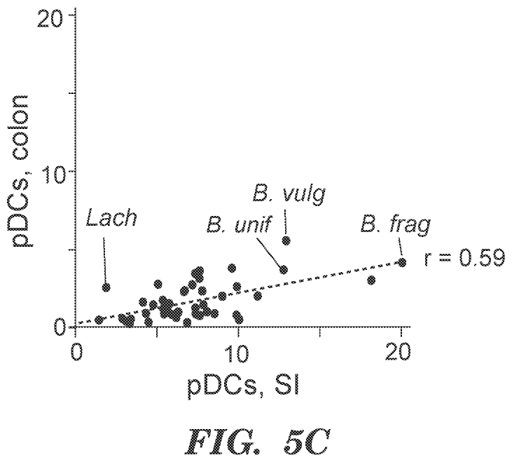

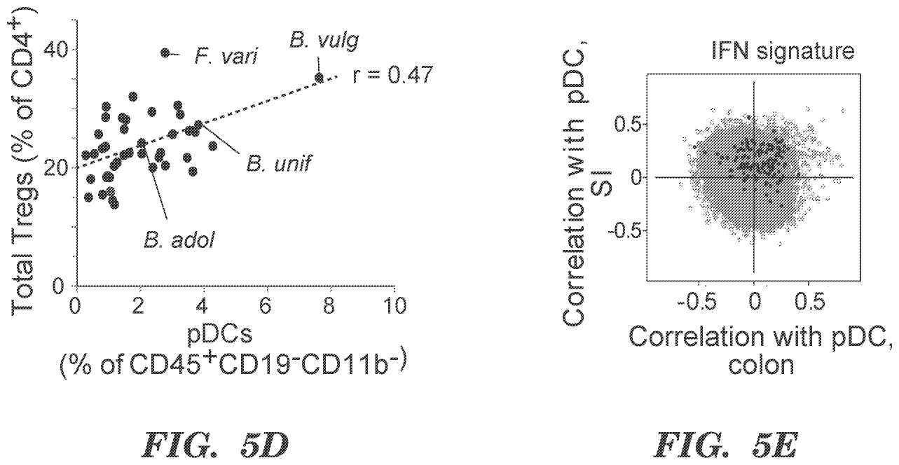

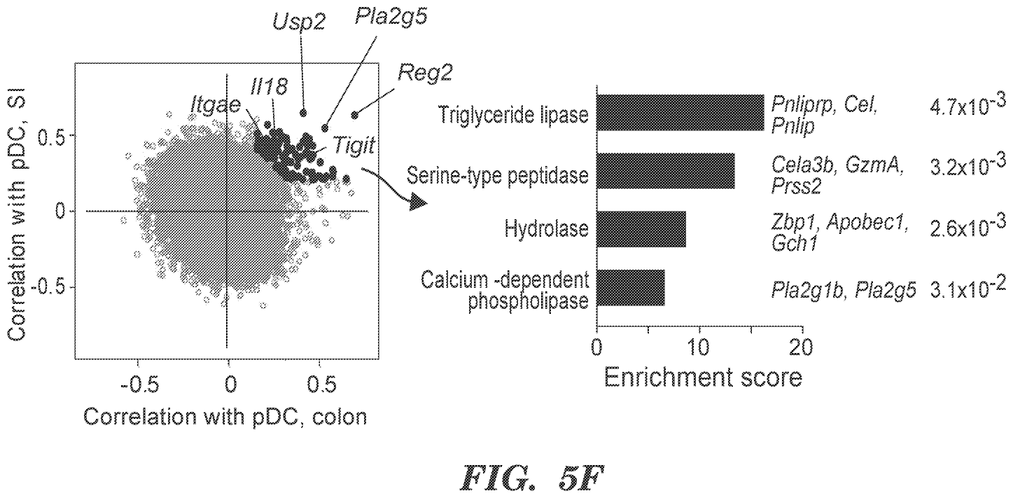

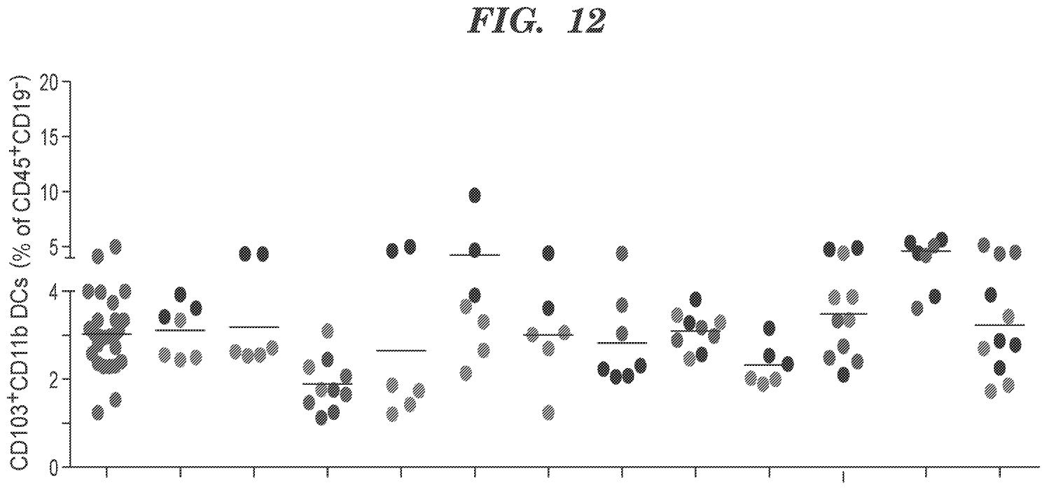

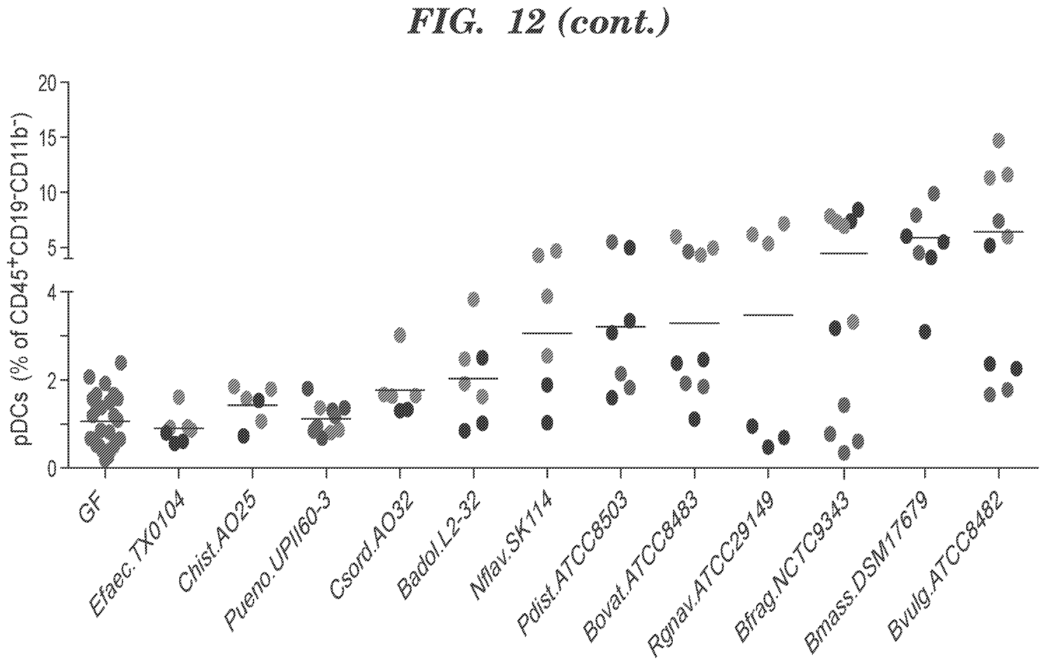

Colonic pDCs are biased by gut bacteria. Plasmacytoid dendritic cells are distinctive players in the innate arm of the immune system, playing a central role in antiviral defenses through their ability to produce copious amounts of type I IFNs. Correspondingly, they have been implicated in several IFN-linked diseases. The influence of the gut microbiota on the pDC pool is largely unknown. Some studies describe a reduction in pDCs in mice with a restricted microbiota distinct from that typical of SPF mice, while other studies reveal induction of pDCs in mLNs by B. fragilis during ongoing colitis. Among the myeloid populations, pDCs had the greatest range of fluctuation in our screen (FIG. 2A), as exemplified by the cytofluorometry profiles in FIG. 5A. These fluctuations were bidirectional (FIG. 5B): 38% of the bacteria tested increased colonic pDC proportions (by ≥2-fold) in monocolonized mice over those in GF mice, while 8% reduced colonic pDC proportions by >2-fold—most extremely in mice colonized with Staphylococcus saprophyticus and Lactobacillus rhamnosus, which harbored almost no pDCs. However, these frequencies were quite variable even in mice colonized by the same organism. For instance, Bacteroides vulgatus (ATCC 8482) was the most potent species at inducing colonic pDCs on average (mean, 6.4% pDCs), but with a range from 1.7% to 14.7%. The recalibration of pDCs in the colon resulting from monocolonization was more variable than the recalibration of CD103+ DCs in the same mice (FIG. 12). Interestingly, the ability of a microbe to induce pDCs in the SI and the colon was significantly correlated (r=0.52, t-test p=0.00061; FIG. 5C); without being bound to any particular theory, this correlation indicates that the same mediators or pathways can be at play in the two organs. pDCs have significant tolerogenic potential and can stimulate Tregs, an ability that has been associated with type I IFN production. Also of interest was the significant correlation between the strains' ability to boost colonic pDCs and total FoxP3+ Treg frequencies (r=0.46, t-test p=0.003; FIG. 5D).

Next, sets of genes whose expression was most correlated with pDC frequencies in the SI or the colon were identified, which provided insight into the molecular pathways through which microbes modulate pDCs and/or the physiological consequences of their pDC levels. No clear cluster of outliers stood out in these correlations. However, a set of IFN-inducible signature transcripts showed an enhanced correlation with pDC frequencies in both the SI and the colon (FIG. 5E, red dots), which was likely a reflection of their characteristically abundant IFN production. This set of genes (FIG. 5F, left panel, green dots; Table 7) included a few interesting transcripts worth highlighting. One transcript, IL18, was noteworthy given that pDCs express high levels of IL18R2 and that IL18 antagonizes their production of type I IFN. These data indicate that IL18 induced by some microbes can promote pDC accumulation rather than effector function (Chao et al., 2014). Another transcript was Tigit, an activation marker on T cells whose particular expression on Tregs may relate to the correlation between pDC and Treg proportions. Overall, the transcripts most correlated with pDC frequency were enriched in lipid or protein digestion and metabolic pathways (FIG. 5F, right panel), an observation which, without being bound to any particular theory, indicates a connection between pDCs and the metabolic and nutrient uptake functions of the gut. Table 7 lists genes that are reproducibly correlated to pDC frequency in both small intestine and colon with correlation coefficients.

TABLE 7
Genes reproducibly correlated to pDC
frequency in both SI and colon

| | | Correlation | Correlation |
|---|---|---|---|
| | | with pDC in | with pDC in |
| ProbeSetID | Gene | colon | SI |
| 10346564 | Casp8 | 0.346645 | 0.498719 |
| 10347933 | Sp140 | 0.259032 | 0.387232 |
| 10356601 | Per2 | 0.211345 | 0.420222 |
| 10360173 | Slamf7 | 0.267414 | 0.560423 |
| 10364093 | Derl3 | 0.458536 | 0.290893 |
| 10364950 | Gadd45b | 0.385884 | 0.215789 |
| 10368970 | Prdm1 | 0.279245 | 0.378849 |
| 10371846 | Apaf1 | 0.415562 | 0.208077 |
| 10372410 | Glipr1 | 0.318339 | 0.391511 |
| 10374035 | Xbp1 | 0.256687 | 0.387791 |
| 10374236 | Upp1 | 0.300071 | 0.509047 |
| 10378286 | Itgae | 0.232808 | 0.455385 |
| 10379228 | Nos2 | 0.424223 | 0.402772 |
| 10382492 | Otop3 | 0.364241 | 0.350084 |
| 10389786 | Hlf | 0.285109 | 0.39868 |
| 10393449 | Socs3 | 0.373819 | 0.301883 |
| 10396421 | Hif1a | 0.332519 | 0.293642 |
| 10399924 | Pik3cg | 0.272931 | 0.389514 |
| 10402864 | Ighg | 0.521823 | 0.460325 |
| 10403015 | AI324046 | 0.472454 | 0.416115 |
| 10403018 | IghmAC38.205.12 | 0.510747 | 0.37342 |
| 10403031 | V165-D-J-Cmu | 0.557121 | 0.423053 |
| 10403043 | Ighv172 | 0.537281 | 0.368026 |
| 10403048 | Ighv172 | 0.550906 | 0.345123 |
| 10403060 | Ighv172 | 0.605047 | 0.314407 |
| 10403073 | Ighg | 0.523306 | 0.420653 |
| 10403558 | Ero1lb | 0.353423 | 0.251136 |
| 10404389 | Irf4 | 0.367052 | 0.478287 |
| 10412211 | Gzma | 0.384435 | 0.256284 |
| 10419288 | Gch1 | 0.391716 | 0.451802 |
| 10420308 | Gzmb | 0.373019 | 0.313732 |
| 10424731 | Gsdmd | 0.514588 | 0.252144 |
| 10428998 | Asap1 | 0.287448 | 0.370447 |
| 10430179 | Apol7b | 0.247496 | 0.365874 |
| 10433584 | Tnfrsf17 | 0.39167 | 0.421808 |
| 10435288 | Muc13 | 0.344427 | 0.511887 |
| 10438405 | Igl-V1 | 0.553652 | 0.247211 |
| 10439527 | Tigit | 0.477199 | 0.349885 |
| 10439936 | Nfkbiz | 0.538738 | 0.3354 |
| 10444258 | Psmb8 | 0.32356 | 0.292912 |
| 10448402 | Prss30 | 0.335393 | 0.386393 |
| 10450154 | H2-Aa | 0.337735 | 0.281426 |
| 10450344 | C2 | 0.261285 | 0.355782 |
| 10450800 | Trim15 | 0.268384 | 0.444917 |
| 10450808 | Trim40 | 0.284579 | 0.438955 |
| 10458278 | 2010001M09Rik | 0.386175 | 0.377385 |
| 10458340 | Hbegf | 0.291272 | 0.389074 |
| 10460317 | Pitpnm1 | 0.411275 | 0.395358 |
| 10462390 | Cd274 | 0.264962 | 0.342769 |
| 10464298 | Pnlip | 0.593502 | 0.22215 |
| 10464313 | Pnliprp1 | 0.684444 | 0.265219 |
| 10464761 | Sytl2 | 0.315712 | 0.377985 |
| 10468746 | Hspa12a | 0.207883 | 0.458576 |
| 10468898 | Lax1 | 0.309815 | 0.504814 |
| 10469070 | Nudt5 | 0.201833 | 0.426516 |
| 10472514 | Nostrin | 0.382751 | 0.228096 |
| 10475448 | Duoxa2 | 0.53598 | 0.466442 |
| 10477169 | Id1 | 0.36672 | 0.356027 |
| 10480035 | Pfkfb3 | 0.243618 | 0.384942 |
| 10481278 | Cel | 0.644311 | 0.222475 |
| 10486956 | Duox2 | 0.387938 | 0.472042 |
| 10490150 | Zbp1 | 0.546959 | 0.36078 |
| 10490632 | BC006779 | 0.353295 | 0.260611 |
| 10493794 | S100a14 | 0.491765 | 0.20197 |
| 10494978 | Ptpn22 | 0.27292 | 0.393292 |
| 10495186 | AI504432 | 0.300401 | 0.410083 |
| 10495967 | Tifa | 0.309916 | 0.45809 |
| 10497463 | Cpb1 | 0.551402 | 0.232406 |
| 10501494 | Amy2b | 0.435759 | 0.219462 |
| 10501500 | Amy2a5 | 0.451442 | 0.219802 |
| 10501544 | Amy2a5 | 0.447158 | 0.220132 |
| 10502606 | Clca6 | 0.369887 | 0.254824 |
| 10504757 | BC005685 | 0.303221 | 0.407388 |
| 10505623 | D4Bwg0951e | 0.2173 | 0.433333 |
| 10507218 | Mknk1 | 0.398221 | 0.325598 |
| 10516266 | Zc3h12a | 0.300508 | 0.433271 |
| 10517573 | Cela3b | 0.684321 | 0.221534 |
| 10517655 | Pla2g5 | 0.634706 | 0.539383 |
| 10518050 | Cela2a | 0.601016 | 0.221343 |
| 10519949 | A630072M18Rik | 0.238882 | 0.370209 |
| 10523182 | Areg | 0.321547 | 0.281287 |
| 10524698 | Pla2g1b | 0.771336 | 0.205541 |
| 10524844 | Taok3 | 0.256407 | 0.397111 |
| 10527565 | Pdx1 | 0.202491 | 0.399613 |
| 10528527 | Fam126a | 0.408628 | 0.316738 |
| 10531126 | Igj | 0.463838 | 0.347343 |
| 10531972 | Gbp8 | 0.41637 | 0.315868 |
| 10533603 | Rhof | 0.273544 | 0.438676 |
| 10534303 | Lat2 | 0.347774 | 0.284231 |
| 10534909 | Sp110 | 0.292899 | 0.416224 |
| 10537014 | Cpa2 | 0.627869 | 0.204953 |
| 10537627 | Prss2 | 0.68895 | 0.25192 |
| 10538901 | BC005685 | 0.24361 | 0.36908 |
| 10538903 | Igk-V28 | 0.495059 | 0.45008 |
| 10539194 | Reg2 | 0.824988 | 0.623835 |
| 10542677 | Etnk1 | 0.360159 | 0.257281 |
| 10544326 | 2210010C04Rik | 0.56942 | 0.204525 |
| 10545194 | Rprl1 | 0.46329 | 0.333779 |
| 10545198 | Igkv4-71 | 0.426508 | 0.238759 |
| 10545215 | Igk-V28 | 0.467621 | 0.220977 |
| 10545242 | Igk-V19-20 | 0.513754 | 0.482844 |
| 10545247 | Igk-V19-14 | 0.446378 | 0.367115 |
| 10545252 | Igk-V21-2 | 0.55886 | 0.360408 |
| 10545865 | Cml3 | 0.379881 | 0.235882 |
| 10545869 | Cml3 | 0.370989 | 0.263822 |
| 10547621 | Apobec1 | 0.28719 | 0.432022 |
| 10553092 | Dbp | 0.278645 | 0.449649 |
| 10563615 | Hps5 | 0.256838 | 0.421852 |
| 10567366 | Gp2 | 0.554846 | 0.201971 |
| 10567702 | Arhgap17 | 0.261719 | 0.467299 |
| 10569168 | Slc25a22 | 0.313131 | 0.343052 |
| 10581355 | Ctrl | 0.6183 | 0.207553 |
| 10582862 | Arhgef12 | 0.285532 | 0.44116 |
| 10584634 | Usp2 | 0.495373 | 0.638087 |
| 10585194 | Il18 | 0.295246 | 0.343781 |
| 10587194 | Gnb5 | 0.415412 | 0.222715 |
| 10590620 | Ccr9 | 0.201598 | 0.503039 |
| 10591022 | 4931406C07Rik | 0.257904 | 0.358737 |
| 10592126 | Fam118b | 0.335069 | 0.430701 |
| 10606792 | Nxf7 | 0.345013 | 0.449816 |

### Antimicrobial Peptide Expression Upon Microbial Colonization

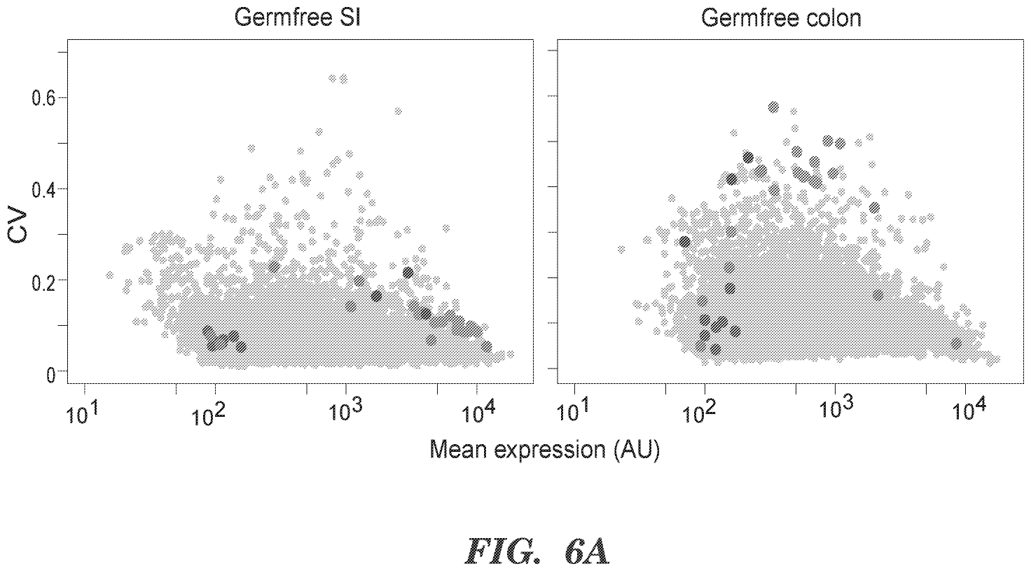

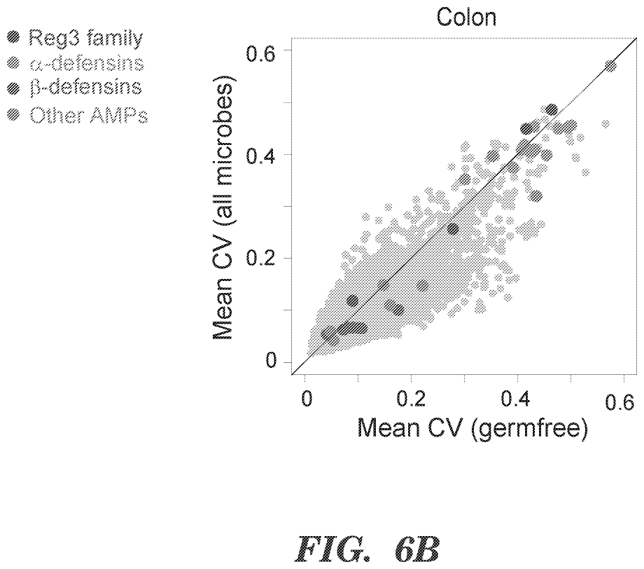

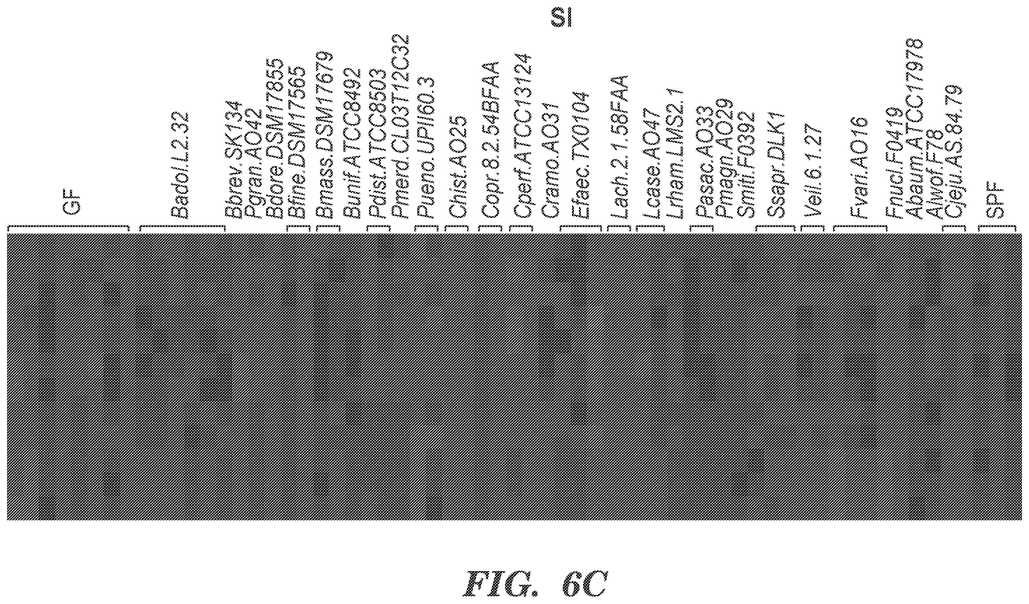

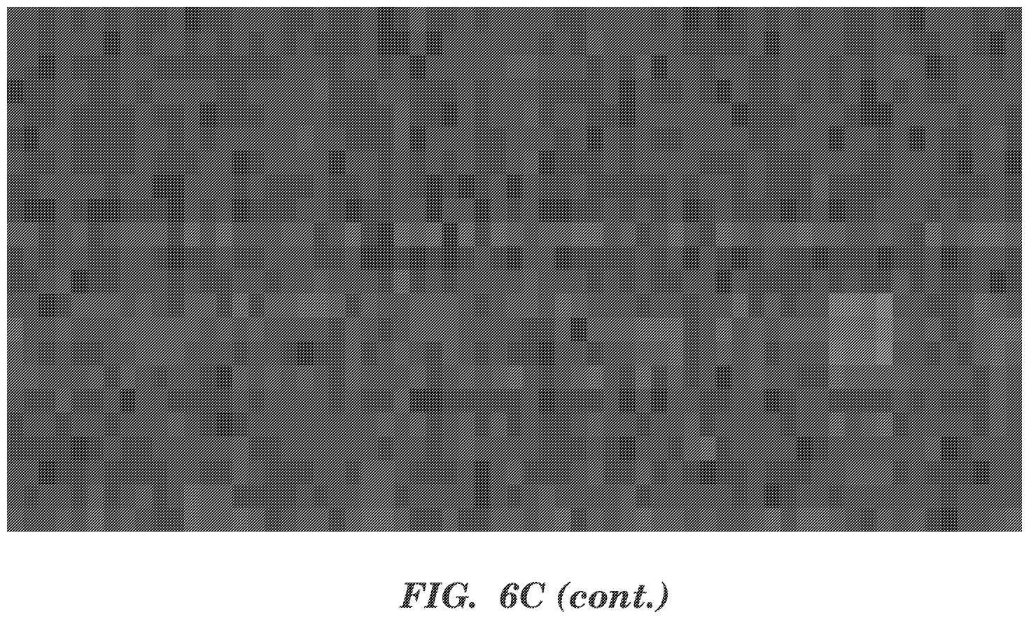

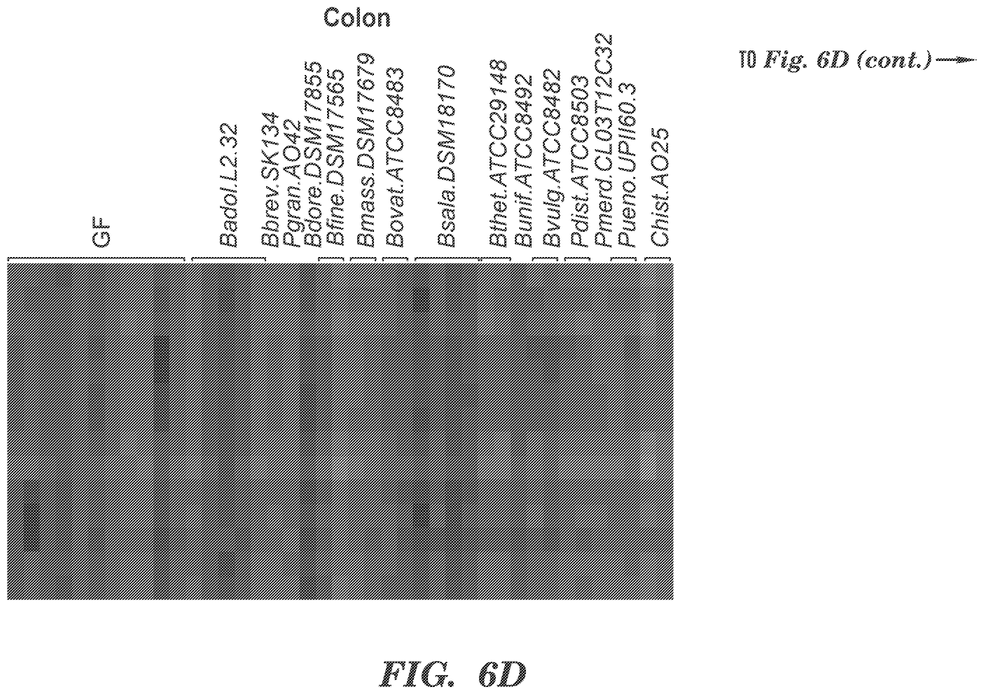

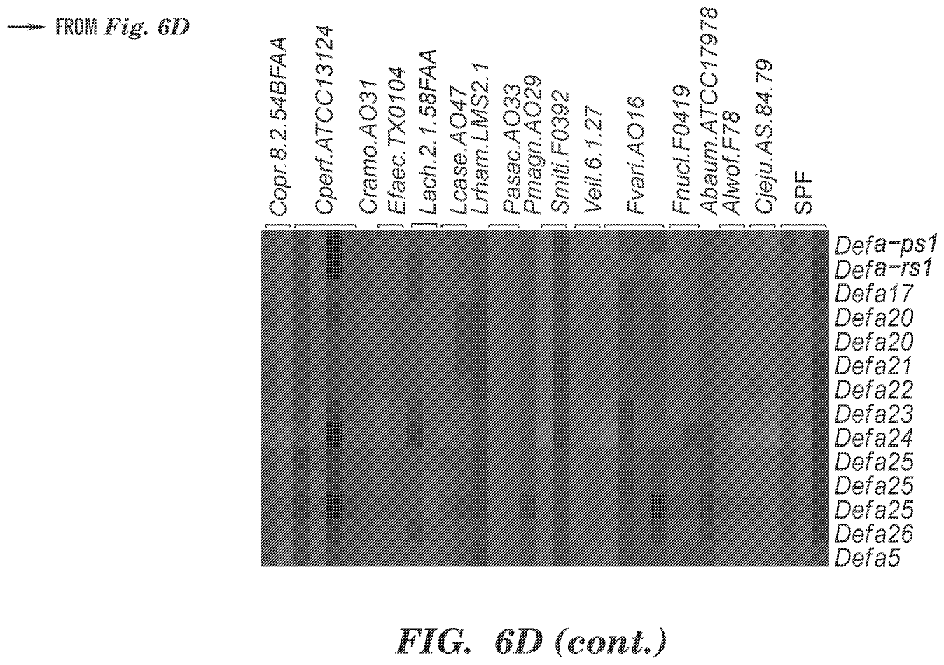

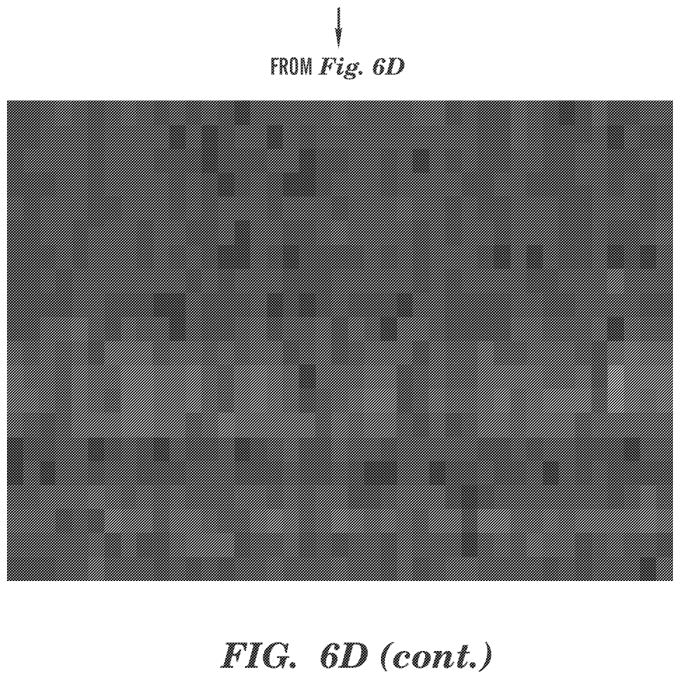

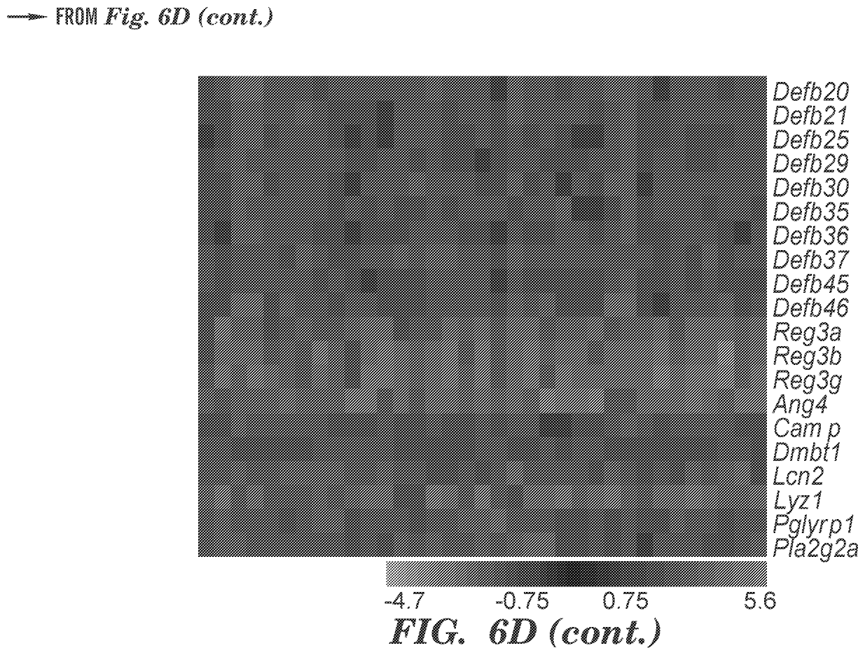

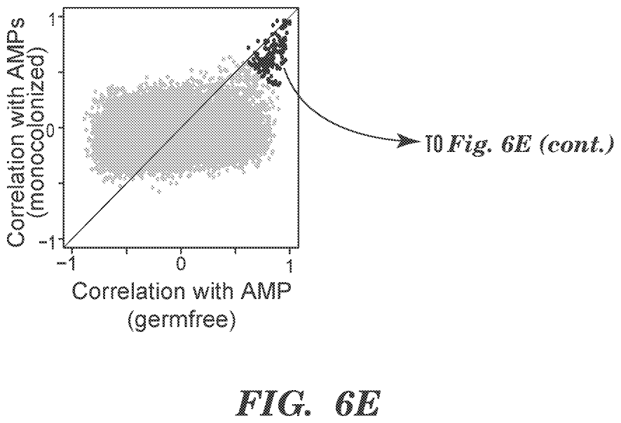

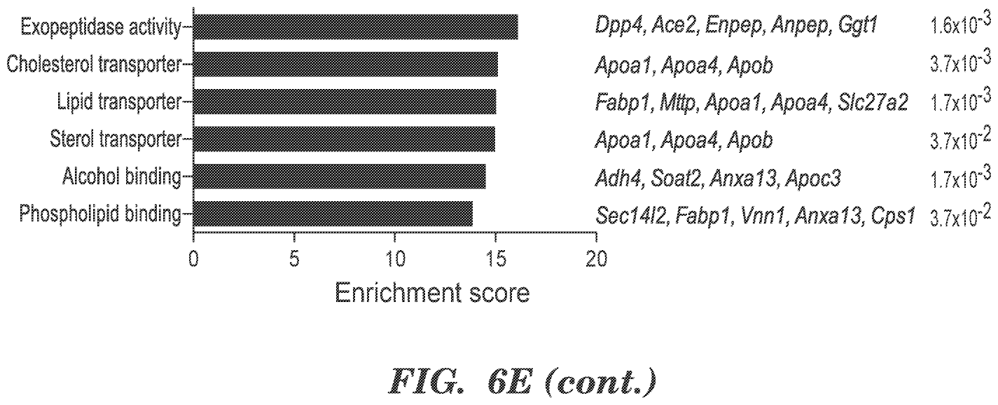

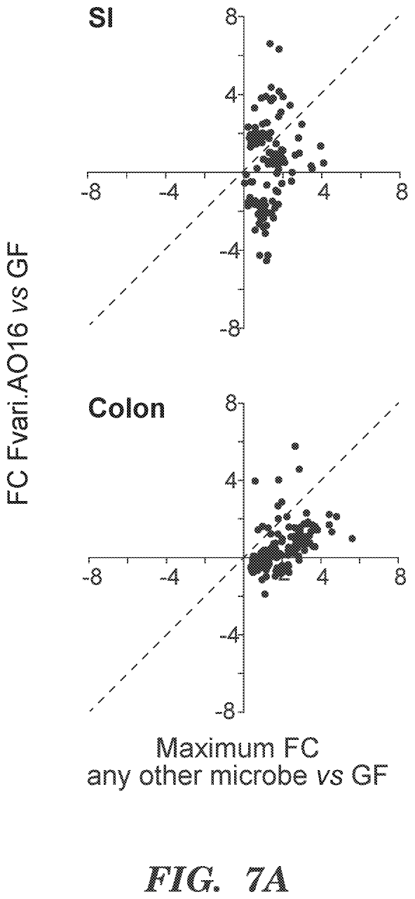

Expression of many gut AMPs is constitutive, although bacterial colonization can induce a subset of these peptides in SI Paneth cells. It was next assessed whether AMPs respond similarly to different bacterial species and whether they are coordinately regulated in the SI and the colon. In GF mice, α-defensins, Reg3 family members, and other Paneth cell-derived products (such as Ang4) were expressed at reproducibly high levels in the SI but at 20-fold lower levels in the colon, (FIG. 6A), where they were among the most variably expressed transcripts genome-wide (as indicated by their reproducibly high CV, FIG. 6A-B) In contrast, β-defensins, which are produced by many types of epithelial cells, were expressed at comparable levels in the SI and the colon.

The impact of bacterial exposure on AMP transcription was then assessed in the intestines. The property of high variability in the GF colon was maintained upon microbial exposure (FIG. 6B). Expression of most AMPs was not substantially affected in the SI of any of the monocolonized mice, with only a modest induction of Reg3 family transcripts by a few bacterial species (FIG. 6C). The most profound change in the SI was a down-regulation of all three Reg3 genes by F. varium. In marked contrast, AMP expression was more responsive in the colon, with changes extending significantly beyond the baseline fluctuation in GF colons (FIG. 6D). Many α-defensin (but not β-defensin) transcripts were coordinately induced by a few phylogenetically diverse species (e.g., Parabacteroides merdae, Porphyromonas uenonis), with a similar pattern for the Reg3 family.

As denoted by the high CV of AMP transcripts in the colon (FIG. 6A), individual GF mice manifested substantial differences in the expression of α-defensin and Reg3 genes. Without being bound to any particular theory, this fluctuation in AMP levels, even in the absence of microbes, indicated that other triggers were affecting their expression. To elucidate the source of this variability, it was sought to detect other genes whose expression correlated with AMPs across the colons of either GF or monocolonized mice (FIG. 6E, left panel). There was no correlation with the expression of IFN signature genes, which would have indicated enteric viral infections, or with IL22 transcripts, which would have suggested stimulation of epithelial cells by ILCs via IL22. A group of genes stood out as most strongly correlated with AMPs in both GF and colonized mice; pathway analysis of these transcripts revealed a significant enrichment in a number of nutrient transport and lipid metabolism pathways, which without being bound to any particular theory indicates a link among nutrition, enterocyte function, and AMP production (FIG. 6E, right panel; and data not shown—see supplemental materials of Geva-Zatorsky et al., Cell 2017, incorporated by reference herein below). Thus, without being bound to any particular theory, colonization by some symbionts elicits highly coordinated AMP expression in the colon over a fluctuating background that appears to reflect intestinal function rather than microbial stimulation. Genes are correlated with AMP scores in GF and monocolonized mice with Spearman correlation coefficients (data not shown—see supplemental materials of Geva-Zatorsky et al., Cell 2017, incorporated by reference herein below).

Fusobacterium varium Elicits an Unusually Strong Host Response Signature

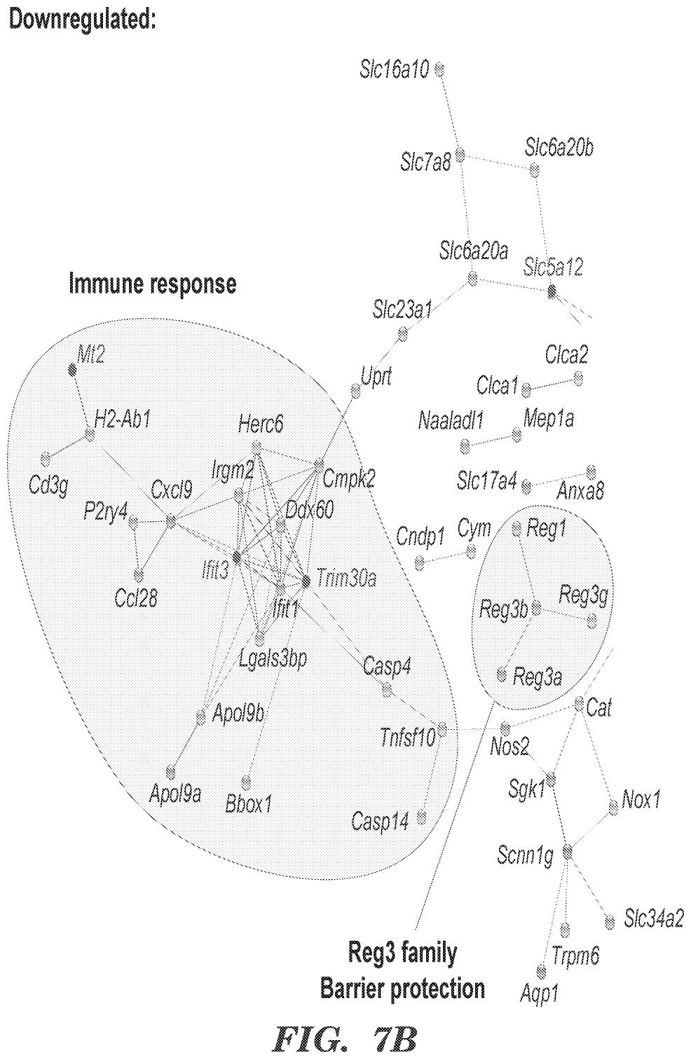

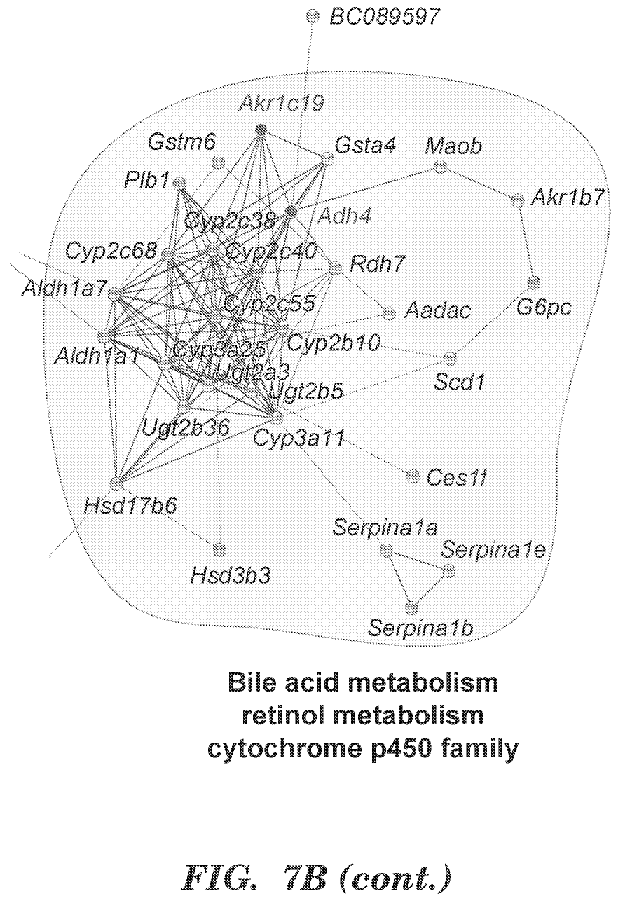

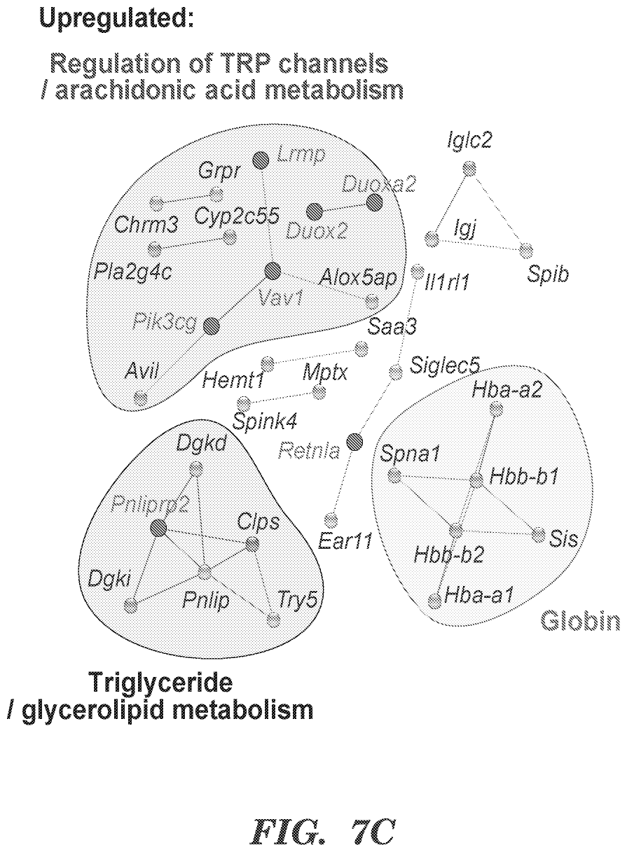

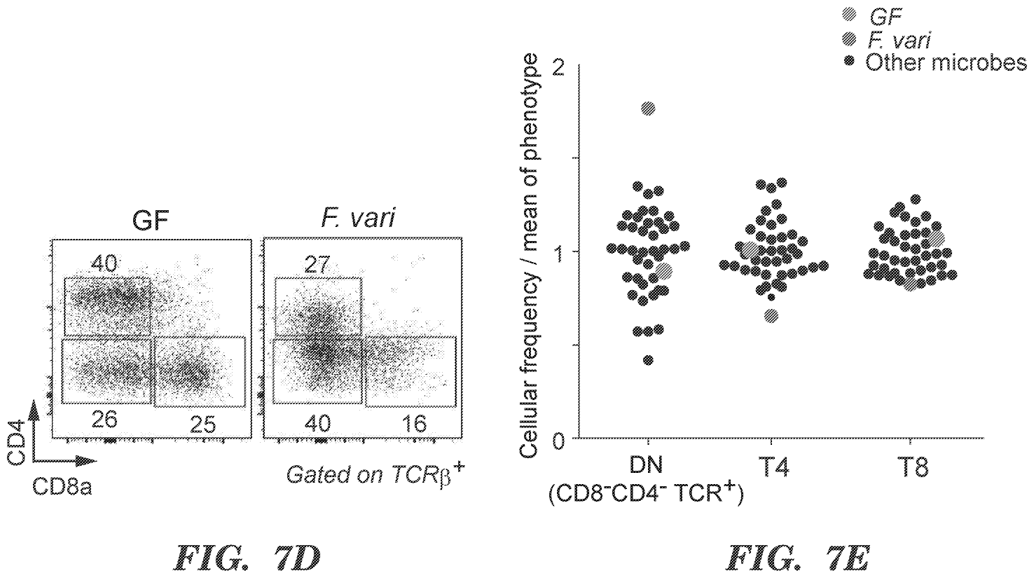

The gene-expression data of FIGS. 4 and 6 indicate that F. varium was one of the more stimulatory bacteria. F. varium also influenced many immune cell populations in the colon (FIG. 2C, especially DN T cells). F. varium is a gram-negative obligate anaerobe in the phylum Fusobacteria. In the SI, monocolonization with this species stood out, with a concentrated suppression of genes within cluster 2 and a strong up-regulation of cluster 7 (FIG. 4C). In the colon, its effects were also strong, albeit less unusual (FIG. 4D). When the SI transcriptomes of mice colonized with F. varium (AO16) were compared with the transcriptomes of any other monocolonized mice, 35% of the genes were more strongly induced (FIG. 7A). Seven percent of this set of genes were also more intensely induced in the colon by F. varium than by other bacteria. (FIG. 7A).

The functional nature of the response to F. varium was investigated by clustering (in the String database) the sets of transcripts down- or up-regulated by F. varium in either the SI or the colon (FIGS. 7B and 7C). Overall, there were a few altered genes related to immune function. Repressed transcripts included a large set related to bile acid metabolism, with a sizable cluster of the Cytochrome p450 gene family (e.g., Cyp3a25, Cyp2b10) and retinol metabolism genes (e.g., Rdh7, Aldh1a1) (FIG. 7B). Cytochrome p450 controls mechanisms of xenobiotic metabolism in the gut and, together with other members of this cluster (e.g., Rdh7 or Aldh1), influences the metabolism of all trans-retinoic acid. F. varium also strongly repressed the Reg3 antimicrobial family, particularly in the SI (FIG. 6C). Without being bound to any particular theory, an advantage is gained by F. varium in suppressing these AMPs, an important role in barrier integrity usually induced by microbes. Without wishing to be bound by theory, F. varium suppresses Reg3 to avoid death induced by AMPs, creating a more favorable milieu for itself. Up-regulated genes include those involved in arachidonic acid metabolism (e.g., Alox5ap) (FIG. 7C), the essential precursor for lipid mediators of inflammation. Table 8 depicts a complete list of genes that are up- or down-regulated in the small intestine and colon of Fusobacterium varium-colonized mice. FC (Fvari.AO16/GF)≤0.5 (repressed) and ≥2 (induced) for SI and FC Fvari.AO16/GF)≤0.67 (repressed) and ≥1.5 (induced) for colon. Table 9 depicts a list of F. varium-preferential genes. These genes are most strongly altered in F. varium-colonized mice compared with mice colonized with any other microbe [FC (varium.AO16/other microbes) cut off 1.5].

TABLE 8
Complete list of genes that are down-regulated and up-regulated
in the SI and colon of F. varium colonized mice

| Down-regulated | | | Up-regulated | | |
|---|---|---|---|---|---|
| ProbeSetID | GeneSymbol | Tissue | ProbeSetID | GeneSymbol | Tissue |
| 10347481 | Cyp27a1 | SI | 10345791 | Il1rl1 | SI |
| 10348896 | Gal3st2 | SI | 10345807 | Il18r1 | SI |
| 10351347 | Creg1 | SI | 10348321 | Dgkd | SI |
| 10354777 | Satb2 | SI | 10351515 | Rnu1b1 | SI |
| 10362073 | Sgk1 | SI | 10351959 | 1810030J14Rik | SI |
| 10367059 | BC089597 | SI | 10353034 | Snord87 | SI |
| 10368720 | Slc16a10 | SI | 10354647 | Pgap1 | SI |
| 10369932 | Susd2 | SI | 10354739 | Atp5l | SI |
| 10370054 | Slc5a4b | SI | 10355403 | Fn1 | SI |
| 10373330 | Rdh7 | SI | 10357488 | Cd55 | SI |
| 10373334 | Hsd17b6 | SI | 10357516 | C4bp | SI |
| 10376326 | Irgm2 | SI | 10358399 | Rgs13 | SI |
| 10376376 | 1810065E05Rik | SI | 10360145 | B930036N10Rik | SI |
| 10379184 | Slc46a1 | SI | 10360149 | Itln1 | SI |
| 10379228 | Nos2 | SI | 10362674 | Rnu3a | SI |
| 10381387 | G6pc | SI | 10365559 | Igf1 | SI |
| 10383047 | Enpp7 | SI | 10366774 | Avil | SI |
| 10386197 | 2210407C18Rik | SI | 10367582 | Vip | SI |
| 10388834 | Slc13a2 | SI | 10367691 | Iyd | SI |
| 10389261 | Gm11437 | SI | 10376887 | Snord49a | SI |
| 10393573 | Lgals3bp | SI | 10377429 | Snord118 | SI |
| 10394060 | Sectm1b | SI | 10379866 | Car4 | SI |
| 10395039 | Cmpk2 | SI | 10380059 | Rnu3b1 | SI |
| 10397145 | Acot2 | SI | 10382316 | Kcnj16 | SI |
| 10399365 | Slc7a15 | SI | 10390022 | Rsad1 | SI |
| 10402390 | Serpina1b | SI | 10390032 | Acsf2 | SI |
| 10402399 | Serpina1a | SI | 10390860 | Krt23 | SI |
| 10402409 | Serpina1e | SI | 10392845 | Cd300lf | SI |
| 10403312 | Akr1c19 | SI | 10398075 | Serpina3n | SI |
| 10403821 | Tcrg-V3 | SI | 10399924 | Pik3cg | SI |
| 10406564 | Acot12 | SI | 10400304 | Egln3 | SI |
| 10407940 | Tcrg-V2 | SI | 10400844 | Pygl | SI |
| 10408251 | Slc17a4 | SI | 10401109 | Gpx2 | SI |
| 10409592 | Lect2 | SI | 10403069 | Igh-6 | SI |
| 10410007 | Fbp1 | SI | 10405211 | Gadd45g | SI |
| 10412607 | Abhd6 | SI | 10405753 | Me1 | SI |
| 10419854 | Slc7a8 | SI | 10406176 | Slc9a3 | SI |
| 10420308 | Gzmb | SI | 10413014 | Chchd1 | SI |
| 10421186 | Gm10002 | SI | 10414262 | Ear2 | SI |
| 10424781 | Grina | SI | 10414269 | Bnip3 | SI |
| 10425049 | Apol9b | SI | 10416503 | Snora31 | SI |
| 10429160 | St3gal1 | SI | 10418341 | Il17rb | SI |
| 10430174 | Apol9a | SI | 10419156 | Ear10 | SI |
| 10433241 | Dnase1 | SI | 10419568 | Ear11 | SI |
| 10438423 | Olfr165 | SI | 10419575 | Ang4 | SI |
| 10440433 | Tmprss15 | SI | 10420668 | Mir15a | SI |
| 10444291 | H2-Ab1 | SI | 10422608 | Oxct1 | SI |
| 10451016 | Mep1a | SI | 10424430 | Gsdmcl-ps | SI |
| 10458262 | Slc23a1 | SI | 10424670 | Hemt1 | SI |
| 10460072 | Cndp1 | SI | 10425799 | Rnu12 | SI |
| 10460746 | Naaladl1 | SI | 10428124 | Rgs22 | SI |
| 10461934 | Trpm6 | SI | 10428943 | Gsdmc | SI |
| 10461979 | Aldh1a1 | SI | 10428955 | Gsdmc2 | SI |
| 10462618 | Ifit3 | SI | 10428973 | Gsdmc4 | SI |
| 10462623 | Ifit1 | SI | 10429588 | 9030619P08Rik | SI |
| 10463005 | Cyp2c55 | SI | 10430794 | Pmm1 | SI |
| 10466624 | Aldh1a7 | SI | 10432176 | Snora34 | SI |
| 10467372 | Cyp2c38 | SI | 10433114 | Itga5 | SI |
| 10467385 | Cyp2c68 | SI | 10436087 | Retnlb | SI |
| 10467390 | Cyp2c40 | SI | 10436095 | Retnla | SI |
| 10467979 | Scd1 | SI | 10445241 | Tnfrsf21 | SI |
| 10469353 | Fam23a | SI | 10446253 | Vav1 | SI |
| 10474450 | Slc5a12 | SI | 10449467 | Clps | SI |
| 10477935 | Gm1332 | SI | 10450948 | 9130008F23Rik | SI |
| 10480155 | Cubn | SI | 10452770 | Capn13 | SI |
| 10485466 | Cat | SI | 10454807 | Snora74a | SI |
| 10485700 | Bbox1 | SI | 10455015 | Vaultrc5 | SI |
| 10490903 | Car13 | SI | 10457091 | Neto1 | SI |
| 10490913 | Car3 | SI | 10458090 | Reep5 | SI |
| 10491091 | Tnfsf1O | SI | 10462473 | Mbl2 | SI |
| 10492300 | Aadac | SI | 10463112 | Ccnj | SI |
| 10496466 | Adh4 | SI | 10464298 | Pnlip | SI |
| 10500555 | Hsd3b3 | SI | 10464313 | Pnliprp1 | SI |
| 10501074 | Cym | SI | 10464328 | Pnliprp2 | SI |
| 10501208 | Gstm6 | SI | 10465831 | 5730408K05Rik | SI |
| 10502552 | Clca1 | SI | 10475448 | Duoxa2 | SI |
| 10502565 | Clca2 | SI | 10475517 | AA467197 | SI |
| 10502575 | Clca4 | SI | 10475990 | Slc20a1 | SI |
| 10506594 | Acot11 | SI | 10477250 | Hck | SI |
| 10514912 | Dio1 | SI | 10480275 | Nebl | SI |
| 10520869 | Plb1 | SI | 10481278 | Cel | SI |
| 10521892 | Slc34a2 | SI | 10486956 | Duox2 | SI |
| 10525989 | Gpr133 | SI | 10488482 | Acss1 | SI |
| 10527494 | Cyp3a25 | SI | 10492136 | Dclk1 | SI |
| 10531051 | Ugt2b36 | SI | 10492306 | Sucnr1 | SI |
| 10531057 | Ugt2b5 | SI | 10493820 | S100a6 | SI |
| 10531066 | Ugt2a3 | SI | 10497463 | Cpb1 | SI |
| 10531407 | Cxcl9 | SI | 10497590 | Mecom | SI |
| 10535524 | Ocm | SI | 10499130 | Rnu73b | SI |
| 10535704 | Cyp3a11 | SI | 10501494 | Amy2b | SI |
| 10537169 | Akr1b7 | SI | 10501500 | Amy2a5 | SI |
| 10537545 | Gm7254 | SI | 10501922 | Snhg8 | SI |
| 10538459 | Aqp1 | SI | 10502638 | Clca5 | SI |
| 10538590 | Herc6 | SI | 10502845 | Fam73a | SI |
| 10539179 | Reg3b | SI | 10504002 | Spink4 | SI |
| 10539186 | Reg3a | SI | 10504450 | Glipr2 | SI |
| 10539200 | Reg1 | SI | 10508719 | Snora16a | SI |
| 10542857 | Far2 | SI | 10508723 | Snora61 | SI |
| 10543017 | Pdk4 | SI | 10517573 | Cela3b | SI |
| 10545569 | Reg3g | SI | 10517682 | 2310028O11Rik | SI |
| 10551197 | Cyp2b10 | SI | 10519905 | Gnat3 | SI |
| 10566358 | Trim30a | SI | 10527638 | Alox5ap | SI |
| 10571984 | Ddx60 | SI | 10530772 | Nmu | SI |
| 10574023 | Mt2 | SI | 10531126 | Igj | SI |
| 10580663 | Ces1f | SI | 10531342 | U90926 | SI |
| 10580678 | Ces1g | SI | 10534395 | Cldn4 | SI |
| 10582997 | Casp4 | SI | 10536949 | Fam40b | SI |
| 10587315 | Gsta4 | SI | 10537014 | Cpa2 | SI |
| 10593015 | Cd3g | SI | 10537051 | Cpa1 | SI |
| 10597949 | Slc6a20b | SI | 10538214 | D330028D13Rik | SI |
| 10597960 | Slc6a20a | SI | 10538871 | Gm4964 | SI |
| 10598138 | Spry3 | SI | 10538880 | Igk-V1 | SI |
| 10598203 | Ccl28 | SI | 10538882 | Gm5571 | SI |
| 10601326 | Uprt | SI | 10538887 | Gm5153 | SI |
| 10603746 | Maob | SI | 10538924 | LOC100046496 | SI |
| 10605938 | P2ry4 | SI | 10542050 | Efcab4b | SI |
| 10606640 | Nox1 | SI | 10542691 | Lrmp | SI |
| 10349648 | Ctse | colon | 10543967 | Dgki | SI |
| 10370522 | Caspl4 | colon | 10544326 | 2210010C04Rik | SI |
| 10414065 | Anxa8 | colon | 10544333 | Try5 | SI |
| 10438423 | Olfr165 | colon | 10544523 | Rny1 | SI |
| 10440655 | 2310079G19Rik | colon | 10545233 | Gm10883 | SI |
| 10557111 | Scnn1g | colon | 10545425 | Sh2d6 | SI |
| 10574780 | Hsd11b2 | colon | 10547153 | Alox5 | SI |
| | | | 10550131 | Pla2g4c | SI |
| | | | 10551531 | Sycn | SI |
| | | | 10551966 | Hspb6 | SI |
| | | | 10552369 | Siglec5 | SI |
| | | | 10553967 | Pcsk6 | SI |
| | | | 10555510 | Pde2a | SI |
| | | | 10556244 | Snora23 | SI |
| | | | 10562812 | Spib | SI |
| | | | 10563099 | Snord35b | SI |
| | | | 10563350 | Fut2 | SI |
| | | | 10564161 | Snord116 | SI |
| | | | 10565811 | Snord15b | SI |
| | | | 10565813 | Snord15a | SI |
| | | | 10566254 | Hbb-b1 | SI |
| | | | 10566258 | Hbb-b2 | SI |
| | | | 10569399 | Trpm5 | SI |
| | | | 10569618 | Ano1 | SI |
| | | | 10570741 | Defb1 | SI |
| | | | 10580624 | Ces1c | SI |
| | | | 10581882 | Ctrb1 | SI |
| | | | 10585410 | Sh2d7 | SI |
| | | | 10587616 | Prss35 | SI |
| | | | 10590267 | Snora62 | SI |
| | | | 10594289 | Glce | SI |
| | | | 10602756 | Smpx | SI |
| | | | 10604076 | Snora69 | SI |
| | | | 10606609 | Tspan6 | SI |
| | | | 10607705 | S100g | SI |
| | | | 10607752 | Bmx | SI |
| | | | 10351905 | Spna1 | colon |
| | | | 10353034 | Snord87 | colon |
| | | | 10358399 | Rgs13 | colon |
| | | | 10358408 | Rgs1 | colon |
| | | | 10360149 | Itln1 | colon |
| | | | 10362674 | Rnu3a | colon |
| | | | 10375051 | Hba-a1 | colon |
| | | | 10375058 | Hba-a2 | colon |
| | | | 10376887 | Snord49a | colon |
| | | | 10377429 | Snord118 | colon |
| | | | 10378793 | Tmigd1 | colon |
| | | | 10379535 | Ccl8 | colon |
| | | | 10380059 | Rnu3b1 | colon |
| | | | 10382316 | Kcnj16 | colon |
| | | | 10402512 | Scarnal13 | colon |
| | | | 10402991 | Gm16970 | colon |
| | | | 10403028 | LOC382693 | colon |
| | | | 10403034 | LOC100046275 | colon |
| | | | 10403043 | Ighv1-72 | colon |
| | | | 10403069 | Igh-6 | colon |
| | | | 10407591 | Chrm3 | colon |
| | | | 10413014 | Chchd1 | colon |
| | | | 10416503 | Snora31 | colon |
| | | | 10419568 | Ear11 | colon |
| | | | 10419575 | Ang4 | colon |
| | | | 10424430 | Gsdmcl-ps | colon |
| | | | 10425799 | Rnu12 | colon |
| | | | 10428955 | Gsdmc2 | colon |
| | | | 10429588 | 9030619P08Rik | colon |
| | | | 10430425 | Lgals2 | colon |
| | | | 10432176 | Snora34 | colon |
| | | | 10436087 | Retnlb | colon |
| | | | 10436095 | Retnla | colon |
| | | | 10438405 | Igl-V1 | colon |
| | | | 10439068 | Osta | colon |
| | | | 10439790 | Trat1 | colon |
| | | | 10446713 | Snord53 | colon |
| | | | 10449467 | Clps | colon |
| | | | 10453231 | Slc8a1 | colon |
| | | | 10454807 | Snora74a | colon |
| | | | 10455015 | Vaultrc5 | colon |
| | | | 10463005 | Cyp2c55 | colon |
| | | | 10464328 | Pnliprp2 | colon |
| | | | 10465831 | 5730408K05Rik | colon |
| | | | 10467319 | Rbp4 | colon |
| | | | 10475448 | Duoxa2 | colon |
| | | | 10486956 | Duox2 | colon |
| | | | 10494636 | Reg4 | colon |
| | | | 10498024 | Slc7a11 | colon |
| | | | 10498653 | 1110032A04Rik | colon |
| | | | 10498659 | Sis | colon |
| | | | 10501922 | Snhg8 | colon |
| | | | 10502638 | Clca5 | colon |
| | | | 10502845 | Fam73a | colon |
| | | | 10504002 | Spink4 | colon |
| | | | 10508721 | Snora44 | colon |
| | | | 10508723 | Snora61 | colon |
| | | | 10516906 | Snora73b | colon |
| | | | 10516908 | Snora73a | colon |
| | | | 10531126 | Igj | colon |
| | | | 10532164 | Atp5k | colon |
| | | | 10538214 | D330028D13Rik | colon |
| | | | 10538871 | Gm4964 | colon |
| | | | 10538880 | Igk-V1 | colon |
| | | | 10538882 | Gm5571 | colon |
| | | | 10538887 | Gm5153 | colon |
| | | | 10538903 | Igk-V28 | colon |
| | | | 10538924 | LOC100046496 | colon |
| | | | 10542050 | Efcab4b | colon |
| | | | 10544523 | Rny1 | colon |
| | | | 10545173 | LOC672291 | colon |
| | | | 10545180 | Gm10879 | colon |
| | | | 10545182 | Gm459 | colon |
| | | | 10545184 | Gm10880 | colon |
| | | | 10545187 | Gm1502 | colon |
| | | | 10545196 | Gm1419 | colon |
| | | | 10545198 | Igkv4-71 | colon |
| | | | 10545210 | Gm1524 | colon |
| | | | 10545233 | Gm10883 | colon |
| | | | 10545247 | Igk-V19-14 | colon |
| | | | 10550131 | Pla2g4c | colon |
| | | | 10556244 | Snora23 | colon |
| | | | 10563597 | Saa3 | colon |
| | | | 10565811 | Snord15b | colon |
| | | | 10565813 | Snord15a | colon |
| | | | 10566254 | Hbb-b1 | colon |
| | | | 10566258 | Hbb-b2 | colon |
| | | | 10567589 | Usp31 | colon |
| | | | 10570656 | Defa23 | colon |
| | | | 10570660 | Gm10104 | colon |
| | | | 10570663 | Defa25 | colon |
| | | | 10570668 | Gm15284 | colon |
| | | | 10570683 | Defa-ps1 | colon |
| | | | 10570690 | Defa17 | colon |
| | | | 10570693 | Defa5 | colon |
| | | | 10570706 | Defa20 | colon |
| | | | 10570717 | Gm14850 | colon |
| | | | 10570726 | Defa26 | colon |
| | | | 10570732 | Gm15315 | colon |
| | | | 10570735 | Defa24 | colon |
| | | | 10574427 | Impdh2 | colon |
| | | | 10580624 | Ces1c | colon |
| | | | 10588132 | A4gnt | colon |
| | | | 10607705 | S100g | colon |
| | | | 10607712 | Grpr | colon |

TABLE 9
List of F. varium-preferential genes. Bold marks
upregulated and italicized marks downregulated genes.

| | | | min of | max of | Fvari.AO16/ |
|---|---|---|---|---|---|
| | | | any other | any other | min or max |
| Gene | | Fvari.AO16/GF | microbes/GF | microbe/GF | of any other |
| Symbol | Tissue | (log2) | (log2) | (log2) | microbe (log2) |
| KCnj16 | colon | 3.9675 | −0.3985 | 0.598643333 | 6.6 |
| Ang4 | colon | 4.03 | −2.365 | 1.817475 | 2.2 |
| Retn1b | colon | 5.775 | −2.19 | 2.675 | 2.2 |
| Pnliprp2 | SI | 2.3325 | −1.570375 | 0.227715 | 10.2 |
| Capn13 | SI | 3.31 | −0.6172 | 0.5554 | 6.0 |
| Pmm1 | SI | 1.935 | −0.157 | 0.3402 | 5.7 |
| Pla2g4c | SI | 6.605 | −1.08 | 1.34385 | 4.9 |
| Pik3cg | SI | 1.65 | −0.2222 | 0.3789 | 4.4 |
| Ccnj | SI | 1.6775 | −0.56905 | 0.3896 | 4.3 |
| Duox2 | SI | 3.8175 | −1.03 | 0.903 | 4.2 |
| Lrmp | SI | 1.84 | −0.3745 | 0.4651 | 4.0 |
| Nmu | SI | 2.3025 | −0.2447 | 0.5914 | 3.9 |
| Pcsk6 | SI | 2.0125 | −0.4605 | 0.5537 | 3.6 |
| Retn1b | SI | 6.3325 | −0.14605 | 1.809806667 | 3.5 |
| Fam40b | SI | 2.095 | −0.3248 | 0.6009 | 3.5 |
| Tspan6 | SI | 2.045 | −0.7906 | 0.5941 | 3.4 |
| Gsdmc2 | SI | 3.9025 | −1.00775 | 1.14 | 3.4 |
| Dclk1 | SI | 1.5 | −0.37286 | 0.4486 | 3.3 |
| Ces1c | SI | 1.66 | −0.6814 | 0.5107 | 3.3 |
| Mecom | SI | 1.5925 | −0.7144 | 0.4934725 | 3.2 |
| Duoxa2 | SI | 4.3725 | −0.6665 | 1.41 | 3.1 |
| Retn1a | SI | 3.6875 | −0.3385 | 1.387625 | 2.7 |
| Cd55 | SI | 2.485 | −0.5882 | 0.9705 | 2.6 |
| Gsdmc2 | SI | 3.805 | −1.25 | 1.51 | 2.5 |
| Egln3 | SI | 1.725 | −0.6354 | 0.6897 | 2.5 |
| Vav1 | SI | 1.5675 | −0.4211 | 0.6322 | 2.5 |
| Gsdmc4 | SI | 4.1525 | −0.9741 | 1.82 | 2.3 |
| Me1 | SI | 2.5625 | −0.343795 | 1.17 | 2.2 |
| Ear2 | SI | 2.025 | −0.5681 | 0.9424 | 2.1 |
| Ugt2b5 | SI | −1.815 | −0.602325 | 1.595 | 3.0 |
| Tcrg-V2 | SI | −2.079175 | −0.7834 | 1.3 | 2.7 |
| Trim30a | SI | −1.641 | −0.73625 | 0.244 | 2.2 |
| Adh4 | SI | −1.0175 | −2.0125 | 1.131 | 0.5 |
| Ces1g | SI | −2.1075 | −1.12 | 1.43 | 1.9 |
| Olfr165 | SI | −2.625 | −1.53 | 0.79815 | 1.7 |
| Slc5a12 | SI | −4.2575 | −2.4875 | 0.825 | 1.7 |
| Akr1c19 | SI | −1.72 | −1.0331 | 0.8237 | 1.7 |
| Mt2 | SI | −2.105 | −1.3 | 0.7861 | 1.6 |
| Dnase1 | SI | −2.1175 | −1.311075 | 0.9769 | 1.6 |
| Ugt2b36 | SI | −1.675 | −1.042355 | 0.73765 | 1.6 |
| Ifit3 | SI | −1.71575 | −1.112375 | 0.82285 | 1.5 |

In accordance with the transcriptional effects, F. varium had one of the largest phenotypic impacts (FIG. 2D). Specifically, it had the strongest effect on αβT cells, reducing both T4 (CD4+) and T8 (CD8+) populations and causing a higher frequency of colonic DN (CD4−CD8−TCRβ+) cells than any other microbe (FIGS. 7D and 7E).

Fusobacterium spp. are among the few intestinal symbionts that can be found in both vertebrates and in free-living bacterial communities, rendering them potent to introduce evolutionarily honed functions. Relatively little is known about the Fusobacterium genus and human health, but Fusobacterium nucleatum is prevalent among patients with colorectal carcinoma and among some patients with inflammatory bowel disease. The virulence and invasiveness of F. nucleatum strains vary via unknown mechanisms that do not fit subspecies classifications, and the strain of F. nucleatum used here (F0419) elicited no outstanding phenotypes in our study. Without being bound to any particular theory, F. varium's prominent signature supports the notion that members of this genus may have unique interactions with the host.

### Example 2

The driving concept of this study was that the gut microbiota hosts a largely untapped wealth of immunomodulatory activities. To provide proof of concept, the inventors devised a sensitive, broad-ranging screen that entailed monocolonization of mice with human gut symbionts followed by extensive, unsupervised immunophenotyping and transcriptomics. Indeed, a screen of 53 bacterial species yielded a number of activities, both anticipated and unanticipated. For example, individual microbes were identified that are capable of inducing Th17 cells in the SI to a level similar to that driven by SFB. Unexpected, was the observation that about one-quarter of the bacteria examined, encompassing a diversity of species, could induce RORγ+Helios− Tregs in the colon, given claims that a consortium of 17 Clostridium species or several limited individual members of the microbiota are needed for Treg induction. Other potentially interesting immunomodulatory activities have not been reported previously—e.g., the augmentation of IL10-producing CD4+ T cells and the parallel reduction of IL22-producing ILCs in the colon by Veillonella; the impressive reduction of pDC numbers by L. rhamnosus; and the unusually strong and broad immunoperturbing activity of F. varium.

Without being bound to any particular theory, this approach has the potential to yield an apothecary of immunomodulatory agents tailored to modulate the immune system in a chosen manner. While local gut effects are the most straightforward to achieve, it is contemplated herein that microbiota manipulations can also regulate gut-distal immune responses-both protective and pathogenic. Data on RORγ+Helios− Tregs and Th17 cells argue that at least some of the observed activities can be recapitulated in SPF mice.

Beyond these practical considerations, the data provide several insights into immune system-microbiota interactions in the gut. The enormous complexity of the intestinal microbiota means that isolating the impact of a particular bacterial species on the intestinal or systemic immune system is a rather daunting task. Reliance on gnotobiotic conditions aids such deconvolution. Importantly, it was found that, in the absence of competition, most of the tested bacteria were able to robustly colonize the mouse intestine and that the great majority of them elicited immunophenotypic and/or transcriptomic changes, while few were stealth to the parameters measured. It was previously demonstrated that mice colonized with a complex human microbiota had small intestinal immune systems characteristic of GF mice. In contrast, the study described herein shows that colonization with single microbes derived from the human intestine does influence the immune system in the gut of host mice. Without being bound to any particular theory, these different outcomes are attributed to the much higher load of any one bacterium (up to 10,000× higher in monocolonized mice than in “human microbiota” mice), providing much greater antigen or metabolite stimuli.

Without being bound to any particular theory, the data convey that immune system recalibration to the microbiota shows substantial diversity and redundancy. On one hand, most microbes elicited a distinct immunophenotype in the host; on the other hand, many immunologic alterations were induced by more than one microbe, and bacteria could be found with opposite effects in most parameters. Without being bound to any particular theory, these adaptations might explain why microbial communities are so vast, providing balance to both the community and the host. A sufficiently large community of diverse genomic inputs allows buffering in case certain community members are lost. The broad diversity and redundancy of immunologic alterations permit many different microbes to provide the balance needed to promote overall host health. Importantly, both the diversity and the redundancy can be provided by organisms from the same or different phyla. Similarly, none of the transcriptional effects were induced by all of the microbes. In fact, different bacteria often had opposing impacts on the gut transcriptome, for example AMP gene expression. There did not appear to be a phylogenetic relationship in either the immunologic or the genomic changes. The lack of a relation between microbe-induced immune recalibration and microbial phylogeny would also contribute to stabilization of the microbiota's influence even if specific taxa were lost. The bacteria examined induced both shared and unique responses in different tissues at both the transcriptional and the cellular levels. For example, for Tregs and pDCs, a strong correlation existed between the SI and the colon (and other tissues). However, for IL17, IL22, and ILCs, recalibration and transcriptional responses to bacteria were mostly restricted to the SI. Interestingly, without being bound to any particular theory, the finding of greater variability between gene-expression profiles in GF mice than in monocolonized mice supports the contention that the presence of microbial communities stabilizes both immunologic and transcriptional phenotypes and provides resistance to perturbation. This notion of coupled diversity and redundancy may also explain why it is so often difficult to distill a designated microbiota influence or state of dysbiosis down to a single (or a single set of) bacterial species.

Without being bound to any particular theory, the absence of outcomes shared by all species within a phylum, or even a genus, suggests that this interspecies diversification might have occurred through horizontal transfer and/or that the corresponding mechanisms/pathways are common in the bacterial world. Moreover, this study shows differences even among the strains of the same species. This highlights the importance of strain specificity being associated with immunophenotypes. Even in parallel colonizations with the same microbes, some differences were observed. It is certainly possible that the bacterial and host transcriptomes adapt at different rates and that factors other than the ones we controlled for, such as microbial load, host age, and duration of colonization, are important in stabilizing responses.

This study demonstrates that manipulation of the gut microbiota presents many opportunities to impact the host immune system. It is clear that multiple individual microbes have important effects on the host, and that a balance of the microbiota is necessary for homeostasis. The combinatorial effects of immunomodulatory microbes can be further assessed both in a gnotobiotic setting and under SPF conditions. Determining the minimal consortium of microbes that can maintain a stable balance between the microbiota and the host immune system will likely now be possible. By identifying individual effector strains, studies on the mechanisms of host/microbial interactions (pathway interactions and key molecules) raise vital questions. Without being bound to any particular theory, the advantage of using specific molecules which can be dosed and regulated as any drug, would yield host responses that are more reproducible and therefore advantageous over using viable bacteria to modify or regulate a given host response

### Example 3

### Methods, Experimental Model and Subject Details

### Bacteria

Bacteria were purchased or obtained from several sources: the ATCC (atcc.org), BEI, (beiresources.org), or DSMZ (dsmz.de) repository or were obtained from BWH clinical labs or Harvard-affiliated labs (Table 1). Anaerobic bacteria were cultured in PYG broth under strictly anaerobic conditions (80% N2, 10% H2, 10% CO2) at 37° C. in an anaerobic chamber. All bacteria (Bacteroides, Clostridium, Bifidobacterium, Lactobacillus, Enterococcus, Fusobacterium, Propionibacterium, and Peptostreptococcus spp.) were grown in peptone-yeast-glucose medium supplemented with hemin and vitamin K or on brucella blood agar plates and TSA blood agar plates (BBL). Acinetobacter spp. were grown in Super Broth (SB) medium and on LB agar plates. Lachnospiraceae, Veillonella spp., and Coprobacillus spp. were grown in chopped meat broth. Staphylococcus spp. were grown aerobically at 37° C. in L-broth and on LB agar plates. Campylobacter and Helicobacter spp. were grown on brucella blood agar plates (VWR) and kept in microaerophilic conditions (CampyPak EZ in an anaerobic container system) at 37° C. The cladogram was generated using Human Microbiome Project data in GraPhlAn (http://huttenhower.sph.harvard.edu/galaxy/) and MetaPhlAn version 1.1.0 (http://www.hmpdacc.org/HMSMCP/healthy/#data). The overall mean diversity calculated by MEGA6 was 0.472. The total mean abundance was 62.6 and the prevalence ranged from 1.4 to 100 with a median of 64.4.

All strains of bacteria that were not from international repositories (Table 1) were deposited to BEI resources (https://www.beiresources.org/).

### Mice

GF C57BL/6J mice, originally purchased from the National Gnotobiotic Rodent Resource Center of the University of North Carolina at Chapel Hill, and bred in our lab facility, were used at Harvard Medical School in GF flexible film isolators (Class Biologically Clean®) throughout this study. Sterility tests (culture and PCR) were done every week, ensuring that mice remained GF. Mice food was autoclaved at 128° C. for 30 min at 26 PSI. Water was autoclaved at 121° C. for 1 h. SPF mice were housed under the same conditions in the same facility with the same food (autoclaved to ensure comparable nutrients) for 2 weeks. Animals of both genders were used as available. Littermates were randomly assigned to experimental groups, to avoid any bias, whenever possible. Animal protocol IS00000187 and COMS protocol 07-267 were approved by Harvard Medical School's Institutional Animal Care and Use Committee and the Committee on Microbiological Safety, respectively. This study adheres to the ARRIVE guidelines.

### Generation and Processing of Monocolonized Mice

GF C57BL/6 mice were orally inoculated by gavage with a broth grown single bacterial strain at 4 weeks of age and kept in gnotobiotic isolators. Each group of mice was housed in gnotobiotic isolators under sterile conditions for 2 weeks. Fecal material was collected and plated at 1 week and 2 weeks after bacterial inoculation to ensure monocolonization by a single bacterial strain. The identity of all colonizing microbial species was confirmed by 16S sequencing using the 27F (AGAGTTTGATCMTGGCTCAG—SEQ ID NO: 1) and 1492R (TACGGYTACCTTGTTACGACTT—SEQ ID NO: 2) primers and Sanger sequencing at the Harvard Biopolymers Facility. All colonizations were done and processed at the same time of the day to reduce diurnal variability. Processing was undertaken by the same individuals throughout these studies to minimize person-to-person variability.

### Preparation of Lymphocytes and Flow Cytometry

Intestinal tissues were treated with 30 mL of RPMI containing 1 mM dithiothreitol, 20 mM EDTA, and 2% FBS at 37° C. for 15 min to remove epithelial cells. The intestinal tissues and Peyer's patches were then minced and dissociated in RPMI containing collagenase II (1.5 mg/mL; Gibco), dispase (0.5 mg/mL), and 1% FBS, with constant stirring at 37° C. (45 min for colons and small intestines; 15 min for Peyer's patches). Single-cell suspensions were then filtered and washed with 4% RPMI solution.

Mesenteric lymph nodes (mLN), and Systemic lymphoid organs (SLO) were mechanically disrupted. Subcutaneous (inguinal and axillary) lymph nodes and spleens were pooled and red blood cells were lysed. To minimize variability and reagent drift, collagenase II and dispase were purchased in bulk and tested for consistency in digestion and viability of cells before use. Single-cell suspensions were stained for surface and intracellular markers and analyzed with BD LSRII.

Single-cell suspensions were stained with three constant panels of antibodies for consistency. The first panel included antibodies against CD4, CD8, TCRβ, CD45, TCRγδ, CD19, Foxp3, Helios and Rorγ. The second panel included antibodies against CD45, CD4, TCRβ, TCRγ, I117a, IFNγ, IL22, and IL10. The third panel included antibodies against CD45, CD19, CD11c, CD11b, Ly6c, PDCA-1, F4/80, and CD103. For cytokine analysis (second antibody panel), cells were treated with RMPI containing 10% FBS, phorbol 12-myristate 13-acetate (10 ng/mL; Sigma), and ionomycin (1 μM; Sigma) in the presence of GolgiStop (BD Biosciences) at 37° C. for 3.5 h. For intracellular staining of cytokines and transcription factors (first and second antibody panels), cells were stained for surface markers and fixed in eBioscience Fix/Perm buffer overnight, with subsequent permeabilization in eBioscience permeabilization buffer at room temperature for 45 min in the presence of antibodies. Cells stained with the third panel of markers were fixed in 1% formalin diluted in DMEM overnight. Great care was taken to reduce variability and reagent drift in all enzymes, reagents and antibodies. Cells were acquired with a BD LSRII, and analysis was performed with FlowJo (Tree Star) software.

Compensation for each experiment was adjusted with Rainbow Calibration particles to ensure consistency in data collection. The concentration, clone, and source of antibodies were kept constant to ensure consistency in staining. Occasionally, the entire set of data was sampled and reanalyzed blindly to ensure equal gating criteria and scoring. The raw data were independently analyzed by two individuals, and an average value was reported. Each analyst used the same version of FlowJo Software and the same bio-exponential settings previously determined for each experiment. When independent scoring differed by ≥25%, the scoring was re-determined by the two analysts together in order to understand and resolve the variation. If the analysts were unable to agree on how the experiment should be scored, the data were excluded from the final reports. Any strong discrepancies in staining due to reagent drift (e.g., enzymes, antibodies) were noted, and the data in question were excluded from the final reports. Frequencies of each cell type were averaged for each microbial colonization condition.

### IgA ELISA

IgA levels in feces of monocolonized mice were measured with a Mouse IgA Elisa Kit (eBioscience, 88-50450-88) according to the manufacturer's instructions.

### Gene-Expression Profiling

Data collection: The same segments of the distal colon and (0.5 cm long and 3 cm away from rectum) and three segments (each 0.3 cm long) from the same midsection of the duodenum, jejunum, and ileum of the small intestine were collected from mice. These segments were then homogenized in TRIzol and stored at −80° C. until RNA isolation. GF samples were collected throughout the duration of the screen. Samples were collected from both female or and male mice. Colon profiling included a total of four batches of samples totaling in 56 samples from male mice and 16 samples from female mice. SI profiling included a total of four batches of samples totaling in 51 samples from male mice and 7 samples from female mice. Each batch of microbially colonized intestines was profiled together with at least two replicates of GF control samples. Profiling was performed on Affymetrix Mouse Genome M1.0 ST arrays as previously described (Cipolletta et al., Nature 2012; 486, 549-553), nearly always at least in duplicates (singletons in rare instances).

### Quantification and Statistical Analysis

### Immunophenotypes

Fold-change values were calculated by dividing the frequencies of a given cell type for each microbial colonization by the average frequency obtained from GF mice, To control for multiple testing, a false discovery rate was calculated by the Benjamini-Hochberg procedure (Benjamini and Hochberg, Roy. Stat.Soc. B. 1995; 57, 289-300) was calculated and; the thresholds used are indicated in the text and figures where relevant.

Pearson correlations (for normalized mean immunophenotypes) and Euclidean distances (either per mouse or per normalized mean) within phyla, genera, species or strains were calculated by GeneE. To normalize per cell type, each frequency was divided by the mean of the cell type of interest across all microbes.

### Gene Expression Profiling

Data normalization and batch correction: Microarray data were background-corrected and normalized with the robust multi-array average algorithm. Gender and batch effects were corrected in a linear model with the feature as dependent variable and technical variables (batches) as regressors (implemented by R package “swamp”).

CV calculation: Microarrays for each microbe were typically performed in duplicate or triplicate. Thus, the CV per transcript for GF intestines was determined by (1) calculating the CV per transcript for randomly sampled GF pairs from a total of 8 (SI) or 12 (colon) GF replicates, and (2) iterating the random sampling 250 times and taking the average of the 250 CV values as the final CV value for GF mice. CV values for microbially colonized samples were calculated as per normal, without random sampling.

Selection of differentially expressed genes: Analysis on the whole tissue transcriptome focused on a select set of genes with a fold change relative to GF of >2.5 (or <0.4) and uncorrected p(−log 10)>2.5. Scatter analysis for most extreme effects on transcripts (both as fold change and as t-test p-value) was performed in R-Project or Multiplot Studio.

AMP aggregate score and correlation with gene expression: Aggregate AMP scores were calculated as follows: (1) RNA levels for each transcript belonging to the α-defensin and Reg3 family of AMPs, for which changes in expression levels were most dynamic, were normalized to the mean expression level across all samples; and (2) the normalized transcript levels were then summed and averaged for each sample to derive an aggregate AMP score. The correlation of all other transcripts with the respective AMP scores was determined with the Spearman correlation test. Correlations were calculated separately for GF and colonized mice, with use of six randomly sampled replicates for either group and iteration of the sampling and correlation test 50 times. The mean of the 50 correlation coefficients was taken to be the final coefficient value. RNAs with a correlation coefficient of >0.6 for both GF and monocolonized mice were extracted for pathway enrichment analysis.

Clustering and enrichment analysis: Hierarchical clustering and K-means clustering were performed on these selected genes in GeneE. Pathway analysis was done with STRING (www.string-db.org), and Enrichr (Chen et al., BMC. Bioinformatics 2013; 14, 128; Kuleshov et al., Nucleic Acid Res. 2016; 44, W90-W97, http://amp.pharm.mssm.edu/Enrichr/). Enrichment for cell types was verified in ImmGen and GNF databases.

### Data and Software Availability

The extensive dataset presented in FIGS. 1-4, is included in Tables 1-5 and in data not shown—see supplemental materials of Geva-Zatorsky et al., Cell 2017, incorporated by reference herein below. Phylogenetic identity of all bacteria is detailed in Table 1. The immunophenotypes as frequencies of cell types per an individual mouse basis were assessed (data not shown—see supplemental materials of Geva-Zatorsky et al., Cell 2017, incorporated by reference herein below). The gene expression raw data are in the Gene Expression Omnibus (GEO) database with accession number GSE88919.

Various embodiments of the methods and compositions are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

References cited herein are hereby individually incorporated by reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the descriptions, definition, and/or the use of a term associated with any of the incorporated material and that associated with the present document, the description, definition, and/or the use of the term in the present document shall prevail. The reference Geva-Zatorsky et al., Mining the Human Gut Microbiota for Immunomodulatory Organisms, Cell (2017), http://dx.doi.org/10.1016/j.cell.2017.01.022 including the supplemental materials referenced therein, are incorporated by reference herein in their entirety.

1. A method for manipulating a selected population of immune cells in a subject, the method comprising administering to the subject at least one bacterial strain selected from the group consisting of: Clostridium sordellii, Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella, Coprobacillus, Bacteroides uniformis, Clostridium perfringens, Bacteroides fragilis, Bacteroides vulgatus, Lactobacillus rhamnosus, Staphylococcus saprophyticus, Parabacteroides distasonis, Fusobacterium nucleatum, Propionibacterium granulosum, Bifidobacterium longum, Bacteroides ovatus, Bacteroides thetaiotaomicron, Enterococcus faecium, Helicobacter pylori, Ruminococcus gnavus, Peptostreptococus asaccharolyticus, Streptococcus mitis, and a combination thereof.

2. The method of claim 1, wherein the bacterial strain is administered to the GI tract of the subject.

3. The method of claim 2, wherein the manipulation comprises a change in an immune cell population in a tissue of the colon or small intestine.

4. The method of claim 1, wherein the manipulation comprises an expansion of:
(i) a monocyte population, and the bacterial strain is Clostridium sordellii,
(ii) a population of dendritic cells, and the bacterial strain is selected from the group consisting of Bifidobacterium breve, Bacteroides uniformis, Lachnospiraceae, and combinations thereof,
(iii) a population of plasmacytoid dendritic cells, and the bacterial strain is selected from the group consisting of Bacteroides fragilis, Bacteroides vulgatus, and a combination thereof, or
(iv) a population of IL22+ innate lymphoid cells, and the bacterial strain is selected from the group consisting of Bacteroides uniformis, Lactobacillus casei, and a combination thereof.

5. (canceled)

6. The method of claim 1, wherein the manipulation comprises a contraction of:
(i) a population of macrophages, and the bacterial strain is selected from the group consisting of Acinetobacter baumannii, Acinetobacter lwoffii, Bifidobacterium breve, Bacteroides dorei, Collinsella aerofaciens, Clostridium ramosum, Lachnospiraceae, Lactobacillus casei, Veillonella or a combination thereof,
(ii) a population of mononuclear phagocytes, and the bacterial strain is selected from the group consisting of Acinetobacter lwoffii, Collinsella aerofaciens, Coprobacillus, and combinations thereof,
(iii) a population of plasmacytoid dendritic cells, and the bacterial strain is selected from the group consisting of Lactobacillus rhamnosus, Staphylococcus saprophyticus, and a combination thereof, or
(iv) a population of type 3 innate lymphoid cells, and the bacterial strain is selected from the group consisting of Coprobacillus, Parabacteroides distasonis, Veillonella, and combinations thereof.

7.-25. (canceled)

26. The method of claim 1, wherein the manipulation comprises a contraction of:
(i) a population of IL22+ innate lymphoid cells, and the bacterial strain is selected from the group consisting of Acinetobacter lwoffii, Coprobacillus, Clostridium sordellii, Veillonella, and combinations thereof,
(ii) a population of IL22+ innate lymphoid cells, and the bacterial strain is selected from the group consisting of Acinetobacter lwoffii, Fusobacterium nucleatum, Propionibacterium granulosum, Veillonella, and combinations thereof,
(iii) a population of CD4 T cells, and the bacterial strain is selected from the group consisting of Bacteroides thetaiotaomicron, Peptostreptococus asaccharolyticus, Streptococcus mitis, and combinations thereof, or
(iv) a population of CD4 T cells, and the bacterial strain is selected from the group consisting of Clostridium perfringens, Peptostreptococus asaccharolyticus, and a combination thereof.

27. (canceled)

28. The method of claim 1, wherein the manipulation comprises an expansion of:
(i) a population of IL22+ innate lymphoid cells, and the bacterial strain is selected from the group consisting of Acinetobacter baumannii, Bacteroides dorei, and a combination thereof, or
(ii) a population of CD4 T cells, and the bacterial strain is selected from the group consisting of Acinetobacter lwoffii, Bifidobacterium longum, Bacteroides ovatus, Bacteroides thetaiotaomicron, Bacteroides vulgatus, Coprobacillus, Enterococcus faecium, Helicobacter pylori, Ruminococcus gnavus, Veillonella and combinations thereof.

29.-39. (canceled)

40.-49. (canceled)

50. A method of promoting expansion in a population of CD8−, CD4−, TCRγ+ T cells in a tissue of the gastrointestinal tract of a mammal, the method comprising administering a composition comprising a Fusobacterium varium bacterium to the gastrointestinal tract (GI) tract of the mammal.

51. The method of claim 50, wherein the tissue of the gastrointestinal tract comprises the small intestine.

52. The method of claim 50, wherein the tissue of the gastrointestinal tract comprises the colon.

53.-67. (canceled)

68. A method of sustained, localized delivery of a bioactive molecule to the oral cavity of a mammal, the method comprising administering a composition comprising a Porphyromonas gingivalis, Prevotella intermedia or Prevotella melaninogenica bacterium to the mammal.

69. The method of claim 68, wherein the bioactive molecule is expressed by the administered bacterium.

70. The method of claim 68, wherein the administered bacterium is engineered to express the bioactive molecule.

71.-77. (canceled)

78. A method of sustained, localized delivery of a bioactive molecule to the stomach of a mammal, the method comprising administering a composition comprising a Lactobacillus johnsonii bacterium to the mammal.

79. (canceled)

80. The method of claim 78, wherein the bioactive molecule is expressed by the administered bacterium.

81. The method of claim 78, wherein the administered bacterium is engineered to express the bioactive molecule.

82.-96. (canceled)

97. The method of claim 68, wherein the sustained delivery of the bioactive molecule treats an oral disease or disorder selected from the group consisting of: caries, periodontal disease, thrush, aphthous ulcer and halitosis.
